(19) United States
(12) Patent Application Publication
(10) Pub. No.: US 2023/0084899 A1
(43) Pub. Date: Mar. 16, 2023

(54) COMBINATION OF AN AHR-INHIBITOR AND AN PD1-INHIBITOR ANTIBODY AND ITS USE IN THE TREATMENT OF CANCER

(71) Applicant: Bayer Aktiengesellschaft, Leverkusen (DE)

(72) Inventor: Ilona GUTCHER, Berlin (DE)

(73) Assignee: Bayer Aktiengesellschaft, Leverkusen (DE)

(21) Appl. No.: 17/785,860

(22) PCT Filed: Dec. 14, 2020
(86) PCT No.: PCT/EP2020/085938

§ 371 Date: Jun. 15, 2022

Related U.S. Application Data
(60) Provisional application No. 62/948,430, filed on Dec. 16, 2019.

(30) Foreign Application Priority Data
Dec. 16, 2019 (EP) 19216672.6

(51) Int. Cl. A61K 31/501; C07K 16/28; A61K 31/506; A61K 31/381; A61P 35/00

(52) U.S. Cl. CPC A61K 31/501; C07K 16/2827; A61K 31/506; A61K 31/381; A61P 35/00

(57) ABSTRACT

The present invention relates to combinations of at least two components, component A and component B, component A being an AhR inhibitor, and component B being pembrolizumab or nivolumab. A further aspect of the present invention relates to combinations of three components, component A, component B, and component C; component A being an AhR inhibitor, component B being pembrolizumab or nivolumab, and component C being a further pharmaceutical agent. The present invention further relates to the use of such combinations as described herein for the preparation of a medicament for the treatment or prophylaxis of a disease, particularly for the treatment or prophylaxis of cancers of the breast, respiratory tract, brain, reproductive organs, digestive tract, urinary tract, eye, liver, skin, kidney, head and neck, thyroid, parathyroid, and their distant metastases, lymphomas, sarcomas and leukemias.

The present invention relates to combinations of at least two components, component A and component B, component A being an Aryl Hydrocarbon Receptor (AhR) inhibitor, and component B being a PD-1 inhibitor.

The term “Aryl Hydrocarbon Receptor inhibitor” is following abbreviated as “AhR inhibitor”.

Another aspect of the present invention relates to the use of such combinations as described herein as medicament for the treatment or prophylaxis of a disease, particularly for the treatment of cancer or tumors; respectively to the use of such combinations as described herein for the preparation of a medicament for the treatment or prophylaxis of a disease, particularly for the treatment of cancer or tumors;

Yet another aspect of the present invention relates to methods of treatment or prophylaxis of a cancer in a subject, comprising administering to said subject a therapeutically effective amount of a combination as described herein.

Further, the present invention relates to a combination, respectively a kit comprising:
one or more components A, as defined herein, or polymorphs, enantiomeres, diastereomeres, racemates, tautomeres, N-oxides, hydrates and solvates, as well as their physiological acceptable salts and solvates of these salts, as well as mixtures of the same, thereof;
a component B, as defined herein;
and, optionally
one or more pharmaceutical agents C, as defined herein;
   in which optionally either or both of said components A and B are in the form of a pharmaceutical formulation which is ready for use to be administered simultaneously, concurrently, separately or sequentially.

Component A may be administered by the oral, intravenous, topical, local installations, intraperitoneal or nasal route.

Component B may be administered by the oral, intravenous, topical, local installations, intraperitoneal or nasal route.

### BACKGROUND TO THE INVENTION

Cancer is the second most prevalent cause of death in the United States, causing 450,000 deaths per year. While substantial progress has been made in identifying some of the likely environmental and hereditary causes of cancer, there is a need for additional therapeutic modalities that target cancer and related diseases.

Particularly, there is a need for therapeutic methods for treating diseases associated with dysregulated growth/proliferation.

Cancer is a complex disease arising after a selection process for cells with acquired functional capabilities like enhanced survival/resistance towards apoptosis and a limitless proliferative potential.

Thus, it is preferred to develop drugs for cancer therapy addressing distinct features of established tumors.

The AhR inhibitors as described supra can target tumors that have increased expression of AhR ligands resulting in increased AhR activation. The inhibitors relieve AhR-induced immunosuppression and can further inhibit the intrinsic AhR activation in cancer cells. Thus, the AhR inhibitors that are described here in the inventive combination as component A can be used as a therapeutic tool in a variety of situations with aberrant AhR activation, e.g. cell proliferative disorders such as cancer. Disorders and conditions suitable for treatment with an antibody of the inventions can be, but are not limited to solid tumors, such as for example cancers of the breast, respiratory tract, brain, reproductive organs, digestive tract, urinary tract, eye, liver, skin, head and neck, thyroid, parathyroid, and their distant metastases. Those disorders also include lymphomas, sarcomas and leukemias.

The AhR (Aryl Hydrocarbon Receptor) is a ligand-activated transcription factor, belonging to the basic helix-loop-helix/Per-Arnt-Sim (bHLH/PAS) family, and is located in the cytosol. Upon ligand binding, the AhR translocates to the nucleus where it heterodimerises with ARNT (AHR Nuclear Translocator) upon which it interacts with DREs (Dioxin Response Elements) of AhR-responsive genes to regulate their transcription. The AhR is best known for binding to environmental toxins and inducing the metabolic machinery, such as cytochrome P 450 enzymes (eg. CYP1A1, CYP1A2 and CYP1B1), required for their elimination (Reyes et al., Science, 1992, 256(5060):1193-5). Activation of AhR by xenobiotics has demonstrated its role in numerous cellular processes such as embryogenesis, tumourigenesis and inflammation.

AhR is expressed in many cells of the immune system, including dendritic cells (DCs), macrophages, T cells and NK cells, and plays an important role in immunoregulation (Nguyen et al., Front Immunol, 2014, 5:551). The classic exogenous AhR ligands TCDD and 3-methylcholanthrene, for example, are known to induce profound immunosuppression, promote carcinogenesis and induce tumour growth (Gramatzki et al., Oncogene, 2009, 28(28):2593-605; Bui et al., Oncogene, 2009, 28(41):3642-51; Esser et al., Trends Immunol, 2009, 30:447-454).

In the context of immunosuppression, AhR activation promotes regulatory T cell generation, inhibits Th1 and Th17 differentiation, directly and indirectly, and decreases the activation and maturation of DCs (Wang et al., Clin Exp Immunol, 2014, 177(2):521-30; Mezrich et al., J Immunol, 2010, 185(6): 3190-8; Wei et al., Lab Invest, 2014, 94(5):528-35; Nguyen et al., PNAS, 2010, 107(46):19961-6). AhR activation modulates the innate immune response and constitutive AhR expression has been shown to negatively regulate the type-I interferon response to viral infection (Yamada et al., Nat Immunol, 2016). Additionally, mice with a constitutively active AhR spontaneously develop tumours (Andersson et al., PNAS, 2002, 99(15):9990-5).

In addition to xenobiotics, the AhR can also bind metabolic products of tryptophan degradation. Tryptophan metabolites, such as kynurenine and kynurenic acid, are endogenous AhR ligands that activate the AhR under physiological conditions (DiNatale et al., Toxicol Sci, 2010, 115(1):89-97; Mezrich et al., J Immunol, 2010, 185(6):3190-8; Opitz et al., Nature, 2011, 478(7368):197-203). Other endogenous ligands are known to bind the AhR although their physiological roles are currently unknown (Nguyen & Bradfield, Chem Res Toxicol, 2008, 21(1):102-116).

The immunosuppressive properties of kynurenine and tryptophan degradation are well described and are implicated in cancer-associated immunosuppression. The enzymes indoleamine-2,3-dioxygenases 1 and 2 (IDO1/IDO2) as well as tryptophan-2,3-dioxygenase 2 (TDO2) are responsible for catalysing the first and rate-limiting step of tryptophan metabolism. IDO1/2-mediated degradation of tryptophan in tumours and tumour-draining lymph nodes reduces anti-tumour immune responses and inhibition of IDO can suppress tumour formation in animal models (Uyttenhove et al., Nat Med, 2003, 9(10):1269-74; Liu et al., Blood, 2005, 115(17): 3520-30; Muller et al., Nat Med, 11(3):312-9; Metz, Cancer Res, 2007, 67(15):7082-7).

TDO2 is also strongly expressed in cancer and can lead to the production of immunosuppressive kynurenine. In glioma, activation of the AHR by kynurenine, downstream of TDO-mediated tryptophan degradation, enhances tumour growth as a consequence of inhibiting anti-tumour immune responses as well as directly promoting tumour cell survival and motility (Opitz et al., Nature, 2011, 478(7368):197-203). AhR ligands generated by tumour cells therefore act in both an autocrine and paracrine fashion on tumour cells and lymphocytes, respectively, to promote tumour growth.

Recently, the PD-1/PD-L1 signalling pathway has emerged as an important regulator of the activity of the immune system. In cancer, tumor cells express PD-L1, the ligand of PD-1, by which they can evade their killing by the host immune system. Inhibitors against PD-1 and its ligands PD-L1 and PD-L2 have recently been developed which interfere with this immune-suppressive mechanism and have shown amazing clinical efficacy, by extension of the overall survival of patients with various types of cancer. Some of these inhibitors have been approved for various cancer indications such as melanoma, NSCLC, HNSCC, RCC, bladder cancer and NHL. A large number of additional clinical trials are in progress in other indications and/or in combination with a variety of other antitumor agents in order to improve the therapeutic activity.

PD-1 inhibitors are biologics, primarily immunoglobulins of the G subclass, which bind to programmed cell death protein 1 also known as PD-1 and block its activity. Known PD-1 inhibitors are nivolumab (Opdivo, BMS-936558, MDX1106), pembrolizumab (Keytruda, MK-3475, lambrolizumab), PDR-001 (Novartis), JS001 (Shanghai Junshi Biosciences), STI-A1110, pidilizumab (Cure Tech), AMP-224 (GlaxoSmithKline), AMP-514 (GlaxoSmithKline), cemiplimab (Regeneron and Sanofi), BGB-A317 (BeiGene, China), SHR-1210 (Jiangsu Hengrui Medicine).

PD-1 (also known as CD279) is a receptor protein which is expressed as monomer on the surface of various immune cells mainly on activated CD4⁺ and CD8⁺ T cells, on macrophages and on activated B cells, but was also found on natural killer (NK) cells and antigen presenting cells (APC). Upon binding to its ligand PD-L1 or PD-L2, the phosphatase SHP-2 is recruited which dephosphorylates the kinase ZAP70, a major component of the T cell receptor (TCR) signaling complex. This shuts down TCR signaling and inhibits the cytotoxic activity of the T cells, their interferon gamma production and proliferation. In addition, PD-1 ligation up-regulates E3-ubiquitin ligases CBL-b and c-CBL that trigger T cell receptor down-modulation. PD-1 is encoded by the Pdcd1 gene in humans and is transcriptionally activated by transcription factors NFATc1, IRF9 and FoxO1, which are activated upon TCR activation and by T cell exhaustion signals such as transforming growth factor B and eomesodermin. The activation induced expression of PD-1 suggests that this receptor regulates rather the later phase of the immune response in the peripheral tissue (effector phase, memory response and chronic infection). However, despite the great success of the above identified approaches, it has turned out that some of them are either not sustainable in their efficacy, i.e., a recurrence of the disease occurs, and/or are only efficacious in some types of cancers.

Therefor there is a great need in the field of immune checkpoint inhibitor therapy for providing new and improved therapies as well as for improving existing therapies.

Pembrolizumab is a humanized monoclonal antibody and is an immune-checkpoint-inhibitor (PD-1 inhibitor) that is used for the treatment of different tumors.

The treatment of cancer using immune-checkpoint-inhibitors is a major new development in oncology. Pembrolizumab specifically targets the communication between tumor cells and T-cells which results in a better recognition of the tumor cells via the immune system. Pembrolizumab blocks the interaction between PD-1 and the programmed cell death ligands (PD-L) PD-L1, respectively PD-L2.

The PD-L-receptor normally switches off the activity of several cells of the immune system, respectively the T-cells. By blocking these PD-1-receptors pembrolizumab prevents said receptor to switch off these immune cells and thus increases the ability to kill the tumor cells. Thus, pembrolizumab can re-activate the natural anti-tumor immune response.

Especially blocking PD-1 shows an effective result in several malignant diseases.

Pembrolizumab already received approval in mono-therapeutically treatment of inoperable metastatic melanomas.

Of further interest for the inventive combination is nivolumab that is as well a humanized monoclonal antibody and is as well an immune-checkpoint-inhibitor (PD-1 inhibitor) that is used for the treatment of different tumors.

Now, there is a need for more effective therapeutic methods and medicaments for treating diseases associated with dysregulated growth, respectively proliferation.

### SUMMARY OF THE INVENTION

Surprisingly, it was observed that by administering an AhR inhibitor as component A of compounds of general formula (I):

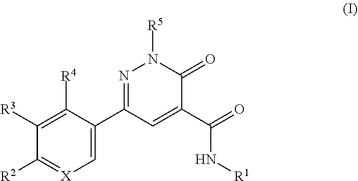

in which
R¹ represents C₂-C₆-hydroxyalkyl, wherein said C₂-C₆-hydroxyalkyl groups are optionally substituted once with cyano, —COOR¹⁰, —CONR¹¹R¹², C₁-C₄-alkoxy or C₃-C₆-cycloalkyl and optionally one to three times with halogen, or C₃-C₆-cycloalkyl substituted once with hydroxy and optionally once with C₁-C₃-alkyl and/or one to three times with halogen, or C₃-C₆-cycloalkyl-C₁-C₃-alkyl substituted once with hydroxy and optionally once with C₁-C₃-alkyl and/or one to three times with halogen, or (C₃-C₆-cycloalkyl)₂-C₁-C₃-alkyl substituted once with hydroxy and optionally once with C₁-C₃-alkyl and/or one to three times with halogen, or 4- to 6-membered heterocycloalkyl substituted once with hydroxy and optionally once with C₁-C₃-alkyl and/or one to three times with halogen;
R² represents chloro, cyano, dimethylamino, methyl, fluoromethyl, difluoromethyl, trifluoromethyl, methoxy, difluoromethoxy or trifluoromethoxy;
R³ represents hydrogen, fluoro, chloro or methyl;
R⁴ represents hydrogen or fluoro;
R⁵ represents monocyclic heteroaryl, which is optionally substituted one to three times, independently from each other, with R⁶;
R⁶ represents C₁-C₄-alkyl, C₁-C₄-haloalkyl, C₃-C₆-cycloalkyl, C₁-C₄-alkoxy, halogen or cyano;
X represents CH or N;
R¹⁰ represents C₁-C₄-alkyl;
R¹¹ and R¹² are the same or different and represent, independently from each other, hydrogen or C₁-C₃-alkyl, or together with the nitrogen atom to which they are attached form a 4- to 6-membered nitrogen containing heterocyclic ring, said ring optionally containing one additional heteroatom selected from O, S, NH, NR^{a} in which R^{a} represents a C₁-C₄-alkyl group;
   their polymorphs, enantiomeres, diastereomeres, racemates, tautomeres, N-oxides, hydrates and solvates, as well as their physiological acceptable salts and solvates of these salts, as well as mixtures of the same, in combination with pembrolizumab or nivolumab as component B, a synergistic anti-proliferative effect could be achieved in cell lines.

### Definitions

The term “substituted” means that one or more hydrogen atoms on the designated atom or group are replaced with a selection from the indicated group, provided that the designated atom's normal valency under the existing circumstances is not exceeded. Combinations of substituents and/or variables are permissible.

The term “optionally substituted” means that the number of substituents can be equal to or different from zero. Unless otherwise indicated, it is possible that optionally substituted groups are substituted with as many optional substituents as can be accommodated by replacing a hydrogen atom with a non-hydrogen substituent on any available carbon atom. Commonly, it is possible for the number of optional substituents, when present, to be 1, 2 or 3.

The term “comprising” when used in the specification includes “consisting of”.

If within the present text any item is referred to as “as mentioned herein”, it means that it may be mentioned anywhere in the present text.

The terms as mentioned in the present text have the following meanings:

The term “halogen” means a fluorine, chlorine, bromine or iodine, particularly a fluorine, chlorine or bromine atom.

The term “C₁-C₆-alkyl” means a linear or branched, saturated, monovalent hydrocarbon group having 1, 2, 3, 4, 5 or 6 carbon atoms, e.g. a methyl, ethyl, propyl, isopropyl, butyl, sec-butyl, isobutyl, tert-butyl, pentyl, isopentyl, 2-methylbutyl, 1-methylbutyl, 1-ethylpropyl, 1,2-dimethylpropyl, neo-pentyl, 1,1-dimethylpropyl, hexyl, 1-methylpentyl, 2-methylpentyl, 3-methylpentyl, 4-methylpentyl, 1-ethylbutyl, 2-ethylbutyl, 1,1-dimethylbutyl, 2,2-dimethylbutyl, 3,3-dimethylbutyl, 2,3-dimethylbutyl, 1,2-dimethylbutyl or 1,3-dimethylbutyl group, or an isomer thereof. Particularly, said group has 1, 2, 3 or 4 carbon atoms (“C₁-C₄-alkyl”), e.g. a methyl, ethyl, propyl, isopropyl, butyl, sec-butyl isobutyl, or tert-butyl group, more particularly 1, 2 or 3 carbon atoms (“C₁-C₃-alkyl”), e.g. a methyl, ethyl, n-propyl or isopropyl group.

The term “C₁-C₆-haloalkyl” means a linear or branched, saturated, monovalent hydrocarbon group in which the term “C₁-C₆-alkyl” is as defined supra, and in which one or more of the hydrogen atoms are replaced, identically or differently, with a halogen atom. Particularly, said halogen atom is a fluorine atom. Said C₁-C₆-haloalkyl group is, for example, fluoromethyl, difluoromethyl, trifluoromethyl, 2-fluoroethyl, 2,2-difluoroethyl, 2,2,2-trifluoroethyl, pentafluoroethyl, 3,3,3-trifluoropropyl or 1,3-difluoropropan-2-yl. Particularly, said group has 1, 2, 3 or 4 carbon atoms (“C₁-C₄-haloalkyl”), more particularly 1, 2 or 3 carbon atoms (“C₁-C₃-haloalkyl”), e.g. a fluoromethyl, difluoromethyl or trifluoromethyl group.

The term “C₂-C₆-hydroxyalkyl” means a linear or branched, saturated, monovalent hydrocarbon group in which the term “C₂-C₆-alkyl” is defined supra, and in which 1 or 2 hydrogen atoms are replaced with a hydroxy group, e.g. a 1-hydroxyethyl, 2-hydroxyethyl, 1,2-dihydroxyethyl, 3-hydroxypropyl, 2-hydroxypropyl, 1-hydroxypropyl, 1-hydroxypropan-2-yl, 2-hydroxypropan-2-yl, 2,3-dihydroxypropyl, 1,3-dihydroxypropan-2-yl, 3-hydroxy-2-methyl-propyl, 2-hydroxy-2-methyl-propyl, 1-hydroxy-2-methyl-propyl group.

The term “C₁-C₄-alkoxy” means a linear or branched, saturated, monovalent group of formula (C₁-C₄-alkyl)-O—, which means methoxy, ethoxy, n-propoxy, isopropoxy, n-butoxy, sec-butoxy, isobutoxy or tert-butoxy.

The term “C₃-C₆-cycloalkyl” means a saturated, monovalent, monocyclic hydrocarbon ring which contains 3, 4, 5 or 6 carbon atoms (“C₃-C₆-cycloalkyl”). Said C₃-C₆-cycloalkyl group is a monocyclic hydrocarbon ring, e.g. a cyclopropyl, cyclobutyl, cyclopentyl or cyclohexyl.

The term “C₂-C₇-alkylene” means a linear or branched, saturated, bivalent hydrocarbon group in which the term “C₂-C₇-alkyl” is as defined supra, and in which 2 hydrogen atoms from different carbon atoms are removed to form a biradical group.

The term “4- to 6-membered heterocycloalkyl” means a monocyclic, saturated heterocycle with 4, 5 or 6 ring atoms in total, which contains one or two identical or different ring heteroatoms from the series N and O, it being possible for said heterocycloalkyl group to be attached to the rest of the molecule via any one of the carbon atoms or, if present, a nitrogen atom.

Said heterocycloalkyl group, without being limited thereto, can be a 4-membered ring, such as azetidinyl or oxetanyl, for example; or a 5-membered ring, such as tetrahydrofuranyl, 1,3-dioxolanyl, pyrrolidinyl, imidazolidinyl, pyrazolidinyl, 1,2-oxazolidinyl or 1,3-oxazolidinyl, for example; or a 6-membered ring, such as tetrahydropyranyl, piperidinyl, morpholinyl, piperazinyl, 1,3-dioxanyl, 1,4-dioxanyl or 1,2-oxazinanyl, for example.

Particularly, “4- to 6-membered heterocycloalkyl” means a 4- to 6-membered heterocycloalkyl as defined supra containing one ring oxygen atom and optionally one further ring heteroatom from the series: N, O. More particularly, “5- or 6-membered heterocycloalkyl” means a monocyclic, saturated heterocycle with 5 or 6 ring atoms in total, containing one ring oxygen atom.

The term “monocyclic heteroaryl” means a monovalent, aromatic ring having 5 or 6 ring atoms (a “5- or 6-membered heteroaryl” group), which contains at least one ring heteroatom and optionally one or two further ring heteroatoms from the series: N, O and/or S, and which is bound via a ring carbon atom or optionally via a ring nitrogen atom (if allowed by valency).

Said heteroaryl group can be a 5-membered heteroaryl group, such as, for example, thienyl, furanyl, pyrrolyl, oxazolyl, thiazolyl, imidazolyl, pyrazolyl, isoxazolyl, isothiazolyl, oxadiazolyl, triazolyl, thiadiazolyl or tetrazolyl; or a 6-membered heteroaryl group, such as, for example, pyridinyl, pyridazinyl, pyrimidinyl, pyrazinyl or triazinyl.

In general, and unless otherwise mentioned, the heteroaryl or heteroarylene groups include all possible isomeric forms thereof, e.g.: tautomers and positional isomers with respect to the point of linkage to the rest of the molecule. Thus, for some illustrative non-restricting examples, the term pyridinyl includes pyridin-2-yl, pyridin-3-yl and pyridin-4-yl; or the term thienyl includes thien-2-yl and thien-3-yl.

Particularly, the heteroaryl group is an isothiazolyl, pyrazolyl, pyridinyl, pyridazinyl or pyrimidinyl group.

Where the plural form of the word compounds, salts, polymorphs, hydrates, solvates and the like, is used herein, this is taken to mean also a single compound, salt, polymorph, isomer, hydrate, solvate or the like.

By “stable compound” or “stable structure” is meant a compound that is sufficiently robust to survive isolation to a useful degree of purity from a reaction mixture, and formulation into an efficacious therapeutic agent.

The compounds of the present invention optionally contain one or more asymmetric centres, depending upon the location and nature of the various substituents desired. It is possible that one or more asymmetric carbon atoms are present in the (R) or (S) configuration, which can result in racemic mixtures in the case of a single asymmetric centre, and in diastereomeric mixtures in the case of multiple asymmetric centres. In certain instances, it is possible that asymmetry also be present due to restricted rotation about a given bond, for example, the central bond adjoining two substituted aromatic rings of the specified compounds.

Preferred compounds are those which produce the more desirable biological activity. Separated, pure or partially purified isomers and stereoisomers or racemic or diastereomeric mixtures of the compounds of the present invention are also included within the scope of the present invention. The purification and the separation of such materials can be accomplished by standard techniques known in the art.

Preferred isomers are those which produce the more desirable biological activity. These separated, pure or partially purified isomers or racemic mixtures of the compounds of this invention are also included within the scope of the present invention. The purification and the separation of such materials can be accomplished by standard techniques known in the art.

The optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid. Mixtures of diastereoisomers can be separated into their individual diastereomers on the basis of their physical and/or chemical differences by methods known in the art, for example, by chromatography or fractional crystallisation. The optically active bases or acids are then liberated from the separated diastereomeric salts. A different process for separation of optical isomers involves the use of chiral chromatography (e.g., HPLC columns using a chiral phase), with or without conventional derivatisation, optimally chosen to maximise the separation of the enantiomers. Suitable HPLC columns using a chiral phase are commercially available, such as those manufactured by Daicel, e.g., Chiracel OD and Chiracel OJ, for example, among many others, which are all routinely selectable. Enzymatic separations, with or without derivatisation, are also useful. The optically active compounds of the present invention can likewise be obtained by chiral syntheses utilizing optically active starting materials.

In order to distinguish different types of isomers from each other reference is made to IUPAC Rules Section E (Pure Appl Chem 45, 11-30, 1976).

The present invention includes all possible stereoisomers of the compounds of the present invention as single stereoisomers, or as any mixture of said stereoisomers, e.g. (R)- or (S)-isomers, in any ratio. Isolation of a single stereoisomer, e.g. a single enantiomer or a single diastereomer, of a compound of the present invention is achieved by any suitable state of the art method, such as chromatography, especially chiral chromatography, for example.

Further, the compounds of the present invention can exist as N-oxides, which are defined in that at least one nitrogen of the compounds of the present invention is oxidised. The present invention includes all such possible N-oxides.

The present invention also covers useful forms of the compounds of the present invention, such as metabolites, hydrates, solvates, prodrugs, salts, in particular pharmaceutically acceptable salts, and/or co-precipitates.

The compounds of the present invention can exist as a hydrate, or as a solvate, wherein the compounds of the present invention contain polar solvents, in particular water, methanol or ethanol for example, as structural element of the crystal lattice of the compounds. It is possible for the amount of polar solvents, in particular water, to exist in a stoichiometric or non-stoichiometric ratio. In the case of stoichiometric solvates, e.g. a hydrate, hemi-, (semi-), mono-, sesqui-, di-, tri-, tetra-, penta- etc. solvates or hydrates, respectively, are possible. The present invention includes all such hydrates or solvates.

Further, it is possible for the compounds of the present invention to exist in free form, e.g. as a free base, or as a free acid, or as a zwitterion, or to exist in the form of a salt. Said salt may be any salt, either an organic or inorganic addition salt, particularly any pharmaceutically acceptable organic or inorganic addition salt, which is customarily used in pharmacy, or which is used, for example, for isolating or purifying the compounds of the present invention.

The term “pharmaceutically acceptable salt” refers to an inorganic or organic acid addition salt of a compound of the present invention. For example, see S. M. Berge, et al. “Pharmaceutical Salts,” J. Pharm. Sci. 1977, 66, 1-19.

A suitable pharmaceutically acceptable salt of the compounds of the present invention may be, for example, an acid-addition salt of a compound of the present invention bearing a nitrogen atom, in a chain or in a ring, for example, which is sufficiently basic, such as an acid-addition salt with an inorganic acid, or “mineral acid”, such as hydrochloric, hydrobromic, hydroiodic, sulfuric, sulfamic, bisulfuric, phosphoric, or nitric acid, for example, or with an organic acid, such as formic, acetic, acetoacetic, pyruvic, trifluoroacetic, propionic, butyric, hexanoic, heptanoic, undecanoic, lauric, benzoic, salicylic, 2-(4-hydroxybenzoyl)-benzoic, camphoric, cinnamic, cyclopentanepropionic, digluconic, 3-hydroxy-2-naphthoic, nicotinic, pamoic, pectinic, 3-phenylpropionic, pivalic, 2-hydroxyethanesulfonic, itaconic, trifluoromethanesulfonic, dodecylsulfuric, ethanesulfonic, benzenesulfonic, para-toluenesulfonic, methanesulfonic, 2-naphthalenesulfonic, naphthalinedisulfonic, camphorsulfonic acid, citric, tartaric, stearic, lactic, oxalic, malonic, succinic, malic, adipic, alginic, maleic, fumaric, D-gluconic, mandelic, ascorbic, glucoheptanoic, glycerophosphoric, aspartic, sulfosalicylic, or thiocyanic acid, for example.

Further, another suitably pharmaceutically acceptable salt of a compound of the present invention which is sufficiently acidic, is an alkali metal salt, for example a sodium or potassium salt, an alkaline earth metal salt, for example a calcium, magnesium or strontium salt, or an aluminium or a zinc salt, or an ammonium salt derived from ammonia or from an organic primary, secondary or tertiary amine having 1 to 20 carbon atoms, such as ethylamine, diethylamine, triethylamine, ethyldiisopropylamine, monoethanolamine, diethanolamine, triethanolamine, dicyclohexylamine, dimethylaminoethanol, diethylaminoethanol, tris(hydroxymethyl)aminomethane, procaine, dibenzylamine, N-methylmorpholine, arginine, lysine, 1,2-ethylenediamine, N-methylpiperidine, N-methyl-glucamine, N,N-dimethyl-glucamine, N-ethyl-glucamine, 1,6-hexanediamine, glucosamine, sarcosine, serinol, 2-amino-1,3-propanediol, 3-amino-1,2-propanediol, 4-amino-1,2,3-butanetriol, or a salt with a quarternary ammonium ion having 1 to 20 carbon atoms, such as tetramethylammonium, tetraethylammonium, tetra(n-propyl)ammonium, tetra(n-butyl)ammonium, N-benzyl-N,N,N-trimethylammonium, choline or benzalkonium.

Those skilled in the art will further recognise that it is possible for acid addition salts of the claimed compounds to be prepared by reaction of the compounds with the appropriate inorganic or organic acid via any of a number of known methods. Alternatively, alkali and alkaline earth metal salts of acidic compounds of the present invention are prepared by reacting the compounds of the present invention with the appropriate base via a variety of known methods.

The present invention includes all possible salts of the compounds of the present invention as single salts, or as any mixture of said salts, in any ratio.

In the present text, in particular in the Experimental Section, for the synthesis of intermediates and of examples of the present invention, when a compound is mentioned as a salt form with the corresponding base or acid, the exact stoichiometric composition of said salt form, as obtained by the respective preparation and/or purification process, is, in most cases, unknown.

Unless specified otherwise, suffixes to chemical names or structural formulae relating to salts, such as “hydrochloride”, “trifluoroacetate”, “sodium salt”, or “x HCl”, “x CF₃COOH”, “x Na⁺”, for example, mean a salt form, the stoichiometry of which salt form not being specified.

This applies analogously to cases in which synthesis intermediates or example compounds or salts thereof have been obtained, by the preparation and/or purification processes described, as solvates, such as hydrates, with (if defined) unknown stoichiometric composition.

Furthermore, the present invention includes all possible crystalline forms, or polymorphs, of the compounds of the present invention, either as single polymorph, or as a mixture of more than one polymorph, in any ratio.

Moreover, the present invention also includes prodrugs of the compounds according to the invention. The term “prodrugs” here designates compounds which themselves can be biologically active or inactive but are converted (for example metabolically or hydrolytically) into compounds according to the invention during their residence time in the body.

The invention further includes all possible crystallized and polymorphic forms of the inventive compounds, whereby the polymorphs are existing either as a single polymorph form or are existing as a mixture of several polymorphs in all concentrations.

The term “combination” in the present invention is used as known to persons skilled in the art, it being possible for said combination to be a fixed combination, a non-fixed combination or a kit-of-parts.

A “fixed combination” in the present invention is used as known to persons skilled in the art and is defined as a combination wherein, for example, a first active ingredient, such as an AhR-inhibitor of the present invention, and an antibody (i.e. pembrolizumab, nivolumab, etc.) are present together in one unit dosage or in one single entity. One example of a “fixed combination” is a pharmaceutical composition wherein a first active ingredient and a further active ingredient are present in admixture for simultaneous administration, such as in a formulation. Another example of a “fixed combination” is a pharmaceutical combination wherein a first active ingredient and a further active ingredient are present in one unit without being in admixture.

A non-fixed combination or “kit-of-parts” in the present invention is used as known to persons skilled in the art and is defined as a combination wherein a first active ingredient and a further active ingredient are present in more than one unit. One example of a non-fixed combination or kit-of-parts is a combination wherein the first active ingredient and the further active ingredient are present separately. It is possible for the components of the non-fixed combination or kit-of-parts to be administered separately, sequentially, simultaneously, concurrently or chronologically staggered.

“Antibodies”, also synonymously called “immunoglobulins” (Ig), are generally comprising four polypeptide chains, two heavy (H) chains and two light (L) chains, and are therefore multimeric proteins, or an equivalent Ig homologue thereof (e.g., a camelid nanobody, which comprises only a heavy chain, single domain antibodies (dAbs) which can be either be derived from a heavy or light chain); including full length functional mutants, variants, or derivatives thereof (including, but not limited to, murine, chimeric, humanized and fully human antibodies, which retain the essential epitope binding features of an Ig molecule (or, if necessary, undergo affinity maturation or deiimuization), and including dual specific, bispecific, multispecific, and dual variable domain immunoglobulins. Immunoglobulin molecules can be of any class (e.g., IgG, IgE, IgM, IgD, IgA, and IgY), or subclass (e.g., IgG1, IgG2, IgG3, IgG4, IgA1, and IgA2) and allotype.

In accordance with a second embodiment of the first aspect, the present invention covers combinations of component A of compounds of general formula (I), supra, in which:
R¹ represents C₂-C₆-hydroxyalkyl, wherein said C₂-C₆-hydroxyalkyl groups are optionally substituted once with cyano, —COOR¹⁰, —CONR¹¹R¹², C₁-C₄-alkoxy or C₃-C₆-cycloalkyl and optionally one to three times with halogen, or
   C₃-C₆-cycloalkyl substituted once with hydroxy and optionally one to three times with halogen, or
   C₃-C₆-cycloalkyl-C₁-C₃-alkyl substituted once with hydroxy and optionally one to three times with halogen, or
   (C₃-C₆-cycloalkyl)₂-C₁-C₃-alkyl substituted once with hydroxy and optionally one to three times with halogen, or
   4- to 6-membered heterocycloalkyl substituted once with hydroxy and optionally one to three times with halogen;
R² represents chloro, cyano, dimethylamino, methyl, fluoromethyl, difluoromethyl, trifluoromethyl, methoxy, difluoromethoxy or trifluoromethoxy;
R³ represents hydrogen, fluoro, chloro or methyl;
R⁴ represents hydrogen or fluoro;
R⁵ represents monocyclic heteroaryl, which is optionally substituted one to three times, independently from each other, with R⁶;
R⁶ represents C₁-C₄-alkyl, C₁-C₄-haloalkyl, C₃-C₆-cycloalkyl, C₁-C₄-alkoxy, halogen or cyano;
X represents CH or N;
R¹⁰ represents C₁-C₄-alkyl;
R¹¹ and R¹² are the same or different and represent, independently from each other, hydrogen or C₁-C₃-alkyl, or together with the nitrogen atom to which they are attached form a 4- to 6-membered nitrogen containing heterocyclic ring, said ring optionally containing one additional heteroatom selected from O, S, NH, NR^{a} in which R^{a} represents a C₁-C₄-alkyl group;
   their polymorphs, enantiomeres, diastereomeres, racemates, tautomeres, N-oxides, hydrates and solvates, as well as their physiological acceptable salts and solvates of these salts, as well as mixtures of the same with pembrolizumab or with nivolumab as component B.

In accordance with a third embodiment of the first aspect, the present invention covers combinations of component A of compounds of general formula (I), supra, in which:
R¹ represents C₂-C₆-hydroxyalkyl, wherein said C₂-C₆-hydroxyalkyl groups are optionally substituted once with cyano, —COOR¹⁰, —CONR¹¹R¹², C₁-C₄-alkoxy or C₃-C₆-cycloalkyl and optionally one to three times with halogen, or
   C₃-C₆-cycloalkyl substituted once with hydroxy and optionally once with C₁-C₃-alkyl and/or one to three times with halogen, or
   C₃-C₆-cycloalkyl-C₁-C₃-alkyl substituted once with hydroxy and optionally once with C₁-C₃-alkyl and/or one to three times with halogen, or
   (C₃-C₆-cycloalkyl)₂-C₁-C₃-alkyl substituted once with hydroxy and optionally once with C₁-C₃-alkyl and/or one to three times with halogen, or
   4- to 6-membered heterocycloalkyl substituted once with hydroxy and optionally once with C₁-C₃-alkyl and/or one to three times with halogen;
R² represents chloro, cyano, dimethylamino, methyl, fluoromethyl, difluoromethyl, trifluoromethyl, methoxy, difluoromethoxy or trifluoromethoxy;
R³ represents hydrogen, fluoro, chloro or methyl;
R⁴ represents hydrogen or fluoro;
R⁵ represents monocyclic heteroaryl, which is optionally substituted one to three times, independently from each other, with R⁶;
R⁶ represents C₁-C₄-alkyl, C₃-C₆-cycloalkyl, C₁-C₄-alkoxy, halogen or cyano;
X represents CH or N;
   their polymorphs, enantiomeres, diastereomeres, racemates, tautomeres, N-oxides, hydrates and solvates, as well as their physiological acceptable salts and solvates of these salts, as well as mixtures of the same with pembrolizumab or with nivolumab as component B.

In accordance with a fourth embodiment of the first aspect, the present invention covers combinations of component A of compounds of general formula (I), supra, in which:
R¹ represents C₂-C₅-hydroxyalkyl, wherein said C₂-C₅-hydroxyalkyl groups are optionally substituted once with cyano, —COOCH₃, —CONH₂, methoxy or cyclopropyl and optionally one to three times with fluoro, or
   C₄-C₆-cycloalkyl substituted once with hydroxy and optionally once with methyl and/or one to two times with fluoro, or
   C₃-C₄-cycloalkyl-methyl substituted once with hydroxy, or
   5- or 6-membered heterocycloalkyl substituted once with hydroxy, said heterocycloalkyl contains one oxygen atom;
R² represents chloro, cyano, dimethylamino, methyl, fluoromethyl, difluoromethyl, trifluoromethyl, methoxy, difluoromethoxy or trifluoromethoxy;
R³ represents hydrogen or fluoro;
R⁴ represents hydrogen or fluoro;
R⁵ represents monocyclic heteroaryl, which is optionally substituted one to three times, independently from each other, with R⁶;
R⁶ represents methyl, difluoromethyl, methoxy, halogen or cyano;
X represents CH or N;
R¹⁰ represents C₁-C₄-alkyl;
R¹¹ and R¹² are the same or different and represent, independently from each other, hydrogen or C₁-C₃-alkyl, or
   together with the nitrogen atom to which they are attached form a 4- to 6-membered nitrogen containing heterocyclic ring, said ring optionally containing one additional heteroatom selected from O, S, NH, NR^{a} in which R^{a} represents a C₁-C₄-alkyl group;
      their polymorphs, enantiomeres, diastereomeres, racemates, tautomeres, N-oxides, hydrates and solvates, as well as their physiological acceptable salts and solvates of these salts, as well as mixtures of the same with pembrolizumab or with nivolumab as component B.

In accordance with a fifth embodiment of the first aspect, the present invention covers combinations of component A of compounds of general formula (I), supra, in which:
R¹ represents C₂-C₅-hydroxyalkyl, wherein said C₂-C₅-hydroxyalkyl groups are optionally substituted once with cyano, —COOCH₃, —CONH₂, methoxy or cyclopropyl and optionally one to three times with fluoro, or
   C₄-C₆-cycloalkyl substituted once with hydroxy and optionally once with methyl and/or one to two times with fluoro, or
   C₃-C₄-cycloalkyl-methyl substituted once with hydroxy, or
   5- or 6-membered heterocycloalkyl substituted once with hydroxy, said heterocycloalkyl contains one oxygen atom;
R² represents chloro, dimethylamino, methyl, fluoromethyl, difluoromethyl, trifluoromethyl, difluoromethoxy or trifluoromethoxy;
R³ represents hydrogen;
R⁴ represents hydrogen or fluoro;
R⁵ represents a group selected from:

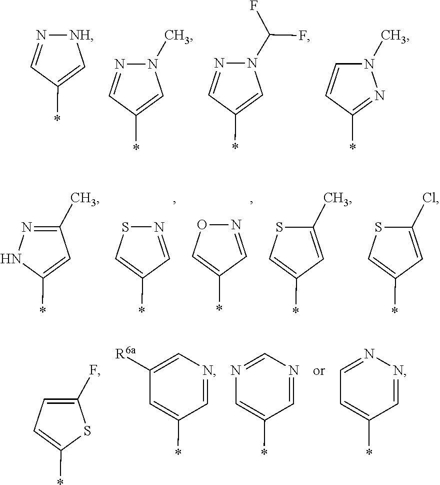

wherein * indicates the point of attachment of said group with the rest of the molecule;
R^{6a} represents hydrogen, methyl, fluoro or chloro;
X represents CH or N;
   their polymorphs, enantiomeres, diastereomeres, racemates, tautomeres, N-oxides, hydrates and solvates, as well as their physiological acceptable salts and solvates of these salts, as well as mixtures of the same with pembrolizumab or with nivolumab as component B

In accordance with a sixth embodiment of the first aspect, the present invention covers combinations of component A of compounds of general formula (Ia):

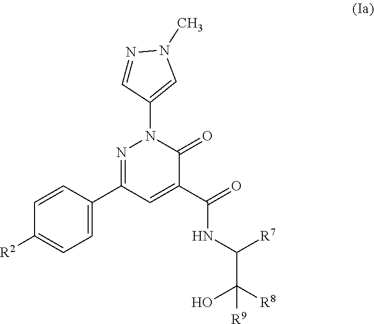

in which
R² represents chloro, dimethylamino, methyl, fluoromethyl, difluoromethyl, trifluoromethyl, difluoromethoxy or trifluoromethoxy;
R⁷ represents hydrogen, methyl, fluoromethyl, difluoromethyl, trifluoromethyl, hydroxymethyl, methoxymethyl, ethyl, isopropyl, cyclopropyl, cyano, —COOCH₃ or —CONH₂;
R³ represents hydrogen, methyl, fluoromethyl, difluoromethyl, trifluoromethyl, hydroxymethyl or methoxymethyl,
   wherein one of R⁷ and R⁸ is different from hydrogen, or
   together R⁷ and R⁸ form a cyclopentyl or cyclohexyl ring, which is optionally substituted one to two times with fluoro, or a heterocycloalkyl ring, which contains one oxygen atom;
R⁹ represents hydrogen or methyl, or
   together R⁸ and R⁹ form a cyclopropyl or cyclobutyl ring;
      their polymorphs, enantiomeres, diastereomeres, racemates, tautomeres, N-oxides, hydrates and solvates, as well as their physiological acceptable salts and solvates of these salts, as well as mixtures of the same with pembrolizumab or with nivolumab as component B.

In accordance with a seventh embodiment of the first aspect, the present invention covers combinations of component A of compounds of general formula (Ib):

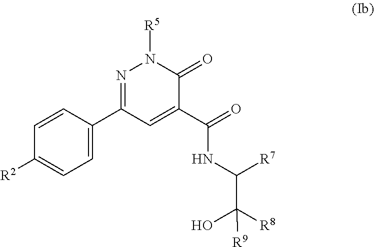

in which
R² represents chloro, dimethylamino, methyl, fluoromethyl, difluoromethyl, trifluoromethyl, difluoromethoxy or trifluoromethoxy;
R⁵ represents a group selected from:

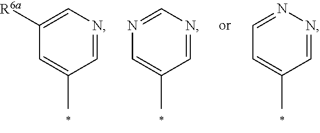

wherein * indicates the point of attachment of said group with the rest of the molecule;
R^{6a} represents hydrogen, methyl, fluoro or chloro;
R⁷ represents hydrogen, methyl, fluoromethyl, difluoromethyl, trifluoromethyl, hydroxymethyl, methoxymethyl, ethyl, isopropyl or cyclopropyl;
R⁸ represents hydrogen, methyl, fluoromethyl, difluoromethyl, trifluoromethyl, hydroxymethyl or methoxymethyl,
   wherein one of R⁷ and R⁸ is different from hydrogen, or
   together R⁷ and R⁸ form a cyclopentyl or cyclohexyl ring, which is optionally substituted one to two times with fluoro, or a heterocycloalkyl ring, which contains one oxygen or sulphur atom;
R⁹ represents hydrogen or methyl, or
   together R⁸ and R⁹ form a cyclopropyl or cyclobutyl ring;
      their polymorphs, enantiomeres, diastereomeres, racemates, tautomeres, N-oxides, hydrates and solvates, as well as their physiological acceptable salts and solvates of these salts, as well as mixtures of the same with pembrolizumab or with nivolumab as component B.

In accordance with a further embodiment of the first aspect, the present invention covers combinations of component A of compounds of general formula (I), supra, in which:
R¹ represents C₂-C₆-hydroxyalkyl, wherein said C₂-C₆-hydroxyalkyl groups are optionally substituted once with cyano, —COOR¹⁰, —CONR¹¹R¹², C₁-C₄-alkoxy or C₃-C₆-cycloalkyl and optionally one to three times with halogen, or
   C₃-C₆-cycloalkyl substituted once with hydroxy and optionally once with C₁-C₃-alkyl and/or one to three times with halogen, or
   C₃-C₆-cycloalkyl-C₁-C₃-alkyl substituted once with hydroxy and optionally once with C₁-C₃-alkyl and/or one to three times with halogen, or
   (C₃-C₆-cycloalkyl)₂-C₁-C₃-alkyl substituted once with hydroxy and optionally once with C₁-C₃-alkyl and/or one to three times with halogen, or
   4- to 6-membered heterocycloalkyl substituted once with hydroxy and optionally once with C₁-C₃-alkyl and/or one to three times with halogen;
      their polymorphs, enantiomeres, diastereomeres, racemates, tautomeres, N-oxides, hydrates and solvates, as well as their physiological acceptable salts and solvates of these salts, as well as mixtures of the same with pembrolizumab or with nivolumab as component B.

In accordance with a further embodiment of the first aspect, the present invention covers combinations of component A of compounds of general formula (I), supra, in which:
R¹ represents C₂-C₅-hydroxyalkyl, wherein said C₂-C₅-hydroxyalkyl groups are optionally substituted once with cyano, —COOCH₃, —CONH₂, methoxy or cyclopropyl and optionally one to three times with fluoro, or
   C₄-C₆-cycloalkyl substituted once with hydroxy and optionally once with methyl and/or one to two times with fluoro, or
   C₃-C₄-cycloalkyl-methyl substituted once with hydroxy, or
   5- or 6-membered heterocycloalkyl substituted once with hydroxy, said heterocycloalkyl contains one oxygen atom;
      their polymorphs, enantiomeres, diastereomeres, racemates, tautomeres, N-oxides, hydrates and solvates, as well as their physiological acceptable salts and solvates of these salts, as well as mixtures of the same with pembrolizumab or with nivolumab as component B.

In accordance with a further embodiment of the first aspect, the present invention covers combinations of component A of compounds of general formula (I), supra, in which:
R¹ represents C₂-C₆-hydroxyalkyl, wherein said C₂-C₆-hydroxyalkyl groups are optionally substituted once with cyano, —COOR¹⁰, —CONR¹¹R¹², C₁-C₄-alkoxy or C₃-C₆-cycloalkyl and optionally one to three times with halogen, or
   C₃-C₆-cycloalkyl substituted once with hydroxy and optionally one to three times with halogen, or
   C₃-C₆-cycloalkyl-C₁-C₃-alkyl substituted once with hydroxy and optionally one to three times with halogen, or
   (C₃-C₆-cycloalkyl)₂-C₁-C₃-alkyl substituted once with hydroxy and optionally one to three times with halogen, or
   4- to 6-membered heterocycloalkyl substituted once with hydroxy and optionally one to three times with halogen;
      their polymorphs, enantiomeres, diastereomeres, racemates, tautomeres, N-oxides, hydrates and solvates, as well as their physiological acceptable salts and solvates of these salts, as well as mixtures of the same with pembrolizumab or with nivolumab as component B.

In accordance with a further embodiment of the first aspect, the present invention covers combinations of component A of compounds of general formula (I), supra, in which:
R¹ represents C₂-C₆-hydroxyalkyl, wherein said C₂-C₆-hydroxyalkyl groups are optionally substituted once with C₁-C₄-alkoxy or C₃-C₆-cycloalkyl and optionally one to three times with halogen, or
   C₃-C₆-cycloalkyl substituted once with hydroxy and optionally once with methyl and/or one to three times with halogen, or
   C₃-C₆-cycloalkyl-C₁-C₃-alkyl substituted once with hydroxy and optionally one to three times with halogen, or
   (C₃-C₆-cycloalkyl)₂-C₁-C₃-alkyl substituted once with hydroxy and optionally one to three times with halogen, or
   4- to 6-membered heterocycloalkyl substituted once with hydroxy and optionally one to three times with halogen;
      their polymorphs, enantiomeres, diastereomeres, racemates, tautomeres, N-oxides, hydrates and solvates, as well as their physiological acceptable salts and solvates of these salts, as well as mixtures of the same with pembrolizumab or with nivolumab as component B.

In accordance with a further embodiment of the first aspect, the present invention covers combinations of component A of compounds of general formula (I), supra, in which:
R¹ represents C₂-C₆-hydroxyalkyl, wherein said C₂-C₆-hydroxyalkyl groups are optionally substituted once with C₁-C₂-alkoxy or cyclopropyl and optionally one to three times with halogen, or
   C₄-C₆-cycloalkyl substituted once with hydroxy and optionally once with methyl and/or one to three times with halogen, or
   C₃-C₆-cycloalkyl-methyl substituted once with hydroxy and optionally one to three times with halogen, or
   (C₃-C₆-cycloalkyl)₂-methyl substituted once with hydroxy and optionally one to three times with halogen, or
   5- or 6-membered heterocycloalkyl substituted once with hydroxy and optionally one to three times with halogen;
      their polymorphs, enantiomeres, diastereomeres, racemates, tautomeres, N-oxides, hydrates and solvates, as well as their physiological acceptable salts and solvates of these salts, as well as mixtures of the same with pembrolizumab or with nivolumab as component B.

In accordance with a further embodiment of the first aspect, the present invention covers combinations of component A of compounds of general formula (I), supra, in which:
R¹ represents C₂-C₅-hydroxyalkyl, wherein said C₂-C₅-hydroxyalkyl groups are optionally substituted once with methoxy or cyclopropyl and optionally one to three times with fluoro, or
   C₄-C₆-cycloalkyl substituted once with hydroxy and optionally once with methyl and/or one to two times with fluoro, or
   C₃-C₄-cycloalkyl-methyl substituted once with hydroxy, or
   5- or 6-membered heterocycloalkyl substituted once with hydroxy, said heterocycloalkyl contains one oxygen atom;
      their polymorphs, enantiomeres, diastereomeres, racemates, tautomeres, N-oxides, hydrates and solvates, as well as their physiological acceptable salts and solvates of these salts, as well as mixtures of the same with pembrolizumab or with nivolumab as component B.

In accordance with a further embodiment of the first aspect, the present invention covers combinations of component A of compounds of general formula (I), supra, in which:
R¹ represents C₂-C₅-hydroxyalkyl, wherein said C₂-C₅-hydroxyalkyl groups are optionally substituted once with methoxy or cyclopropyl and optionally one to three times with fluoro, or
   C₄-C₆-cycloalkyl substituted once with hydroxy and optionally once with methyl or one to two times with fluoro, or
   C₃-C₄-cycloalkyl-methyl substituted once with hydroxy, or
   5- or 6-membered heterocycloalkyl substituted once with hydroxy, said heterocycloalkyl contains one oxygen atom;
      their polymorphs, enantiomeres, diastereomeres, racemates, tautomeres, N-oxides, hydrates and solvates, as well as their physiological acceptable salts and solvates of these salts, as well as mixtures of the same with pembrolizumab or with nivolumab as component B.

In accordance with a further embodiment of the first aspect, the present invention covers combinations of component A of compounds of general formula (I), supra, in which:
R² represents chloro, cyano, dimethylamino, methyl, fluoromethyl, difluoromethyl, trifluoromethyl, methoxy, difluoromethoxy or trifluoromethoxy;
   their polymorphs, enantiomeres, diastereomeres, racemates, tautomeres, N-oxides, hydrates and solvates, as well as their physiological acceptable salts and solvates of these salts, as well as mixtures of the same with pembrolizumab or with nivolumab as component B.

In accordance with a further embodiment of the first aspect, the present invention covers combinations of component A of compounds of general formula (I), supra, in which:
R² represents chloro, dimethylamino, methyl, fluoromethyl, difluoromethyl, trifluoromethyl, difluoromethoxy or trifluoromethoxy;
   their polymorphs, enantiomeres, diastereomeres, racemates, tautomeres, N-oxides, hydrates and solvates, as well as their physiological acceptable salts and solvates of these salts, as well as mixtures of the same with pembrolizumab or with nivolumab as component B.

In accordance with a further embodiment of the first aspect, the present invention covers combinations of component A of compounds of general formula (I), supra, in which:
R³ represents hydrogen, fluoro, chloro or methyl;
   their polymorphs, enantiomeres, diastereomeres, racemates, tautomeres, N-oxides, hydrates and solvates, as well as their physiological acceptable salts and solvates of these salts, as well as mixtures of the same with pembrolizumab or with nivolumab as compound B.

In accordance with a further embodiment of the first aspect, the present invention covers combinations of compound A of general formula (I), supra, in which:
R³ represents hydrogen or fluoro;
   their polymorphs, enantiomeres, diastereomeres, racemates, tautomeres, N-oxides, hydrates and solvates, as well as their physiological acceptable salts and solvates of these salts, as well as mixtures of the same with pembrolizumab or with nivolumab as component B.

In accordance with a further embodiment of the first aspect, the present invention covers combinations of component A of compounds of general formula (I), supra, in which:
R³ represents hydrogen;
   their polymorphs, enantiomeres, diastereomeres, racemates, tautomeres, N-oxides, hydrates and solvates, as well as their physiological acceptable salts and solvates of these salts, as well as mixtures of the same with pembrolizumab or with nivolumab as component B.

In accordance with a further embodiment of the first aspect, the present invention covers combinations of component A of compounds of general formula (I), supra, in which:
R⁴ represents hydrogen or fluoro;
   their polymorphs, enantiomeres, diastereomeres, racemates, tautomeres, N-oxides, hydrates and solvates, as well as their physiological acceptable salts and solvates of these salts, as well as mixtures of the same with pembrolizumab or with nivolumab as component B.

In accordance with a further embodiment of the first aspect, the present invention covers combinations of component A of compounds of general formula (I), supra, in which:
R⁴ represents hydrogen;
   their polymorphs, enantiomeres, diastereomeres, racemates, tautomeres, N-oxides, hydrates and solvates, as well as their physiological acceptable salts and solvates of these salts, as well as mixtures of the same with pembrolizumab or with nivolumab as component B.

In accordance with a further embodiment of the first aspect, the present invention covers combinations of component A of compounds of general formula (I), supra, in which:
R⁵ represents monocyclic heteroaryl, which is optionally substituted one to three times, independently from each other, with R⁶;
   their polymorphs, enantiomeres, diastereomeres, racemates, tautomeres, N-oxides, hydrates and solvates, as well as their physiological acceptable salts and solvates of these salts, as well as mixtures of the same with pembrolizumab or with nivolumab as component B.

In accordance with a further embodiment of the first aspect, the present invention covers combinations of component A of compounds of general formula (I), supra, in which:
R⁵ represents a group selected from:

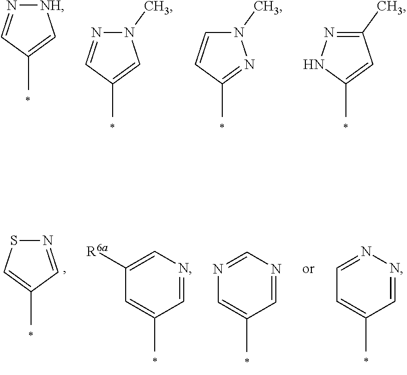

wherein * indicates the point of attachment of said group with the rest of the molecule;
their polymorphs, enantiomeres, diastereomeres, racemates, tautomeres, N-oxides, hydrates and solvates, as well as their physiological acceptable salts and solvates of these salts, as well as mixtures of the same with pembrolizumab or with nivolumab as component B.

In accordance with a further embodiment of the first aspect, the present invention covers combinations of component A of compounds of general formula (I), supra, in which:
R⁵ represents a group selected from:

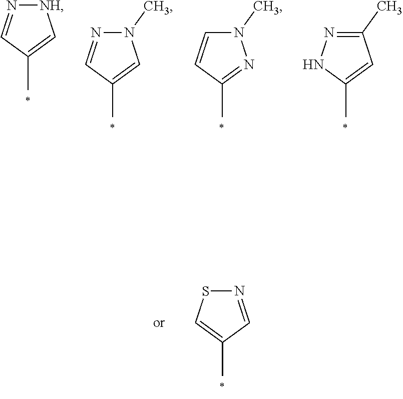

wherein * indicates the point of attachment of said group with the rest of the molecule;
their polymorphs, enantiomeres, diastereomeres, racemates, tautomeres, N-oxides, hydrates and solvates, as well as their physiological acceptable salts and solvates of these salts, as well as mixtures of the same with pembrolizumab or with nivolumab as component B.

In accordance with a further embodiment of the first aspect, the present invention covers combinations of component A of compounds of general formula (I), supra, in which:
R⁵ represents a group selected from:

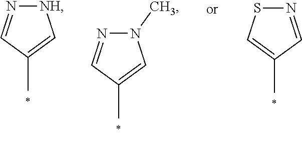

wherein * indicates the point of attachment of said group with the rest of the molecule;
their polymorphs, enantiomeres, diastereomeres, racemates, tautomeres, N-oxides, hydrates and solvates, as well as their physiological acceptable salts and solvates of these salts, as well as mixtures of the same with pembrolizumab or with nivolumab as component B.

In accordance with a further embodiment of the first aspect, the present invention covers combinations of component A of compounds of general formula (I), supra, in which:
R⁵ represents a group selected from:

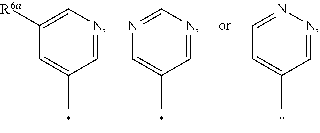

wherein * indicates the point of attachment of said group with the rest of the molecule;
their polymorphs, enantiomeres, diastereomeres, racemates, tautomeres, N-oxides, hydrates and solvates, as well as their physiological acceptable salts and solvates of these salts, as well as mixtures of the same with pembrolizumab or with nivolumab as component B.

In accordance with a further embodiment of the first aspect, the present invention covers combinations of component A of compounds of general formula (I), supra, in which:
R⁶ represents C₁-C₄-alkyl, C₁-C₄-haloalkyl, C₃-C₆-cycloalkyl, C₁-C₄-alkoxy, halogen or cyano;
   their polymorphs, enantiomeres, diastereomeres, racemates, tautomeres, N-oxides, hydrates and solvates, as well as their physiological acceptable salts and solvates of these salts, as well as mixtures of the same with pembrolizumab or with nivolumab as component B.

In accordance with a further embodiment of the first aspect, the present invention covers combinations of component A of compounds of general formula (I), supra, in which:
R⁶ represents C₁-C₄-alkyl, C₃-C₆-cycloalkyl, C₁-C₄-alkoxy, halogen or cyano;
   their polymorphs, enantiomeres, diastereomeres, racemates, tautomeres, N-oxides, hydrates and solvates, as well as their physiological acceptable salts and solvates of these salts, as well as mixtures of the same with pembrolizumab or with nivolumab as component B.

In accordance with a further embodiment of the first aspect, the present invention covers combinations of component A of compounds of general formula (I), supra, in which:
R⁶ represents methyl, methoxy, halogen or cyano;
   their polymorphs, enantiomeres, diastereomeres, racemates, tautomeres, N-oxides, hydrates and solvates, as well as their physiological acceptable salts and solvates of these salts, as well as mixtures of the same with pembrolizumab or with nivolumab as component B.

In accordance with a further embodiment of the first aspect, the present invention covers combinations of component A of compounds of general formula (I), supra, in which:
R^{6a} represents hydrogen, methyl, fluoro or chloro;
   their polymorphs, enantiomeres, diastereomeres, racemates, tautomeres, N-oxides, hydrates and solvates, as well as their physiological acceptable salts and solvates of these salts, as well as mixtures of the same with pembrolizumab or with nivolumab as component B.

In accordance with a further embodiment of the first aspect, the present invention covers combinations of component A of compounds of general formula (I), supra, in which:
R¹⁰ represents C₁-C₄-alkyl;
   their polymorphs, enantiomeres, diastereomeres, racemates, tautomeres, N-oxides, hydrates and solvates, as well as their physiological acceptable salts and solvates of these salts, as well as mixtures of the same with pembrolizumab or with nivolumab as component B.

In accordance with a further embodiment of the first aspect, the present invention covers combinations of component A of compounds of general formula (I), supra, in which:
R¹⁰ represents methyl;
   their polymorphs, enantiomeres, diastereomeres, racemates, tautomeres, N-oxides, hydrates and solvates, as well as their physiological acceptable salts and solvates of these salts, as well as mixtures of the same with pembrolizumab or with nivolumab as component B.

In accordance with a further embodiment of the first aspect, the present invention covers combinations of component A of compounds of general formula (I), supra, in which:
R¹¹ and R¹² are the same or different and represent, independently from each other, hydrogen or C₁-C₃-alkyl, or together with the nitrogen atom to which they are attached form a 4- to 6-membered nitrogen containing heterocyclic ring, said ring optionally containing one additional heteroatom selected from O, S, NH, NR^{a} in which R^{a} represents a C₁-C₄-alkyl group;
   their polymorphs, enantiomeres, diastereomeres, racemates, tautomeres, N-oxides, hydrates and solvates, as well as their physiological acceptable salts and solvates of these salts, as well as mixtures of the same with pembrolizumab or with nivolumab as component B.

In accordance with a further embodiment of the first aspect, the present invention covers combinations of component A of compounds of general formula (I), supra, in which:
R¹¹ and R¹² are the same or different and represent, independently from each other, hydrogen or C₁-C₃-alkyl, or together with the nitrogen atom to which they are attached form a 4- to 6-membered nitrogen containing heterocyclic ring;
   their polymorphs, enantiomeres, diastereomeres, racemates, tautomeres, N-oxides, hydrates and solvates, as well as their physiological acceptable salts and solvates of these salts, as well as mixtures of the same with pembrolizumab or with nivolumab as component B.

In accordance with a further embodiment of the first aspect, the present invention covers combinations of component A of compounds of general formula (I), supra, in which:
R¹¹ and R¹² are the same or different and represent, independently from each other, hydrogen or C₁-C₃-alkyl;
   their polymorphs, enantiomeres, diastereomeres, racemates, tautomeres, N-oxides, hydrates and solvates, as well as their physiological acceptable salts and solvates of these salts, as well as mixtures of the same with pembrolizumab or with nivolumab as component B.

In accordance with a further embodiment of the first aspect, the present invention covers combinations of component A of compounds of general formula (I), supra, in which:
R¹¹ and R¹² are the same or different and represent, independently from each other, hydrogen or methyl;
   their polymorphs, enantiomeres, diastereomeres, racemates, tautomeres, N-oxides, hydrates and solvates, as well as their physiological acceptable salts and solvates of these salts, as well as mixtures of the same with pembrolizumab or with nivolumab as component B.

In accordance with a further embodiment of the first aspect, the present invention covers combinations of component A of compounds of general formula (I), supra, in which:
X represents CH or N;
   their polymorphs, enantiomeres, diastereomeres, racemates, tautomeres, N-oxides, hydrates and solvates, as well as their physiological acceptable salts and solvates of these salts, as well as mixtures of the same with pembrolizumab or with nivolumab as component B.

In accordance with a further embodiment of the first aspect, the present invention covers combinations of component A of compounds of general formula (I), supra, in which:

X represents CH;
their polymorphs, enantiomeres, diastereomeres, racemates, tautomeres, N-oxides, hydrates and solvates, as well as their physiological acceptable salts and solvates of these salts, as well as mixtures of the same with pembrolizumab or with nivolumab as component B.

Especially, as compound A those AhR inhibitors are of interest for the combination as disclosed in the International Patent Application No. PCT/EP2018/052627, published as WO 2018/146010 A1:
N-[(2S)-1-hydroxybutan-2-yl]-6-(4-methylphenyl)-2-(1-methyl-1H-pyrazol-4-yl)-3-oxo-2,3-dihydropyridazine-4-carboxamide;
N-(1-hydroxypropan-2-yl)-6-(4-methylphenyl)-2-(1-methyl-1H-pyrazol-4-yl)-3-oxo-2,3-dihydropyridazine-4-carboxamide;
N-[(2R)-1-hydroxypropan-2-yl]-6-(4-methylphenyl)-2-(1-methyl-1H-pyrazol-4-yl)-3-oxo-2,3-dihydropyridazine-4-carboxamide;
N-[(2S)-1-hydroxypropan-2-yl]-6-(4-methylphenyl)-2-(1-methyl-1H-pyrazol-4-yl)-3-oxo-2,3-dihydropyridazine-4-carboxamide;
6-(4-methylphenyl)-2-(1-methyl-1H-pyrazol-4-yl)-3-oxo-N-(3,3,3-trifluoro-2-hydroxypropyl)-2,3-dihydropyridazine-4-carboxamide;
6-(4-methylphenyl)-2-(1-methyl-1H-pyrazol-4-yl)-3-oxo-N-[(2R)-3,3,3-trifluoro-2-hydroxypropyl]-2,3-dihydropyridazine-4-carboxamide;
6-(4-methylphenyl)-2-(1-methyl-1H-pyrazol-4-yl)-3-oxo-N-[(2S)-3,3,3-trifluoro-2-hydroxypropyl]-2,3-dihydropyridazine-4-carboxamide;
N-(3,3-difluoro-2-hydroxypropyl)-6-(4-methylphenyl)-2-(1-methyl-1H-pyrazol-4-yl)-3-oxo-2,3-dihydropyridazine-4-carboxamide;
N-[(2R)-3,3-difluoro-2-hydroxypropyl]-6-(4-methylphenyl)-2-(1-methyl-1H-pyrazol-4-yl)-3-oxo-2,3-dihydropyridazine-4-carboxamide;
N-[(2S)-3,3-difluoro-2-hydroxypropyl]-6-(4-methylphenyl)-2-(1-methyl-1H-pyrazol-4-yl)-3-oxo-2,3-dihydropyridazine-4-carboxamide;
6-(4-chlorophenyl)-N-(1-hydroxy-3-methylbutan-2-yl)-2-(1-methyl-1H-pyrazol-4-yl)-3-oxo-2,3-dihydropyridazine-4-carboxamide;
6-(4-chlorophenyl)-N-[(2S)-1-hydroxy-3-methylbutan-2-yl]-2-(1-methyl-1H-pyrazol-4-yl)-3-oxo-2,3-dihydropyridazine-4-carboxamide;
6-(4-chlorophenyl)-N-(1-hydroxypropan-2-yl)-2-(1-methyl-1H-pyrazol-4-yl)-3-oxo-2,3-dihydropyridazine-4-carboxamide;
6-(4-chlorophenyl)-N-[(2S)-1-hydroxypropan-2-yl]-2-(1-methyl-1H-pyrazol-4-yl)-3-oxo-2,3-dihydropyridazine-4-carboxamide;
6-(4-chlorophenyl)-2-(1-methyl-1H-pyrazol-4-yl)-3-oxo-N-(3,3,3-trifluoro-2-hydroxypropyl)-2,3-dihydropyridazine-4-carboxamide;
6-(4-chlorophenyl)-2-(1-methyl-1H-pyrazol-4-yl)-3-oxo-N-[(2R)-3,3,3-trifluoro-2-hydroxypropyl]-2,3-dihydropyridazine-4-carboxamide;
6-(4-chlorophenyl)-2-(1-methyl-1H-pyrazol-4-yl)-3-oxo-N-[(2S)-3,3,3-trifluoro-2-hydroxypropyl]-2,3-dihydropyridazine-4-carboxamide;
6-(4-chlorophenyl)-N-(3,3-difluoro-2-hydroxypropyl)-2-(1-methyl-1H-pyrazol-4-yl)-3-oxo-2,3-dihydropyridazine-4-carboxamide;
6-(4-chlorophenyl)-N-[(2R)-3,3-difluoro-2-hydroxypropyl]-2-(1-methyl-1H-pyrazol-4-yl)-3-oxo-2,3-dihydropyridazine-4-carboxamide;
6-(4-chlorophenyl)-N-[(2S)-3,3-difluoro-2-hydroxypropyl]-2-(1-methyl-1H-pyrazol-4-yl)-3-oxo-2,3-dihydropyridazine-4-carboxamide;
6-(4-chlorophenyl)-N-(2-hydroxy-3-methoxypropyl)-2-(1-methyl-1H-pyrazol-4-yl)-3-oxo-2,3-dihydropyridazine-4-carboxamide;
6-(4-chlorophenyl)-N-[(2R)-2-hydroxy-3-methoxypropyl]-2-(1-methyl-1H-pyrazol-4-yl)-3-oxo-2,3-dihydropyridazine-4-carboxamide;
6-(4-chlorophenyl)-N-[(2S)-2-hydroxy-3-methoxypropyl]-2-(1-methyl-1H-pyrazol-4-yl)-3-oxo-2,3-dihydropyridazine-4-carboxamide;
6-(4-chlorophenyl)-N-(2,3-dihydroxypropyl)-2-(1-methyl-1H-pyrazol-4-yl)-3-oxo-2,3-dihydropyridazine-4-carboxamide;
6-(4-chlorophenyl)-N-[(2R)-2,3-dihydroxypropyl]-2-(1-methyl-1H-pyrazol-4-yl)-3-oxo-2,3-dihydropyridazine-4-carboxamide;
6-(4-chlorophenyl)-N-[(2S)-2,3-dihydroxypropyl]-2-(1-methyl-1H-pyrazol-4-yl)-3-oxo-2,3-dihydropyridazine-4-carboxamide;
6-(4-chlorophenyl)-N-(1-hydroxy-3-methoxypropan-2-yl)-2-(1-methyl-1H-pyrazol-4-yl)-3-oxo-2,3-dihydropyridazine-4-carboxamide;
6-(4-chlorophenyl)-N-[(2R)-1-hydroxy-3-methoxypropan-2-yl]-2-(1-methyl-1H-pyrazol-4-yl)-3-oxo-2,3-dihydropyridazine-4-carboxamide;
6-(4-chlorophenyl)-N-[(2S)-1-hydroxy-3-methoxypropan-2-yl]-2-(1-methyl-1H-pyrazol-4-yl)-3-oxo-2,3-dihydropyridazine-4-carboxamide;
6-(4-chlorophenyl)-N-(1,3-dihydroxypropan-2-yl)-2-(1-methyl-1H-pyrazol-4-yl)-3-oxo-2,3-dihydropyridazine-4-carboxamide;
6-(4-chlorophenyl)-N-(2-hydroxy-2-methylpropyl)-2-(1-methyl-1H-pyrazol-4-yl)-3-oxo-2,3-dihydropyridazine-4-carboxamide;
6-(4-chlorophenyl)-2-(1-methyl-1H-pyrazol-4-yl)-3-oxo-N-(1,1,1-trifluoro-3-hydroxypropan-2-yl)-2,3-dihydropyridazine-4-carboxamide;
6-(4-chlorophenyl)-2-(1-methyl-1H-pyrazol-4-yl)-3-oxo-N-[(2R)-1,1,1-trifluoro-3-hydroxypropan-2-yl]-2,3-dihydropyridazine-4-carboxamide;
6-(4-chlorophenyl)-2-(1-methyl-1H-pyrazol-4-yl)-3-oxo-N-[(2S)-1,1,1-trifluoro-3-hydroxypropan-2-yl]-2,3-dihydropyridazine-4-carboxamide;
2-(1-methyl-1H-pyrazol-4-yl)-3-oxo-N-(1,1,1-trifluoro-3-hydroxypropan-2-yl)-6-[4-(trifluoromethyl)phenyl]-2,3-dihydropyridazine-4-carboxamide;
2-(1-methyl-1H-pyrazol-4-yl)-3-oxo-N-[(2R)-1,1,1-trifluoro-3-hydroxypropan-2-yl]-6-[4-(trifluoromethyl)phenyl]-2,3-dihydropyridazine-4-carboxamide;
2-(1-methyl-1H-pyrazol-4-yl)-3-oxo-N-(3,3,3-trifluoro-2-hydroxypropyl)-6-[4-(trifluoromethyl)phenyl]-2,3-dihydropyridazine-4-carboxamide;
2-(1-methyl-1H-pyrazol-4-yl)-3-oxo-N-[(2S)-3,3,3-trifluoro-2-hydroxypropyl]-6-[4-(trifluoromethyl)phenyl]-2,3-dihydropyridazine-4-carboxamide;
N-(1-hydroxypropan-2-yl)-2-(1-methyl-1H-pyrazol-4-yl)-3-oxo-6-[4-(trifluoromethyl)phenyl]-2,3-dihydropyridazine-4-carboxamide;
N-[(2R)-1-hydroxypropan-2-yl]-2-(1-methyl-1H-pyrazol-4-yl)-3-oxo-6-[4-(trifluoromethyl)phenyl]-2,3-dihydropyridazine-4-carboxamide;
N-[(2S)-1-hydroxypropan-2-yl]-2-(1-methyl-1H-pyrazol-4-yl)-3-oxo-6-[4-(trifluoromethyl)phenyl]-2,3-dihydropyridazine-4-carboxamide;
6-(4-chlorophenyl)-2-(1-cyclobutyl-1H-pyrazol-4-yl)-3-oxo-N-(3,3,3-trifluoro-2-hydroxypropyl)-2,3-dihydropyridazine-4-carboxamide;
6-(4-chlorophenyl)-2-(1-cyclobutyl-1H-pyrazol-4-yl)-3-oxo-N-[(2S)-3,3,3-trifluoro-2-hydroxypropyl]-2,3-dihydropyridazine-4-carboxamide;
6-(4-chlorophenyl)-2-(1-cyclobutyl-1H-pyrazol-4-yl)-3-oxo-N-(1,1,1-trifluoro-3-hydroxypropan-2-yl)-2,3-dihydropyridazine-4-carboxamide;
6-(4-chlorophenyl)-2-(1-cyclobutyl-1H-pyrazol-4-yl)-3-oxo-N-[(2R)-1,1,1-trifluoro-3-hydroxypropan-2-yl]-2,3-dihydropyridazine-4-carboxamide;
6-(4-chlorophenyl)-2-(1-cyclobutyl-1H-pyrazol-4-yl)-N-(1-hydroxypropan-2-yl)-3-oxo-2,3-dihydropyridazine-4-carboxamide;
6-(4-chlorophenyl)-2-(1-cyclobutyl-1H-pyrazol-4-yl)-N-[(2S)-1-hydroxypropan-2-yl]-3-oxo-2,3-dihydropyridazine-4-carboxamide;
6-(4-chlorophenyl)-N-[(cis)-4-hydroxytetrahydrofuran-3-yl]-2-(1-methyl-1H-pyrazol-4-yl)-3-oxo-2,3-dihydropyridazine-4-carboxamide;
6-(4-chlorophenyl)-N-[(3R,4R)-4-hydroxytetrahydrofuran-3-yl]-2-(1-methyl-1H-pyrazol-4-yl)-3-oxo-2,3-dihydropyridazine-4-carboxamide;
6-(4-chlorophenyl)-N-[(3S,4S)-4-hydroxytetrahydrofuran-3-yl]-2-(1-methyl-1H-pyrazol-4-yl)-3-oxo-2,3-dihydropyridazine-4-carboxamide;
N-[(cis)-2-hydroxycyclohexyl]-2-(1-methyl-1H-pyrazol-4-yl)-3-oxo-6-[4-(trifluoromethoxy)phenyl]-2,3-dihydropyridazine-4-carboxamide;
N-[(1S,2R)-2-hydroxycyclohexyl]-2-(1-methyl-1H-pyrazol-4-yl)-3-oxo-6-[4-(trifluoromethoxy)phenyl]-2,3-dihydropyridazine-4-carboxamide;
2-(1-methyl-1H-pyrazol-4-yl)-3-oxo-N-(1,1,1-trifluoro-3-hydroxypropan-2-yl)-6-[4-(trifluoromethoxy)phenyl]-2,3-dihydropyridazine-4-carboxamide;
2-(1-methyl-1H-pyrazol-4-yl)-3-oxo-N-[(2R)-1,1,1-trifluoro-3-hydroxypropan-2-yl]-6-[4-(trifluoromethoxy)phenyl]-2,3-dihydropyridazine-4-carboxamide;
6-(4-chlorophenyl)-N-[(cis)-2-hydroxycyclohexyl]-2-(1-methyl-1H-pyrazol-4-yl)-3-oxo-2,3-dihydropyridazine-4-carboxamide;
6-(4-chlorophenyl)-N-[(1S,2R)-2-hydroxycyclohexyl]-2-(1-methyl-1H-pyrazol-4-yl)-3-oxo-2,3-dihydropyridazine-4-carboxamide;
6-(4-chlorophenyl)-N-[(trans)-2-hydroxycyclopentyl]-2-(1-methyl-1H-pyrazol-4-yl)-3-oxo-2,3-dihydropyridazine-4-carboxamide;
6-(4-chlorophenyl)-N-[(1S,2S)-2-hydroxycyclopentyl]-2-(1-methyl-1H-pyrazol-4-yl)-3-oxo-2,3-dihydropyridazine-4-carboxamide;
6-(4-chlorophenyl)-N-[(cis)-2-hydroxycyclopentyl]-2-(1-methyl-1H-pyrazol-4-yl)-3-oxo-2,3-dihydropyridazine-4-carboxamide;
6-(4-chlorophenyl)-N-[(1S,2R)-2-hydroxycyclopentyl]-2-(1-methyl-1H-pyrazol-4-yl)-3-oxo-2,3-dihydropyridazine-4-carboxamide;
6-[4-(difluoromethyl)phenyl]-N-[(trans)-2-hydroxycyclopentyl]-2-(1-methyl-1H-pyrazol-4-yl)-3-oxo-2,3-dihydropyridazine-4-carboxamide;
N-(1-hydroxypropan-2-yl)-2-(1-methyl-1H-pyrazol-4-yl)-3-oxo-6-[4-(trifluoromethoxy)phenyl]-2,3-dihydropyridazine-4-carboxamide;
N-[(2S)-1-hydroxypropan-2-yl]-2-(1-methyl-1H-pyrazol-4-yl)-3-oxo-6-[4-(trifluoromethoxy)phenyl]-2,3-dihydropyridazine-4-carboxamide;
6-[6-(difluoromethyl)pyridin-3-yl]-N-[(cis)-2-hydroxycyclohexyl]-2-(1-methyl-1H-pyrazol-4-yl)-3-oxo-2,3-dihydropyridazine-4-carboxamide;
6-[6-(difluoromethyl)pyridin-3-yl]-N-[(1S,2R)-2-hydroxycyclohexyl]-2-(1-methyl-1H-pyrazol-4-yl)-3-oxo-2,3-dihydropyridazine-4-carboxamide;
N-[(cis)-4-hydroxytetrahydrofuran-3-yl]-2-(1-methyl-1H-pyrazol-4-yl)-3-oxo-6-[4-(trifluoromethoxy)phenyl]-2,3-dihydropyridazine-4-carboxamide;
N-[(3R,4R)-4-hydroxytetrahydrofuran-3-yl]-2-(1-methyl-1H-pyrazol-4-yl)-3-oxo-6-[4-(trifluoromethoxy)phenyl]-2,3-dihydropyridazine-4-carboxamide;
N-[(3S,4S)-4-hydroxytetrahydrofuran-3-yl]-2-(1-methyl-1H-pyrazol-4-yl)-3-oxo-6-[4-(trifluoromethoxy)phenyl]-2,3-dihydropyridazine-4-carboxamide;
6-[4-(difluoromethyl)phenyl]-N-(1-hydroxypropan-2-yl)-2-(1-methyl-1H-pyrazol-4-yl)-3-oxo-2,3-dihydropyridazine-4-carboxamide;
6-[4-(difluoromethyl)phenyl]-N-[(2S)-1-hydroxypropan-2-yl]-2-(1-methyl-1H-pyrazol-4-yl)-3-oxo-2,3-dihydropyridazine-4-carboxamide;
N-(2-hydroxy-2-methylpropyl)-2-(1-methyl-1H-pyrazol-4-yl)-3-oxo-6-[4-(trifluoromethoxy)phenyl]-2,3-dihydropyridazine-4-carboxamide;
6-[4-(difluoromethyl)phenyl]-N-[(cis)-4-hydroxytetrahydrofuran-3-yl]-2-(1-methyl-1H-pyrazol-4-yl)-3-oxo-2,3-dihydropyridazine-4-carboxamide;
1,5-anhydro-2-({[6-(4-chlorophenyl)-2-(1-methyl-1H-pyrazol-4-yl)-3-oxo-2,3-dihydropyridazin-4-yl]carbonyl}amino)-2,4-dideoxy-D-erythro-pentitol;
6-[4-(difluoromethyl)phenyl]-N-(2-hydroxy-2-methylpropyl)-2-(1-methyl-1H-pyrazol-4-yl)-3-oxo-2,3-dihydropyridazine-4-carboxamide;
N-[(trans)-4-hydroxytetrahydrofuran-3-yl]-2-(1-methyl-1H-pyrazol-4-yl)-3-oxo-6-[4-(trifluoromethoxy)phenyl]-2,3-dihydropyridazine-4-carboxamide;
N-[(3R,4S)-4-hydroxytetrahydrofuran-3-yl]-2-(1-methyl-1H-pyrazol-4-yl)-3-oxo-6-[4-(trifluoromethoxy)phenyl]-2,3-dihydropyridazine-4-carboxamide;
N-[(3S,4R)-4-hydroxytetrahydrofuran-3-yl]-2-(1-methyl-1H-pyrazol-4-yl)-3-oxo-6-[4-(trifluoromethoxy)phenyl]-2,3-dihydropyridazine-4-carboxamide;
6-[6-(difluoromethyl)pyridin-3-yl]-N-(1-hydroxypropan-2-yl)-2-(1-methyl-1H-pyrazol-4-yl)-3-oxo-2,3-dihydropyridazine-4-carboxamide;
6-[6-(difluoromethyl)pyridin-3-yl]-N-[(2S)-1-hydroxypropan-2-yl]-2-(1-methyl-1H-pyrazol-4-yl)-3-oxo-2,3-dihydropyridazine-4-carboxamide;
N-(2-hydroxy-2-methylpropyl)-2-(1-methyl-1H-pyrazol-4-yl)-3-oxo-6-[4-(trifluoromethyl)phenyl]-2,3-dihydropyridazine-4-carboxamide;
6-(4-chlorophenyl)-N-(1-hydroxypropan-2-yl)-3-oxo-2-(pyridin-3-yl)-2,3-dihydropyridazine-4-carboxamide;
6-(4-chlorophenyl)-N-[(2R)-1-hydroxypropan-2-yl]-3-oxo-2-(pyridin-3-yl)-2,3-dihydropyridazine-4-carboxamide;
6-(4-chlorophenyl)-N-[(2S)-1-hydroxypropan-2-yl]-3-oxo-2-(pyridin-3-yl)-2,3-dihydropyridazine-4-carboxamide;
6-(4-chlorophenyl)-N-(1,3-dihydroxypropan-2-yl)-3-oxo-2-(pyridin-3-yl)-2,3-dihydropyridazine-4-carboxamide;
6-(4-chlorophenyl)-N-(2-hydroxy-2-methylpropyl)-3-oxo-2-(pyridin-3-yl)-2,3-dihydropyridazine-4-carboxamide;
6-(4-chlorophenyl)-N-(2,3-dihydroxypropyl)-3-oxo-2-(pyridin-3-yl)-2,3-dihydropyridazine-4-carboxamide;
6-(4-chlorophenyl)-N-[(2R)-2,3-dihydroxypropyl]-3-oxo-2-(pyridin-3-yl)-2,3-dihydropyridazine-4-carboxamide;
6-(4-chlorophenyl)-N-[(2S)-2,3-dihydroxypropyl]-3-oxo-2-(pyridin-3-yl)-2,3-dihydropyridazine-4-carboxamide;
6-(4-chlorophenyl)-N-(1-hydroxy-3-methoxypropan-2-yl)-3-oxo-2-(pyridin-3-yl)-2,3-dihydropyridazine-4-carboxamide;
6-(4-chlorophenyl)-N-[(2R)-1-hydroxy-3-methoxypropan-2-yl]-3-oxo-2-(pyridin-3-yl)-2,3-dihydropyridazine-4-carboxamide;
6-(4-chlorophenyl)-N-[(2S)-1-hydroxy-3-methoxypropan-2-yl]-3-oxo-2-(pyridin-3-yl)-2,3-dihydropyridazine-4-carboxamide;
6-(4-chlorophenyl)-3-oxo-2-(pyridin-3-yl)-N-(1,1,1-trifluoro-3-hydroxypropan-2-yl)-2,3-dihydropyridazine-4-carboxamide;
6-(4-chlorophenyl)-3-oxo-2-(pyridin-3-yl)-N-[(2R)-1,1,1-trifluoro-3-hydroxypropan-2-yl]-2,3-dihydropyridazine-4-carboxamide;
6-(4-chlorophenyl)-3-oxo-2-(pyridin-3-yl)-N-[(2S)-1,1,1-trifluoro-3-hydroxypropan-2-yl]-2,3-dihydropyridazine-4-carboxamide;
6-(4-chlorophenyl)-N-(1-cyclopropyl-2-hydroxyethyl)-3-oxo-2-(pyridin-3-yl)-2,3-dihydropyridazine-4-carboxamide;
6-(4-chlorophenyl)-N-[(1R)-1-cyclopropyl-2-hydroxyethyl]-3-oxo-2-(pyridin-3-yl)-2,3-dihydropyridazine-4-carboxamide;
6-(4-chlorophenyl)-N-[(1S)-1-cyclopropyl-2-hydroxyethyl]-3-oxo-2-(pyridin-3-yl)-2,3-dihydropyridazine-4-carboxamide;
6-(4-chlorophenyl)-3-oxo-2-(pyridin-3-yl)-N-(3,3,3-trifluoro-2-hydroxypropyl)-2,3-dihydropyridazine-4-carboxamide;
6-(4-chlorophenyl)-3-oxo-2-(pyridin-3-yl)-N-[(2R)-3,3,3-trifluoro-2-hydroxypropyl]-2,3-dihydropyridazine-4-carboxamide;
6-(4-chlorophenyl)-3-oxo-2-(pyridin-3-yl)-N-[(2S)-3,3,3-trifluoro-2-hydroxypropyl]-2,3-dihydropyridazine-4-carboxamide;
6-(4-chlorophenyl)-N-(3,3-difluoro-2-hydroxypropyl)-3-oxo-2-(pyridin-3-yl)-2,3-dihydropyridazine-4-carboxamide:
6-(4-chlorophenyl)-N-[(2R)-3,3-difluoro-2-hydroxypropyl]-3-oxo-2-(pyridin-3-yl)-2,3-dihydropyridazine-4-carboxamide;
6-(4-chlorophenyl)-N-[(2S)-3,3-difluoro-2-hydroxypropyl]-3-oxo-2-(pyridin-3-yl)-2,3-dihydropyridazine-4-carboxamide;
6-(4-chlorophenyl)-2-(5-fluoropyridin-3-yl)-N-(1-hydroxypropan-2-yl)-3-oxo-2,3-dihydropyridazine-4-carboxamide;
6-(4-chlorophenyl)-2-(5-fluoropyridin-3-yl)-N-[(2R)-1-hydroxypropan-2-yl]-3-oxo-2,3-dihydropyridazine-4-carboxamide;
6-(4-chlorophenyl)-2-(5-fluoropyridin-3-yl)-N-[(2S)-1-hydroxypropan-2-yl]-3-oxo-2,3-dihydropyridazine-4-carboxamide;
6-(4-chlorophenyl)-2-(5-fluoropyridin-3-yl)-N-(2-hydroxy-2-methylpropyl)-3-oxo-2,3-dihydropyridazine-4-carboxamide;
6-(4-chlorophenyl)-2-(5-fluoropyridin-3-yl)-N-(1-hydroxy-3-methylbutan-2-yl)-3-oxo-2,3-dihydropyridazine-4-carboxamide;
6-(4-chlorophenyl)-2-(5-fluoropyridin-3-yl)-N-[(2S)-1-hydroxy-3-methylbutan-2-yl]-3-oxo-2,3-dihydropyridazine-4-carboxamide;
6-(4-chlorophenyl)-N-(1,3-dihydroxypropan-2-yl)-2-(5-fluoropyridin-3-yl)-3-oxo-2,3-dihydropyridazine-4-carboxamide;
6-(4-chlorophenyl)-2-(5-fluoropyridin-3-yl)-N-(1-hydroxy-3-methoxypropan-2-yl)-3-oxo-2,3-dihydropyridazine-4-carboxamide;
6-(4-chlorophenyl)-2-(5-fluoropyridin-3-yl)-N-[(2R)-1-hydroxy-3-methoxypropan-2-yl]-3-oxo-2,3-dihydropyridazine-4-carboxamide;
6-(4-chlorophenyl)-2-(5-fluoropyridin-3-yl)-N-[(2S)-1-hydroxy-3-methoxypropan-2-yl]-3-oxo-2,3-dihydropyridazine-4-carboxamide;
6-(4-chlorophenyl)-N-(1-fluoro-3-hydroxypropan-2-yl)-3-oxo-2-(pyridin-3-yl)-2,3-dihydropyridazine-4-carboxamide;
6-(4-chlorophenyl)-N-[(2R)-1-fluoro-3-hydroxypropan-2-yl]-3-oxo-2-(pyridin-3-yl)-2,3-dihydropyridazine-4-carboxamide;
N-(1-cyclopropyl-2-hydroxyethyl)-3-oxo-2-(pyridin-3-yl)-6-[4-(trifluoromethyl)phenyl]-2,3-dihydropyridazine-4-carboxamide;
N-[(1S)-1-cyclopropyl-2-hydroxyethyl]-3-oxo-2-(pyridin-3-yl)-6-[4-(trifluoromethyl)phenyl]-2,3-dihydropyridazine-4-carboxamide;
6-(4-chlorophenyl)-N-(1-cyclopropyl-2-hydroxyethyl)-2-(5-fluoropyridin-3-yl)-3-oxo-2,3-dihydropyridazine-4-carboxamide;
6-(4-chlorophenyl)-N-(2,3-dihydroxypropyl)-2-(5-fluoropyridin-3-yl)-3-oxo-2,3-dihydropyridazine-4-carboxamide:
6-(4-chlorophenyl)-N-[(2R)-2,3-dihydroxypropyl]-2-(5-fluoropyridin-3-yl)-3-oxo-2,3-dihydropyridazine-4-carboxamide;
6-(4-chlorophenyl)-N-[(2S)-2,3-dihydroxypropyl]-2-(5-fluoropyridin-3-yl)-3-oxo-2,3-dihydropyridazine-4-carboxamide;
6-(4-chlorophenyl)-2-(5-fluoropyridin-3-yl)-3-oxo-N-(1,1,1-trifluoro-3-hydroxypropan-2-yl)-2,3-dihydropyridazine-4-carboxamide;
6-(4-chlorophenyl)-2-(5-fluoropyridin-3-yl)-3-oxo-N-[(2R)-1,1,1-trifluoro-3-hydroxypropan-2-yl]-2,3-dihydropyridazine-4-carboxamide;
N-(3,3-difluoro-2-hydroxypropyl)-3-oxo-2-(pyridin-3-yl)-6-[4-(trifluoromethyl)phenyl]-2,3-dihydropyridazine-4-carboxamide;
N-(2-hydroxycyclopentyl)-3-oxo-2-(pyridin-3-yl)-6-[4-(trifluoromethyl)phenyl]-2,3-dihydropyridazine-4-carboxamide;
N-[(1S,2S)-2-hydroxycyclopentyl]-3-oxo-2-(pyridin-3-yl)-6-[4-(trifluoromethyl)phenyl]-2,3-dihydropyridazine-4-carboxamide;
N-[(1R,2R)-2-hydroxycyclopentyl]-3-oxo-2-(pyridin-3-yl)-6-[4-(trifluoromethyl)phenyl]-2,3-dihydropyridazine-4-carboxamide;
N-[(1R,2S)-2-hydroxycyclopentyl]-3-oxo-2-(pyridin-3-yl)-6-[4-(trifluoromethyl)phenyl]-2,3-dihydropyridazine-4-carboxamide;
N-[(1S,2R)-2-hydroxycyclopentyl]-3-oxo-2-(pyridin-3-yl)-6-[4-(trifluoromethyl)phenyl]-2,3-dihydropyridazine-4-carboxamide;
N-[(cis)-2-hydroxycyclohexyl]-3-oxo-2-(pyridin-3-yl)-6-[4-(trifluoromethyl)phenyl]-2,3-dihydropyridazine-4-carboxamide;
N-[(1S,2R)-2-hydroxycyclohexyl]-3-oxo-2-(pyridin-3-yl)-6-[4-(trifluoromethyl)phenyl]-2,3-dihydropyridazine-4-carboxamide;
N-[(cis)-4-hydroxytetrahydrofuran-3-yl]-3-oxo-2-(pyridin-3-yl)-6-[4-(trifluoromethyl)phenyl]-2,3-dihydropyridazine-4-carboxamide;
3-oxo-2-(pyridin-3-yl)-N-(3,3,3-trifluoro-2-hydroxypropyl)-6-[4-(trifluoromethyl)phenyl]-2,3-dihydropyridazine-4-carboxamide;
3-oxo-2-(pyridin-3-yl)-N-[(2S)-3,3,3-trifluoro-2-hydroxypropyl]-6-[4-(trifluoromethyl)phenyl]-2,3-dihydropyridazine-4-carboxamide;
3-oxo-2-(pyridin-3-yl)-N-(1,1,1-trifluoro-3-hydroxypropan-2-yl)-6-[4-(trifluoromethyl)phenyl]-2,3-dihydropyridazine-4-carboxamide;
3-oxo-2-(pyridin-3-yl)-N-(1,1,1-trifluoro-3-hydroxypropan-2-yl)-6-[4-(trifluoromethyl)phenyl]-2,3-dihydropyridazine-4-carboxamide;
3-oxo-2-(pyridin-3-yl)-N-[(2R)-1,1,1-trifluoro-3-hydroxypropan-2-yl]-6-[4-(trifluoromethyl)phenyl]-2,3-dihydropyridazine-4-carboxamide;
N-(1-hydroxy-3-methoxypropan-2-yl)-3-oxo-2-(pyridin-3-yl)-6-[4-(trifluoromethyl)phenyl]-2,3-dihydropyridazine-4-carboxamide;
N-[(2S)-1-hydroxy-3-methoxypropan-2-yl]-3-oxo-2-(pyridin-3-yl)-6-[4-(trifluoromethyl)phenyl]-2,3-dihydropyridazine-4-carboxamide;
N-(2,3-dihydroxypropyl)-3-oxo-2-(pyridin-3-yl)-6-[4-(trifluoromethyl)phenyl]-2,3-dihydropyridazine-4-carboxamide;
N-[(2R)-2,3-dihydroxypropyl]-3-oxo-2-(pyridin-3-yl)-6-[4-(trifluoromethyl)phenyl]-2,3-dihydropyridazine-4-carboxamide;
N-[(2S)-2,3-dihydroxypropyl]-3-oxo-2-(pyridin-3-yl)-6-[4-(trifluoromethyl)phenyl]-2,3-dihydropyridazine-4-carboxamide;
N-(2-hydroxy-2-methylpropyl)-3-oxo-2-(pyridin-3-yl)-6-[4-(trifluoromethyl)phenyl]-2,3-dihydropyridazine-4-carboxamide;
N-(1,3-dihydroxypropan-2-yl)-3-oxo-2-(pyridin-3-yl)-6-[4-(trifluoromethyl)phenyl]-2,3-dihydropyridazine-4-carboxamide;
N-(1-hydroxypropan-2-yl)-3-oxo-2-(pyridin-3-yl)-6-[4-(trifluoromethyl)phenyl]-2,3-dihydropyridazine-4-carboxamide;
N-[(2R)-1-hydroxypropan-2-yl]-3-oxo-2-(pyridin-3-yl)-6-[4-(trifluoromethyl)phenyl]-2,3-dihydropyridazine-4-carboxamide;
N-[(2S)-1-hydroxypropan-2-yl]-3-oxo-2-(pyridin-3-yl)-6-[4-(trifluoromethyl)phenyl]-2,3-dihydropyridazine-4-carboxamide;
6-(4-chlorophenyl)-N-(1-hydroxypropan-2-yl)-3-oxo-2-(pyrimidin-5-yl)-2,3-dihydropyridazine-4-carboxamide;
6-(4-chlorophenyl)-N-[(2S)-1-hydroxypropan-2-yl]-3-oxo-2-(pyrimidin-5-yl)-2,3-dihydropyridazine-4-carboxamide;
6-(4-chlorophenyl)-3-oxo-2-(pyridin-3-yl)-N-(1,1,1-trifluoro-3-hydroxy-3-methylbutan-2-yl)-2,3-dihydropyridazine-4-carboxamide;
6-(4-chlorophenyl)-3-oxo-2-(pyridin-3-yl)-N-[(2R)-1,1,1-trifluoro-3-hydroxy-3-methylbutan-2-yl]-2,3-dihydropyridazine-4-carboxamide;
6-(4-chlorophenyl)-3-oxo-2-(pyridin-3-yl)-N-[(2S)-1,1,1-trifluoro-3-hydroxy-3-methylbutan-2-yl]-2,3-dihydropyridazine-4-carboxamide;
6-(4-chlorophenyl)-2-(5-fluoropyridin-3-yl)-N-[(cis)-2-hydroxycyclohexyl]-3-oxo-2,3-dihydropyridazine-4-carboxamide;
6-(4-chlorophenyl)-2-(5-fluoropyridin-3-yl)-N-[(1S,2R)-2-hydroxycyclohexyl]-3-oxo-2,3-dihydropyridazine-4-carboxamide;
6-(4-chlorophenyl)-2-(5-fluoropyridin-3-yl)-N-[(cis)-2-hydroxycyclohexyl]-3-oxo-2,3-dihydropyridazine-4-carboxamide;
6-(4-chlorophenyl)-2-(5-fluoropyridin-3-yl)-N-[(1S,2R)-2-hydroxycyclohexyl]-3-oxo-2,3-dihydropyridazine-4-carboxamide;
N-[(cis)-2-hydroxycyclohexyl]-3-oxo-2-(pyridin-3-yl)-6-[4-(trifluoromethoxy)phenyl]-2,3-dihydropyridazine-4-carboxamide;
N-[(1S,2R)-2-hydroxycyclohexyl]-3-oxo-2-(pyridin-3-yl)-6-[4-(trifluoromethoxy)phenyl]-2,3-dihydropyridazine-4-carboxamide;
6-[4-(difluoromethyl)phenyl]-3-oxo-2-(pyridin-3-yl)-N-(1,1,1-trifluoro-3-hydroxypropan-2-yl)-2,3-dihydropyridazine-4-carboxamide;
6-[4-(difluoromethyl)phenyl]-3-oxo-2-(pyridin-3-yl)-N-[(2R)-1,1,1-trifluoro-3-hydroxypropan-2-yl]-2,3-dihydropyridazine-4-carboxamide;
6-(4-chlorophenyl)-2-(5-fluoropyridin-3-yl)-3-oxo-N-(3,3,3-trifluoro-2-hydroxypropyl)-2,3-dihydropyridazine-4-carboxamide;
6-(4-chlorophenyl)-2-(5-fluoropyridin-3-yl)-3-oxo-N-[(2S)-3,3,3-trifluoro-2-hydroxypropyl]-2,3-dihydropyridazine-4-carboxamide;
6-(4-chlorophenyl)-2-(5-fluoropyridin-3-yl)-N-[(cis)-2-hydroxycyclopentyl]-3-oxo-2,3-dihydropyridazine-4-carboxamide;
6-(4-chlorophenyl)-2-(5-fluoropyridin-3-yl)-N-[(1S,2R)-2-hydroxycyclopentyl]-3-oxo-2,3-dihydropyridazine-4-carboxamide;
6-(4-chlorophenyl)-N-[(trans)-2-hydroxycyclopentyl]-3-oxo-2-(pyridin-3-yl)-2,3-dihydropyridazine-4-carboxamide;
6-(4-chlorophenyl)-N-[(1S,2S)-2-hydroxycyclopentyl]-3-oxo-2-(pyridin-3-yl)-2,3-dihydropyridazine-4-carboxamide;
3-oxo-2-(pyridin-3-yl)-N-(3,3,3-trifluoro-2-hydroxypropyl)-6-[4-(trifluoromethoxy)phenyl]-2,3-dihydropyridazine-4-carboxamide;
3-oxo-2-(pyridin-3-yl)-N-[(2S)-3,3,3-trifluoro-2-hydroxypropyl]-6-[4-(trifluoromethoxy)phenyl]-2,3-dihydropyridazine-4-carboxamide;
N-[(trans)-2-hydroxycyclopentyl]-3-oxo-2-(pyridin-3-yl)-6-[4-(trifluoromethoxy)phenyl]-2,3-dihydropyridazine-4-carboxamide;
N-[(1S,2S)-2-hydroxycyclopentyl]-3-oxo-2-(pyridin-3-yl)-6-[4-(trifluoromethoxy)phenyl]-2,3-dihydropyridazine-4-carboxamide;
6-(4-chlorophenyl)-N-[(cis)-2-hydroxycyclohexyl]-3-oxo-2-(pyridin-3-yl)-2,3-dihydropyridazine-4-carboxamide;
3-oxo-2-(pyridin-3-yl)-N-(1,1,1-trifluoro-3-hydroxypropan-2-yl)-6-[4-(trifluoromethoxy)phenyl]-2,3-dihydropyridazine-4-carboxamide;
3-oxo-2-(pyridin-3-yl)-N-[(2R)-1,1,1-trifluoro-3-hydroxypropan-2-yl]-6-[4-(trifluoromethoxy)phenyl]-2,3-dihydropyridazine-4-carboxamide;
3-oxo-2-(pyridin-3-yl)-N-(1,1,1-trifluoro-3-hydroxy-3-methylbutan-2-yl)-6-[4-(trifluoromethoxy)phenyl]-2,3-dihydropyridazine-4-carboxamide;
3-oxo-2-(pyridin-3-yl)-N-[(2R)-1,1,1-trifluoro-3-hydroxy-3-methylbutan-2-yl]-6-[4-(trifluoromethoxy)phenyl]-2,3-dihydropyridazine-4-carboxamide;
3-oxo-2-(pyridin-3-yl)-N-[(2S)-1,1,1-trifluoro-3-hydroxy-3-methylbutan-2-yl]-6-[4-(trifluoromethoxy)phenyl]-2,3-dihydropyridazine-4-carboxamide;
6-(4-chlorophenyl)-N-[(cis)-2-hydroxycyclopentyl]-3-oxo-2-(pyridin-3-yl)-2,3-dihydropyridazine-4-carboxamide;
6-(4-chlorophenyl)-N-[(1R,2S)-2-hydroxycyclopentyl]-3-oxo-2-(pyridin-3-yl)-2,3-dihydropyridazine-4-carboxamide;
6-[4-(difluoromethyl)phenyl]-N-[(trans)-2-hydroxycyclopentyl]-3-oxo-2-(pyridin-3-yl)-2,3-dihydropyridazine-4-carboxamide
6-[4-(difluoromethyl)phenyl]-N-[(1S,2S)-2-hydroxycyclopentyl]-3-oxo-2-(pyridin-3-yl)-2,3-dihydropyridazine-4-carboxamide;
N-[(trans)-3,3-difluoro-2-hydroxycyclohexyl]-3-oxo-2-(pyridin-3-yl)-6-[4-(trifluoromethoxy)phenyl]-2,3-dihydropyridazine-4-carboxamide;
N-[(1S,2R)-3,3-difluoro-2-hydroxycyclohexyl]-3-oxo-2-(pyridin-3-yl)-6-[4-(trifluoromethoxy)phenyl]-2,3-dihydropyridazine-4-carboxamide;
N-[(1R,2S)-3,3-difluoro-2-hydroxycyclohexyl]-3-oxo-2-(pyridin-3-yl)-6-[4-(trifluoromethoxy)phenyl]-2,3-dihydropyridazine-4-carboxamide;
6-(4-chlorophenyl)-N-[(trans)-3,3-difluoro-2-hydroxycyclohexyl]-3-oxo-2-(pyridin-3-yl)-2,3-dihydropyridazine-4-carboxamide;
6-(4-chlorophenyl)-N-[(1S,2R)-3,3-difluoro-2-hydroxycyclohexyl]-3-oxo-2-(pyridin-3-yl)-2,3-dihydropyridazine-4-carboxamide;
6-(4-chlorophenyl)-N-[(1R,2S)-3,3-difluoro-2-hydroxycyclohexyl]-3-oxo-2-(pyridin-3-yl)-2,3-dihydropyridazine-4-carboxamide;
N-[(cis)-2-hydroxycyclopentyl]-3-oxo-2-(pyridin-3-yl)-6-[4-(trifluoromethoxy)phenyl]-2,3-dihydropyridazine-4-carboxamide;
N-[(1S,2R)-2-hydroxycyclopentyl]-3-oxo-2-(pyridin-3-yl)-6-[4-(trifluoromethoxy)phenyl]-2,3-dihydropyridazine-4-carboxamide;
N-(1-cyclopropyl-2-hydroxyethyl)-6-[4-(difluoromethyl)phenyl]-3-oxo-2-(pyridin-3-yl)-2,3-dihydropyridazine-4-carboxamide;
N-[(1S)-1-cyclopropyl-2-hydroxyethyl]-6-[4-(difluoromethyl)phenyl]-3-oxo-2-(pyridin-3-yl)-2,3-dihydropyridazine-4-carboxamide;
6-[4-(difluoromethyl)phenyl]-N-(1-hydroxypropan-2-yl)-3-oxo-2-(pyridin-3-yl)-2,3-dihydropyridazine-4-carboxamide;
6-[4-(difluoromethyl)phenyl]-N-[(2S)-1-hydroxypropan-2-yl]-3-oxo-2-(pyridin-3-yl)-2,3-dihydropyridazine-4-carboxamide;
N-(1-hydroxypropan-2-yl)-6-(4-methylphenyl)-3-oxo-2-(pyridin-3-yl)-2,3-dihydropyridazine-4-carboxamide;
N-[(2S)-1-hydroxypropan-2-yl]-6-(4-methylphenyl)-3-oxo-2-(pyridin-3-yl)-2,3-dihydropyridazine-4-carboxamide;
N-(1-hydroxypropan-2-yl)-3-oxo-2-(pyridin-3-yl)-6-[4-(trifluoromethoxy)phenyl]-2,3-dihydropyridazine-4-carboxamide;
N-[(2S)-1-hydroxypropan-2-yl]-3-oxo-2-(pyridin-3-yl)-6-[4-(trifluoromethoxy)phenyl]-2,3-dihydropyridazine-4-carboxamide;
6-(4-chlorophenyl)-2-(5-fluoropyridin-3-yl)-N-[(trans)-4-hydroxytetrahydrofuran-3-yl]-3-oxo-2,3-dihydropyridazine-4-carboxamide;
6-(4-chlorophenyl)-2-(5-fluoropyridin-3-yl)-N-[(3S,4R)-4-hydroxytetrahydrofuran-3-yl]-3-oxo-2,3-dihydropyridazine-4-carboxamide;
6-(4-chlorophenyl)-2-(5-fluoropyridin-3-yl)-N-[(3R,4S)-4-hydroxytetrahydrofuran-3-yl]-3-oxo-2,3-dihydropyridazine-4-carboxamide;
N-[(cis)-4-hydroxytetrahydrofuran-3-yl]-3-oxo-2-(pyridin-3-yl)-6-[4-(trifluoromethoxy)phenyl]-2,3-dihydropyridazine-4-carboxamide;
N-[(3S,4S)-4-hydroxytetrahydrofuran-3-yl]-3-oxo-2-(pyridin-3-yl)-6-[4-(trifluoromethoxy)phenyl]-2,3-dihydropyridazine-4-carboxamide;
N-[(3R,4R)-4-hydroxytetrahydrofuran-3-yl]-3-oxo-2-(pyridin-3-yl)-6-[4-(trifluoromethoxy)phenyl]-2,3-dihydropyridazine-4-carboxamide;
N-[(trans)-4-hydroxytetrahydrofuran-3-yl]-3-oxo-2-(pyridin-3-yl)-6-[4-(trifluoromethoxy)phenyl]-2,3-dihydropyridazine-4-carboxamide;
N-[(3S,4R)-4-hydroxytetrahydrofuran-3-yl]-3-oxo-2-(pyridin-3-yl)-6-[4-(trifluoromethoxy)phenyl]-2,3-dihydropyridazine-4-carboxamide;
N-[(3R,4S)-4-hydroxytetrahydrofuran-3-yl]-3-oxo-2-(pyridin-3-yl)-6-[4-(trifluoromethoxy)phenyl]-2,3-dihydropyridazine-4-carboxamide;
6-(4-chloro-2-fluorophenyl)-N-(1-hydroxypropan-2-yl)-3-oxo-2-(pyridin-3-yl)-2,3-dihydropyridazine-4-carboxamide;
6-(4-chloro-2-fluorophenyl)-N-[(2S)-1-hydroxypropan-2-yl]-3-oxo-2-(pyridin-3-yl)-2,3-dihydropyridazine-4-carboxamide;
3-(4-chlorophenyl)-N-[(cis)-2-hydroxycyclohexyl]-6-oxo-6H-1,4′-bipyridazine-5-carboxamide;
3-(4-chlorophenyl)-N-[(1S,2R)-2-hydroxycyclohexyl]-6-oxo-6H-1,4′-bipyridazine-5-carboxamide
6-[6-(difluoromethyl)pyridin-3-yl]-N-[(trans)-2-hydroxycyclopentyl]-3-oxo-2-(pyridin-3-yl)-2,3-dihydropyridazine-4-carboxamide;
6-[6-(difluoromethyl)pyridin-3-yl]-N-[(1S,2S)-2-hydroxycyclopentyl]-3-oxo-2-(pyridin-3-yl)-2,3-dihydropyridazine-4-carboxamide;
N-(1-hydroxypropan-2-yl)-3-oxo-2-(pyridin-3-yl)-6-[6-(trifluoromethyl)pyridin-3-yl]-2,3-dihydropyridazine-4-carboxamide;
N-[(2S)-1-hydroxypropan-2-yl]-3-oxo-2-(pyridin-3-yl)-6-[6-(trifluoromethyl)pyridin-3-yl]-2,3-dihydropyridazine-4-carboxamide;
3-(4-chlorophenyl)-N-[(cis)-2-hydroxycyclopentyl]-6-oxo-6H-1,4′-bipyridazine-5-carboxamide;
3-(4-chlorophenyl)-N-[(1S,2R)-2-hydroxycyclopentyl]-6-oxo-6H-1,4′-bipyridazine-5-carboxamide;
6-(4-chlorophenyl)-N-[(trans)-4-hydroxytetrahydrofuran-3-yl]-3-oxo-2-(pyridin-3-yl)-2,3-dihydropyridazine-4-carboxamide;
6-(4-chlorophenyl)-N-[(3S,4R)-4-hydroxytetrahydrofuran-3-yl]-3-oxo-2-(pyridin-3-yl)-2,3-dihydropyridazine-4-carboxamide;
6-(4-chlorophenyl)-N-[(3R,4S)-4-hydroxytetrahydrofuran-3-yl]-3-oxo-2-(pyridin-3-yl)-2,3-dihydropyridazine-4-carboxamide;
6-(4-chlorophenyl)-N-[(cis)-4-hydroxytetrahydrofuran-3-yl]-3-oxo-2-(pyridin-3-yl)-2,3-dihydropyridazine-4-carboxamide;
6-(4-chlorophenyl)-N-[(3R,4R)-4-hydroxytetrahydrofuran-3-yl]-3-oxo-2-(pyridin-3-yl)-2,3-dihydropyridazine-4-carboxamide;
6-(4-chlorophenyl)-N-[(3S,4S)-4-hydroxytetrahydrofuran-3-yl]-3-oxo-2-(pyridin-3-yl)-2,3-dihydropyridazine-4-carboxamide;
3-(4-chlorophenyl)-N-[(trans)-2-hydroxycyclopentyl]-6-oxo-6H-1,4′-bipyridazine-5-carboxamide;
3-(4-chlorophenyl)-N-[(1S,2S)-2-hydroxycyclopentyl]-6-oxo-6H-1,4′-bipyridazine-5-carboxamide;
1,5-anhydro-2-({[6-(4-chlorophenyl)-3-oxo-2-(pyridin-3-yl)-2,3-dihydropyridazin-4-yl]carbonyl}amino)-2,4-dideoxy-cis-pentitol;
1,5-anhydro-2-({[6-(4-chlorophenyl)-3-oxo-2-(pyridin-3-yl)-2,3-dihydropyridazin-4-yl]carbonyl}amino)-2,4-dideoxy-D-erythro-pentitol;
1,5-anhydro-2,4-dideoxy-2-[({3-oxo-2-(pyridin-3-yl)-6-[4-(trifluoromethoxy)phenyl]-2,3-dihydropyridazin-4-yl}carbonyl)amino]-cis-pentitol;
1,5-anhydro-2,4-dideoxy-2-[({3-oxo-2-(pyridin-3-yl)-6-[4-(trifluoromethoxy)phenyl]-2,3-dihydropyridazin-4-yl}carbonyl)amino]-D-erythro-pentitol;
3-(4-chlorophenyl)-N-(1-hydroxypropan-2-yl)-6-oxo-6H-1,4′-bipyridazine-5-carboxamide;
3-(4-chlorophenyl)-N-[(2S)-1-hydroxypropan-2-yl]-6-oxo-6H-1,4′-bipyridazine-5-carboxamide;
3-(4-chlorophenyl)-N-(2-hydroxy-2-methylpropyl)-6-oxo-6H-1,4′-bipyridazine-5-carboxamide;
6-(4-cyanophenyl)-N-(1-hydroxypropan-2-yl)-3-oxo-2-(pyridin-3-yl)-2,3-dihydropyridazine-4-carboxamide;
6-(4-cyanophenyl)-N-[(2S)-1-hydroxypropan-2-yl]-3-oxo-2-(pyridin-3-yl)-2,3-dihydropyridazine-4-carboxamide;
6-[6-(difluoromethyl)pyridin-3-yl]-N-(1-hydroxypropan-2-yl)-3-oxo-2-(pyridin-3-yl)-2,3-dihydropyridazine-4-carboxamide;
6-[6-(difluoromethyl)pyridin-3-yl]-N-[(2S)-1-hydroxypropan-2-yl]-3-oxo-2-(pyridin-3-yl)-2,3-dihydropyridazine-4-carboxamide;
3-(4-chlorophenyl)-N-[(cis)-4-hydroxytetrahydrofuran-3-yl]-6-oxo-6H-1,4′-bipyridazine-5-carboxamide;
6-[6-(difluoromethyl)pyridin-3-yl]-N-[(cis)-4-methyltetrahydrofuran-3-yl]-3-oxo-2-(pyridin-3-yl)-2,3-dihydropyridazine-4-carboxamide;
6-[6-(difluoromethyl)pyridin-3-yl]-N-[(3R,4S)-4-methyltetrahydrofuran-3-yl]-3-oxo-2-(pyridin-3-yl)-2,3-dihydropyridazine-4-carboxamide;
6-[6-(difluoromethyl)pyridin-3-yl]-N-[(3S,4R)-4-methyltetrahydrofuran-3-yl]-3-oxo-2-(pyridin-3-yl)-2,3-dihydropyridazine-4-carboxamide;
6-(4-chlorophenyl)-3-oxo-2-(pyrimidin-5-yl)-N-(1,1,1-trifluoro-3-hydroxy-3-methylbutan-2-yl)-2,3-dihydropyridazine-4-carboxamide;
6-(4-chlorophenyl)-3-oxo-2-(pyrimidin-5-yl)-N-[(2R)-1,1,1-trifluoro-3-hydroxy-3-methylbutan-2-yl]-2,3-dihydropyridazine-4-carboxamide;
6-(4-chlorophenyl)-3-oxo-2-(pyrimidin-5-yl)-N-[(2S)-1,1,1-trifluoro-3-hydroxy-3-methylbutan-2-yl]-2,3-dihydropyridazine-4-carboxamide;
N-[(1-hydroxycyclopropyl)methyl]-3-oxo-2-(pyridin-3-yl)-6-[4-(trifluoromethyl)phenyl]-2,3-dihydropyridazine-4-carboxamide;
N-[(1-hydroxycyclobutyl)methyl]-3-oxo-2-(pyridin-3-yl)-6-[4-(trifluoromethyl)phenyl]-2,3-dihydropyridazine-4-carboxamide;
6-(4-chlorophenyl)-N-(1-hydroxypropan-2-yl)-3-oxo-2-(1H-pyrazol-4-yl)-2,3-dihydropyridazine-4-carboxamide;
6-(4-chlorophenyl)-N-[(2S)-1-hydroxypropan-2-yl]-3-oxo-2-(1H-pyrazol-4-yl)-2,3-dihydropyridazine-4-carboxamide;
6-[4-(dimethylamino)phenyl]-N-(1-hydroxypropan-2-yl)-3-oxo-2-(pyridin-3-yl)-2,3-dihydropyridazine-4-carboxamide;
6-[4-(dimethylamino)phenyl]-N-[(2S)-1-hydroxypropan-2-yl]-3-oxo-2-(pyridin-3-yl)-2,3-dihydropyridazine-4-carboxamide;
6-(4-chlorophenyl)-N-(1-hydroxypropan-2-yl)-2-(1-methyl-1H-pyrazol-3-yl)-3-oxo-2,3-dihydropyridazine-4-carboxamide;
6-(4-chlorophenyl)-N-[(2S)-1-hydroxypropan-2-yl]-2-(1-methyl-1H-pyrazol-3-yl)-3-oxo-2,3-dihydropyridazine-4-carboxamide;
6-(4-chlorophenyl)-N-(1-hydroxypropan-2-yl)-2-(3-methyl-1H-pyrazol-5-yl)-3-oxo-2,3-dihydropyridazine-4-carboxamide;
6-(4-chlorophenyl)-N-[(2S)-1-hydroxypropan-2-yl]-2-(3-methyl-1H-pyrazol-5-yl)-3-oxo-2,3-dihydropyridazine-4-carboxamide;
6-(4-chlorophenyl)-N-(1-hydroxypropan-2-yl)-3-oxo-2-(1,2-thiazol-4-yl)-2,3-dihydropyridazine-4-carboxamide;
6-(4-chlorophenyl)-N-[(2S)-1-hydroxypropan-2-yl]-3-oxo-2-(1,2-thiazol-4-yl)-2,3-dihydropyridazine-4-carboxamide;
N-[(1-Hydroxycyclobutyl)methyl]-3-oxo-2-(1,2-thiazol-4-yl)-6-[4-(trifluoromethyl)phenyl]-2,3-dihydropyridazine-4-carboxamide;
N-[(2S)-3-Hydroxy-3-methylbutan-2-yl]-3-oxo-2-(1,2-thiazol-4-yl)-6-[4-(trifluoromethyl)phenyl]-2,3-dihydropyridazine-4-carboxamide;
(+)-3-Oxo-2-(pyridin-3-yl)-N-(1,1,1-trifluoro-3-hydroxy-3-methylbutan-2-yl)-6-[4-(trifluoromethyl)phenyl]-2,3-dihydropyridazine-4-carboxamide enantiomer 2;
6-(4-Chlorophenyl)-3-oxo-2-(1,2-thiazol-4-yl)-N-[(2R)-1,1,1-trifluoro-3-hydroxypropan-2-yl]-2,3-dihydropyridazine-4-carboxamide;
6-(4-Chlorophenyl)-3-oxo-2-(1,2-thiazol-4-yl)-N-[(2S)-3,3,3-trifluoro-2-hydroxypropyl]-2,3-dihydropyridazine-4-carboxamide;
6-(4-Chlorophenyl)-N-[(1-hydroxycyclobutyl)methyl]-3-oxo-2-(1,2-thiazol-4-yl)-2,3-dihydropyridazine-4-carboxamide
6-(4-Chlorophenyl)-N-[(2R)-1-fluoro-3-hydroxypropan-2-yl]-3-oxo-2-(1,2-thiazol-4-yl)-2,3-dihydropyridazine-4-carboxamide;
N-[(2R)-3-Hydroxy-3-methylbutan-2-yl]-2-(1-methyl-1H-pyrazol-4-yl)-3-oxo-6-[4-(trifluoromethyl)phenyl]-2,3-dihydropyridazine-4-carboxamide;
N-[(2R)-1-Fluoro-3-hydroxypropan-2-yl]-3-oxo-2-(1,2-thiazol-4-yl)-6-[4-(trifluoromethyl)phenyl]-2,3-dihydropyridazine-4-carboxamide;
N-[(1S,2R)-2-Hydroxycyclopentyl]-2-(1-methyl-1H-pyrazol-4-yl)-3-oxo-6-[4-(trifluoromethyl)phenyl]-2,3-dihydropyridazine-4-carboxamide,
N-[(2S)-1-Hydroxypropan-2-yl]-3-oxo-2-(1,2-thiazol-4-yl)-6-[4-(trifluoromethyl)phenyl]-2,3-dihydropyridazine-4-carboxamide;
N-[(2S)-3-Hydroxy-3-methylbutan-2-yl]-2-(1-methyl-1H-pyrazol-4-yl)-3-oxo-6-[4-(trifluoromethyl)phenyl]-2,3-dihydropyridazine-4-carboxamide;
N-[(1S)-1-Cyclopropyl-2-hydroxyethyl]-3-oxo-2-(1,2-thiazol-4-yl)-6-[4-(trifluoromethyl)phenyl]-2,3-dihydropyridazine-4-carboxamide;
N-(2-Hydroxy-2-methylpropyl)-3-oxo-2-(1,2-thiazol-4-yl)-6-[4-(trifluoromethyl)phenyl]-2,3-dihydropyridazine-4-carboxamide;
6-(4-Chlorophenyl)-N-[(2S)-3-hydroxy-3-methylbutan-2-yl]-3-oxo-2-(1,2-thiazol-4-yl)-2,3-dihydropyridazine-4-carboxamide;
N-[(1S,2S)-2-Hydroxycyclopentyl]-2-(1-methyl-1H-pyrazol-4-yl)-3-oxo-6-[4-(trifluoromethyl)phenyl]-2,3-dihydropyridazine-4-carboxamide;
N-[(2R)-1-Fluoro-3-hydroxypropan-2-yl]-2-(1-methyl-1H-pyrazol-4-yl)-3-oxo-6-[4-(trifluoromethyl)phenyl]-2,3-dihydropyridazine-4-carboxamide;
3-Oxo-2-(1H-pyrazol-4-yl)-N-[(2R)-1,1,1-trifluoro-3-hydroxypropan-2-yl]-6-[4-(trifluoromethyl)phenyl]-2,3-dihydropyridazine-4-carboxamide;
3-Oxo-2-(pyridin-3-yl)-N-(1,1,1-trifluoro-3-hydroxy-3-methylbutan-2-yl)-6-[4-(trifluoromethyl)phenyl]-2,3-dihydropyridazine-4-carboxamide;
2-[1-(Difluoromethyl)-1H-pyrazol-4-yl]-3-oxo-N-[(2R)-1,1,1-trifluoro-3-hydroxypropan-2-yl]-6-[4-(trifluoromethyl)phenyl]-2,3-dihydropyridazine-4-carboxamide;
N-cis-4-Hydroxytetrahydrofuran-3-yl-2-(1-methyl-1H-pyrazol-4-yl)-3-oxo-6-[4-(trifluoromethyl)phenyl]-2,3-dihydropyridazine-4-carboxamide;
3-Oxo-2-(1H-pyrazol-4-yl)-N-[(2S)-3,3,3-trifluoro-2-hydroxypropyl]-6-[4-(trifluoromethyl)phenyl]-2,3-dihydropyridazine-4-carboxamide;
(+)-N-cis-2-Hydroxycyclobutyl-3-oxo-2-(pyridin-3-yl)-6-[4-(trifluoromethyl)phenyl]-2,3-dihydropyridazine-4-carboxamide enantiomer 1;
(+)-N-cis-4-Hydroxytetrahydrofuran-3-yl-2-(1-methyl-1H-pyrazol-4-yl)-3-oxo-6-[4-(trifluoromethyl)phenyl]-2,3-dihydropyridazine-4-carboxamide enantiomer 2;
N-[(2S)-3,3-Difluoro-2-hydroxypropyl]-2-(1-methyl-1H-pyrazol-4-yl)-3-oxo-6-[4-(trifluoromethyl)phenyl]-2,3-dihydropyridazine-4-carboxamide;
6-(4-Chlorophenyl)-2-(5-fluoro-2-thienyl)-N-[(2S)-1-hydroxypropan-2-yl]-3-oxo-2,3-dihydropyridazine-4-carboxamide;
N-[(2R)-1-Fluoro-3-hydroxypropan-2-yl]-3-oxo-2-(pyridin-3-yl)-6-[4-(trifluoromethyl)phenyl]-2,3-dihydropyridazine-4-carboxamide;
6-(4-Chlorophenyl)-N-(2-hydroxy-2-methylpropyl)-3-oxo-2-(1,2-thiazol-4-yl)-2,3-dihydropyridazine-4-carboxamide;
2-[1-(Difluoromethyl)-1H-pyrazol-4-yl]-3-oxo-N-[(2S)-3,3,3-trifluoro-2-hydroxypropyl]-6-[4-(trifluoromethyl)phenyl]-2,3-dihydropyridazine-4-carboxamide;
N-cis-2-Hydroxycyclobutyl-3-oxo-2-(pyridin-3-yl)-6-[4-(trifluoromethyl)phenyl]-2,3-dihydropyridazine-4-carboxamide
6-(4-Chlorophenyl)-2-(5-methyl-3-thienyl)-3-oxo-N-[(2S)-3,3,3-trifluoro-2-hydroxypropyl]-2,3-dihydropyridazine-4-carboxamide;
N-[(2S)-3-Hydroxy-3-methylbutan-2-yl]-3-oxo-2-(pyridin-3-yl)-6-[4-(trifluoromethyl)phenyl]-2,3-dihydropyridazine-4-carboxamide;
6-(4-Chlorophenyl)-N-[(1S)-1-cyano-2-hydroxyethyl]-2-(1-methyl-1H-pyrazol-4-yl)-3-oxo-2,3-dihydropyridazine-4-carboxamide;
2-[1-(Difluoromethyl)-1H-pyrazol-4-yl]-N-[(2S)-3-hydroxy-3-methylbutan-2-yl]-3-oxo-6-[4-(trifluoromethyl)phenyl]-2,3-dihydropyridazine-4-carboxamide;
(+)-N-cis-4-Hydroxytetrahydrofuran-3-yl-3-oxo-2-(pyridin-3-yl)-6-[4-(trifluoromethyl)phenyl]-2,3-dihydropyridazine-4-carboxamide enantiomer 1;
(−)-N-(3,3-Difluoro-2-hydroxypropyl)-3-oxo-2-(pyridin-3-yl)-6-[4-(trifluoromethyl)phenyl]-2,3-dihydropyridazine-4-carboxamide enantiomer 2;
(−)-N-cis-4-Hydroxytetrahydrofuran-3-yl-2-(1-methyl-1H-pyrazol-4-yl)-3-oxo-6-[4-(trifluoromethyl)phenyl]-2,3-dihydropyridazine-4-carboxamide enantiomer 1;
6-(4-Chlorophenyl)-2-(5-chloro-3-thienyl)-3-oxo-N-[(2S)-3,3,3-trifluoro-2-hydroxypropyl]-2,3-dihydropyridazine-4-carboxamide;
N-[(2S)-3-Hydroxy-3-methylbutan-2-yl]-3-oxo-2-(1,2-thiazol-4-yl)-6-[6-(trifluoromethyl)pyridin-3-yl]-2,3-dihydropyridazine-4-carboxamide;
N-[(2R)-3-Hydroxy-3-methylbutan-2-yl]-3-oxo-2-(pyridin-3-yl)-6-[4-(trifluoromethyl)phenyl]-2,3-dihydropyridazine-4-carboxamide;
6-(4-Chlorophenyl)-2-(1,2-oxazol-4-yl)-3-oxo-N-[(2S)-3,3,3-trifluoro-2-hydroxypropyl]-2,3-dihydropyridazine-4-carboxamide;
2-[1-(Difluoromethyl)-1H-pyrazol-4-yl]-3-oxo-N-[(2S)-3,3,3-trifluoro-2-hydroxypropyl]-6-[4-(trifluoromethyl)phenyl]-2,3-dihydropyridazine-4-carboxamide;
6-(4-Chlorophenyl)-3-oxo-2-(pyrimidin-5-yl)-N-[(2S)-3,3,3-trifluoro-2-hydroxypropyl]-2,3-dihydropyridazine-4-carboxamide;
N-[(1S)-1-Cyclopropyl-2-hydroxyethyl]-2-(1-methyl-1H-pyrazol-4-yl)-3-oxo-6-[4-(trifluoromethyl)phenyl]-2,3-dihydropyridazine-4-carboxamide;
N-[(1R,2S)-2-Hydroxycyclopentyl]-2-(1-methyl-1H-pyrazol-4-yl)-3-oxo-6-[4-(trifluoromethyl)phenyl]-2,3-dihydropyridazine-4-carboxamide;
N-(2-Hydroxy-2-methylpropyl)-3-oxo-2-(1,2-thiazol-4-yl)-6-[6-(trifluoromethyl)pyridin-3-yl]-2,3-dihydropyridazine-4-carboxamide;
(−)-3-Oxo-2-(pyridin-3-yl)-N-(1,1,1-trifluoro-3-hydroxy-3-methylbutan-2-yl)-6-[4-(trifluoromethyl)phenyl]-2,3-dihydropyridazine-4-carboxamide enantiomer 1;
6-(4-Chlorophenyl)-N-[(2S)-1-hydroxypropan-2-yl]-2-(5-methyl-3-thienyl)-3-oxo-2,3-dihydropyridazine-4-carboxamide;
6-(4-Chlorophenyl)-2-(5-chloro-3-thienyl)-N-[(2S)-1-hydroxypropan-2-yl]-3-oxo-2,3-dihydropyridazine-4-carboxamide;
N-[(2S)-1-Hydroxypropan-2-yl]-3-oxo-2-(1H-pyrazol-4-yl)-6-[4-(trifluoromethyl)phenyl]-2,3-dihydropyridazine-4-carboxamide;
N-[(2S)-3-Fluoro-2-hydroxypropyl]-2-(1-methyl-1H-pyrazol-4-yl)-3-oxo-6-[4-(trifluoromethyl)phenyl]-2,3-dihydropyridazine-4-carboxamide;
6-(4-Chlorophenyl)-N-(2-hydroxy-2-methylpropyl)-2-(5-methyl-3-thienyl)-3-oxo-2,3-dihydropyridazine-4-carboxamide;
N-(3,3-Difluoro-2-hydroxypropyl)-3-oxo-2-(pyridin-3-yl)-6-[4-(trifluoromethyl)phenyl]-2,3-dihydropyridazine-4-carboxamide enantiomer 1;
N-[(1R,2R)-2-Hydroxycyclopentyl]-2-(1-methyl-1H-pyrazol-4-yl)-3-oxo-6-[4-(trifluoromethyl)phenyl]-2,3-dihydropyridazine-4-carboxamide;
(+)-6-(4-Chlorophenyl)-N-cis-2-hydroxycyclobutyl-3-oxo-2-(pyridin-3-yl)-2,3-dihydropyridazine-4-carboxamide enantiomer 1;
6-(4-Chlorophenyl)-2-(5-chloro-3-thienyl)-N-(2-hydroxy-2-methylpropyl)-3-oxo-2,3-dihydropyridazine-4-carboxamide;
N-[(2S)-1-Hydroxypropan-2-yl]-3-oxo-2-(1,2-thiazol-4-yl)-6-[6-(trifluoromethyl)pyridin-3-yl]-2,3-dihydropyridazine-4-carboxamide;
(−)-N-cis-4-Hydroxytetrahydrofuran-3-yl-3-oxo-2-(pyridin-3-yl)-6-[4-(trifluoromethyl)phenyl]-2,3-dihydropyridazine-4-carboxamide, enantiomer 2;
6-(4-Chlorophenyl)-N-[(2S)-1-hydroxypropan-2-yl]-2-(1,2-oxazol-4-yl)-3-oxo-2,3-dihydropyridazine-4-carboxamide;
6-(4-Chlorophenyl)-N-[(1S)-1-cyano-2-hydroxyethyl]-3-oxo-2-(pyridin-3-yl)-2,3-dihydropyridazine-4-carboxamide;
N-[(2S)-3-Fluoro-2-hydroxypropyl]-3-oxo-2-(pyridin-3-yl)-6-[4-(trifluoromethyl)phenyl]-2,3-dihydropyridazine-4-carboxamide;
6-(4-Chlorophenyl)-2-[1-(difluoromethyl)-1H-pyrazol-4-yl]-3-oxo-N-[(2S)-3,3,3-trifluoro-2-hydroxypropyl]-2,3-dihydropyridazine-4-carboxamide;
N-[(2R)-1-Hydroxy-3-methoxypropan-2-yl]-3-oxo-2-(pyridin-3-yl)-6-[4-(trifluoromethyl)phenyl]-2,3-dihydropyridazine-4-carboxamide;
6-(4-Chlorophenyl)-N-cis-2-hydroxycyclobutyl-3-oxo-2-(pyridin-3-yl)-2,3-dihydropyridazine-4-carboxamide;
6-(4-Chlorophenyl)-N-[(1S)-1-cyclopropyl-2-hydroxyethyl]-2-(5-fluoropyridin-3-yl)-3-oxo-2,3-dihydropyridazine-4-carboxamide;
N-[(2S)-3-Hydroxy-3-methylbutan-2-yl]-3-oxo-2-(1H-pyrazol-4-yl)-6-[4-(trifluoromethyl)phenyl]-2,3-dihydropyridazine-4-carboxamide;
2-[1-(Difluoromethyl)-1H-pyrazol-4-yl]-N-[(2S)-1-hydroxypropan-2-yl]-3-oxo-6-[4-(trifluoromethyl)phenyl]-2,3-dihydropyridazine-4-carboxamide;
2-[1-(Difluoromethyl)-1H-pyrazol-4-yl]-N-(2-hydroxy-2-methylpropyl)-3-oxo-6-[4-(trifluoromethyl)phenyl]-2,3-dihydropyridazine-4-carboxamide;
6-(4-Chlorophenyl)-N-[(1-hydroxycyclopropyl)methyl]-3-oxo-2-(pyridin-3-yl)-2,3-dihydropyridazine-4-carboxamide;
6-(4-Chlorophenyl)-2-[1-(difluoromethyl)-1H-pyrazol-4-yl]-N-[(2S)-3-hydroxy-3-methylbutan-2-yl]-3-oxo-2,3-dihydropyridazine-4-carboxamide;
6-(4-Chlorophenyl)-N-[(2R)-3-hydroxy-3-methylbutan-2-yl]-3-oxo-2-(pyrimidin-5-yl)-2,3-dihydropyridazine-4-carboxamide;
N-(2-Hydroxy-2-methylpropyl)-3-oxo-2-(1H-pyrazol-4-yl)-6-[4-(trifluoromethyl)phenyl]-2,3-dihydropyridazine-4-carboxamide;
Methyl N-{[6-(4-chlorophenyl)-2-(1-methyl-1H-pyrazol-4-yl)-3-oxo-2,3-dihydropyridazin-4-yl]carbonyl}-D-serinate;
(−)-N-cis-2-Hydroxycyclobutyl-3-oxo-2-(pyridin-3-yl)-6-[4-(trifluoromethyl)phenyl]-2,3-dihydropyridazine-4-carboxamide enantiomer 2;
6-(4-Chlorophenyl)-N-(2-hydroxy-2-methylpropyl)-2-(1,2-oxazol-4-yl)-3-oxo-2,3-dihydropyridazine-4-carboxamide;
6-(4-Chlorophenyl)-N-[(2S)-3-fluoro-2-hydroxypropyl]-2-(1-methyl-1H-pyrazol-4-yl)-3-oxo-2,3-dihydropyridazine-4-carboxamide;
6-(4-Chlorophenyl)-3-oxo-2-(pyrimidin-5-yl)-N-[(2R)-1,1,1-trifluoro-3-hydroxypropan-2-yl]-2,3-dihydropyridazine-4-carboxamide;
6-(4-Chlorophenyl)-2-[1-(difluoromethyl)-1H-pyrazol-4-yl]-N-[(2S)-1-hydroxypropan-2-yl]-3-oxo-2,3-dihydropyridazine-4-carboxamide;
6-(4-Chlorophenyl)-N-[(1S)-1-cyclopropyl-2-hydroxyethyl]-3-oxo-2-(pyrimidin-5-yl)-2,3-dihydropyridazine-4-carboxamide;
Methyl N-{[6-(4-chlorophenyl)-3-oxo-2-(pyridin-3-yl)-2,3-dihydropyridazin-4-yl]carbonyl}-D-serinate;
6-[4-(Fluoromethyl)phenyl]-2-(1-methyl-1H-pyrazol-4-yl)-3-oxo-N-[(2R)-1,1,1-trifluoro-3-hydroxypropan-2-yl]-2,3-dihydropyridazine-4-carboxamide;
6-(4-Chlorophenyl)-N-[(2S)-3-hydroxy-3-methylbutan-2-yl]-3-oxo-2-(1H-pyrazol-4-yl)-2,3-dihydropyridazine-4-carboxamide;
6-(4-Chlorophenyl)-N-(2-hydroxy-2-methylpropyl)-3-oxo-2-(pyrimidin-5-yl)-2,3-dihydropyridazine-4-carboxamide;
6-(4-Chlorophenyl)-2-[1-(difluoromethyl)-1H-pyrazol-4-yl]-N-(2-hydroxy-2-methylpropyl)-3-oxo-2,3-dihydropyridazine-4-carboxamide;
6-(4-Chlorophenyl)-N-[(2R)-1-hydroxypropan-2-yl]-3-oxo-2-(pyrimidin-5-yl)-2,3-dihydropyridazine-4-carboxamide;
6-[4-(Fluoromethyl)phenyl]-3-oxo-2-(pyridin-3-yl)-N-[(2S)-3,3,3-trifluoro-2-hydroxypropyl]-2,3-dihydropyridazine-4-carboxamide;
(−)-6-(4-Chlorophenyl)-N-cis-2-hydroxycyclobutyl-3-oxo-2-(pyridin-3-yl)-2,3-dihydropyridazine-4-carboxamide, enantiomer 2;
6-[4-(Fluoromethyl)phenyl]-3-oxo-2-(pyridin-3-yl)-N-[(2R)-1,1,1-trifluoro-3-hydroxypropan-2-yl]-2,3-dihydropyridazine-4-carboxamide;
6-[4-(Fluoromethyl)phenyl]-2-(1-methyl-1H-pyrazol-4-yl)-3-oxo-N-[(2S)-3,3,3-trifluoro-2-hydroxypropyl]-2,3-dihydropyridazine-4-carboxamide;
6-[4-(Fluoromethyl)phenyl]-N-[(2S)-3-hydroxy-3-methylbutan-2-yl]-2-(1-methyl-1H-pyrazol-4-yl)-3-oxo-2,3-dihydropyridazine-4-carboxamide;
6-[4-(Fluoromethyl)phenyl]-N-[(2S)-1-hydroxypropan-2-yl]-3-oxo-2-(pyridin-3-yl)-2,3-dihydropyridazine-4-carboxamide;
6-[4-(Fluoromethyl)phenyl]-N-(2-hydroxy-2-methylpropyl)-3-oxo-2-(pyridin-3-yl)-2,3-dihydropyridazine-4-carboxamide;
6-[4-(Fluoromethyl)phenyl]-N-[(2S)-1-hydroxypropan-2-yl]-2-(1-methyl-1H-pyrazol-4-yl)-3-oxo-2,3-dihydropyridazine-4-carboxamide;
6-[4-(Fluoromethyl)phenyl]-N-[(2S)-3-hydroxy-3-methylbutan-2-yl]-3-oxo-2-(pyridin-3-yl)-2,3-dihydropyridazine-4-carboxamide;
N-[(2R)-1-Amino-3-hydroxy-1-oxopropan-2-yl]-6-(4-chlorophenyl)-2-(1-methyl-1H-pyrazol-4-yl)-3-oxo-2,3-dihydropyridazine-4-carboxamide;
N-cis-4-Hydroxytetrahydrothiophen-3-yl-3-oxo-2-(pyridin-3-yl)-6-[4-(trifluoromethyl)phenyl]-2,3-dihydropyridazine-4-carboxamide;
(−)-N-cis-4-Hydroxytetrahydrothiophen-3-yl-3-oxo-2-(pyridin-3-yl)-6-[4-(trifluoromethyl)phenyl]-2,3-dihydropyridazine-4-carboxamide, enantiomer 2;
N-(2-Hydroxy-2-methylpropyl)-3-oxo-2-(pyridin-3-yl)-6-[4-(trifluoromethoxy)phenyl]-2,3-dihydropyridazine-4-carboxamide;
3-Oxo-2-(pyridin-3-yl)-N-[(2R)-3,3,3-trifluoro-2-hydroxypropyl]-6-[4-(trifluoromethoxy)-phenyl]-2,3-dihydropyridazine-4-carboxamide;
N-[(2R)-3-Hydroxy-3-methylbutan-2-yl]-3-oxo-2-(pyridin-3-yl)-6-[4-(trifluoromethoxy)-phenyl]-2,3-dihydropyridazine-4-carboxamide;
N-[(2S)-3,3-Difluoro-2-hydroxypropyl]-3-oxo-2-(pyridin-3-yl)-6-[4-(trifluoromethoxy)phenyl]-2,3-dihydropyridazine-4-carboxamide;
N-[(2S)-3-Fluoro-2-hydroxypropyl]-3-oxo-2-(pyridin-3-yl)-6-[4-(trifluoromethoxy)phenyl]-2,3-dihydropyridazine-4-carboxamide;
6-(4-Chlorophenyl)-3-oxo-2-(pyridin-3-yl)-N-(4,4,4-trifluoro-3-hydroxybutan-2-yl)-2,3-dihydropyridazine-4-carboxamide Isomer 1;
6-(4-Chlorophenyl)-3-oxo-2-(pyridin-3-yl)-N-(4,4,4-trifluoro-3-hydroxybutan-2-yl)-2,3-dihydropyridazine-4-carboxamide Isomer 2;
6-(4-Chlorophenyl)-3-oxo-2-(pyridin-3-yl)-N-(4,4,4-trifluoro-3-hydroxybutan-2-yl)-2,3-dihydropyridazine-4-carboxamide Isomer 3;
6-(4-Chlorophenyl)-3-oxo-2-(pyridin-3-yl)-N-(4,4,4-trifluoro-3-hydroxybutan-2-yl)-2,3-dihydropyridazine-4-carboxamide Isomer 4;
6-(4-Chlorophenyl)-2-(5-fluoropyridin-3-yl)-N-[(cis)-4-hydroxytetrahydrofuran-3-yl]-3-oxo-2,3-dihydropyridazine-4-carboxamide;
6-(4-Chlorophenyl)-N-[(2R)-3-hydroxy-3-methylbutan-2-yl]-3-oxo-2-(pyridin-3-yl)-2,3-dihydropyridazine-4-carboxamide;
6-(4-Chlorophenyl)-2-(5-chloropyridin-3-yl)-N-[(2S)-1-hydroxypropan-2-yl]-3-oxo-2,3-dihydropyridazine-4-carboxamide;
6-(4-Chlorophenyl)-N-[(2S)-1-hydroxypropan-2-yl]-2-(5-methylpyridin-3-yl)-3-oxo-2,3-dihydropyridazine-4-carboxamide;
6-[4-(Difluoromethoxy)phenyl]-2-(1-methyl-1H-pyrazol-4-yl)-3-oxo-N-(1,1,1-trifluoro-3-hydroxy-3-methylbutan-2-yl)-2,3-dihydropyridazine-4-carboxamide;
6-[4-(Difluoromethoxy)phenyl]-2-(1-methyl-1H-pyrazol-4-yl)-3-oxo-N-(1,1,1-trifluoro-3-hydroxy-3-methylbutan-2-yl)-2,3-dihydropyridazine-4-carboxamide Isomer 1;
6-[4-(Difluoromethoxy)phenyl]-2-(1-methyl-1H-pyrazol-4-yl)-3-oxo-N-(1,1,1-trifluoro-3-hydroxy-3-methylbutan-2-yl)-2,3-dihydropyridazine-4-carboxamide Isomer 2;
6-[4-(Difluoromethoxy)phenyl]-N-[(2S)-1-hydroxypropan-2-yl]-2-(1-methyl-1H-pyrazol-4-yl)-3-oxo-2,3-dihydropyridazine-4-carboxamide;
6-[4-(Difluoromethoxy)phenyl]-N-[(2S)-3-hydroxy-3-methylbutan-2-yl]-2-(1-methyl-1H-pyrazol-4-yl)-3-oxo-2,3-dihydropyridazine-4-carboxamide;
6-[4-(Dimethylamino)phenyl]-N-[(2S)-3-hydroxy-3-methylbutan-2-yl]-2-(1-methyl-1H-pyrazol-4-yl)-3-oxo-2,3-dihydropyridazine-4-carboxamide;
6-[4-(Dimethylamino)phenyl]-N-[(2S)-1-hydroxypropan-2-yl]-2-(1-methyl-1H-pyrazol-4-yl)-3-oxo-2,3-dihydropyridazine-4-carboxamide;
N-[(2S)-3-Hydroxy-3-methylbutan-2-yl]-3-oxo-2-(1,2-thiazol-4-yl)-6-[4-(trifluoromethoxy)-phenyl]-2,3-dihydropyridazine-4-carboxamide;
6-[4-(Difluoromethyl)phenyl]-3-oxo-2-(pyridin-3-yl)-N-[(2S)-3,3,3-trifluoro-2-hydroxy-propyl]-2,3-dihydropyridazine-4-carboxamide;
6-[4-(Difluoromethoxy)phenyl]-3-oxo-2-(pyridin-3-yl)-N-[(2S)-3,3,3-trifluoro-2-hydroxypropyl]-2,3-dihydropyridazine-4-carboxamide;
6-[4-(Difluoromethyl)phenyl]-N-[(cis)-4-hydroxytetrahydrofuran-3-yl]-2-(1-methyl-1H-pyrazol-4-yl)-3-oxo-2,3-dihydropyridazine-4-carboxamide Isomere 1;
6-(4-Chlorophenyl)-N-[(2S)-3-hydroxy-3-methylbutan-2-yl]-2-(1-methyl-1H-pyrazol-4-yl)-3-oxo-2,3-dihydropyridazine-4-carboxamide;
6-(4-Chlorophenyl)-N-(2-hydroxy-2-methylcyclopentyl)-2-(1-methyl-1H-pyrazol-4-yl)-3-oxo-2,3-dihydropyridazine-4-carboxamide;
6-[4-(Difluoromethyl)phenyl]-N-[(cis)-4-hydroxytetrahydrofuran-3-yl]-3-oxo-2-(pyridin-3-yl)-2,3-dihydropyridazine-4-carboxamide Isomere 1;
6-[4-(Difluoromethyl)phenyl]-N-[(2S)-3-hydroxy-3-methylbutan-2-yl]-3-oxo-2-(pyridin-3-yl)-2,3-dihydropyridazine-4-carboxamide;
6-[4-(Difluoromethyl)phenyl]-N-[(cis)-4-hydroxytetrahydrofuran-3-yl]-2-(1-methyl-1H-pyrazol-4-yl)-3-oxo-2,3-dihydropyridazine-4-carboxamide Isomere 2;
3-(4-Chlorophenyl)-6-oxo-N-[(2S)-3,3,3-trifluoro-2-hydroxypropyl]-6H-1,4′-bipyridazine-5-carboxamide;
6-[4-(Difluoromethyl)phenyl]-N-(2-hydroxy-2-methylpropyl)-3-oxo-2-(pyridin-3-yl)-2,3-dihydropyridazine-4-carboxamide;
6-[4-(Difluoromethyl)phenyl]-N-[(cis)-4-hydroxytetrahydrofuran-3-yl]-3-oxo-2-(pyridin-3-yl)-2,3-dihydropyridazine-4-carboxamide Isomere 2;
6-(4-Chlorophenyl)-N-[(2R)-1-hydroxypropan-2-yl]-2-(1-methyl-1H-pyrazol-4-yl)-3-oxo-2,3-dihydropyridazine-4-carboxamide;
6-(4-Chlorophenyl)-N-[(2R)-3-hydroxy-3-methylbutan-2-yl]-3-oxo-2-(1H-pyrazol-4-yl)-2,3-dihydropyridazine-4-carboxamide;
6-(4-Chlorophenyl)-N-[(2R)-3-hydroxy-3-methylbutan-2-yl]-3-oxo-2-(1,2-thiazol-4-yl)-2,3-dihydropyridazine-4-carboxamide;
N-[(2R)-3-Hydroxy-3-methylbutan-2-yl]-3-oxo-2-(1,2-thiazol-4-yl)-6-[4-(trifluoromethoxy)-phenyl]-2,3-dihydropyridazine-4-carboxamide;
N-[(cis)-2-hydroxy-2-methylcyclopentyl]-3-oxo-2-(pyridin-3-yl)-6-[4-(trifluoromethoxy)-phenyl]-2,3-dihydropyridazine-4-carboxamide;
N-[(cis)-2-hydroxy-2-methylcyclopentyl]-3-oxo-2-(pyridin-3-yl)-6-[4-(trifluoromethoxy)-phenyl]-2,3-dihydropyridazine-4-carboxamide, isomer 1;
N-[(cis)-2-hydroxy-2-methylcyclopentyl]-3-oxo-2-(pyridin-3-yl)-6-[4-(trifluoromethoxy)-phenyl]-2,3-dihydropyridazine-4-carboxamide, isomer 2;
6-[4-(difluoromethoxy)phenyl]-N-(2-hydroxy-2-methylpropyl)-3-oxo-2-(pyridin-3-yl)-2,3-dihydropyridazine-4-carboxamide;
6-[4-(difluoromethoxy)phenyl]-N-[(2S)-1-hydroxypropan-2-yl]-3-oxo-2-(pyridin-3-yl)-2,3-dihydropyridazine-4-carboxamide;
6-[4-(difluoromethoxy)phenyl]-N-[(2S)-3-hydroxy-3-methylbutan-2-yl]-3-oxo-2-(pyridin-3-yl)-2,3-dihydropyridazine-4-carboxamide;
6-[4-(difluoromethoxy)phenyl]-N-[(2S,3S)-3-hydroxybutan-2-yl]-3-oxo-2-(pyridin-3-yl)-2,3-dihydropyridazine-4-carboxamide;
6-(4-chlorophenyl)-N-[(cis)-2-hydroxy-2-methylcyclopentyl]-2-(1-methyl-1H-pyrazol-4-yl)-3-oxo-2,3-dihydropyridazine-4-carboxamide; isomer 1;
6-(4-Chlorophenyl)-N-[(cis)-2-hydroxy-2-methylcyclopentyl]-2-(1-methyl-1H-pyrazol-4-yl)-3-oxo-2,3-dihydropyridazine-4-carboxamide; Isomer 2;
2-(5-fluoropyridin-3-yl)-N-[(2S)-1-hydroxy-3-methylbutan-2-yl]-3-oxo-6-[6-(trifluoro-methyl)pyridin-3-yl]-2,3-dihydropyridazine-4-carboxamide;
2-(5-fluoropyridin-3-yl)-N-[(1S,2R)-2-hydroxycyclopentyl]-3-oxo-6-[6-(trifluoromethyl)-pyridin-3-yl]-2,3-dihydropyridazine-4-carboxamide;
N-[(1S)-1-cyclopropyl-2-hydroxyethyl]-2-(5-fluoropyridin-3-yl)-3-oxo-6-[6-(trifluoromethyl)-pyridin-3-yl]-2,3-dihydropyridazine-4-carboxamide;
2-(5-fluoropyridin-3-yl)-N-[(2S)-3-hydroxy-3-methylbutan-2-yl]-3-oxo-6-[6-(trifluoro-methyl)pyridin-3-yl]-2,3-dihydropyridazine-4-carboxamide;
N-[(1S)-1-cyano-2-hydroxyethyl]-2-(5-fluoropyridin-3-yl)-3-oxo-6-[6-(trifluoromethyl)-pyridin-3-yl]-2,3-dihydropyridazine-4-carboxamide;
2-(5-fluoropyridin-3-yl)-N-[(2R)-3-hydroxy-3-methylbutan-2-yl]-3-oxo-6-[6-(trifluoro-methyl)pyridin-3-yl]-2,3-dihydropyridazine-4-carboxamide;
1,5-anhydro-2,4-dideoxy-2-[({2-(5-fluoropyridin-3-yl)-3-oxo-6-[6-(trifluoromethyl)pyridin-3-yl]-2,3-dihydropyridazine-4-yl}carbonyl)amino]-D-erythro-pentitol;
2-(5-fluoropyridin-3-yl)-N-(2-hydroxy-2-methylpropyl)-3-oxo-6-[6-(trifluoromethyl)pyridin-3-yl]-2,3-dihydropyridazine-4-carboxamide;
   and
N-[(2S)-1-hydroxypropan-2-yl]-3-oxo-2-(pyridin-3-yl)-6-[5-(trifluoromethyl)pyridin-2-yl]-2,3-dihydropyridazine-4-carboxamide,
   their polymorphs, enantiomeres, diastereomeres, racemates, tautomeres, N-oxides, hydrates and solvates, as well as their physiological acceptable salts and solvates of these salts, as well as mixtures of the same.

More especially, as compound A those AhR inhibitors are of interest for the combination as disclosed in the International Patent Application No. PCT/EP2018/052627, published as WO 2018/146010 A1:
N-(1-Hydroxy-3-methylbutan-2-yl)-6-(4-methylphenyl)-2-(1-methyl-1H-pyrazol-4-yl)-3-oxo-2,3-dihydropyridazine-4-carboxamide

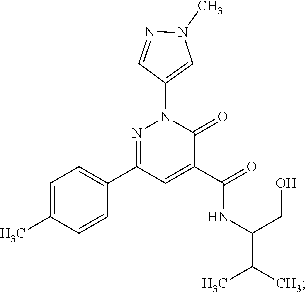 [SMILES: Cc1ccc(-c2cc(C(=O)NC(CO)C(C)C)c(=O)n(-c3cnn(C)c3)n2)cc1]

N-(1-Hydroxy-3-methylbutan-2-yl)-6-(4-methylphenyl)-2-(1-methyl-1H-pyrazol-4-yl)-3-oxo-2,3-dihydropyridazine-4-carboxamide, Enantiomer 1

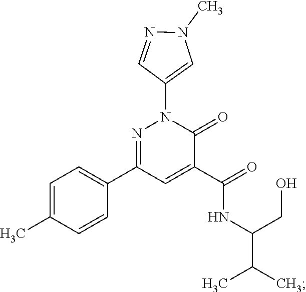 [SMILES: Cc1ccc(-c2cc(C(=O)NC(CO)C(C)C)c(=O)n(-c3cnn(C)c3)n2)cc1]

N-(1-Hydroxy-3-methylbutan-2-yl)-6-(4-methylphenyl)-2-(1-methyl-1H-pyrazol-4-yl)-3-oxo-2,3-dihydropyridazine-4-carboxamide, Enantiomer 2

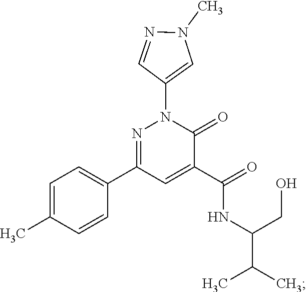 [SMILES: Cc1ccc(-c2cc(C(=O)NC(CO)C(C)C)c(=O)n(-c3cnn(C)c3)n2)cc1]

N-(1-Hydroxybutan-2-yl)-6-(4-methylphenyl)-2-(1-methyl-1H-pyrazol-4-yl)-3-oxo-2,3-dihydropyridazine-4-carboxamide

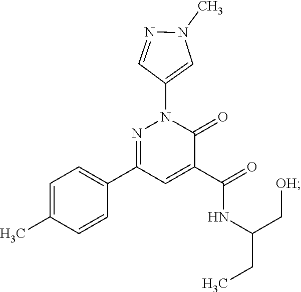 [SMILES: CCC(CO)NC(=O)c1cc(-c2ccc(C)cc2)nn(-c2cnn(C)c2)c1=O]

N-(1-Hydroxybutan-2-yl)-6-(4-methylphenyl)-2-(1-methyl-1H-pyrazol-4-yl)-3-oxo-2,3-dihydropyridazine-4-carboxamide, Enantiomer 1

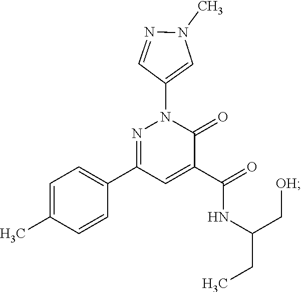 [SMILES: CCC(CO)NC(=O)c1cc(-c2ccc(C)cc2)nn(-c2cnn(C)c2)c1=O]

N-(1-Hydroxybutan-2-yl)-6-(4-methylphenyl)-2-(1-methyl-1H-pyrazol-4-yl)-3-oxo-2,3-dihydropyridazine-4-carboxamide, Enantiomer 2

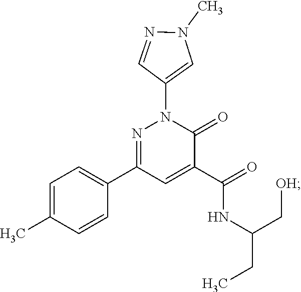 [SMILES: CCC(CO)NC(=O)c1cc(-c2ccc(C)cc2)nn(-c2cnn(C)c2)c1=O]

N-(1-Hydroxypropan-2-yl)-6-(4-methylphenyl)-2-(1-methyl-1H-pyrazol-4-yl)-3-oxo-2,3-dihydropyridazine-4-carboxamide

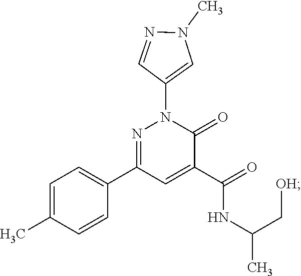 [SMILES: Cc1ccc(-c2cc(C(=O)NC(C)CO)c(=O)n(-c3cnn(C)c3)n2)cc1]

N-[(2S)-1-Hydroxypropan-2-yl]-6-(4-methylphenyl)-2-(1-methyl-1H-pyrazol-4-yl)-3-oxo-2,3-dihydropyridazine-4-carboxamide

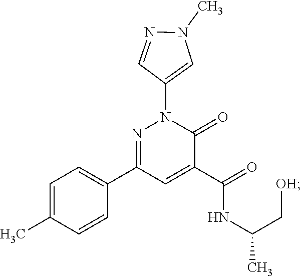 [SMILES: Cc1ccc(-c2cc(C(=O)N[C@@H](C)CO)c(=O)n(-c3cnn(C)c3)n2)cc1]

N-[(2R)-1-Hydroxypropan-2-yl]-6-(4-methylphenyl)-2-(1-methyl-1H-pyrazol-4-yl)-3-oxo-2,3-dihydropyridazine-4-carboxamide;

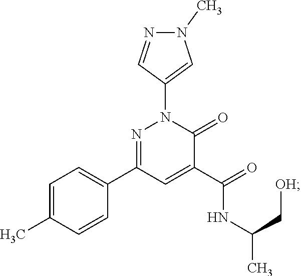 [SMILES: Cc1ccc(-c2cc(C(=O)N[C@H](C)CO)c(=O)n(-c3cnn(C)c3)n2)cc1]

6-(4-Methylphenyl)-2-(1-methyl-1H-pyrazol-4-yl)-3-oxo-N-(3,3,3-trifluoro-2-hydroxypropyl)-2,3-dihydropyridazine-4-carboxamide

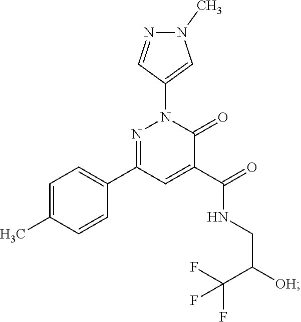 [SMILES: Cc1ccc(-c2cc(C(=O)NCC(O)C(F)(F)F)c(=O)n(-c3cnn(C)c3)n2)cc1]

(−)-6-(4-Methylphenyl)-2-(1-methyl-1H-pyrazol-4-yl)-3-oxo-N-(3,3,3-trifluoro-2-hydroxypropyl)-2,3-dihydropyridazine-4-carboxamide, Enantiomer 1

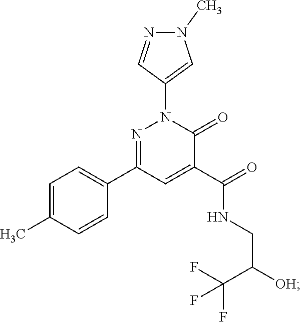 [SMILES: Cc1ccc(-c2cc(C(=O)NCC(O)C(F)(F)F)c(=O)n(-c3cnn(C)c3)n2)cc1]

(+)-6-(4-Methylphenyl)-2-(1-methyl-1H-pyrazol-4-yl)-3-oxo-N-(3,3,3-trifluoro-2-hydroxypropyl)-2,3-dihydropyridazine-4-carboxamide, Enantiomer 2

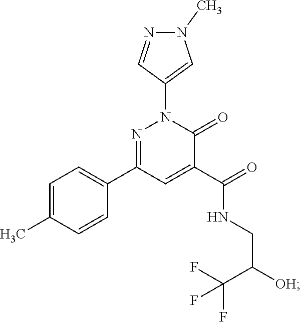 [SMILES: Cc1ccc(-c2cc(C(=O)NCC(O)C(F)(F)F)c(=O)n(-c3cnn(C)c3)n2)cc1]

N-(3,3-Difluoro-2-hydroxypropyl)-6-(4-methylphenyl)-2-(1-methyl-1H-pyrazol-4-yl)-3-oxo-2,3-dihydropyridazine-4-carboxamide

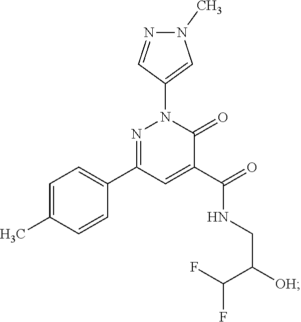 [SMILES: Cc1ccc(-c2cc(C(=O)NCC(O)C(F)F)c(=O)n(-c3cnn(C)c3)n2)cc1]

N-(3,3-Difluoro-2-hydroxypropyl)-6-(4-methylphenyl)-2-(1-methyl-1H-pyrazol-4-yl)-3-oxo-2,3-dihydropyridazine-4-carboxamide, Enantiomer 1

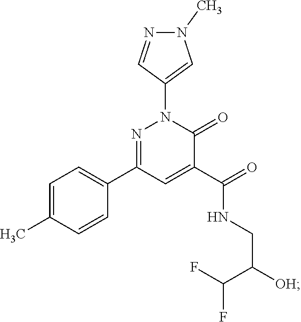 [SMILES: Cc1ccc(-c2cc(C(=O)NCC(O)C(F)F)c(=O)n(-c3cnn(C)c3)n2)cc1]

N-(3,3-Difluoro-2-hydroxypropyl)-6-(4-methylphenyl)-2-(1-methyl-1H-pyrazol-4-yl)-3-oxo-2,3-dihydropyridazine-4-carboxamide, Enantiomer 2

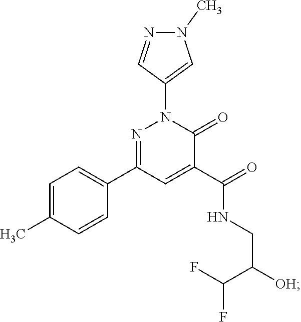 [SMILES: Cc1ccc(-c2cc(C(=O)NCC(O)C(F)F)c(=O)n(-c3cnn(C)c3)n2)cc1]

6-(4-Chlorophenyl)-N-[(2S)-1-hydroxy-3-methylbutan-2-yl]-2-(1-methyl-1H-pyrazol-4-yl)-3-oxo-2,3-dihydropyridazine-4-carboxamide

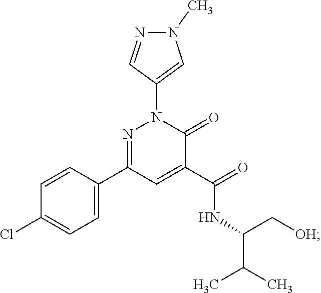 [SMILES: CC(C)[C@@H](CO)NC(=O)c1cc(-c2ccc(Cl)cc2)nn(-c2cnn(C)c2)c1=O]

6-(4-Chlorophenyl)-N-[(2S)-1-hydroxypropan-2-yl]-2-(1-methyl-1H-pyrazol-4-yl)-3-oxo-2,3-dihydropyridazine-4-carboxamide

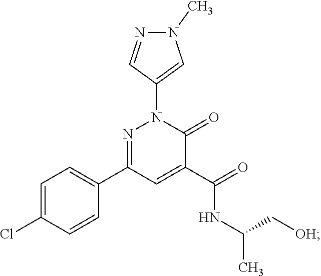 [SMILES: C[C@@H](CO)NC(=O)c1cc(-c2ccc(Cl)cc2)nn(-c2cnn(C)c2)c1=O]

6-(4-Chlorophenyl)-2-(1-methyl-1H-pyrazol-4-yl)-3-oxo-N-(3,3,3-trifluoro-2-hydroxypropyl)-2,3-dihydropyridazine-4-carboxamide

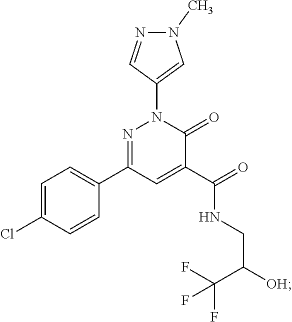 [SMILES: Cn1cc(-n2nc(-c3ccc(Cl)cc3)cc(C(=O)NCC(O)C(F)(F)F)c2=O)cn1]

(−)-6-(4-Chlorophenyl)-2-(1-methyl-1H-pyrazol-4-yl)-3-oxo-N-(3,3,3-trifluoro-2-hydroxypropyl)-2,3-dihydropyridazine-4-carboxamide

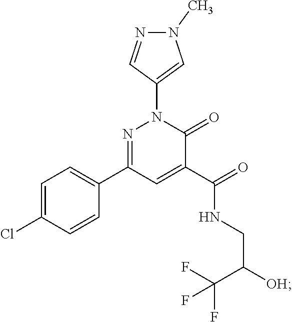 [SMILES: Cn1cc(-n2nc(-c3ccc(Cl)cc3)cc(C(=O)NCC(O)C(F)(F)F)c2=O)cn1]

(+)-6-(4-Chlorophenyl)-2-(1-methyl-1H-pyrazol-4-yl)-3-oxo-N-(3,3,3-trifluoro-2-hydroxypropyl)-2,3-dihydropyridazine-4-carboxamide

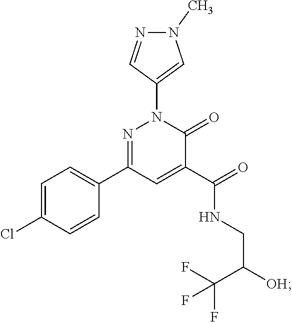 [SMILES: Cn1cc(-n2nc(-c3ccc(Cl)cc3)cc(C(=O)NCC(O)C(F)(F)F)c2=O)cn1]

6-(4-Chlorophenyl)-N-(3,3-difluoro-2-hydroxypropyl)-2-(1-methyl-1H-pyrazol-4-yl)-3-oxo-2,3-dihydropyridazine-4-carboxamide

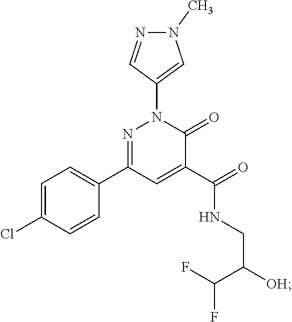 [SMILES: Cn1cc(-n2nc(-c3ccc(Cl)cc3)cc(C(=O)NCC(O)C(F)F)c2=O)cn1]

(−)-6-(4-Chlorophenyl)-N-(3,3-difluoro-2-hydroxypropyl)-2-(1-methyl-1H-pyrazol-4-yl)-3-oxo-2,3-dihydropyridazine-4-carboxamide

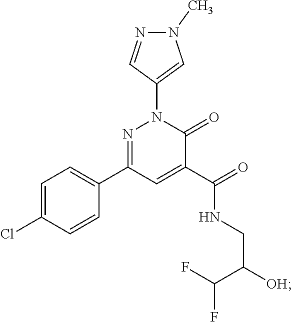 [SMILES: Cn1cc(-n2nc(-c3ccc(Cl)cc3)cc(C(=O)NCC(O)C(F)F)c2=O)cn1]

(+)-6-(4-Chlorophenyl)-N-(3,3-difluoro-2-hydroxypropyl)-2-(1-methyl-1H-pyrazol-4-yl)-3-oxo-2,3-dihydropyridazine-4-carboxamide

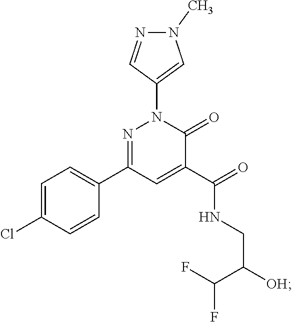 [SMILES: Cn1cc(-n2nc(-c3ccc(Cl)cc3)cc(C(=O)NCC(O)C(F)F)c2=O)cn1]

6-(4-Chlorophenyl)-N-(2-hydroxy-3-methoxypropyl)-2-(1-methyl-1H-pyrazol-4-yl)-3-oxo-2,3-dihydropyridazine-4-carboxamide

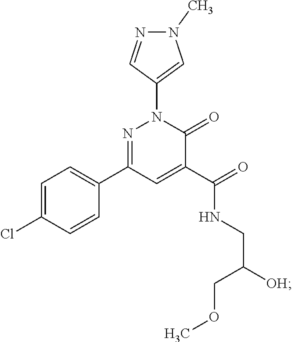 [SMILES: COCC(O)CNC(=O)c1cc(-c2ccc(Cl)cc2)nn(-c2cnn(C)c2)c1=O]

(−)-6-(4-Chlorophenyl)-N-(2-hydroxy-3-methoxypropyl)-2-(1-methyl-1H-pyrazol-4-yl)-3-oxo-2,3-dihydropyridazine-4-carboxamide

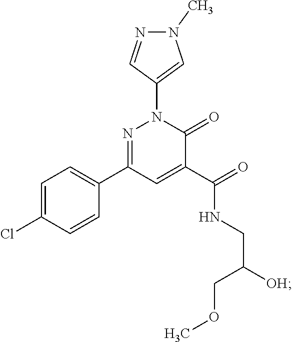 [SMILES: COCC(O)CNC(=O)c1cc(-c2ccc(Cl)cc2)nn(-c2cnn(C)c2)c1=O]

(+)-6-(4-Chlorophenyl)-N-(2-hydroxy-3-methoxypropyl)-2-(1-methyl-1H-pyrazol-4-yl)-3-oxo-2,3-dihydropyridazine-4-carboxamide

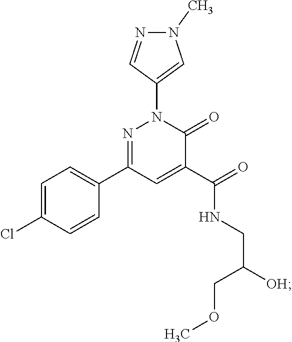 [SMILES: COCC(O)CNC(=O)c1cc(-c2ccc(Cl)cc2)nn(-c2cnn(C)c2)c1=O]

6-(4-Chlorophenyl)-N-[(2S)-2,3-dihydroxypropyl]-2-(1-methyl-1H-pyrazol-4-yl)-3-oxo-2,3-dihydropyridazine-4-carboxamide

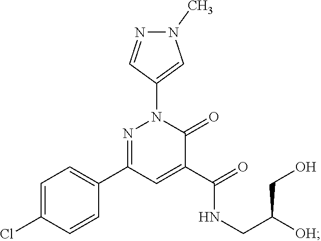 [SMILES: Cn1cc(-n2nc(-c3ccc(Cl)cc3)cc(C(=O)NC[C@H](O)CO)c2=O)cn1]

6-(4-Chlorophenyl)-N-[(2R)-2,3-dihydroxypropyl]-2-(1-methyl-1H-pyrazol-4-yl)-3-oxo-2,3-dihydropyridazine-4-carboxamide

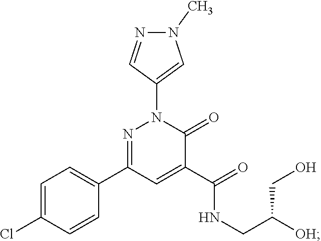 [SMILES: Cn1cc(-n2nc(-c3ccc(Cl)cc3)cc(C(=O)NC[C@@H](O)CO)c2=O)cn1]

6-(4-Chlorophenyl)-N-[(2S)-1-hydroxy-3-methoxypropan-2-yl]-2-(1-methyl-1H-pyrazol-4-yl)-3-oxo-2,3-dihydropyridazine-4-carboxamide

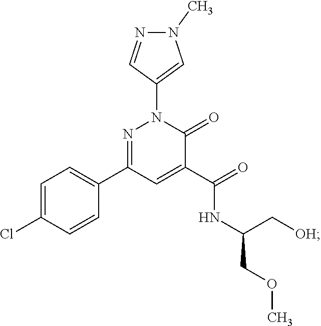 [SMILES: COC[C@H](CO)NC(=O)c1cc(-c2ccc(Cl)cc2)nn(-c2cnn(C)c2)c1=O]

6-(4-Chlorophenyl)-N-[(2R)-1-hydroxy-3-methoxypropan-2-yl]-2-(1-methyl-1H-pyrazol-4-yl)-3-oxo-2,3-dihydropyridazine-4-carboxamide

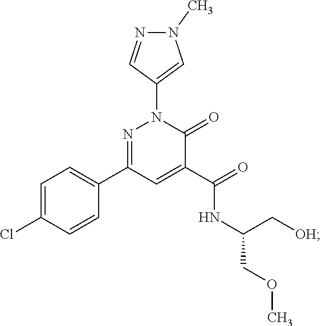 [SMILES: COC[C@@H](CO)NC(=O)c1cc(-c2ccc(Cl)cc2)nn(-c2cnn(C)c2)c1=O]

6-(4-Chlorophenyl)-N-(1,3-dihydroxypropan-2-yl)-2-(1-methyl-1H-pyrazol-4-yl)-3-oxo-2,3-dihydropyridazine-4-carboxamide

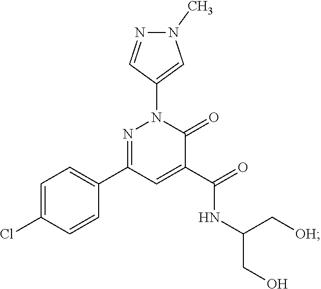 [SMILES: Cn1cc(-n2nc(-c3ccc(Cl)cc3)cc(C(=O)NC(CO)CO)c2=O)cn1]

6-(4-Chlorophenyl)-N-(2-hydroxy-2-methylpropyl)-2-(1-methyl-1H-pyrazol-4-yl)-3-oxo-2,3-dihydropyridazine-4-carboxamide

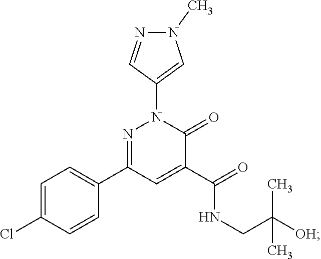 [SMILES: Cn1cc(-n2nc(-c3ccc(Cl)cc3)cc(C(=O)NCC(C)(C)O)c2=O)cn1]

6-(4-Chlorophenyl)-2-(1-methyl-1H-pyrazol-4-yl)-3-oxo-N-[(2RS)-1,1,1-trifluoro-3-hydroxypropan-2-yl]-2,3-dihydropyridazine-4-carboxamide

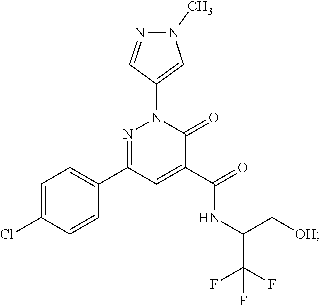 [SMILES: Cn1cc(-n2nc(-c3ccc(Cl)cc3)cc(C(=O)NC(CO)C(F)(F)F)c2=O)cn1]

(−)-6-(4-Chlorophenyl)-2-(1-methyl-1H-pyrazol-4-yl)-3-oxo-N-[(2S)-1,1,1-trifluoro-3-hydroxypropan-2-yl]-2,3-dihydropyridazine-4-carboxamide

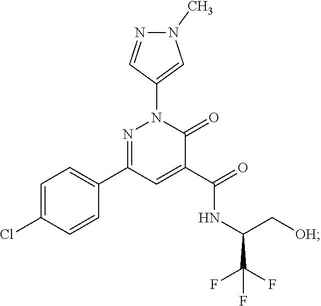 [SMILES: Cn1cc(-n2nc(-c3ccc(Cl)cc3)cc(C(=O)N[C@@H](CO)C(F)(F)F)c2=O)cn1]

(+)-6-(4-Chlorophenyl)-2-(1-methyl-1H-pyrazol-4-yl)-3-oxo-N-[(2R)-1,1,1-trifluoro-3-hydroxypropan-2-yl]-2,3-dihydropyridazine-4-carboxamide

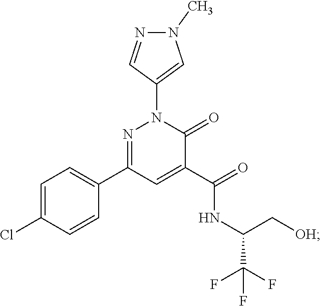 [SMILES: Cn1cc(-n2nc(-c3ccc(Cl)cc3)cc(C(=O)N[C@H](CO)C(F)(F)F)c2=O)cn1]

2-(1-Methyl-1H-pyrazol-4-yl)-3-oxo-N-[(2R)-1,1,1-trifluoro-3-hydroxypropan-2-yl]-6-[4-(trifluoromethyl)phenyl]-2,3-dihydropyridazine-4-carboxamide

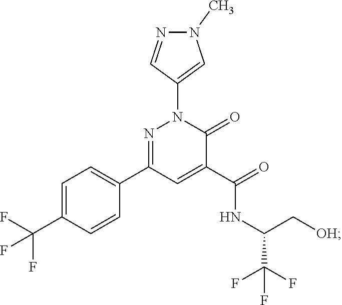 [SMILES: Cn1cc(-n2nc(-c3ccc(C(F)(F)F)cc3)cc(C(=O)N[C@H](CO)C(F)(F)F)c2=O)cn1]

2-(1-Methyl-1H-pyrazol-4-yl)-3-oxo-N-[(2S)-3,3,3-trifluoro-2-hydroxypropyl]-6-[4-(trifluoromethyl)phenyl]-2,3-dihydropyridazine-4-carboxamide

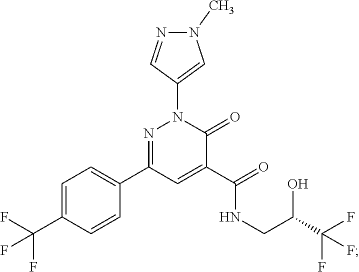 [SMILES: Cn1cc(-n2nc(-c3ccc(C(F)(F)F)cc3)cc(C(=O)NC[C@H](O)C(F)(F)F)c2=O)cn1]

(−)-N-[(2R)-1-Hydroxypropan-2-yl]-2-(1-methyl-1H-pyrazol-4-yl)-3-oxo-6-[4-(trifluoromethyl)phenyl]-2,3-dihydropyridazine-4-carboxamide

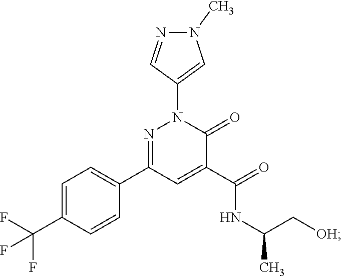 [SMILES: C[C@H](CO)NC(=O)c1cc(-c2ccc(C(F)(F)F)cc2)nn(-c2cnn(C)c2)c1=O]

(+)-N-[(2S)-1-Hydroxypropan-2-yl]-2-(1-methyl-1H-pyrazol-4-yl)-3-oxo-6-[4-(trifluoromethyl)phenyl]-2,3-dihydropyridazine-4-carboxamide

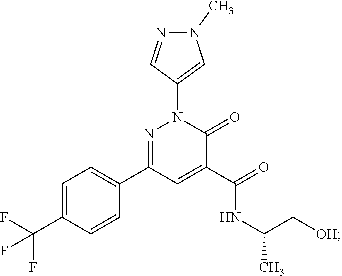 [SMILES: C[C@@H](CO)NC(=O)c1cc(-c2ccc(C(F)(F)F)cc2)nn(-c2cnn(C)c2)c1=O]

6-(4-Chlorophenyl)-2-(1-cyclobutyl-1H-pyrazol-4-yl)-3-oxo-N-[(2S)-3,3,3-trifluoro-2-hydroxypropyl]-2,3-diihydropyridazine-4-carboxamide

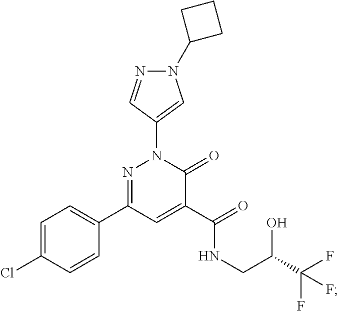 [SMILES: O=C(NC[C@H](O)C(F)(F)F)c1cc(-c2ccc(Cl)cc2)nn(-c2cnn(C3CCC3)c2)c1=O]

6-(4-Chlorophenyl)-2-(1-cyclobutyl-1H-pyrazol-4-yl)-3-oxo-N-[(2R)-1,1,1-trifluoro-3-hydroxypropan-2-yl]-2,3-dihydropyridazine-4-carboxamide

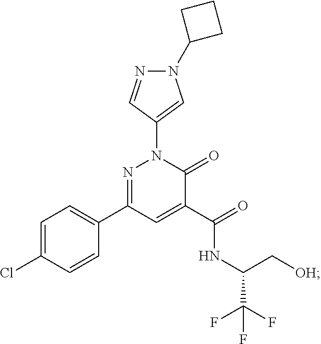 [SMILES: O=C(N[C@H](CO)C(F)(F)F)c1cc(-c2ccc(Cl)cc2)nn(-c2cnn(C3CCC3)c2)c1=O]

6-(4-Chlorophenyl)-2-(1-cyclobutyl-1H-pyrazol-4-yl)-N-[(2S)-1-hydroxypropan-2-yl]-3-oxo-2,3-dihydropyridazine-4-carboxamide

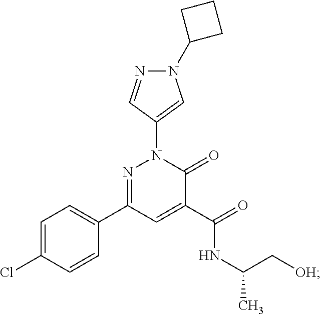 [SMILES: C[C@@H](CO)NC(=O)c1cc(-c2ccc(Cl)cc2)nn(-c2cnn(C3CCC3)c2)c1=O]

6-(4-Chlorophenyl)-N-[(cis)-4-hydroxytetrahydrofuran-3-yl]-2-(1-methyl-1H-pyrazol-4-yl)-3-oxo-2,3-dihydropyridazine-4-carboxamide

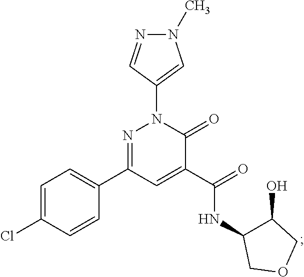 [SMILES: Cn1cc(-n2nc(-c3ccc(Cl)cc3)cc(C(=O)N[C@@H]3COC[C@@H]3O)c2=O)cn1]

6-(4-Chlorophenyl)-N-[(cis)-4-hydroxytetrahydrofuran-3-yl]-2-(1-methyl-1H-pyrazol-4-yl)-3-oxo-2,3-dihydropyridazine-4-carboxamide, Enantiomer 1

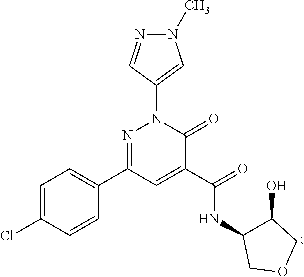 [SMILES: Cn1cc(-n2nc(-c3ccc(Cl)cc3)cc(C(=O)N[C@@H]3COC[C@@H]3O)c2=O)cn1]

6-(4-Chlorophenyl)-N-[(cis)-4-hydroxytetrahydrofuran-3-yl]-2-(1-methyl-1H-pyrazol-4-yl)-3-oxo-2,3-dihydropyridazine-4-carboxamide, Enantiomer 2

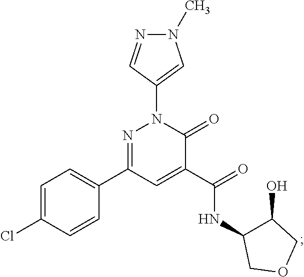 [SMILES: Cn1cc(-n2nc(-c3ccc(Cl)cc3)cc(C(=O)N[C@@H]3COC[C@@H]3O)c2=O)cn1]

N-[(1S,2R)-2-Hydroxycyclohexyl]-2-(1-methyl-1H-pyrazol-4-yl)-3-oxo-6-[4-(trifluoromethoxy)phenyl]-2,3-dihydropyridazine-4-carboxamide

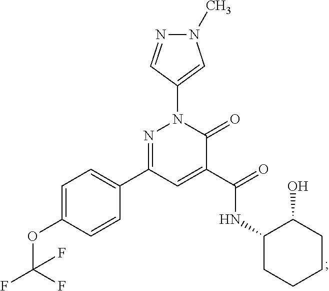 [SMILES: Cn1cc(-n2nc(-c3ccc(OC(F)(F)F)cc3)cc(C(=O)N[C@H]3CCCC[C@H]3O)c2=O)cn1]

2-(1-Methyl-1H-pyrazol-4-yl)-3-oxo-N-[(2R)-1,1,1-trifluoro-3-hydroxypropan-2-yl]-6-[4-(trifluoromethoxy)phenyl]-2,3-dihydropyridazine-4-carboxamide

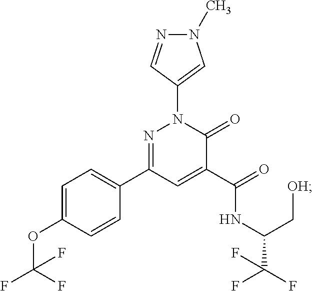 [SMILES: Cn1cc(-n2nc(-c3ccc(OC(F)(F)F)cc3)cc(C(=O)N[C@H](CO)C(F)(F)F)c2=O)cn1]

6-(4-Chlorophenyl)-N-[(1S,2R)-2-hydroxycyclohexyl]-2-(1-methyl-1H-pyrazol-4-yl)-3-oxo-2,3-dihydropyridazine-4-carboxamide

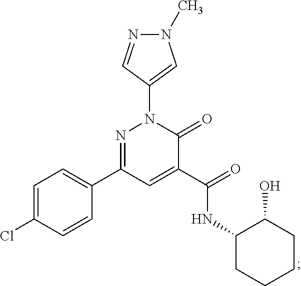 [SMILES: Cn1cc(-n2nc(-c3ccc(Cl)cc3)cc(C(=O)N[C@H]3CCCC[C@H]3O)c2=O)cn1]

6-(4-Chlorophenyl)-N-[(1S,2S)-2-hydroxycyclopentyl]-2-(1-methyl-1H-pyrazol-4-yl)-3-oxo-2,3-dihydropyridazine-4-carboxamide

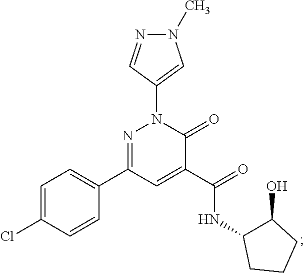 [SMILES: Cn1cc(-n2nc(-c3ccc(Cl)cc3)cc(C(=O)N[C@H]3CCC[C@@H]3O)c2=O)cn1]

6-(4-Chlorophenyl)-N-[(1S,2R)-2-hydroxycyclopentyl]-2-(1-methyl-1H-pyrazol-4-yl)-3-oxo-2,3-dihydropyridazine-4-carboxamide

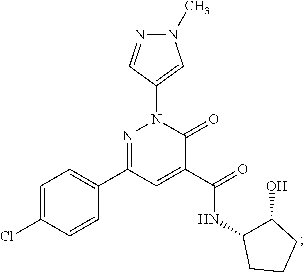 [SMILES: Cn1cc(-n2nc(-c3ccc(Cl)cc3)cc(C(=O)N[C@H]3CCC[C@H]3O)c2=O)cn1]

6-(4-Chlorophenyl)-N-[(trans)-2-hydroxycyclopentyl]-2-(1-methyl-1H-pyrazol-4-yl)-3-oxo-2,3-dihydropyridazine-4-carboxamide

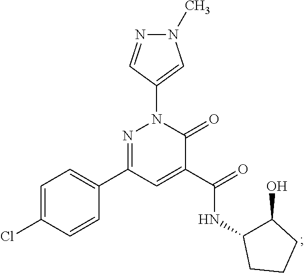 [SMILES: Cn1cc(-n2nc(-c3ccc(Cl)cc3)cc(C(=O)N[C@H]3CCC[C@@H]3O)c2=O)cn1]

6-[4-(Difluoromethyl)phenyl]-N-[(trans)-2-hydroxycyclopentyl]-2-(1-methyl-1H-pyrazol-4-yl)-3-oxo-2,3-dihydropyridazine-4-carboxamide

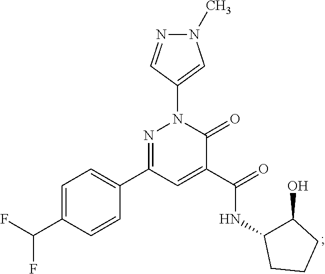 [SMILES: Cn1cc(-n2nc(-c3ccc(C(F)F)cc3)cc(C(=O)N[C@H]3CCC[C@@H]3O)c2=O)cn1]

N-[(2S)-1-Hydroxypropan-2-yl]-2-(1-methyl-1H-pyrazol-4-yl)-3-oxo-6-[4-(trifluoromethoxy)phenyl]-2,3-dihydropyridazine-4-carboxamide

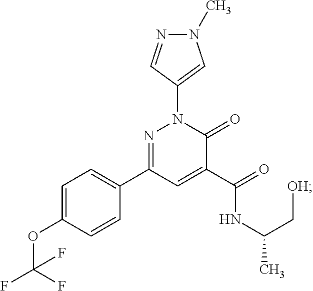 [SMILES: C[C@@H](CO)NC(=O)c1cc(-c2ccc(OC(F)(F)F)cc2)nn(-c2cnn(C)c2)c1=O]

6-[6-(Difluoromethyl)pyridin-3-yl]-N-[(1S,2R)-2-hydroxycyclohexyl]-2-(1-methyl-1H-pyrazol-4-yl)-3-oxo-2,3-dihydropyridazine-4-carboxamide

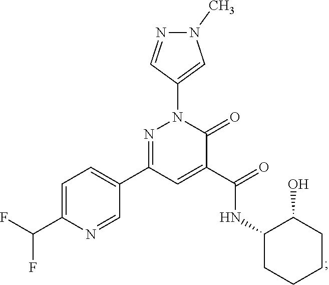 [SMILES: Cn1cc(-n2nc(-c3ccc(C(F)F)nc3)cc(C(=O)N[C@H]3CCCC[C@H]3O)c2=O)cn1]

N-[(cis)-4-Hydroxytetrahydrofuran-3-yl]-2-(1-methyl-1H-pyrazol-4-yl)-3-oxo-6-[4-(trifluoromethoxy)phenyl]-2,3-dihydropyridazine-4-carboxamide

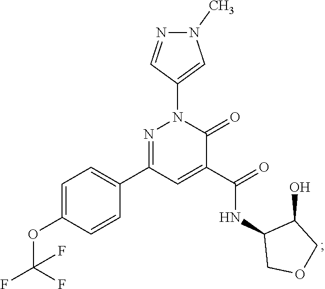 [SMILES: Cn1cc(-n2nc(-c3ccc(OC(F)(F)F)cc3)cc(C(=O)N[C@@H]3COC[C@@H]3O)c2=O)cn1]

N-[(cis)-4-Hydroxytetrahydrofuran-3-yl]-2-(1-methyl-1H-pyrazol-4-yl)-3-oxo-6-[4-(trifluoromethoxy)phenyl]-2,3-dihydropyridazine-4-carboxamide, Enantiomer 1

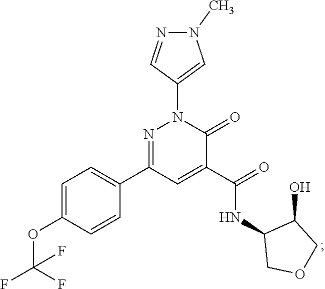 [SMILES: Cn1cc(-n2nc(-c3ccc(OC(F)(F)F)cc3)cc(C(=O)N[C@@H]3COC[C@@H]3O)c2=O)cn1]

N-[(cis)-4-Hydroxytetrahydrofuran-3-yl]-2-(1-methyl-1H-pyrazol-4-yl)-3-oxo-6-[4-(trifluoromethoxy)phenyl]-2,3-dihydropyridazine-4-carboxamide, Enantiomer 2

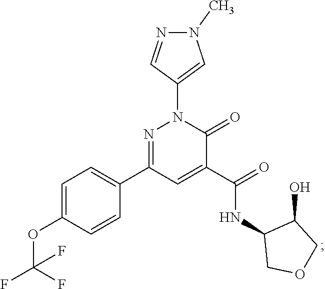 [SMILES: Cn1cc(-n2nc(-c3ccc(OC(F)(F)F)cc3)cc(C(=O)N[C@@H]3COC[C@@H]3O)c2=O)cn1]

6-[4-(Difluoromethyl)phenyl]-N-[(2S)-1-hydroxypropan-2-yl]-2-(1-methyl-1H-pyrazol-4-yl)-3-oxo-2,3-dihydropyridazine-4-carboxamide

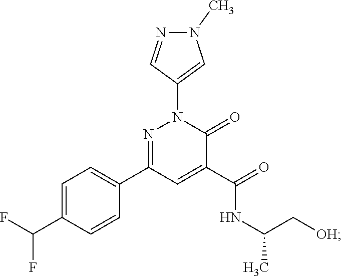 [SMILES: C[C@@H](CO)NC(=O)c1cc(-c2ccc(C(F)F)cc2)nn(-c2cnn(C)c2)c1=O]

N-(2-Hydroxy-2-methylpropyl)-2-(1-methyl-1H-pyrazol-4-yl)-3-oxo-6-[4-(trifluoromethoxy)phenyl]-2,3-dihydropyridazine-4-carboxamide

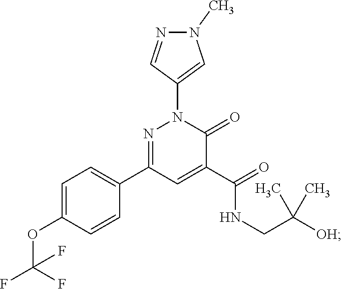 [SMILES: Cn1cc(-n2nc(-c3ccc(OC(F)(F)F)cc3)cc(C(=O)NCC(C)(C)O)c2=O)cn1]

6-[4-(Difluoromethyl)phenyl]-N-[(cis)-4-hydroxytetrahydrofuran-3-yl]-2-(1-methyl-1H-pyrazol-4-yl)-3-oxo-2,3-dihydropyridazine-4-carboxamide

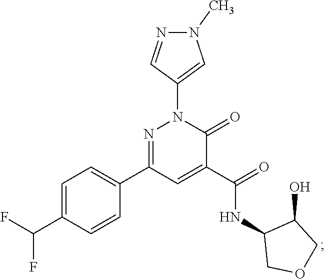 [SMILES: Cn1cc(-n2nc(-c3ccc(C(F)F)cc3)cc(C(=O)N[C@@H]3COC[C@@H]3O)c2=O)cn1]

1,5-Anhydro-2-({[6-(4-chlorophenyl)-2-(1-methyl-1H-pyrazol-4-yl)-3-oxo-2,3-dihydropyridazin-4-yl]carbonyl}amino)-2,4-dideoxy-D-erythro-pentitol

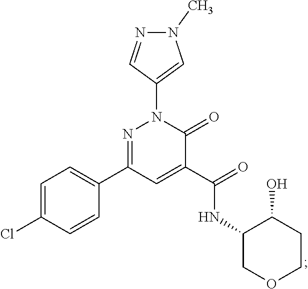 [SMILES: Cn1cc(-n2nc(-c3ccc(Cl)cc3)cc(C(=O)N[C@H]3COCC[C@H]3O)c2=O)cn1]

6-[4-(Difluoromethyl)phenyl]-N-(2-hydroxy-2-methylpropyl)-2-(1-methyl-1H-pyrazol-4-yl)-3-oxo-2,3-dihydropyridazine-4-carboxamide

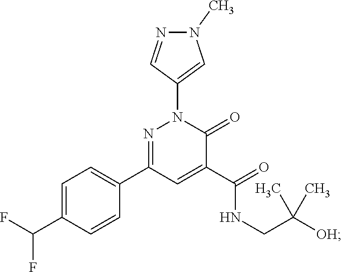 [SMILES: Cn1cc(-n2nc(-c3ccc(C(F)F)cc3)cc(C(=O)NCC(C)(C)O)c2=O)cn1]

N-[(trans)-4-Hydroxytetrahydrofuran-3-yl]-2-(1-methyl-1H-pyrazol-4-yl)-3-oxo-6-[4-(trifluoromethoxy)phenyl]-2,3-dihydropyridazine-4-carboxamide

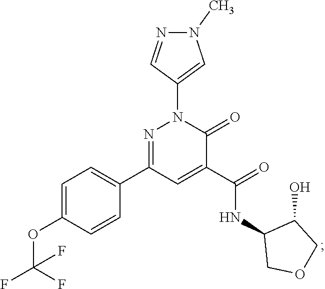 [SMILES: Cn1cc(-n2nc(-c3ccc(OC(F)(F)F)cc3)cc(C(=O)N[C@@H]3COC[C@H]3O)c2=O)cn1]

N-[(trans)-4-Hydroxytetrahydrofuran-3-yl]-2-(1-methyl-1H-pyrazol-4-yl)-3-oxo-6-[4-(trifluoromethoxy)phenyl]-2,3-dihydropyridazine-4-carboxamide, Enantiomer 1

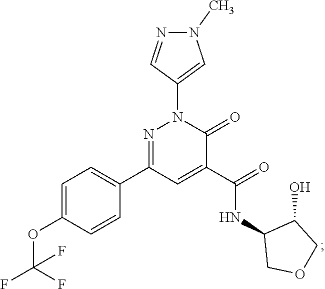 [SMILES: Cn1cc(-n2nc(-c3ccc(OC(F)(F)F)cc3)cc(C(=O)N[C@@H]3COC[C@H]3O)c2=O)cn1]

N-[(trans)-4-Hydroxytetrahydrofuran-3-yl]-2-(1-methyl-1H-pyrazol-4-yl)-3-oxo-6-[4-(trifluoromethoxy)phenyl]-2,3-dihydropyridazine-4-carboxamide, Enantiomer 2

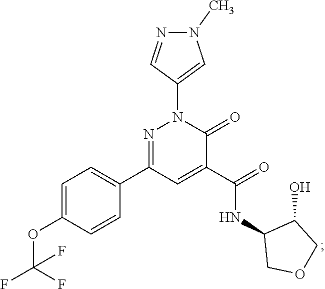 [SMILES: Cn1cc(-n2nc(-c3ccc(OC(F)(F)F)cc3)cc(C(=O)N[C@@H]3COC[C@H]3O)c2=O)cn1]

6-[6-(Difluoromethyl)pyridin-3-yl]-N-[(2S)-1-hydroxypropan-2-yl]-2-(1-methyl-1H-pyrazol-4-yl)-3-oxo-2,3-dihydropyridazine-4-carboxamide

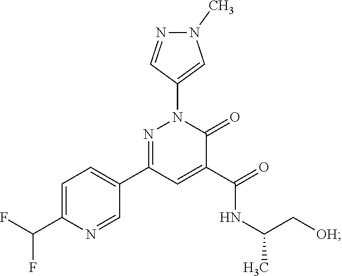 [SMILES: C[C@@H](CO)NC(=O)c1cc(-c2ccc(C(F)F)nc2)nn(-c2cnn(C)c2)c1=O]

N-(2-Hydroxy-2-methylpropyl)-2-(1-methyl-1H-pyrazol-4-yl)-3-oxo-6-[4-(trifluoromethyl)phenyl]-2,3-dihydropyridazine-4-carboxamide

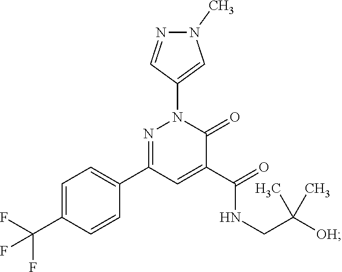 [SMILES: Cn1cc(-n2nc(-c3ccc(C(F)(F)F)cc3)cc(C(=O)NCC(C)(C)O)c2=O)cn1]

6-(4-Chlorophenyl)-N-[(2S)-1-hydroxypropan-2-yl]-3-oxo-2-(pyridin-3-yl)-2,3-dihydropyridazine-4-carboxamide

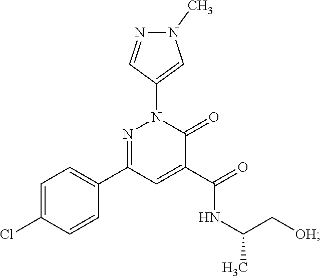 [SMILES: C[C@@H](CO)NC(=O)c1cc(-c2ccc(Cl)cc2)nn(-c2cnn(C)c2)c1=O]

6-(4-Chlorophenyl)-N-[(2R)-1-hydroxypropan-2-yl]-3-oxo-2-(pyridin-3-yl)-2,3-dihydropyridazine-4-carboxamide

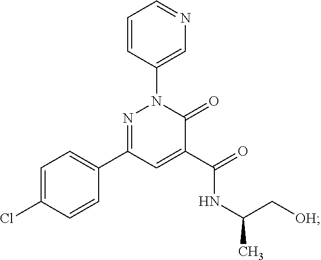 [SMILES: C[C@H](CO)NC(=O)c1cc(-c2ccc(Cl)cc2)nn(-c2cccnc2)c1=O]

6-(4-Chlorophenyl)-N-(1,3-dihydroxypropan-2-yl)-3-oxo-2-(pyridin-3-yl)-2,3-dihydropyridazine-4-carboxamide

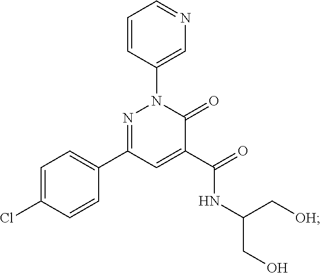 [SMILES: O=C(NC(CO)CO)c1cc(-c2ccc(Cl)cc2)nn(-c2cccnc2)c1=O]

6-(4-Chlorophenyl)-N-(2-hydroxy-2-methylpropyl)-3-oxo-2-(pyridin-3-yl)-2,3-dihydropyridazine-4-carboxamide

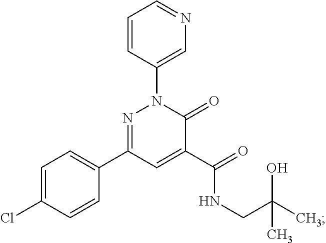 [SMILES: CC(C)(O)CNC(=O)c1cc(-c2ccc(Cl)cc2)nn(-c2cccnc2)c1=O]

(−)-6-(4-Chlorophenyl)-N-[(2S)-2,3-dihydroxypropyl]-3-oxo-2-(pyridin-3-yl)-2,3-dihydropyridazine-4-carboxamide

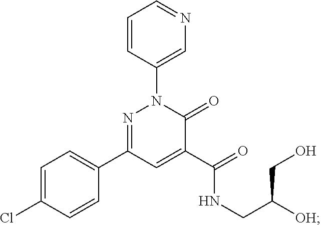 [SMILES: O=C(NC[C@H](O)CO)c1cc(-c2ccc(Cl)cc2)nn(-c2cccnc2)c1=O]

(+)-6-(4-Chlorophenyl)-N-[(2R)-2,3-dihydroxypropyl]-3-oxo-2-(pyridin-3-yl)-2,3-dihydropyridazine-4-carboxamide

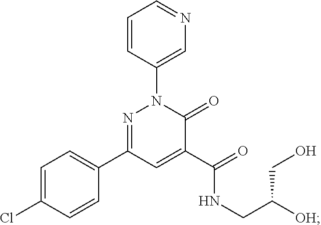 [SMILES: O=C(NC[C@@H](O)CO)c1cc(-c2ccc(Cl)cc2)nn(-c2cccnc2)c1=O]

(+)-6-(4-Chlorophenyl)-N-[(2S)-1-hydroxy-3-methoxypropan-2-yl]-3-oxo-2-(pyridin-3-yl)-2,3-dihydropyridazine-4-carboxamide

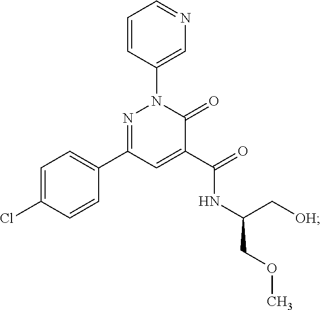 [SMILES: COC[C@H](CO)NC(=O)c1cc(-c2ccc(Cl)cc2)nn(-c2cccnc2)c1=O]

(−)-6-(4-Chlorophenyl)-N-[(2R)-1-hydroxy-3-methoxypropan-2-yl]-3-oxo-2-(pyridin-3-yl)-2,3-dihydropyridazine-4-carboxamide

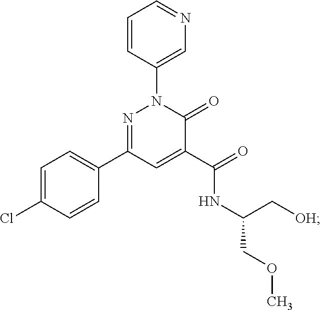 [SMILES: COC[C@@H](CO)NC(=O)c1cc(-c2ccc(Cl)cc2)nn(-c2cccnc2)c1=O]

6-(4-Chlorophenyl)-3-oxo-2-(pyridin-3-yl)-N-[(2RS)-1,1,1-trifluoro-3-hydroxypropan-2-yl]-2,3-dihydropyridazine-4-carboxamide

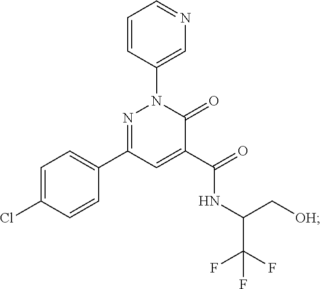 [SMILES: O=C(NC(CO)C(F)(F)F)c1cc(-c2ccc(Cl)cc2)nn(-c2cccnc2)c1=O]

6-(4-Chlorophenyl)-N-[(1 RS)-1-cyclopropyl-2-hydroxyethyl]-3-oxo-2-(pyridin-3-yl)-2,3-dihydropyridazine-4-carboxamide

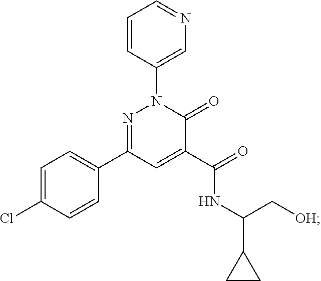 [SMILES: O=C(NC(CO)C1CC1)c1cc(-c2ccc(Cl)cc2)nn(-c2cccnc2)c1=O]

6-(4-Chlorophenyl)-3-oxo-2-(pyridin-3-yl)-N-[(2RS)-3,3,3-trifluoro-2-hydroxypropyl]-2,3-dihydropyridazine-4-carboxamide

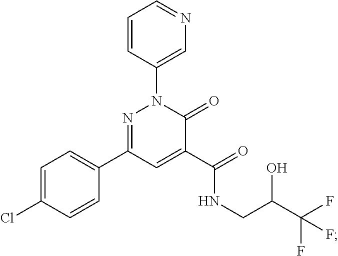 [SMILES: O=C(NCC(O)C(F)(F)F)c1cc(-c2ccc(Cl)cc2)nn(-c2cccnc2)c1=O]

6-(4-Chlorophenyl)-N-[(2 RS)-3,3-difluoro-2-hydroxypropyl]-3-oxo-2-(pyridin-3-yl)-2,3-dihydropyridazine-4-carboxamide

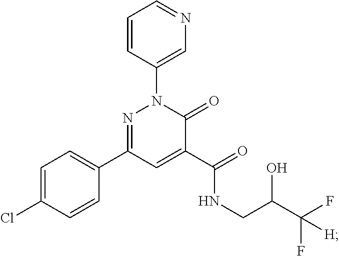

6-(4-Chlorophenyl)-2-(5-fluoropyridin-3-yl)-N-[(2S)-1-hydroxypropan-2-yl]-3-oxo-2,3-dihydropyridazine-4-carboxamide

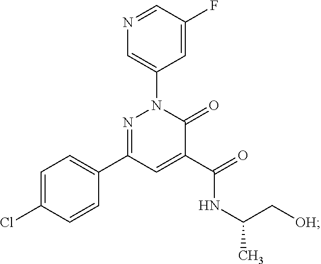 [SMILES: C[C@@H](CO)NC(=O)c1cc(-c2ccc(Cl)cc2)nn(-c2cncc(F)c2)c1=O]

6-(4-Chlorophenyl)-2-(5-fluoropyridin-3-yl)-N-[(2R)-1-hydroxypropan-2-yl]-3-oxo-2,3-dihydropyridazine-4-carboxamide

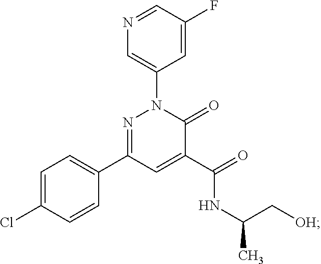 [SMILES: C[C@H](CO)NC(=O)c1cc(-c2ccc(Cl)cc2)nn(-c2cncc(F)c2)c1=O]

6-(4-Chlorophenyl)-2-(5-fluoropyridin-3-yl)-N-(2-hydroxy-2-methylpropyl)-3-oxo-2,3-dihydropyridazine-4-carboxamide

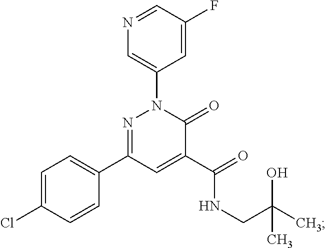 [SMILES: CC(C)(O)CNC(=O)c1cc(-c2ccc(Cl)cc2)nn(-c2cncc(F)c2)c1=O]

6-(4-Chlorophenyl)-2-(5-fluoropyridin-3-yl)-N-[(2S)-1-hydroxy-3-methylbutan-2-yl]-3-oxo-2,3-dihydropyridazine-4-carboxamide

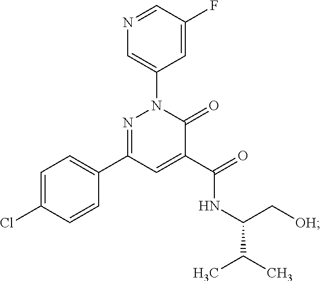 [SMILES: CC(C)[C@@H](CO)NC(=O)c1cc(-c2ccc(Cl)cc2)nn(-c2cncc(F)c2)c1=O]

6-(4-Chlorophenyl)-N-(1,3-dihydroxypropan-2-yl)-2-(5-fluoropyridin-3-yl)-3-oxo-2,3-dihydropyridazine-4-carboxamide

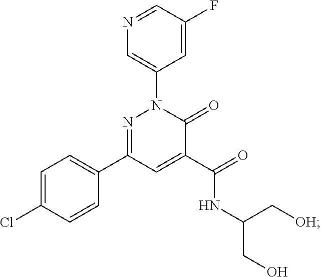 [SMILES: O=C(NC(CO)CO)c1cc(-c2ccc(Cl)cc2)nn(-c2cncc(F)c2)c1=O]

6-(4-Chlorophenyl)-2-(5-fluoropyridin-3-yl)-N-[(2R)-1-hydroxy-3-methoxypropan-2-yl]-3-oxo-2,3-dihydropyridazine-4-carboxamide

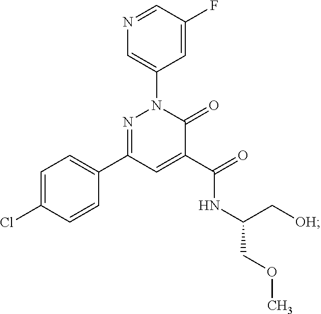 [SMILES: COC[C@@H](CO)NC(=O)c1cc(-c2ccc(Cl)cc2)nn(-c2cncc(F)c2)c1=O]

6-(4-Chlorophenyl)-2-(5-fluoropyridin-3-yl)-N-[(2S)-1-hydroxy-3-methoxypropan-2-yl]-3-oxo-2,3-dihydropyridazine-4-carboxamide

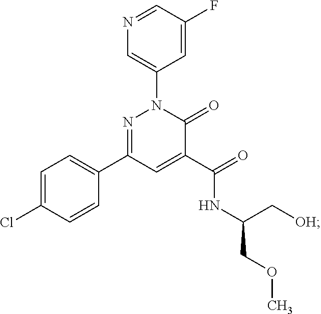 [SMILES: COC[C@H](CO)NC(=O)c1cc(-c2ccc(Cl)cc2)nn(-c2cncc(F)c2)c1=O]

(−)-6-(4-Chlorophenyl)-N-(3,3-difluoro-2-hydroxypropyl)-3-oxo-2-(pyridin-3-yl)-2,3-dihydropyridazine-4-carboxamide

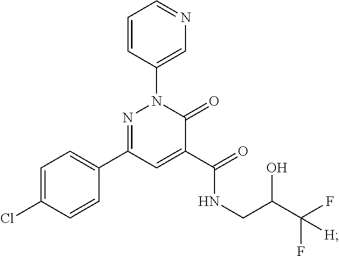

(+)-6-(4-Chlorophenyl)-N-(3,3-difluoro-2-hydroxypropyl)-3-oxo-2-(pyridin-3-yl)-2,3-dihydropyridazine-4-carboxamide

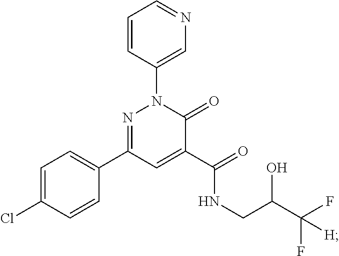

(−)-6-(4-Chlorophenyl)-3-oxo-2-(pyridin-3-yl)-N-(3,3,3-trifluoro-2-hydroxypropyl)-2,3-dihydropyridazine-4-carboxamide

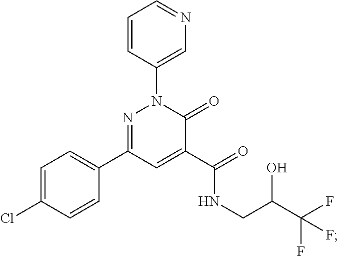 [SMILES: O=C(NCC(O)C(F)(F)F)c1cc(-c2ccc(Cl)cc2)nn(-c2cccnc2)c1=O]

(+)-6-(4-Chlorophenyl)-3-oxo-2-(pyridin-3-yl)-N-(3,3,3-trifluoro-2-hydroxypropyl)-2,3-dihydropyridazine-4-carboxamide

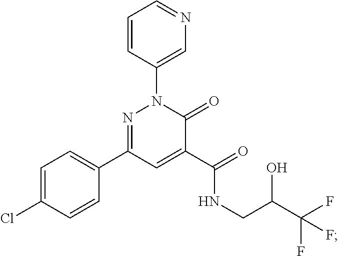 [SMILES: O=C(NCC(O)C(F)(F)F)c1cc(-c2ccc(Cl)cc2)nn(-c2cccnc2)c1=O]

(+)-6-(4-Chlorophenyl)-N-(1-cyclopropyl-2-hydroxyethyl)-3-oxo-2-(pyridin-3-yl)-2,3-dihydropyridazine-4-carboxamide

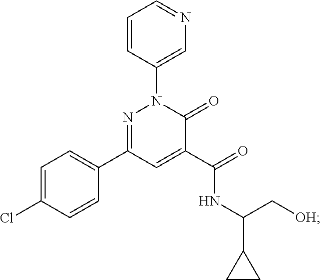 [SMILES: O=C(NC(CO)C1CC1)c1cc(-c2ccc(Cl)cc2)nn(-c2cccnc2)c1=O]

(−)-6-(4-Chlorophenyl)-N-(1-cyclopropyl-2-hydroxyethyl)-3-oxo-2-(pyridin-3-yl)-2,3-dihydropyridazine-4-carboxamide

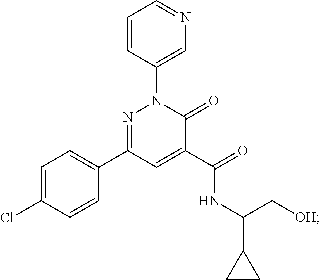 [SMILES: O=C(NC(CO)C1CC1)c1cc(-c2ccc(Cl)cc2)nn(-c2cccnc2)c1=O]

(+)-6-(4-Chlorophenyl)-N-[(2R)-1-fluoro-3-hydroxypropan-2-yl]-3-oxo-2-(pyridin-3-yl)-2,3-dihydropyridazine-4-carboxamide

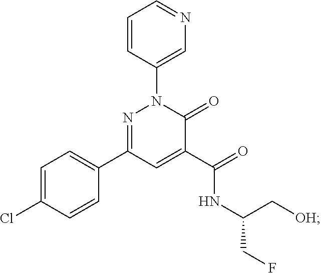 [SMILES: O=C(N[C@H](CO)CF)c1cc(-c2ccc(Cl)cc2)nn(-c2cccnc2)c1=O]

N-[(1S)-1-Cyclopropyl-2-hydroxyethyl]-3-oxo-2-(pyridin-3-yl)-6-[4-(trifluoromethyl)phenyl]-2,3-dihydropyridazine-4-carboxamide

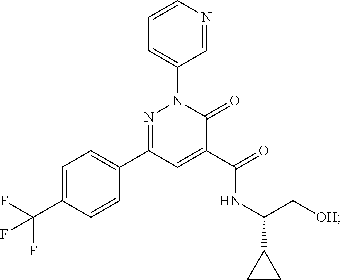 [SMILES: O=C(N[C@H](CO)C1CC1)c1cc(-c2ccc(C(F)(F)F)cc2)nn(-c2cccnc2)c1=O]

(−)-6-(4-Chlorophenyl)-3-oxo-2-(pyridin-3-yl)-N-[(2S)-1,1,1-trifluoro-3-hydroxypropan-2-yl]-2,3-dihydropyridazine-4-carboxamide

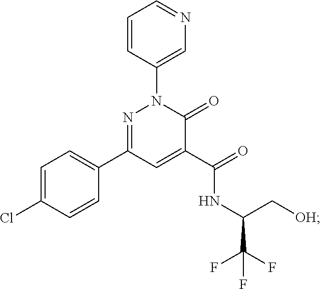 [SMILES: O=C(N[C@@H](CO)C(F)(F)F)c1cc(-c2ccc(Cl)cc2)nn(-c2cccnc2)c1=O]

(+)-6-(4-Chlorophenyl)-3-oxo-2-(pyridin-3-yl)-N-[(2R)-1,1,1-trifluoro-3-hydroxypropan-2-yl]-2,3-dihydropyridazine-4-carboxamide

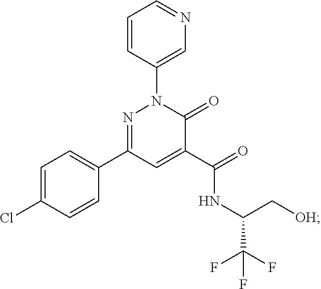 [SMILES: O=C(N[C@H](CO)C(F)(F)F)c1cc(-c2ccc(Cl)cc2)nn(-c2cccnc2)c1=O]

6-(4-Chlorophenyl)-N-[(1 RS)-1-cyclopropyl-2-hydroxyethyl]-2-(5-fluoropyridin-3-yl)-3-oxo-2,3-dihydropyridazine-4-carboxamide

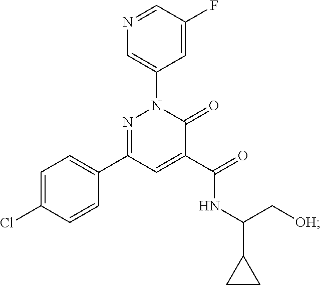 [SMILES: O=C(NC(CO)C1CC1)c1cc(-c2ccc(Cl)cc2)nn(-c2cncc(F)c2)c1=O]

6-(4-Chlorophenyl)-N-[(2R)-2,3-dihydroxypropyl]-2-(5-fluoropyridin-3-yl)-3-oxo-2,3-dihydropyridazine-4-carboxamide

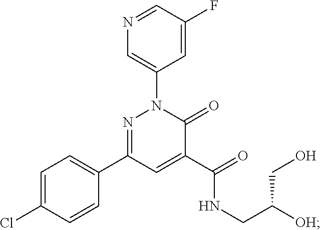 [SMILES: O=C(NC[C@@H](O)CO)c1cc(-c2ccc(Cl)cc2)nn(-c2cncc(F)c2)c1=O]

6-(4-Chlorophenyl)-N-[(2S)-2,3-dihydroxypropyl]-2-(5-fluoropyridin-3-yl)-3-oxo-2,3-dihydropyridazine-4-carboxamide

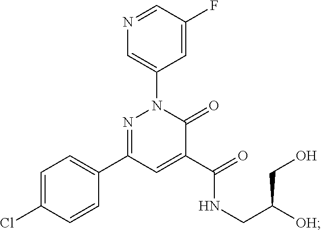 [SMILES: O=C(NC[C@H](O)CO)c1cc(-c2ccc(Cl)cc2)nn(-c2cncc(F)c2)c1=O]

6-(4-Chlorophenyl)-2-(5-fluoropyridin-3-yl)-3-oxo-N-[(2R)-1,1,1-trifluoro-3-hydroxypropan-2-yl]-2,3-dihydropyridazine-4-carboxamide

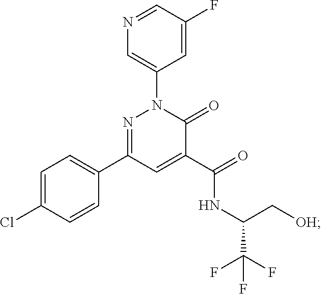 [SMILES: O=C(N[C@H](CO)C(F)(F)F)c1cc(-c2ccc(Cl)cc2)nn(-c2cncc(F)c2)c1=O]

N-[(2RS)-3,3-Difluoro-2-hydroxypropyl]-3-oxo-2-(pyridin-3-yl)-6-[4-(trifluoromethyl)phenyl]-2,3-dihydropyridazine-4-carboxamide

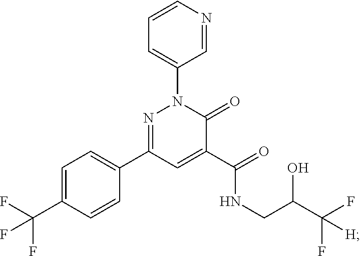

N-[(1S,2S)-2-Hydroxycyclopentyl]-3-oxo-2-(pyridin-3-yl)-6-[4-(trifluoromethyl)phenyl]-2,3-dihydropyridazine-4-carboxamide

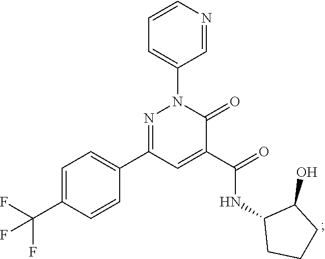 [SMILES: O=C(N[C@H]1CCC[C@@H]1O)c1cc(-c2ccc(C(F)(F)F)cc2)nn(-c2cccnc2)c1=O]

N-[(1R,2S)-2-Hydroxycyclopentyl]-3-oxo-2-(pyridin-3-yl)-6-[4-(trifluoromethyl)phenyl]-2,3-dihydropyridazine-4-carboxamide

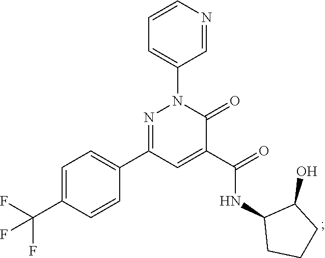 [SMILES: O=C(N[C@@H]1CCC[C@@H]1O)c1cc(-c2ccc(C(F)(F)F)cc2)nn(-c2cccnc2)c1=O]

N-[(1R,2R)-2-Hydroxycyclopentyl]-3-oxo-2-(pyridin-3-yl)-6-[4-(trifluoromethyl)phenyl]-2,3-dihydropyridazine-4-carboxamide

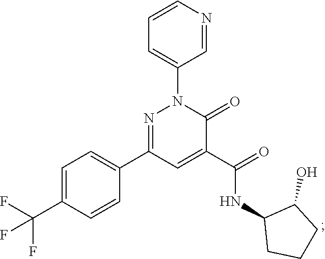 [SMILES: O=C(N[C@@H]1CCC[C@H]1O)c1cc(-c2ccc(C(F)(F)F)cc2)nn(-c2cccnc2)c1=O]

N-[(1S,2R)-2-Hydroxycyclopentyl]-3-oxo-2-(pyridin-3-yl)-6-[4-(trifluoromethyl)phenyl]-2,3-dihydropyridazine-4-carboxamide

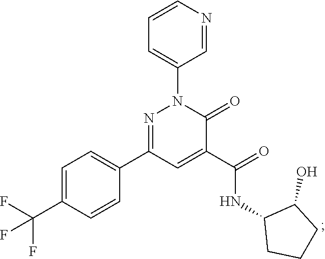 [SMILES: O=C(N[C@H]1CCC[C@H]1O)c1cc(-c2ccc(C(F)(F)F)cc2)nn(-c2cccnc2)c1=O]

N-[(1S,2R)-2-Hydroxycyclohexyl]-3-oxo-2-(pyridin-3-yl)-6-[4-(trifluoromethyl)phenyl]-2,3-dihydropyridazine-4-carboxamide

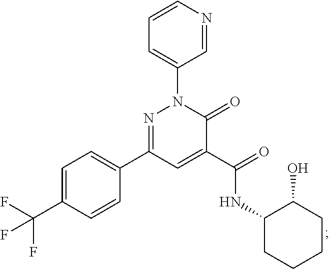 [SMILES: O=C(N[C@H]1CCCC[C@H]1O)c1cc(-c2ccc(C(F)(F)F)cc2)nn(-c2cccnc2)c1=O]

N-[(3RS,4RS)-4-Hydroxytetrahydrofuran-3-yl]-3-oxo-2-(pyridin-3-yl)-6-[4-(trifluoromethyl)-phenyl]-2,3-dihydropyridazine-4-carboxamide

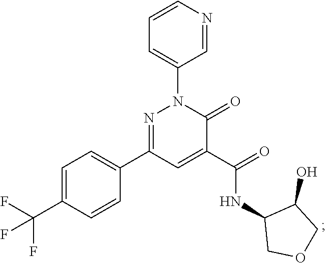 [SMILES: O=C(N[C@@H]1COC[C@@H]1O)c1cc(-c2ccc(C(F)(F)F)cc2)nn(-c2cccnc2)c1=O]

3-Oxo-2-(pyridin-3-yl)-N-[(2RS)-3,3,3-trifluoro-2-hydroxypropyl]-6-[4-(trifluoromethyl)phenyl]-2,3-dihydropyridazine-4-carboxamide

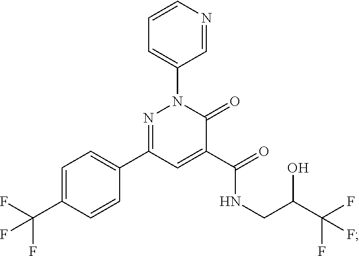 [SMILES: O=C(NCC(O)C(F)(F)F)c1cc(-c2ccc(C(F)(F)F)cc2)nn(-c2cccnc2)c1=O]

N-[(1RS)-1-Cyclopropyl-2-hydroxyethyl]-3-oxo-2-(pyridin-3-yl)-6-[4-(trifluoromethyl)phenyl]-2,3-dihydropyridazine-4-carboxamide

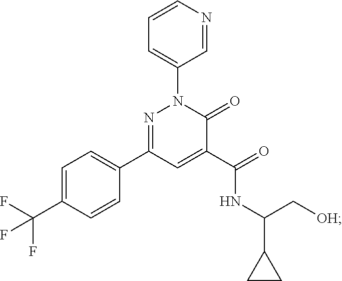 [SMILES: O=C(NC(CO)C1CC1)c1cc(-c2ccc(C(F)(F)F)cc2)nn(-c2cccnc2)c1=O]

3-Oxo-2-(pyridin-3-yl)-N-[(2RS)-1,1,1-trifluoro-3-hydroxypropan-2-yl]-6-[4-(trifluoromethyl)phenyl]-2,3-dihydropyridazine-4-carboxamide

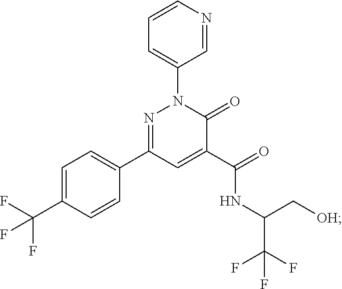 [SMILES: O=C(NC(CO)C(F)(F)F)c1cc(-c2ccc(C(F)(F)F)cc2)nn(-c2cccnc2)c1=O]

N-[(2S)-1-Hydroxy-3-methoxypropan-2-yl]-3-oxo-2-(pyridin-3-yl)-6-[4-(trifluoromethyl)phenyl]-2,3-dihydropyridazine-4-carboxamide

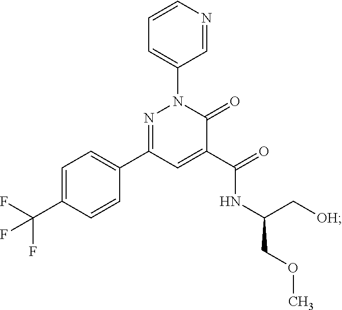 [SMILES: COC[C@H](CO)NC(=O)c1cc(-c2ccc(C(F)(F)F)cc2)nn(-c2cccnc2)c1=O]

3-Oxo-2-(pyridin-3-yl)-N-[(2R)-1,1,1-trifluoro-3-hydroxypropan-2-yl]-6-[4-(trifluoromethyl)phenyl]-2,3-dihydropyridazine-4-carboxamide

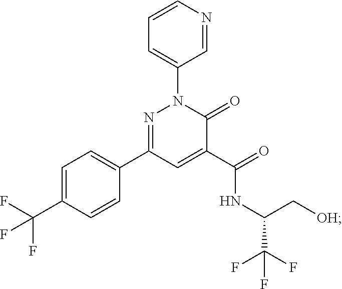 [SMILES: O=C(N[C@H](CO)C(F)(F)F)c1cc(-c2ccc(C(F)(F)F)cc2)nn(-c2cccnc2)c1=O]

N-[(2R)-2,3-Dihydroxypropyl]-3-oxo-2-(pyridin-3-yl)-6-[4-(trifluoromethyl)phenyl]-2,3-dihydropyridazine-4-carboxamide

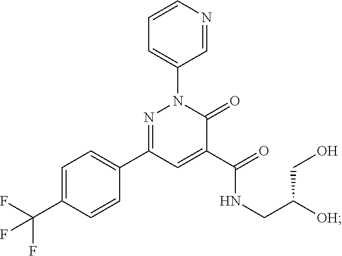 [SMILES: O=C(NC[C@@H](O)CO)c1cc(-c2ccc(C(F)(F)F)cc2)nn(-c2cccnc2)c1=O]

N-[(2S)-2,3-Dihydroxypropyl]-3-oxo-2-(pyridin-3-yl)-6-[4-(trifluoromethyl)phenyl]-2,3-dihydropyridazine-4-carboxamide

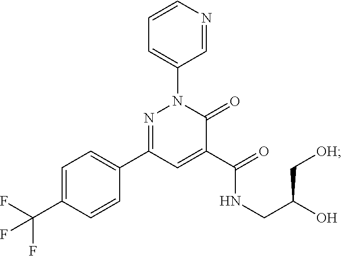 [SMILES: O=C(NC[C@H](O)CO)c1cc(-c2ccc(C(F)(F)F)cc2)nn(-c2cccnc2)c1=O]

N-(2-Hydroxy-2-methylpropyl)-3-oxo-2-(pyridin-3-yl)-6-[4-(trifluoromethyl)phenyl]-2,3-dihydropyridazine-4-carboxamide

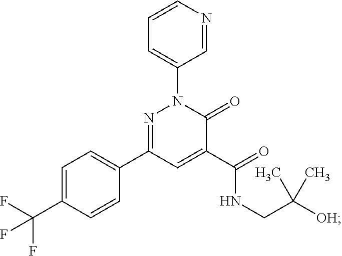 [SMILES: CC(C)(O)CNC(=O)c1cc(-c2ccc(C(F)(F)F)cc2)nn(-c2cccnc2)c1=O]

N-(1,3-Dihydroxypropan-2-yl)-3-oxo-2-(pyridin-3-yl)-6-[4-(trifluoromethyl)phenyl]-2,3-dihydropyridazine-4-carboxamide

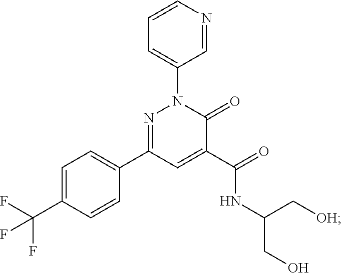 [SMILES: O=C(NC(CO)CO)c1cc(-c2ccc(C(F)(F)F)cc2)nn(-c2cccnc2)c1=O]

3-Oxo-2-(pyridin-3-yl)-N-[(2S)-3,3,3-trifluoro-2-hydroxypropyl]-6-[4-(trifluoromethyl)phenyl]-2,3-dihydropyridazine-4-carboxamide

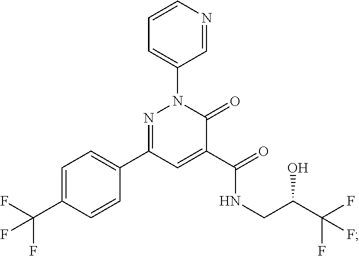 [SMILES: O=C(NC[C@H](O)C(F)(F)F)c1cc(-c2ccc(C(F)(F)F)cc2)nn(-c2cccnc2)c1=O]

N-[(2R)-1-Hydroxypropan-2-yl]-3-oxo-2-(pyridin-3-yl)-6-[4-(trifluoromethyl)phenyl]-2,3-dihydropyridazine-4-carboxamide

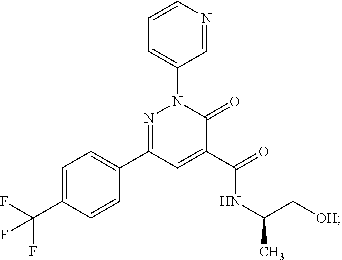 [SMILES: C[C@H](CO)NC(=O)c1cc(-c2ccc(C(F)(F)F)cc2)nn(-c2cccnc2)c1=O]

N-[(2S)-1-Hydroxypropan-2-yl]-3-oxo-2-(pyridin-3-yl)-6-[4-(trifluoromethyl)phenyl]-2,3-dihydropyridazine-4-carboxamide

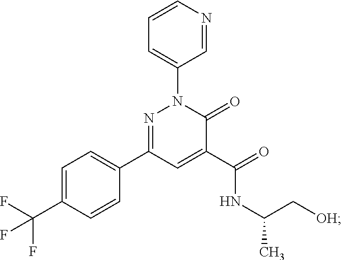 [SMILES: C[C@@H](CO)NC(=O)c1cc(-c2ccc(C(F)(F)F)cc2)nn(-c2cccnc2)c1=O]

6-(4-Chlorophenyl)-N-[(2S)-1-hydroxypropan-2-yl]-3-oxo-2-(pyrimidin-5-yl)-2,3-dihydropyridazine-4-carboxamide

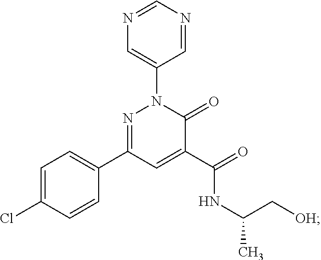 [SMILES: C[C@@H](CO)NC(=O)c1cc(-c2ccc(Cl)cc2)nn(-c2cncnc2)c1=O]

6-(4-Chlorophenyl)-3-oxo-2-(pyridin-3-yl)-N-(1,1,1-trifluoro-3-hydroxy-3-methylbutan-2-yl)-2,3-dihydropyridazine-4-carboxamide

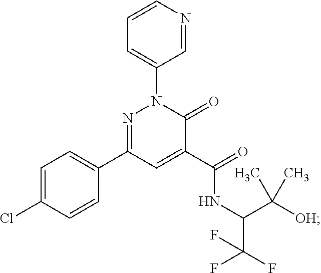 [SMILES: CC(C)(O)C(NC(=O)c1cc(-c2ccc(Cl)cc2)nn(-c2cccnc2)c1=O)C(F)(F)F]

6-(4-Chlorophenyl)-3-oxo-2-(pyridin-3-yl)-N-(1,1,1-trifluoro-3-hydroxy-3-methylbutan-2-yl)-2,3-dihydropyridazine-4-carboxamide, Enantiomer 1

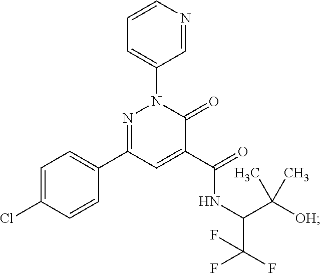 [SMILES: CC(C)(O)C(NC(=O)c1cc(-c2ccc(Cl)cc2)nn(-c2cccnc2)c1=O)C(F)(F)F]

6-(4-Chlorophenyl)-3-oxo-2-(pyridin-3-yl)-N-(1,1,1-trifluoro-3-hydroxy-3-methylbutan-2-yl)-2,3-dihydropyridazine-4-carboxamide, Enantiomer 2

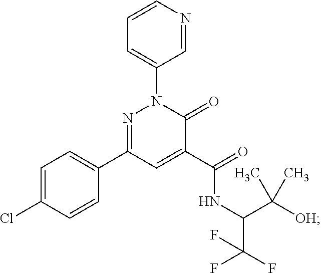 [SMILES: CC(C)(O)C(NC(=O)c1cc(-c2ccc(Cl)cc2)nn(-c2cccnc2)c1=O)C(F)(F)F]

6-(4-Chlorophenyl)-2-(5-fluoropyridin-3-yl)-N-[(1S,2R)-2-hydroxycyclohexyl]-3-oxo-2,3-dihydropyridazine-4-carboxamide

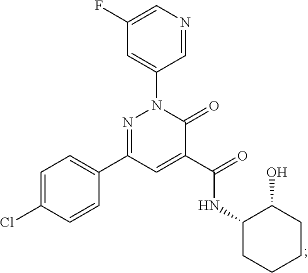 [SMILES: O=C(N[C@H]1CCCC[C@H]1O)c1cc(-c2ccc(Cl)cc2)nn(-c2cncc(F)c2)c1=O]

6-(4-Chlorophenyl)-N-[(1S,2R)-2-hydroxycyclohexyl]-3-oxo-2-(pyridin-3-yl)-2,3-dihydropyridazine-4-carboxamide

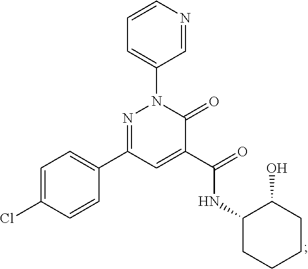 [SMILES: O=C(N[C@H]1CCCC[C@H]1O)c1cc(-c2ccc(Cl)cc2)nn(-c2cccnc2)c1=O]

N-[(1S,2R)-2-Hydroxycyclohexyl]-3-oxo-2-(pyridin-3-yl)-6-[4-(trifluoromethoxy)phenyl]-2,3-dihydropyridazine-4-carboxamide

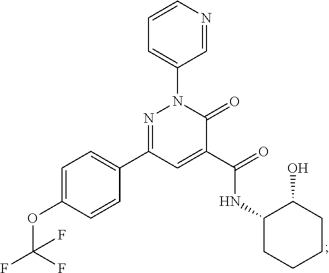 [SMILES: O=C(N[C@H]1CCCC[C@H]1O)c1cc(-c2ccc(OC(F)(F)F)cc2)nn(-c2cccnc2)c1=O]

6-[4-(Difluoromethyl)phenyl]-3-oxo-2-(pyridin-3-yl)-N-[(2R)-1,1,1-trifluoro-3-hydroxypropan-2-yl]-2,3-dihydropyridazine-4-carboxamide

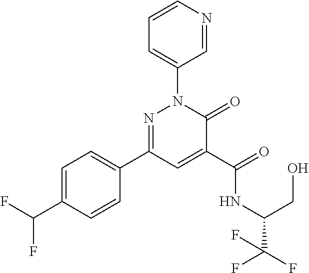 [SMILES: O=C(N[C@H](CO)C(F)(F)F)c1cc(-c2ccc(C(F)F)cc2)nn(-c2cccnc2)c1=O]

6-(4-Chlorophenyl)-2-(5-fluoropyridin-3-yl)-3-oxo-N-[(2S)-3,3,3-trifluoro-2-hydroxypropyl]-2,3-dihydropyridazine-4-carboxamide

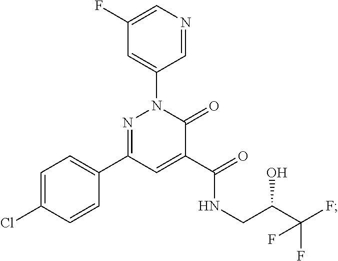 [SMILES: O=C(NC[C@H](O)C(F)(F)F)c1cc(-c2ccc(Cl)cc2)nn(-c2cncc(F)c2)c1=O]

6-(4-Chlorophenyl)-2-(5-fluoropyridin-3-yl)-N-[(1S,2R)-2-hydroxycyclopentyl]-3-oxo-2,3-dihydropyridazine-4-carboxamide

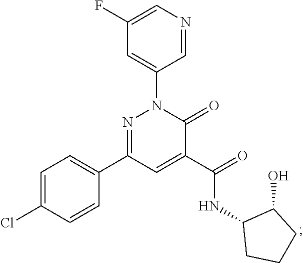 [SMILES: O=C(N[C@H]1CCC[C@H]1O)c1cc(-c2ccc(Cl)cc2)nn(-c2cncc(F)c2)c1=O]

6-(4-Chlorophenyl)-N-[(1S,2S)-2-hydroxycyclopentyl]-3-oxo-2-(pyridin-3-yl)-2,3-dihydropyridazine-4-carboxamide

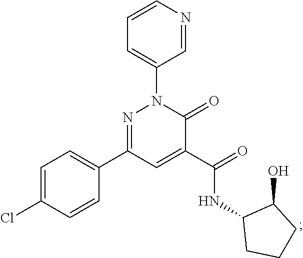 [SMILES: O=C(N[C@H]1CCC[C@@H]1O)c1cc(-c2ccc(Cl)cc2)nn(-c2cccnc2)c1=O]

3-Oxo-2-(pyridin-3-yl)-N-[(2S)-3,3,3-trifluoro-2-hydroxypropyl]-6-[4-(trifluoromethoxy)phenyl]-2,3-dihydropyridazine-4-carboxamide

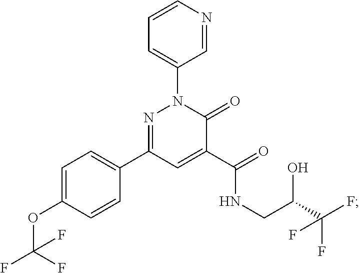 [SMILES: O=C(NC[C@H](O)C(F)(F)F)c1cc(-c2ccc(OC(F)(F)F)cc2)nn(-c2cccnc2)c1=O]

N-[(1S,2S)-2-Hydroxycyclopentyl]-3-oxo-2-(pyridin-3-yl)-6-[4-(trifluoromethoxy)phenyl]-2,3-dihydropyridazine-4-carboxamide

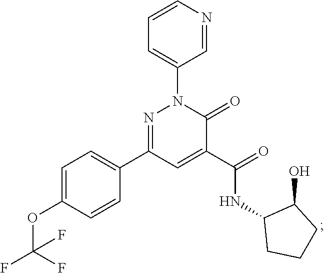 [SMILES: O=C(N[C@H]1CCC[C@@H]1O)c1cc(-c2ccc(OC(F)(F)F)cc2)nn(-c2cccnc2)c1=O]

6-(4-Chlorophenyl)-N-[(cis)-2-hydroxycyclohexyl]-3-oxo-2-(pyridin-3-yl)-2,3-dihydropyridazine-4-carboxamide

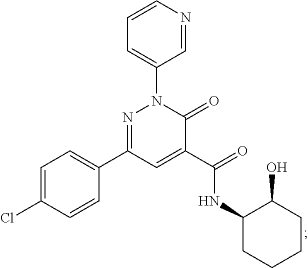 [SMILES: O=C(N[C@@H]1CCCC[C@@H]1O)c1cc(-c2ccc(Cl)cc2)nn(-c2cccnc2)c1=O]

3-Oxo-2-(pyridin-3-yl)-N-[(2R)-1,1,1-trifluoro-3-hydroxypropan-2-yl]-6-[4-(trifluoromethoxy)phenyl]-2,3-dihydropyridazine-4-carboxamide

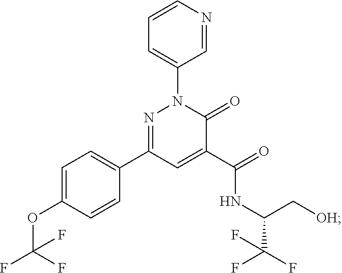 [SMILES: O=C(N[C@H](CO)C(F)(F)F)c1cc(-c2ccc(OC(F)(F)F)cc2)nn(-c2cccnc2)c1=O]

3-Oxo-2-(pyridin-3-yl)-N-[1,1,1-trifluoro-3-hydroxy-3-methylbutan-2-yl]-6-[4-(trifluoromethoxy)phenyl]-2,3-dihydropyridazine-4-carboxamide

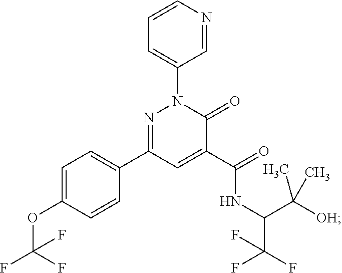 [SMILES: CC(C)(O)C(NC(=O)c1cc(-c2ccc(OC(F)(F)F)cc2)nn(-c2cccnc2)c1=O)C(F)(F)F]

3-Oxo-2-(pyridin-3-yl)-N-[1,1,1-trifluoro-3-hydroxy-3-methylbutan-2-yl]-6-[4-(trifluoromethoxy)phenyl]-2,3-dihydropyridazine-4-carboxamide, Enantiomer 1

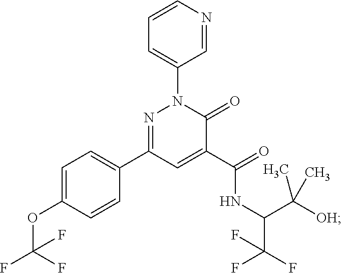 [SMILES: CC(C)(O)C(NC(=O)c1cc(-c2ccc(OC(F)(F)F)cc2)nn(-c2cccnc2)c1=O)C(F)(F)F]

3-Oxo-2-(pyridin-3-yl)-N-[1,1,1-trifluoro-3-hydroxy-3-methylbutan-2-yl]-6-[4-(trifluoromethoxy)phenyl]-2,3-dihydropyridazine-4-carboxamide, Enantiomer 2

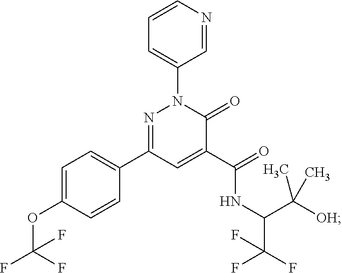 [SMILES: CC(C)(O)C(NC(=O)c1cc(-c2ccc(OC(F)(F)F)cc2)nn(-c2cccnc2)c1=O)C(F)(F)F]

6-(4-Chlorophenyl)-N-[(1R,2S)-2-hydroxycyclopentyl]-3-oxo-2-(pyridin-3-yl)-2,3-dihydropyridazine-4-carboxamide

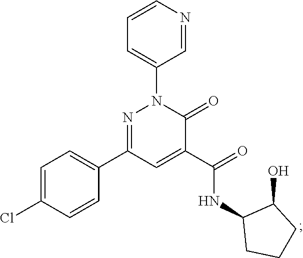 [SMILES: O=C(N[C@@H]1CCC[C@@H]1O)c1cc(-c2ccc(Cl)cc2)nn(-c2cccnc2)c1=O]

6-[4-(Difluoromethyl)phenyl]-N-[(1S,2S)-2-hydroxycyclopentyl]-3-oxo-2-(pyridin-3-yl)-2,3-dihydropyridazine-4-carboxamide

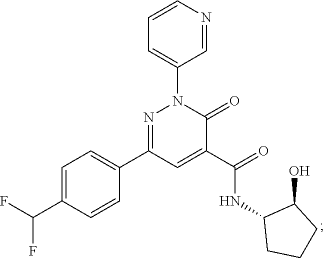 [SMILES: O=C(N[C@H]1CCC[C@@H]1O)c1cc(-c2ccc(C(F)F)cc2)nn(-c2cccnc2)c1=O]

N-[(trans)-3,3-Difluoro-2-hydroxycyclohexyl]-3-oxo-2-(pyridin-3-yl)-6-[4-(trifluoromethoxy)phenyl]-2,3-dihydropyridazine-4-carboxamide

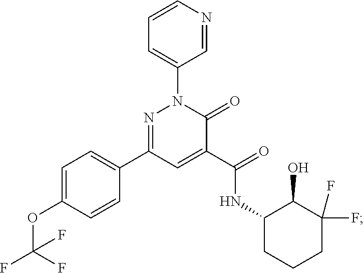 [SMILES: O=C(N[C@H]1CCCC(F)(F)[C@@H]1O)c1cc(-c2ccc(OC(F)(F)F)cc2)nn(-c2cccnc2)c1=O]

N-[(trans)-3,3-Difluoro-2-hydroxycyclohexyl]-3-oxo-2-(pyridin-3-yl)-6-[4-(trifluoromethoxy)phenyl]-2,3-dihydropyridazine-4-carboxamide, Enantiomer 1

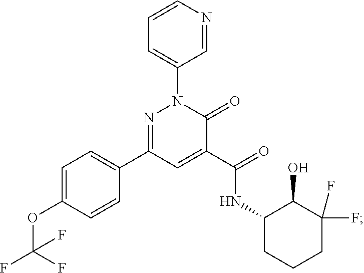 [SMILES: O=C(N[C@H]1CCCC(F)(F)[C@@H]1O)c1cc(-c2ccc(OC(F)(F)F)cc2)nn(-c2cccnc2)c1=O]

N-[(trans)-3,3-Difluoro-2-hydroxycyclohexyl]-3-oxo-2-(pyridin-3-yl)-6-[4-(trifluoromethoxy)phenyl]-2,3-dihydropyridazine-4-carboxamide, Enantiomer 2

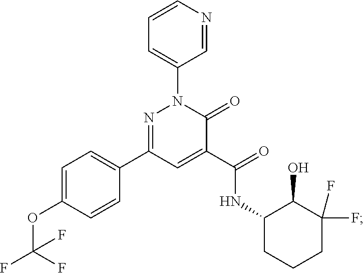 [SMILES: O=C(N[C@H]1CCCC(F)(F)[C@@H]1O)c1cc(-c2ccc(OC(F)(F)F)cc2)nn(-c2cccnc2)c1=O]

6-(4-Chlorophenyl)-N-[(trans)-3,3-difluoro-2-hydroxycyclohexyl]-3-oxo-2-(pyridin-3-yl)-2,3-dihydropyridazine-4-carboxamide

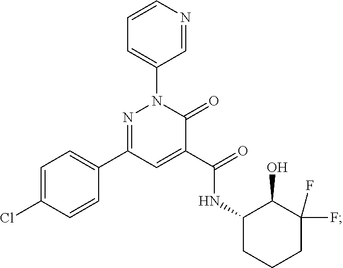 [SMILES: O=C(N[C@H]1CCCC(F)(F)[C@@H]1O)c1cc(-c2ccc(Cl)cc2)nn(-c2cccnc2)c1=O]

6-(4-Chlorophenyl)-N-[(trans)-3,3-difluoro-2-hydroxycyclohexyl]-3-oxo-2-(pyridin-3-yl)-2,3-dihydropyridazine-4-carboxamide, Enantiomer 1

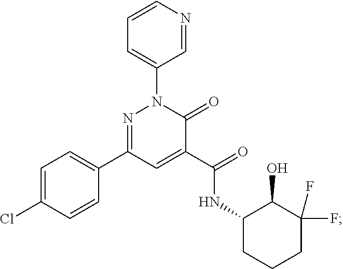 [SMILES: O=C(N[C@H]1CCCC(F)(F)[C@@H]1O)c1cc(-c2ccc(Cl)cc2)nn(-c2cccnc2)c1=O]

6-(4-Chlorophenyl)-N-[(trans)-3,3-difluoro-2-hydroxycyclohexyl]-3-oxo-2-(pyridin-3-yl)-2,3-dihydropyridazine-4-carboxamide, Enantiomer 2

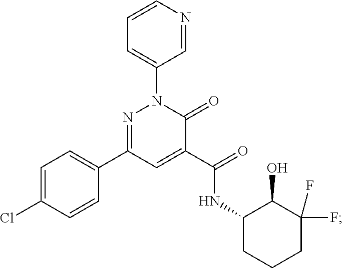 [SMILES: O=C(N[C@H]1CCCC(F)(F)[C@@H]1O)c1cc(-c2ccc(Cl)cc2)nn(-c2cccnc2)c1=O]

3-Oxo-2-(pyridin-3-yl)-N-[3,3,3-trifluoro-2-hydroxypropyl]-6-[4-(trifluoromethoxy)phenyl]-2,3-dihydropyridazine-4-carboxamide

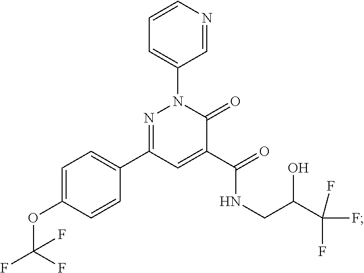 [SMILES: O=C(NCC(O)C(F)(F)F)c1cc(-c2ccc(OC(F)(F)F)cc2)nn(-c2cccnc2)c1=O]

N-[(1S,2R)-2-Hydroxycyclopentyl]-3-oxo-2-(pyridin-3-yl)-6-[4-(trifluoromethoxy)phenyl]-2,3-dihydropyridazine-4-carboxamide

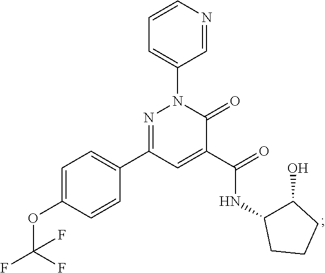 [SMILES: O=C(N[C@H]1CCC[C@H]1O)c1cc(-c2ccc(OC(F)(F)F)cc2)nn(-c2cccnc2)c1=O]

6-(4-Chlorophenyl)-N-[(trans)-2-hydroxycyclopentyl]-3-oxo-2-(pyridin-3-yl)-2,3-dihydropyridazine-4-carboxamide

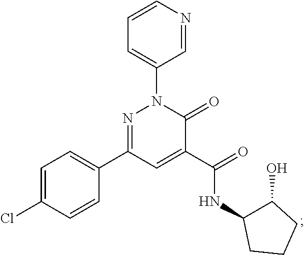 [SMILES: O=C(N[C@@H]1CCC[C@H]1O)c1cc(-c2ccc(Cl)cc2)nn(-c2cccnc2)c1=O]

3-Oxo-2-(pyridin-3-yl)-N-(1,1,1-trifluoro-3-hydroxypropan-2-yl)-6-[4-(trifluoromethoxy)phenyl]-2,3-dihydropyridazine-4-carboxamide

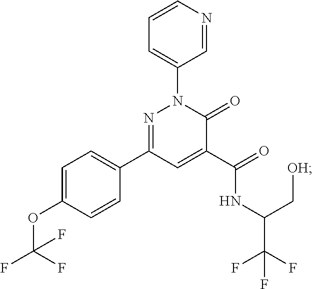 [SMILES: O=C(NC(CO)C(F)(F)F)c1cc(-c2ccc(OC(F)(F)F)cc2)nn(-c2cccnc2)c1=O]

N-[(1S)-1-Cyclopropyl-2-hydroxyethyl]-6-[4-(difluoromethyl)phenyl]-3-oxo-2-(pyridin-3-yl)-2,3-dihydropyridazine-4-carboxamide

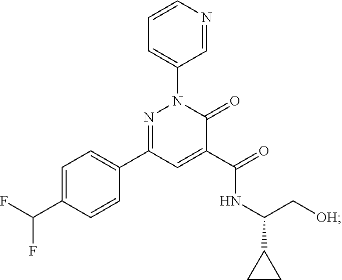 [SMILES: O=C(N[C@H](CO)C1CC1)c1cc(-c2ccc(C(F)F)cc2)nn(-c2cccnc2)c1=O]

6-[4-(Difluoromethyl)phenyl]-N-[(2S)-1-hydroxypropan-2-yl]-3-oxo-2-(pyridin-3-yl)-2,3-dihydropyridazine-4-carboxamide

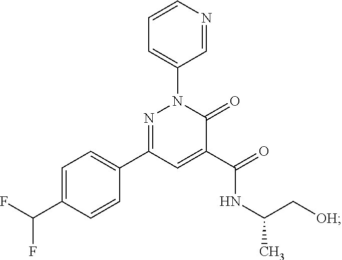 [SMILES: C[C@@H](CO)NC(=O)c1cc(-c2ccc(C(F)F)cc2)nn(-c2cccnc2)c1=O]

N-[(2S)-1-Hydroxypropan-2-yl]-6-(4-methylphenyl)-3-oxo-2-(pyridin-3-yl)-2,3-dihydropyridazine-4-carboxamide

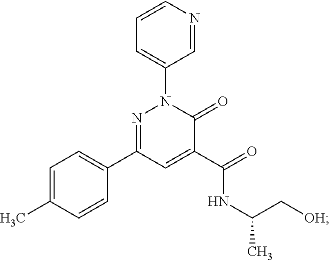 [SMILES: Cc1ccc(-c2cc(C(=O)N[C@@H](C)CO)c(=O)n(-c3cccnc3)n2)cc1]

N-[(2S)-1-Hydroxypropan-2-yl]-3-oxo-2-(pyridin-3-yl)-6-[4-(trifluoromethoxy)phenyl]-2,3-dihydropyridazine-4-carboxamide

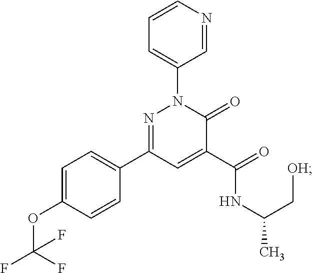 [SMILES: C[C@@H](CO)NC(=O)c1cc(-c2ccc(OC(F)(F)F)cc2)nn(-c2cccnc2)c1=O]

6-(4-Chlorophenyl)-2-(5-fluoropyridin-3-yl)-N-[(trans)-4-hydroxytetrahydrofuran-3-yl]-3-oxo-2,3-dihydropyridazine-4-carboxamide

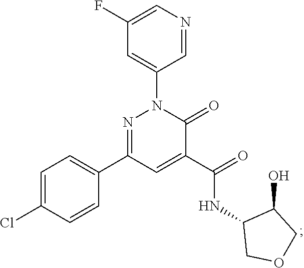 [SMILES: O=C(N[C@H]1COC[C@@H]1O)c1cc(-c2ccc(Cl)cc2)nn(-c2cncc(F)c2)c1=O]

6-(4-Chlorophenyl)-2-(5-fluoropyridin-3-yl)-N-[(trans)-4-hydroxytetrahydrofuran-3-yl]-3-oxo-2,3-dihydropyridazine-4-carboxamide, Enantiomer 1

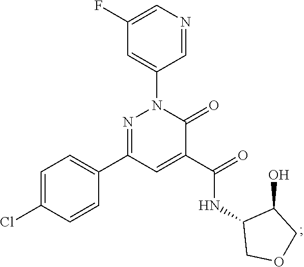 [SMILES: O=C(N[C@H]1COC[C@@H]1O)c1cc(-c2ccc(Cl)cc2)nn(-c2cncc(F)c2)c1=O]

6-(4-Chlorophenyl)-2-(5-fluoropyridin-3-yl)-N-[(trans)-4-hydroxytetrahydrofuran-3-yl]-3-oxo-2,3-dihydropyridazine-4-carboxamide, Enantiomer 2

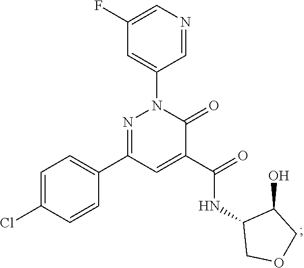 [SMILES: O=C(N[C@H]1COC[C@@H]1O)c1cc(-c2ccc(Cl)cc2)nn(-c2cncc(F)c2)c1=O]

N-[(cis)-4-Hydroxytetrahydrofuran-3-yl]-3-oxo-2-(pyridin-3-yl)-6-[4-(trifluoromethoxy)phenyl]-2,3-dihydropyridazine-4-carboxamide

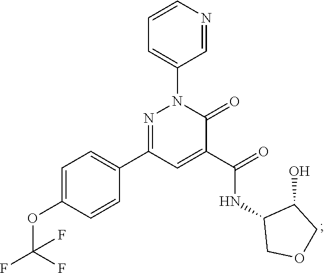 [SMILES: O=C(N[C@H]1COC[C@H]1O)c1cc(-c2ccc(OC(F)(F)F)cc2)nn(-c2cccnc2)c1=O]

N-[(cis)-4-Hydroxytetrahydrofuran-3-yl]-3-oxo-2-(pyridin-3-yl)-6-[4-(trifluoromethoxy)phenyl]-2,3-dihydropyridazine-4-carboxamide, Enantiomer 1

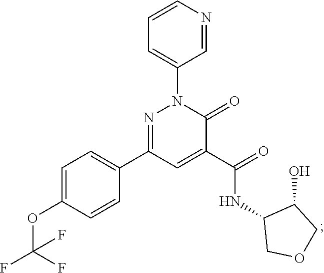 [SMILES: O=C(N[C@H]1COC[C@H]1O)c1cc(-c2ccc(OC(F)(F)F)cc2)nn(-c2cccnc2)c1=O]

N-[(cis)-4-Hydroxytetrahydrofuran-3-yl]-3-oxo-2-(pyridin-3-yl)-6-[4-(trifluoromethoxy)phenyl]-2,3-dihydropyridazine-4-carboxamide, Enantiomer 2

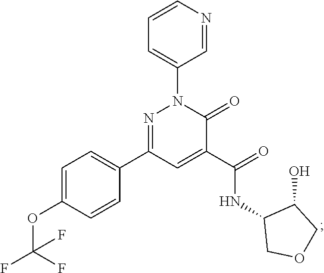 [SMILES: O=C(N[C@H]1COC[C@H]1O)c1cc(-c2ccc(OC(F)(F)F)cc2)nn(-c2cccnc2)c1=O]

N-[(trans)-4-Hydroxytetrahydrofuran-3-yl]-3-oxo-2-(pyridin-3-yl)-6-[4-(trifluoromethoxy)phenyl]-2,3-dihydropyridazine-4-carboxamide

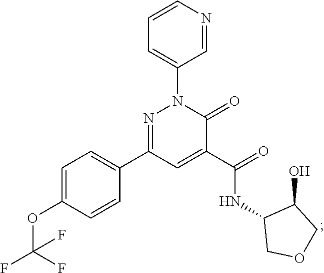 [SMILES: O=C(N[C@H]1COC[C@@H]1O)c1cc(-c2ccc(OC(F)(F)F)cc2)nn(-c2cccnc2)c1=O]

N-[(trans)-4-Hydroxytetrahydrofuran-3-yl]-3-oxo-2-(pyridin-3-yl)-6-[4-(trifluoromethoxy)phenyl]-2,3-dihydropyridazine-4-carboxamide, Enantiomer 1

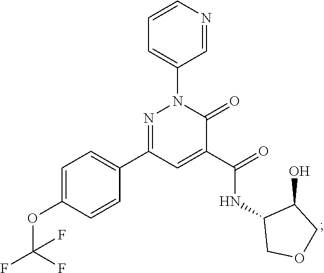 [SMILES: O=C(N[C@H]1COC[C@@H]1O)c1cc(-c2ccc(OC(F)(F)F)cc2)nn(-c2cccnc2)c1=O]

N-[(trans)-4-Hydroxytetrahydrofuran-3-yl]-3-oxo-2-(pyridin-3-yl)-6-[4-(trifluoromethoxy)phenyl]-2,3-dihydropyridazine-4-carboxamide, Enantiomer 2

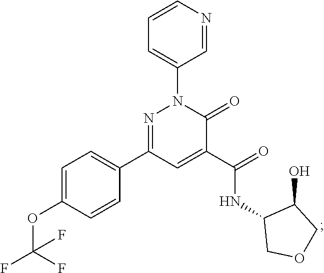 [SMILES: O=C(N[C@H]1COC[C@@H]1O)c1cc(-c2ccc(OC(F)(F)F)cc2)nn(-c2cccnc2)c1=O]

6-(4-Chloro-2-fluorophenyl)-N-[(2S)-1-hydroxypropan-2-yl]-3-oxo-2-(pyridin-3-yl)-2,3-dihydropyridazine-4-carboxamide

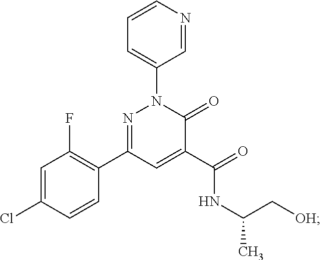 [SMILES: C[C@@H](CO)NC(=O)c1cc(-c2ccc(Cl)cc2F)nn(-c2cccnc2)c1=O]

3-(4-Chlorophenyl)-N-[(1S,2R)-2-hydroxycyclohexyl]-6-oxo-6H-1,4′-bipyridazine-5-carboxamide

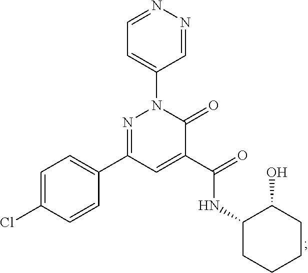 [SMILES: O=C(N[C@H]1CCCC[C@H]1O)c1cc(-c2ccc(Cl)cc2)nn(-c2ccnnc2)c1=O]

6-[6-(Difluoromethyl)pyridin-3-yl]-N-[(1S,2S)-2-hydroxycyclopentyl]-3-oxo-2-(pyridin-3-yl)-2,3-dihydropyridazine-4-carboxamide

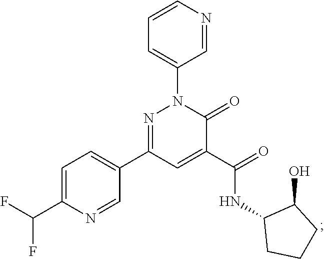 [SMILES: O=C(N[C@H]1CCC[C@@H]1O)c1cc(-c2ccc(C(F)F)nc2)nn(-c2cccnc2)c1=O]

N-[(2S)-1-Hydroxypropan-2-yl]-3-oxo-2-(pyridin-3-yl)-6-[6-(trifluoromethyl)pyridin-3-yl]-2,3-dihydropyridazine-4-carboxamide

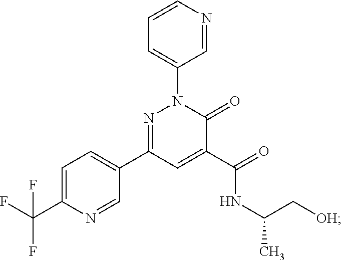 [SMILES: C[C@@H](CO)NC(=O)c1cc(-c2ccc(C(F)(F)F)nc2)nn(-c2cccnc2)c1=O]

3-(4-Chlorophenyl)-N-[(1 S,2R)-2-hydroxycyclopentyl]-6-oxo-6H-1,4′-bipyridazine-5-carboxamide

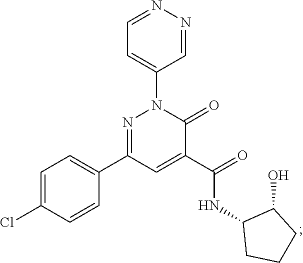 [SMILES: O=C(N[C@H]1CCC[C@H]1O)c1cc(-c2ccc(Cl)cc2)nn(-c2ccnnc2)c1=O]

6-(4-Chlorophenyl)-N-[(trans)-4-hydroxytetrahydrofuran-3-yl]-3-oxo-2-(pyridin-3-yl)-2,3-dihydropyridazine-4-carboxamide

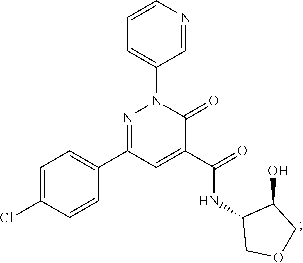 [SMILES: O=C(N[C@H]1COC[C@@H]1O)c1cc(-c2ccc(Cl)cc2)nn(-c2cccnc2)c1=O]

6-(4-Chlorophenyl)-N-[(trans)-4-hydroxytetrahydrofuran-3-yl]-3-oxo-2-(pyridin-3-yl)-2,3-dihydropyridazine-4-carboxamide, Enantiomer 1

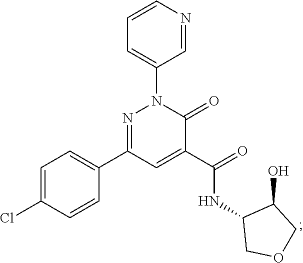 [SMILES: O=C(N[C@H]1COC[C@@H]1O)c1cc(-c2ccc(Cl)cc2)nn(-c2cccnc2)c1=O]

6-(4-Chlorophenyl)-N-[(trans)-4-hydroxytetrahydrofuran-3-yl]-3-oxo-2-(pyridin-3-yl)-2,3-dihydropyridazine-4-carboxamide, Enantiomer 2

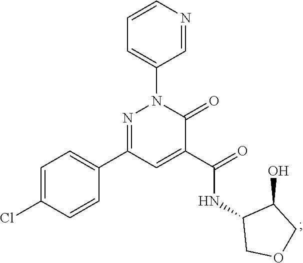 [SMILES: O=C(N[C@H]1COC[C@@H]1O)c1cc(-c2ccc(Cl)cc2)nn(-c2cccnc2)c1=O]

6-(4-Chlorophenyl)-N-[(cis)-4-hydroxytetrahydrofuran-3-yl]-3-oxo-2-(pyridin-3-yl)-2,3-dihydropyridazine-4-carboxamide

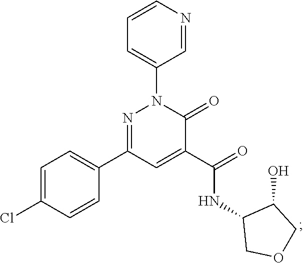 [SMILES: O=C(N[C@H]1COC[C@H]1O)c1cc(-c2ccc(Cl)cc2)nn(-c2cccnc2)c1=O]

6-(4-Chlorophenyl)-N-[(cis)-4-hydroxytetrahydrofuran-3-yl]-3-oxo-2-(pyridin-3-yl)-2,3-dihydropyridazine-4-carboxamide, Enantiomer 1

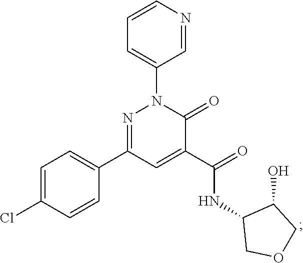 [SMILES: O=C(N[C@H]1COC[C@H]1O)c1cc(-c2ccc(Cl)cc2)nn(-c2cccnc2)c1=O]

6-(4-Chlorophenyl)-N-[(cis)-4-hydroxytetrahydrofuran-3-yl]-3-oxo-2-(pyridin-3-yl)-2,3-dihydropyridazine-4-carboxamide, Enantiomer 2

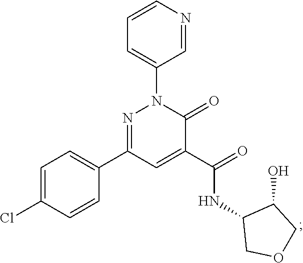 [SMILES: O=C(N[C@H]1COC[C@H]1O)c1cc(-c2ccc(Cl)cc2)nn(-c2cccnc2)c1=O]

3-(4-Chlorophenyl)-N-[(1S,2S)-2-hydroxycyclopentyl]-6-oxo-6H-1,4′-bipyridazine-5-carboxamide

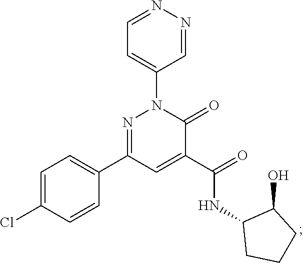 [SMILES: O=C(N[C@H]1CCC[C@@H]1O)c1cc(-c2ccc(Cl)cc2)nn(-c2ccnnc2)c1=O]

1,5-Anhydro-2-({[6-(4-chlorophenyl)-3-oxo-2-(pyridin-3-yl)-2,3-dihydropyridazin-4-yl]carbonyl}amino)-2,4-dideoxy-D-erythro-pentitol

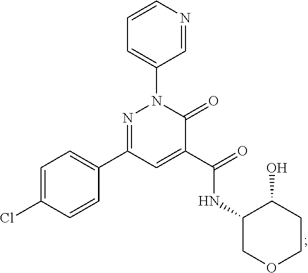 [SMILES: O=C(N[C@H]1COCC[C@H]1O)c1cc(-c2ccc(Cl)cc2)nn(-c2cccnc2)c1=O]

1,5-Anhydro-2,4-dideoxy-2-[({3-oxo-2-(pyridin-3-yl)-6-[4-(trifluoromethoxy)phenyl]-2,3-dihydropyridazin-4-yl}carbonyl)amino]-D-erythro-pentitol

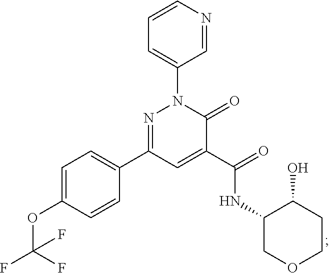 [SMILES: O=C(N[C@H]1COCC[C@H]1O)c1cc(-c2ccc(OC(F)(F)F)cc2)nn(-c2cccnc2)c1=O]

3-(4-Chlorophenyl)-N-[(2S)-1-hydroxypropan-2-yl]-6-oxo-6H-1,4′-bipyridazine-5-carboxamide

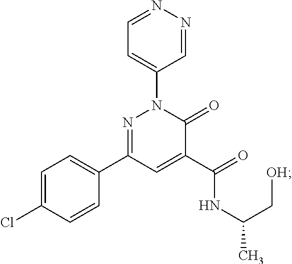 [SMILES: C[C@@H](CO)NC(=O)c1cc(-c2ccc(Cl)cc2)nn(-c2ccnnc2)c1=O]

3-(4-Chlorophenyl)-N-(2-hydroxy-2-methylpropyl)-6-oxo-6H-1,4′-bipyridazine-5-carboxamide

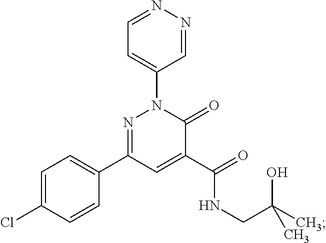 [SMILES: CC(C)(O)CNC(=O)c1cc(-c2ccc(Cl)cc2)nn(-c2ccnnc2)c1=O]

6-(4-Cyanophenyl)-N-[(2S)-1-hydroxypropan-2-yl]-3-oxo-2-(pyridin-3-yl)-2,3-dihydropyridazine-4-carboxamide

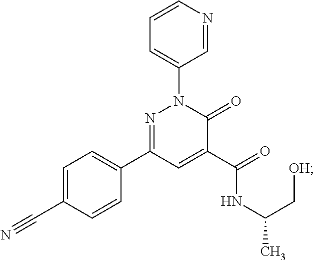 [SMILES: C[C@@H](CO)NC(=O)c1cc(-c2ccc(C#N)cc2)nn(-c2cccnc2)c1=O]

6-[6-(Difluoromethyl)pyridin-3-yl]-N-[(2S)-1-hydroxypropan-2-yl]-3-oxo-2-(pyridin-3-yl)-2,3-dihydropyridazine-4-carboxamide

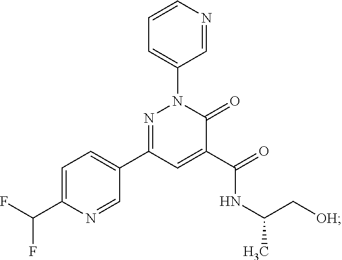 [SMILES: C[C@@H](CO)NC(=O)c1cc(-c2ccc(C(F)F)nc2)nn(-c2cccnc2)c1=O]

3-(4-Chlorophenyl)-N-[(cis)-4-hydroxytetrahydrofuran-3-yl]-6-oxo-6H-1,4′-bipyridazine-5-carboxamide

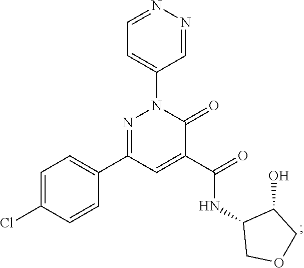 [SMILES: O=C(N[C@H]1COC[C@H]1O)c1cc(-c2ccc(Cl)cc2)nn(-c2ccnnc2)c1=O]

6-[6-(Difluoromethyl) pyridin-3-yl]-N-[(cis)-4-hydroxytetrahydrofuran-3-yl]-3-oxo-2-(pyridin-3-yl)-2,3-dihydropyridazine-4-carboxamide

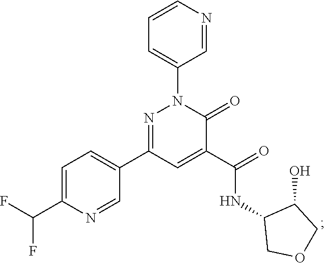 [SMILES: O=C(N[C@H]1COC[C@H]1O)c1cc(-c2ccc(C(F)F)nc2)nn(-c2cccnc2)c1=O]

6-[6-(Difluoromethyl)pyridin-3-yl]-N-[(cis)-4-hydroxytetrahydrofuran-3-yl]-3-oxo-2-(pyridin-3-yl)-2,3-dihydropyridazine-4-carboxamide, Enantiomer 1

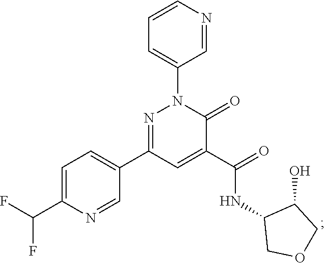 [SMILES: O=C(N[C@H]1COC[C@H]1O)c1cc(-c2ccc(C(F)F)nc2)nn(-c2cccnc2)c1=O]

6-[6-(Difluoromethyl)pyridin-3-yl]-N-[(cis)-4-hydroxytetrahydrofuran-3-yl]-3-oxo-2-(pyridin-3-yl)-2,3-dihydropyridazine-4-carboxamide, Enantiomer 2

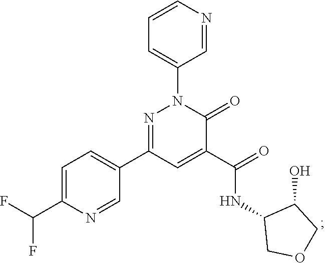 [SMILES: O=C(N[C@H]1COC[C@H]1O)c1cc(-c2ccc(C(F)F)nc2)nn(-c2cccnc2)c1=O]

6-(4-Chlorophenyl)-3-oxo-2-(pyrimidin-5-yl)-N-(1,1,1-trifluoro-3-hydroxy-3-methylbutan-2-yl)-2,3-dihydropyridazine-4-carboxamide

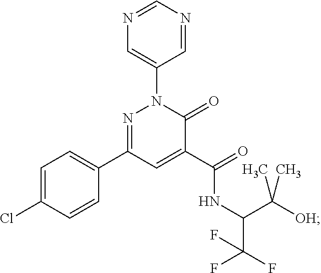 [SMILES: CC(C)(O)C(NC(=O)c1cc(-c2ccc(Cl)cc2)nn(-c2cncnc2)c1=O)C(F)(F)F]

6-(4-Chlorophenyl)-3-oxo-2-(pyrimidin-5-yl)-N-(1,1,1-trifluoro-3-hydroxy-3-methylbutan-2-yl)-2,3-dihydropyridazine-4-carboxamide, Enantiomer 1

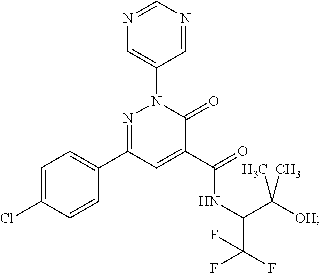 [SMILES: CC(C)(O)C(NC(=O)c1cc(-c2ccc(Cl)cc2)nn(-c2cncnc2)c1=O)C(F)(F)F]

6-(4-Chlorophenyl)-3-oxo-2-(pyrimidin-5-yl)-N-(1,1,1-trifluoro-3-hydroxy-3-methylbutan-2-yl)-2,3-dihydropyridazine-4-carboxamide, Enantiomer 2

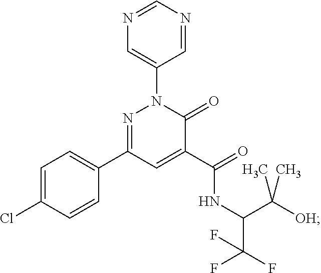 [SMILES: CC(C)(O)C(NC(=O)c1cc(-c2ccc(Cl)cc2)nn(-c2cncnc2)c1=O)C(F)(F)F]

N-[(1-Hydroxycyclopropyl)methyl]-3-oxo-2-(pyridin-3-yl)-6-[4-(trifluoromethyl)phenyl]-2,3-dihydropyridazine-4-carboxamide

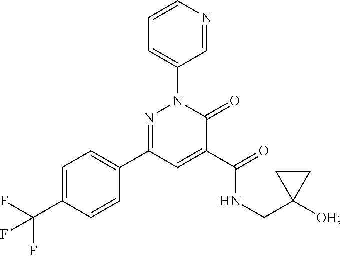 [SMILES: O=C(NCC1(O)CC1)c1cc(-c2ccc(C(F)(F)F)cc2)nn(-c2cccnc2)c1=O]

N-[(1-Hydroxycyclobutyl)methyl]-3-oxo-2-(pyridin-3-yl)-6-[4-(trifluoromethyl)phenyl]-2,3-dihydropyridazine-4-carboxamide

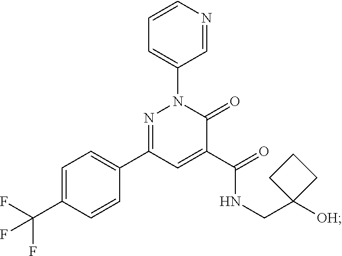 [SMILES: O=C(NCC1(O)CCC1)c1cc(-c2ccc(C(F)(F)F)cc2)nn(-c2cccnc2)c1=O]

(+)-6-(4-Chlorophenyl)-N-[(2S)-1-hydroxypropan-2-yl]-3-oxo-2-(1H-pyrazol-4-yl)-2,3-dihydropyridazine-4-carboxamide

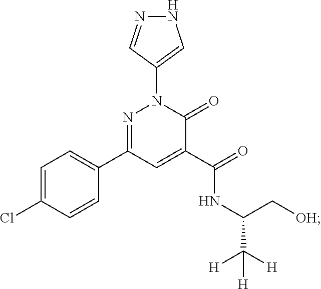 [SMILES: C[C@@H](CO)NC(=O)c1cc(-c2ccc(Cl)cc2)nn(-c2cn[nH]c2)c1=O]

6-[4-(Dimethylamino)phenyl]-N-[(2S)-1-hydroxypropan-2-yl]-3-oxo-2-(pyridin-3-yl)-2,3-dihydropyridazine-4-carboxamide

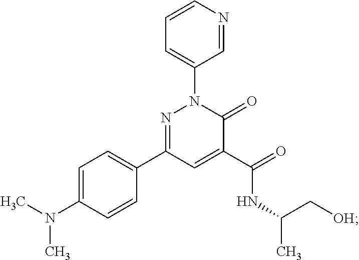 [SMILES: C[C@@H](CO)NC(=O)c1cc(-c2ccc(N(C)C)cc2)nn(-c2cccnc2)c1=O]

6-(4-Chlorophenyl)-N-[(2S)-1-hydroxypropan-2-yl]-2-(1-methyl-1H-pyrazol-3-yl)-3-oxo-2,3-dihydropyridazine-4-carboxamide

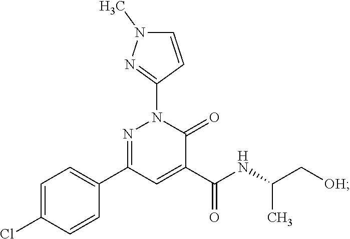 [SMILES: C[C@@H](CO)NC(=O)c1cc(-c2ccc(Cl)cc2)nn(-c2ccn(C)n2)c1=O]

6-(4-Chlorophenyl)-N-[(2S)-1-hydroxypropan-2-yl]-2-(3-methyl-1H-pyrazol-5-yl)-3-oxo-2,3-dihydropyridazine-4-carboxamide

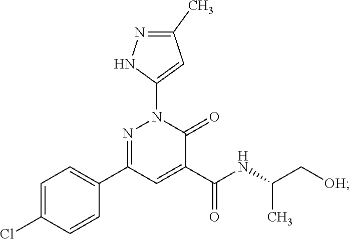 [SMILES: Cc1cc(-n2nc(-c3ccc(Cl)cc3)cc(C(=O)N[C@@H](C)CO)c2=O)[nH]n1]

(+)-6-(4-Chlorophenyl)-N-[(2S)-1-hydroxypropan-2-yl]-3-oxo-2-(1,2-thiazol-4-yl)-2,3-dihydropyridazine-4-carboxamide

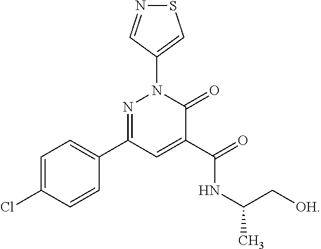 [SMILES: C[C@@H](CO)NC(=O)c1cc(-c2ccc(Cl)cc2)nn(-c2cnsc2)c1=O]

| Structure | IUPAC name |
|---|---|
| 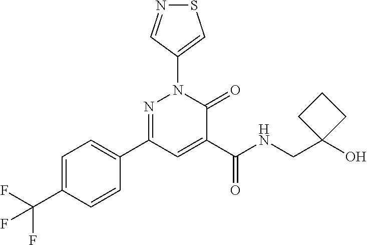 [SMILES: O=C(NCC1(O)CCC1)c1cc(-c2ccc(C(F)(F)F)cc2)nn(-c2cnsc2)c1=O] | N-[(1-Hydroxycyclobutyl)methyl]-3-oxo-2-(1,2-thiazol-4-yl)-6- [4-(trifluoromethyl)phenyl]-2,3-dihydropyridazine-4- carboxamide |
| 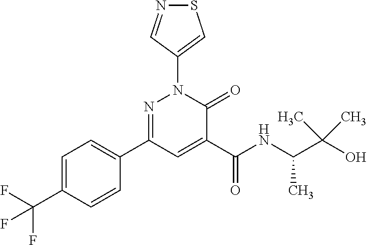 [SMILES: C[C@H](NC(=O)c1cc(-c2ccc(C(F)(F)F)cc2)nn(-c2cnsc2)c1=O)C(C)(C)O] | N-[(2S)-3-Hydroxy-3-methylbutan-2-yl]-3-oxo-2-(1,2-thiazol-4- yl)-6-[4-(trifluoromethyl)phenyl]-2,3-dihydropyridazine-4- carboxamide |
| 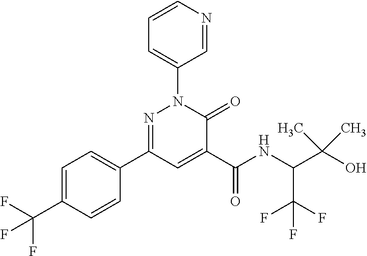 [SMILES: CC(C)(O)C(NC(=O)c1cc(-c2ccc(C(F)(F)F)cc2)nn(-c2cccnc2)c1=O)C(F)(F)F] | (+)-3-Oxo-2-(pyridin-3-yl)-N-(1,1,1-trifluoro-3-hydroxy-3- methylbutan-2-yl)-6-[4-(trifluoromethyl)phenyl]-2,3- dihydropyridazine-4-carboxamide, enantiomer 2 |
| 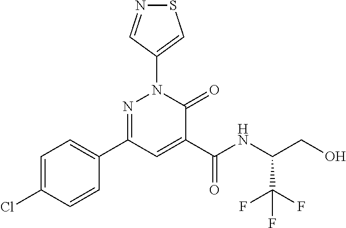 [SMILES: O=C(N[C@H](CO)C(F)(F)F)c1cc(-c2ccc(Cl)cc2)nn(-c2cnsc2)c1=O] | 6-(4-Chlorophenyl)-3-oxo-2-(1,2-thiazol-4-yl)-N-[(2R)-1,1,1- trifluoro-3-hydroxypropan-2-yl]-2,3-dihydropyridazine-4- carboxamide |
| 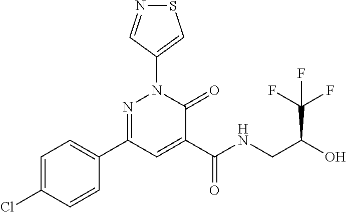 [SMILES: O=C(NC[C@H](O)C(F)(F)F)c1cc(-c2ccc(Cl)cc2)nn(-c2cnsc2)c1=O] | 6-(4-Chlorophenyl)-3-oxo-2-(1,2-thiazol-4-yl)-N-[(2S)-3,3,3- trifluoro-2-hydroxypropyl]-2,3-dihydropyridazine-4- carboxamide |
| 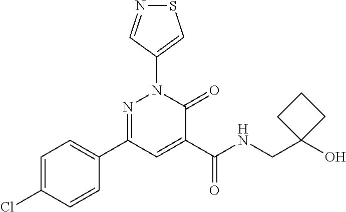 [SMILES: O=C(NCC1(O)CCC1)c1cc(-c2ccc(Cl)cc2)nn(-c2cnsc2)c1=O] | 6-(4-Chlorophenyl)-N-[(1-hydroxycyclobutyl)methyl]-3-oxo-2- (1,2-thiazol-4-yl)-2,3-dihydropyridazine-4-carboxamide |
| 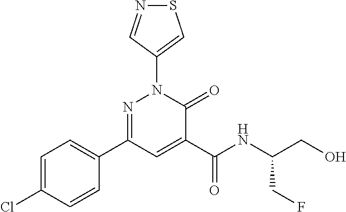 [SMILES: O=C(N[C@H](CO)CF)c1cc(-c2ccc(Cl)cc2)nn(-c2cnsc2)c1=O] | 6-(4-Chlorophenyl)-N-[(2R)-1-fluoro-3-hydroxypropan-2-yl]-3- oxo-2-(1,2-thiazol-4-yl)-2,3-dihydropyridazine-4-carboxamide |
| 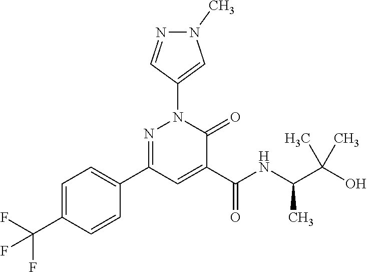 [SMILES: C[C@@H](NC(=O)c1cc(-c2ccc(C(F)(F)F)cc2)nn(-c2cnn(C)c2)c1=O)C(C)(C)O] | N-[(2R)-3-Hydroxy-3-methylbutan-2-yl]-2-(1-methyl-1H- pyrazol-4-yl)-3-oxo-6-[4-(trifluoromethyl)phenyl]-2,3- dihydropyridazine-4-carboxamide |
| 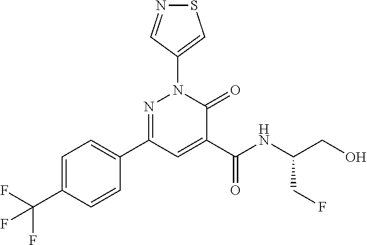 [SMILES: O=C(N[C@H](CO)CF)c1cc(-c2ccc(C(F)(F)F)cc2)nn(-c2cnsc2)c1=O] | N-[(2R)-1-Fluoro-3-hydroxypropan-2-yl]-3-oxo-2-(1,2-thiazol- 4-yl)-6-[4-(trifluoromethyl)phenyl]-2,3-dihydropyridazine-4- carboxamide |
| 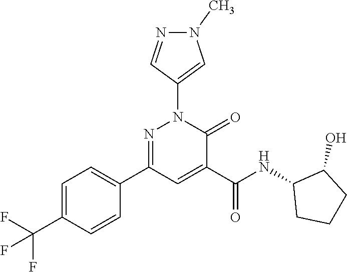 [SMILES: Cn1cc(-n2nc(-c3ccc(C(F)(F)F)cc3)cc(C(=O)N[C@H]3CCC[C@H]3O)c2=O)cn1] | N-[(1S,2R)-2-Hydroxycyclopentyl]-2-(1-methyl-1H-pyrazol-4- yl)-3-oxo-6-[4-(trifluoromethyl)phenyl]-2,3-dihydropyridazine-4- carboxamide |
| 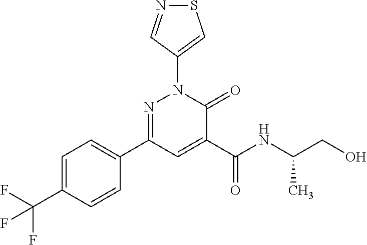 [SMILES: C[C@@H](CO)NC(=O)c1cc(-c2ccc(C(F)(F)F)cc2)nn(-c2cnsc2)c1=O] | N-[(2S)-1-Hydroxypropan-2-yl]-3-oxo-2-(1,2-thiazol-4-yl)-6-[4- (trifluoromethyl)phenyl]-2,3-dihydropyridazine-4-carboxamide |
| 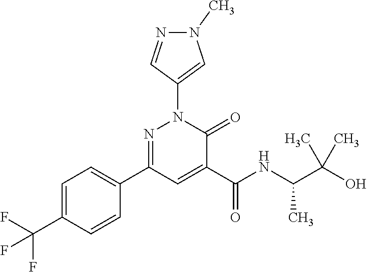 [SMILES: C[C@H](NC(=O)c1cc(-c2ccc(C(F)(F)F)cc2)nn(-c2cnn(C)c2)c1=O)C(C)(C)O] | N-[(2S)-3-Hydroxy-3-methylbutan-2-yl]-2-(1-methyl-1H- pyrazol-4-yl)-3-oxo-6-[4-(trifluoromethyl)phenyl]-2,3- dihydropyridazine-4-carboxamide |
| 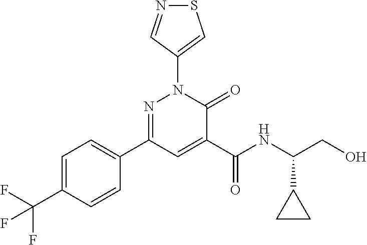 [SMILES: O=C(N[C@H](CO)C1CC1)c1cc(-c2ccc(C(F)(F)F)cc2)nn(-c2cnsc2)c1=O] | N-[(1S)-1-Cyclopropyl-2-hydroxyethyl]-3-oxo-2-(1,2-thiazol-4- yl)-6-[4-(trifluoromethyl)phenyl]-2,3-dihydropyridazine-4- carboxamide |
| 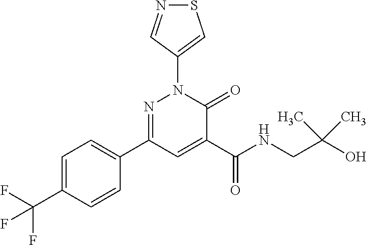 [SMILES: CC(C)(O)CNC(=O)c1cc(-c2ccc(C(F)(F)F)cc2)nn(-c2cnsc2)c1=O] | N-(2-Hydroxy-2-methylpropyl)-3-oxo-2-(1,2-thiazol-4-yl)-6-[4- (trifluoromethyl)phenyl]-2,3-dihydropyridazine-4-carboxamide |
| 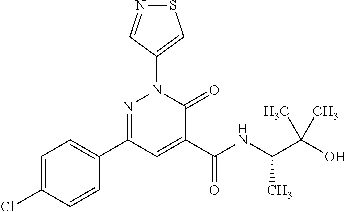 [SMILES: C[C@H](NC(=O)c1cc(-c2ccc(Cl)cc2)nn(-c2cnsc2)c1=O)C(C)(C)O] | 6-(4-Chlorophenyl)-N-[(2S)-3-hydroxy-3-methylbutan-2-yl]-3- oxo-2-(1,2-thiazol-4-yl)-2,3-dihydropyridazine-4-carboxamide |
| 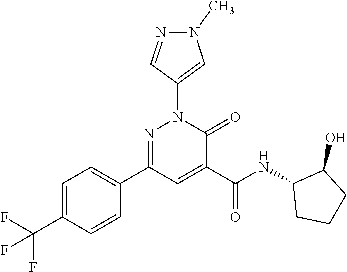 [SMILES: Cn1cc(-n2nc(-c3ccc(C(F)(F)F)cc3)cc(C(=O)N[C@H]3CCC[C@@H]3O)c2=O)cn1] | N-[(1S,2S)-2-Hydroxycyclopentyl]-2-(1-methyl-1H-pyrazol-4- yl)-3-oxo-6-[4-(trifluoromethyl)phenyl]-2,3-dihydropyridazine-4- carboxamide |
| 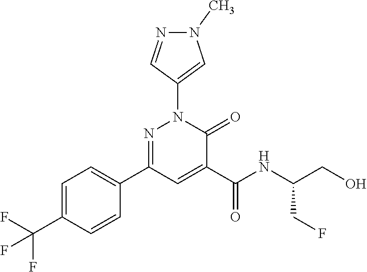 [SMILES: Cn1cc(-n2nc(-c3ccc(C(F)(F)F)cc3)cc(C(=O)N[C@H](CO)CF)c2=O)cn1] | N-[(2R)-1-Fluoro-3-hydroxypropan-2-yl]-2-(1-methyl-1H- pyrazol-4-yl)-3-oxo-6-[4-(trifluoromethyl)phenyl]-2,3- dihydropyridazine-4-carboxamide |
| 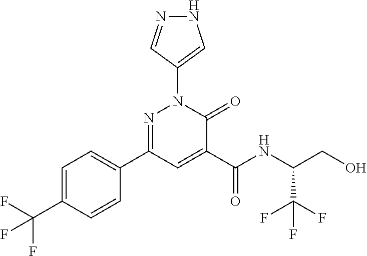 [SMILES: O=C(N[C@H](CO)C(F)(F)F)c1cc(-c2ccc(C(F)(F)F)cc2)nn(-c2cn[nH]c2)c1=O] | 3-Oxo-2-(1H-pyrazol-4-yl)-N-[(2R)-1,1,1-trifluoro-3- hydroxypropan-2-yl]-6-[4-(trifluoromethyl)phenyl]-2,3- dihydropyridazine-4-carboxamide |
| 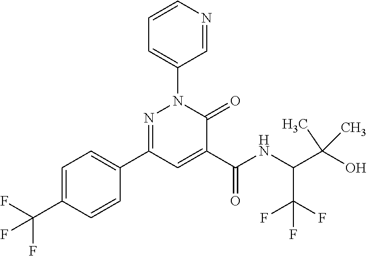 [SMILES: CC(C)(O)C(NC(=O)c1cc(-c2ccc(C(F)(F)F)cc2)nn(-c2cccnc2)c1=O)C(F)(F)F] | 3-Oxo-2-(pyridin-3-yl)-N-(1,1,1-trifluoro-3-hydroxy-3- methylbutan-2-yl)-6-[4-(trifluoromethyl)phenyl]-2,3- dihydropyridazine-4-carboxamide |
| 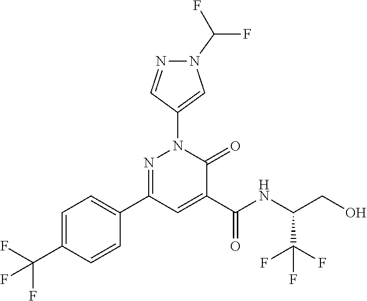 [SMILES: O=C(N[C@H](CO)C(F)(F)F)c1cc(-c2ccc(C(F)(F)F)cc2)nn(-c2cnn(C(F)F)c2)c1=O] | 2-[1-(Difluoromethyl)-1H-pyrazol-4-yl]-3-oxo-N-[(2R)-1,1,1- trifluoro-3-hydroxypropan-2-yl]-6-[4-(trifluoromethyl)phenyl]- 2,3-dihydropyridazine-4-carboxamide |
| 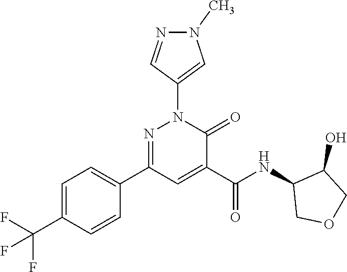 [SMILES: Cn1cc(-n2nc(-c3ccc(C(F)(F)F)cc3)cc(C(=O)N[C@@H]3COC[C@@H]3O)c2=O)cn1] | N-cis-4-Hydroxytetrahydrofuran-3-yl-2-(1-methyl-1H-pyrazol- 4-yl)-3-oxo-6-[4-(trifluoromethyl)phenyl]-2,3-dihydropyridazine- 4-carboxamide |
| 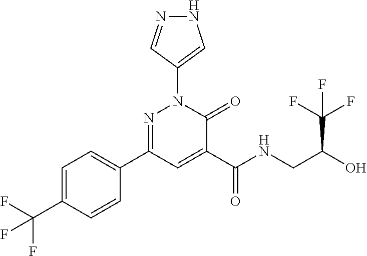 [SMILES: O=C(NC[C@H](O)C(F)(F)F)c1cc(-c2ccc(C(F)(F)F)cc2)nn(-c2cn[nH]c2)c1=O] | 3-Oxo-2-(1H-pyrazol-4-yl)-N-[(2S)-3,3,3-trifluoro-2- hydroxypropyl]-6-[4-(trifluoromethyl)phenyl]-2,3- dihydropyridazine-4-carboxamide |
| 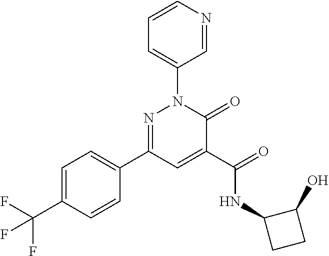 [SMILES: O=C(N[C@@H]1CC[C@@H]1O)c1cc(-c2ccc(C(F)(F)F)cc2)nn(-c2cccnc2)c1=O] | (+)-N-cis-2-Hydroxycyclobutyl-3-oxo-2-(pyridin-3-yl)-6-[4- (trifluoromethyl)phenyl]-2,3-dihydropyridazine-4-carboxamide, enantiomer 1 |
| 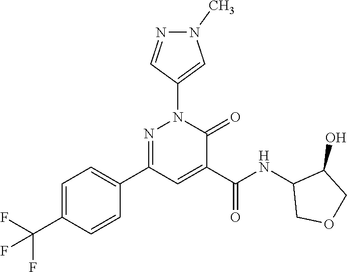 [SMILES: Cn1cc(-n2nc(-c3ccc(C(F)(F)F)cc3)cc(C(=O)NC3COC[C@@H]3O)c2=O)cn1] | (+)-N-cis-4-Hydroxytetrahydrofuran-3-yl-2-(1-methyl-1H- pyrazol-4-yl)-3-oxo-6-[4-(trifluoromethyl)phenyl]-2,3- dihydropyridazine-4-carboxamide, enantiomer 2 |
| 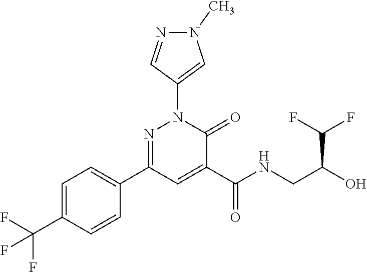 [SMILES: Cn1cc(-n2nc(-c3ccc(C(F)(F)F)cc3)cc(C(=O)NC[C@H](O)C(F)F)c2=O)cn1] | N-[(2S)-3,3-Difluoro-2-hydroxypropyl]-2-(1-methyl-1H-pyrazol- 4-yl)-3-oxo-6-[4-(trifluoromethyl)phenyl]-2,3-dihydropyridazine- 4-carboxamide |
| 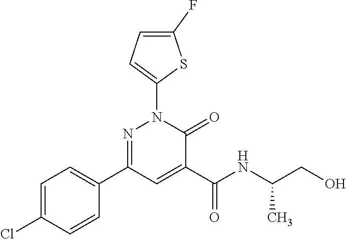 [SMILES: C[C@@H](CO)NC(=O)c1cc(-c2ccc(Cl)cc2)nn(-c2ccc(F)s2)c1=O] | 6-(4-Chlorophenyl)-2-(5-fluoro-2-thienyl)-N-[(2S)-1- hydroxypropan-2-yl]-3-oxo-2,3-dihydropyridazine-4- carboxamide |
| 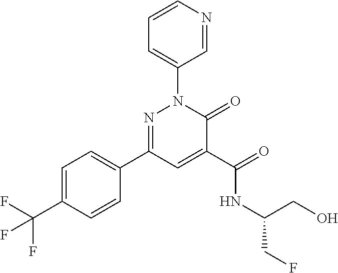 [SMILES: O=C(N[C@H](CO)CF)c1cc(-c2ccc(C(F)(F)F)cc2)nn(-c2cccnc2)c1=O] | N-[(2R)-1-Fluoro-3-hydroxypropan-2-yl]-3-oxo-2-(pyridin-3-yl)- 6-[4-(trifluoromethyl)phenyl]-2,3-dihydropyridazine-4- carboxamide |
| 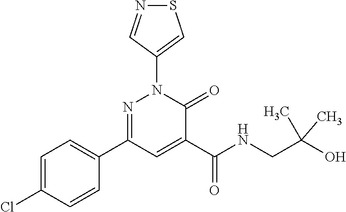 [SMILES: CC(C)(O)CNC(=O)c1cc(-c2ccc(Cl)cc2)nn(-c2cnsc2)c1=O] | 6-(4-Chlorophenyl)-N-(2-hydroxy-2-methylpropyl)-3-oxo-2- (1,2-thiazol-4-yl)-2,3-dihydropyridazine-4-carboxamide |
| 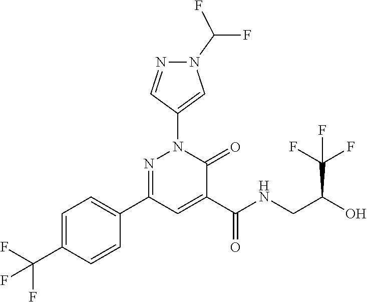 [SMILES: O=C(NC[C@H](O)C(F)(F)F)c1cc(-c2ccc(C(F)(F)F)cc2)nn(-c2cnn(C(F)F)c2)c1=O] | 2-[1-(Difluoromethyl)-1H-pyrazol-4-yl]-3-oxo-N-[(2S)-3,3,3- trifluoro-2-hydroxypropyl]-6-[4-(trifluoromethyl)phenyl]-2,3- dihydropyridazine-4-carboxamide |
| 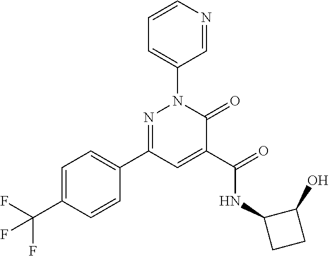 [SMILES: O=C(N[C@@H]1CC[C@@H]1O)c1cc(-c2ccc(C(F)(F)F)cc2)nn(-c2cccnc2)c1=O] | N-cis-2-Hydroxycyclobutyl-3-oxo-2-(pyridin-3-yl)-6-[4- (trifluoromethyl)phenyl]-2,3-dihydropyridazine-4-carboxamide |
| 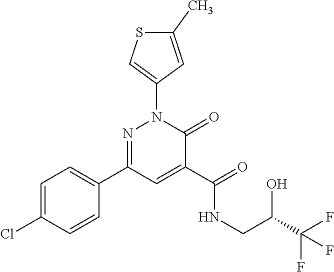 [SMILES: Cc1cc(-n2nc(-c3ccc(Cl)cc3)cc(C(=O)NC[C@H](O)C(F)(F)F)c2=O)cs1] | 6-(4-Chlorophenyl)-2-(5-methyl-3-thienyl)-3-oxo-N-[(2S)-3,3,3- trifluoro-2-hydroxypropyl]-2,3-dihydropyridazine-4- carboxamide |
| 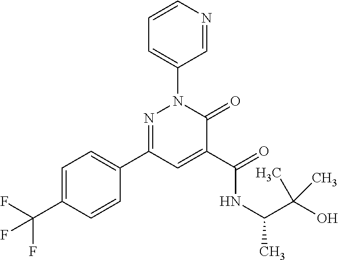 [SMILES: C[C@H](NC(=O)c1cc(-c2ccc(C(F)(F)F)cc2)nn(-c2cccnc2)c1=O)C(C)(C)O] | N-[(2S)-3-Hydroxy-3-methylbutan-2-yl]-3-oxo-2-(pyridin-3-yl)- 6-[4-(trifluoromethyl)phenyl]-2,3-dihydropyridazine-4- carboxamide |
| 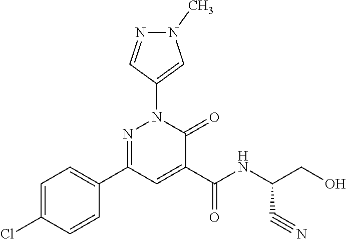 [SMILES: Cn1cc(-n2nc(-c3ccc(Cl)cc3)cc(C(=O)N[C@@H](C#N)CO)c2=O)cn1] | 6-(4-Chlorophenyl)-N-[(1S)-1-cyano-2-hydroxyethyl]-2-(1- methyl-1H-pyrazol-4-yl)-3-oxo-2,3-dihydropyridazine-4- carboxamide |
| 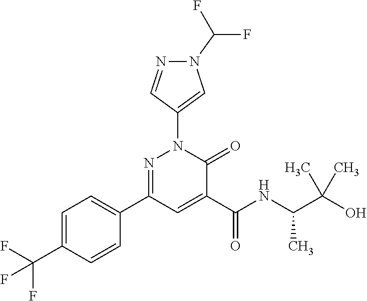 [SMILES: C[C@H](NC(=O)c1cc(-c2ccc(C(F)(F)F)cc2)nn(-c2cnn(C(F)F)c2)c1=O)C(C)(C)O] | 2-[1-(Difluoromethyl)-1H-pyrazol-4-yl]-N-[(2S)-3-hydroxy-3- methylbutan-2-yl]-3-oxo-6-[4-(trifluoromethyl)phenyl]-2,3- dihydropyridazine-4-carboxamide |
| 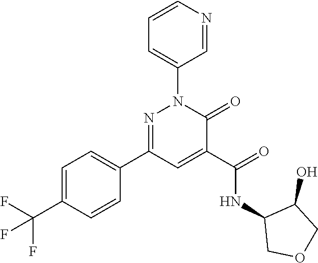 [SMILES: O=C(N[C@@H]1COC[C@@H]1O)c1cc(-c2ccc(C(F)(F)F)cc2)nn(-c2cccnc2)c1=O] | (+)-N-cis-4-Hydroxytetrahydrofuran-3-yl-3-oxo-2-(pyridin-3-yl)- 6-[4-(trifluoromethyl)phenyl]-2,3-dihydropyridazine-4- carboxamide, enantiomer 1 |
| 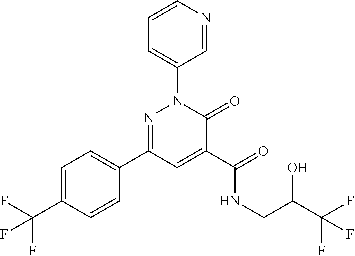 [SMILES: O=C(NCC(O)C(F)(F)F)c1cc(-c2ccc(C(F)(F)F)cc2)nn(-c2cccnc2)c1=O] | (−)-N-(3,3-Difluoro-2-hydroxypropyl)-3-oxo-2-(pyridin-3-yl)-6- [4-(trifluoromethyl)phenyl]-2,3-dihydropyridazine-4- carboxamide, enantiomer 2 |
| 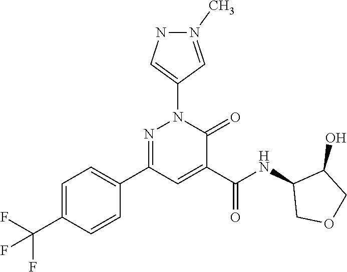 | (−)-N-cis-4-Hydroxytetrahydrofuran-3-yl-2-(1-methyl-1H- pyrazol-4-yl)-3-oxo-6-[4-(trifluoromethyl)phenyl]-2,3- dihydropyridazine-4-carboxamide, enantiomer 1 |
| 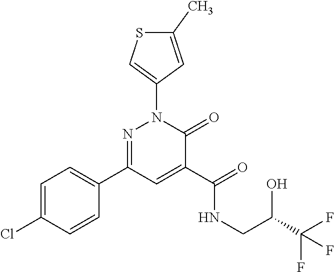 [SMILES: Cc1cc(-n2nc(-c3ccc(Cl)cc3)cc(C(=O)NC[C@H](O)C(F)(F)F)c2=O)cs1] | 6-(4-Chlorophenyl)-2-(5-chloro-3-thienyl)-3-oxo-N-[(2S)-3,3,3- trifluoro-2-hydroxypropyl]-2,3-dihydropyridazine-4- carboxamide |
| 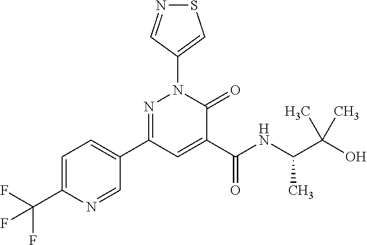 [SMILES: C[C@H](NC(=O)c1cc(-c2ccc(C(F)(F)F)nc2)nn(-c2cnsc2)c1=O)C(C)(C)O] | N-[(2S)-3-Hydroxy-3-methylbutan-2-yl]-3-oxo-2-(1,2-thiazol-4- yl)-6-[6-(trifluoromethyl)pyridin-3-yl]-2,3-dihydropyridazine-4- carboxamide |
| 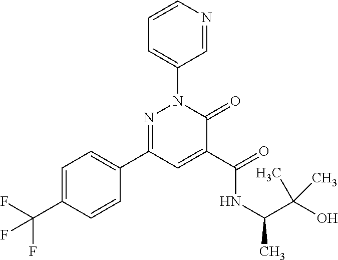 [SMILES: C[C@@H](NC(=O)c1cc(-c2ccc(C(F)(F)F)cc2)nn(-c2cccnc2)c1=O)C(C)(C)O] | N-[(2R)-3-Hydroxy-3-methylbutan-2-yl]-3-oxo-2-(pyridin-3-yl)- 6-[4-(trifluoromethyl)phenyl]-2,3-dihydropyridazine-4- carboxamide |
| 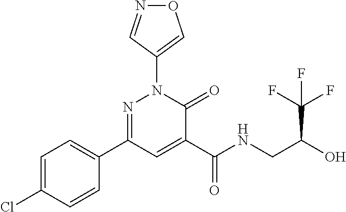 [SMILES: O=C(NC[C@H](O)C(F)(F)F)c1cc(-c2ccc(Cl)cc2)nn(-c2cnoc2)c1=O] | 6-(4-Chlorophenyl)-2-(1,2-oxazol-4-yl)-3-oxo-N-[(2S)-3,3,3- trifluoro-2-hydroxypropyl]-2,3-dihydropyridazine-4- carboxamide |
| 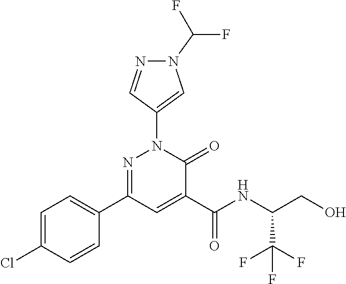 [SMILES: O=C(N[C@H](CO)C(F)(F)F)c1cc(-c2ccc(Cl)cc2)nn(-c2cnn(C(F)F)c2)c1=O] | 6-(4-Chlorophenyl)-2-[1-(difluoromethyl)-1H-pyrazol-4-yl]-3- oxo-N-[(2R)-1,1,1-trifluoro-3-hydroxypropan-2-yl]-2,3- dihydropyridazine-4-carboxamide |
| 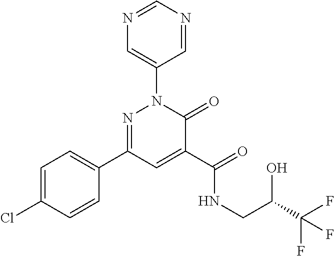 [SMILES: O=C(NC[C@H](O)C(F)(F)F)c1cc(-c2ccc(Cl)cc2)nn(-c2cncnc2)c1=O] | 6-(4-Chlorophenyl)-3-oxo-2-(pyrimidin-5-yl)-N-[(2S)-3,3,3- trifluoro-2-hydroxypropyl]-2,3-dihydropyridazine-4- carboxamide |
| 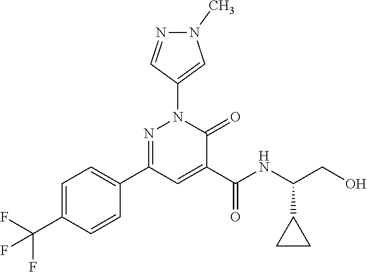 [SMILES: Cn1cc(-n2nc(-c3ccc(C(F)(F)F)cc3)cc(C(=O)N[C@H](CO)C3CC3)c2=O)cn1] | N-[(1S)-1-Cyclopropyl-2-hydroxyethyl]-2-(1-methyl-1H- pyrazol-4-yl)-3-oxo-6-[4-(trifluoromethyl)phenyl]-2,3- dihydropyridazine-4-carboxamide |
| 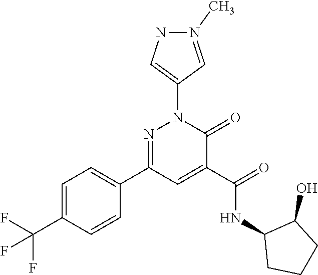 | N-[(1R,2S)-2-Hydroxycyclopentyl]-2-(1-methyl-1H-pyrazol-4- yl)-3-oxo-6-[4-(trifluoromethyl)phenyl]-2,3-dihydropyridazine-4- carboxamide |
| 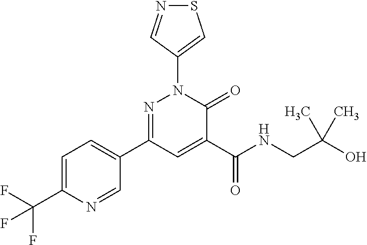 [SMILES: CC(C)(O)CNC(=O)c1cc(-c2ccc(C(F)(F)F)nc2)nn(-c2cnsc2)c1=O] | N-(2-Hydroxy-2-methylpropyl)-3-oxo-2-(1,2-thiazol-4-yl)-6-[6- (trifluoromethyl)pyridin-3-yl]-2,3-dihydropyridazine-4- carboxamide |
| 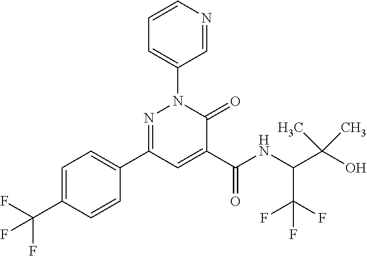 [SMILES: CC(C)(O)C(NC(=O)c1cc(-c2ccc(C(F)(F)F)cc2)nn(-c2cccnc2)c1=O)C(F)(F)F] | (−)-3-Oxo-2-(pyridin-3-yl)-N-(1,1,1-trifluoro-3-hydroxy-3- methylbutan-2-yl)-6-[4-(trifluoromethyl)phenyl]-2,3- dihydropyridazine-4-carboxamide, enantiomer 1 |
| 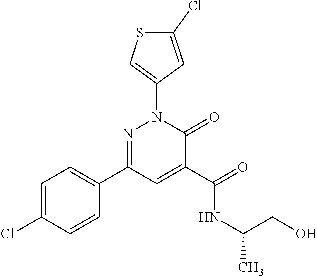 [SMILES: C[C@@H](CO)NC(=O)c1cc(-c2ccc(Cl)cc2)nn(-c2csc(Cl)c2)c1=O] | 6-(4-Chlorophenyl)-N-[(2S)-1-hydroxypropan-2-yl]-2-(5- methyl-3-thienyl)-3-oxo-2,3-dihydropyridazine-4-carboxamide |
| 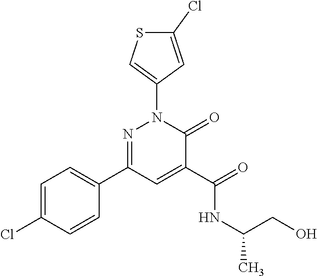 [SMILES: C[C@@H](CO)NC(=O)c1cc(-c2ccc(Cl)cc2)nn(-c2csc(Cl)c2)c1=O] | 6-(4-Chlorophenyl)-2-(5-chloro-3-thienyl)-N-[(2S)-1- hydroxypropan-2-yl]-3-oxo-2,3-dihydropyridazine-4- carboxamide |
| 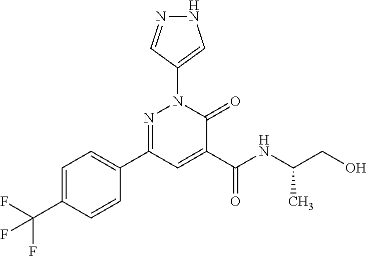 [SMILES: C[C@@H](CO)NC(=O)c1cc(-c2ccc(C(F)(F)F)cc2)nn(-c2cn[nH]c2)c1=O] | N-[(2S)-1-Hydroxypropan-2-yl]-3-oxo-2-(1H-pyrazol-4-yl)-6-[4- (trifluoromethyl)phenyl]-2,3-dihydropyridazine-4-carboxamide |
| 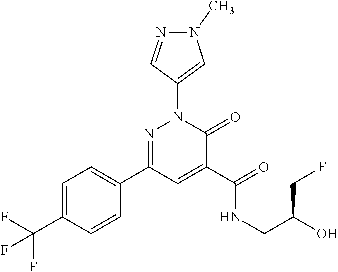 [SMILES: Cn1cc(-n2nc(-c3ccc(C(F)(F)F)cc3)cc(C(=O)NC[C@H](O)CF)c2=O)cn1] | N-[(2S)-3-Fluoro-2-hydroxypropyl]-2-(1-methyl-1H-pyrazol-4- yl)-3-oxo-6-[4-(trifluoromethyl)phenyl]-2,3-dihydropyridazine-4- carboxamide |
| 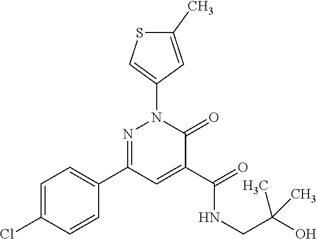 [SMILES: Cc1cc(-n2nc(-c3ccc(Cl)cc3)cc(C(=O)NCC(C)(C)O)c2=O)cs1] | 6-(4-Chlorophenyl)-N-(2-hydroxy-2-methylpropyl)-2-(5-methyl- 3-thienyl)-3-oxo-2,3-dihydropyridazine-4-carboxamide |
| 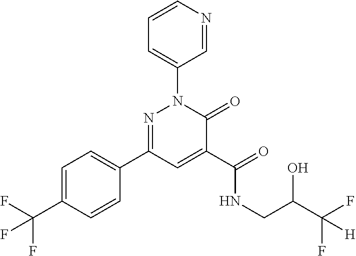 [SMILES: O=C(NCC(O)C(F)F)c1cc(-c2ccc(C(F)(F)F)cc2)nn(-c2cccnc2)c1=O] | (+)-N-(3,3-Difluoro-2-hydroxypropyl)-3-oxo-2-(pyridin-3-yl)-6- [4-(trifluoromethyl)phenyl]-2,3-dihydropyridazine-4- carboxamide, enantiomer 1 |
| 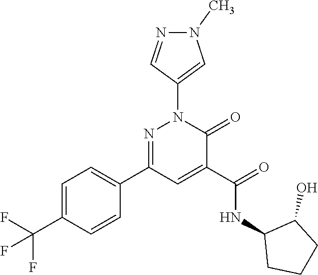 [SMILES: Cn1cc(-n2nc(-c3ccc(C(F)(F)F)cc3)cc(C(=O)N[C@@H]3CCC[C@H]3O)c2=O)cn1] | N-[(1R,2R)-2-Hydroxycyclopentyl]-2-(1-methyl-1H-pyrazol-4- yl)-3-oxo-6-[4-(trifluoromethyl)phenyl]-2,3-dihydropyridazine-4- carboxamide |
| 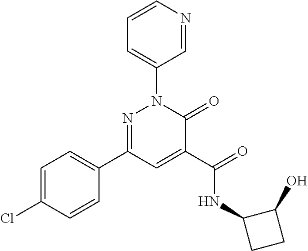 [SMILES: O=C(N[C@@H]1CC[C@@H]1O)c1cc(-c2ccc(Cl)cc2)nn(-c2cccnc2)c1=O] | (+)-6-(4-Chlorophenyl)-N-cis-2-hydroxycyclobutyl-3-oxo-2- (pyridin-3-yl)-2,3-dihydropyridazine-4-carboxamide, enantiomer 1 |
| 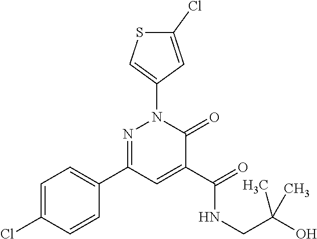 [SMILES: CC(C)(O)CNC(=O)c1cc(-c2ccc(Cl)cc2)nn(-c2csc(Cl)c2)c1=O] | 6-(4-Chlorophenyl)-2-(5-chloro-3-thienyl)-N-(2-hydroxy-2- methylpropyl)-3-oxo-2,3-dihydropyridazine-4-carboxamide |
| 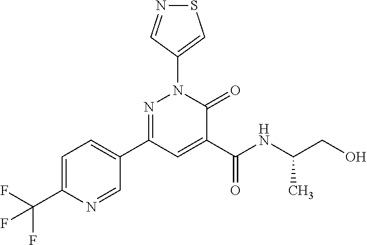 [SMILES: C[C@@H](CO)NC(=O)c1cc(-c2ccc(C(F)(F)F)nc2)nn(-c2cnsc2)c1=O] | N-[(2S)-1-Hydroxypropan-2-yl]-3-oxo-2-(1,2-thiazol-4-yl)-6-[6- (trifluoromethyl)pyridin-3-yl]-2,3-dihydropyridazine-4- carboxamide |
| 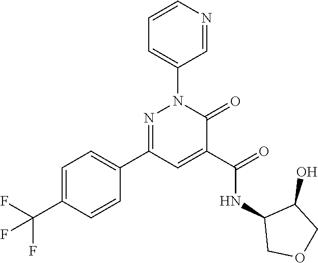 [SMILES: O=C(N[C@@H]1COC[C@@H]1O)c1cc(-c2ccc(C(F)(F)F)cc2)nn(-c2cccnc2)c1=O] | (−)-N-cis-4-Hydroxytetrahydrofuran-3-yl-3-oxo-2-(pyridin-3-yl)- 6-[4-(trifluoromethyl)phenyl]-2,3-dihydropyridazine-4- carboxamide, enantiomer 2 |
| 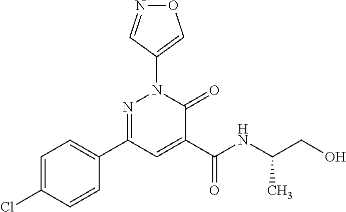 [SMILES: C[C@@H](CO)NC(=O)c1cc(-c2ccc(Cl)cc2)nn(-c2cnoc2)c1=O] | 6-(4-Chlorophenyl)-N-[(2S)-1-hydroxypropan-2-yl]-2-(1,2- oxazol-4-yl)-3-oxo-2,3-dihydropyridazine-4-carboxamide |
| 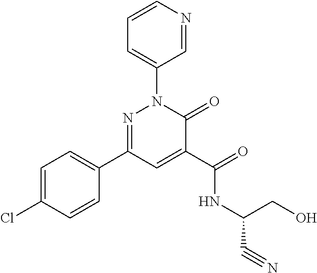 [SMILES: N#C[C@@H](CO)NC(=O)c1cc(-c2ccc(Cl)cc2)nn(-c2cccnc2)c1=O] | 6-(4-Chlorophenyl)-N-[(1S)-1-cyano-2-hydroxyethyl]-3-oxo-2- (pyridin-3-yl)-2,3-dihydropyridazine-4-carboxamide |
| 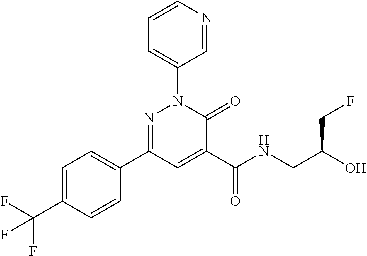 [SMILES: O=C(NC[C@H](O)CF)c1cc(-c2ccc(C(F)(F)F)cc2)nn(-c2cccnc2)c1=O] | N-[(2S)-3-Fluoro-2-hydroxypropyl]-3-oxo-2-(pyridin-3-yl)-6-[4- (trifluoromethyl)phenyl]-2,3-dihydropyridazine-4-carboxamide |
| 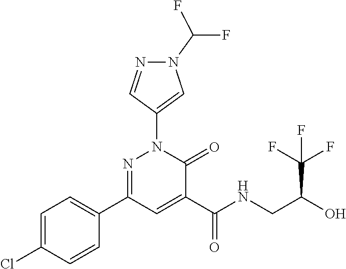 [SMILES: O=C(NC[C@H](O)C(F)(F)F)c1cc(-c2ccc(Cl)cc2)nn(-c2cnn(C(F)F)c2)c1=O] | 6-(4-Chlorophenyl)-2-[1-(difluoromethyl)-1H-pyrazol-4-yl]-3- oxo-N-[(2S)-3,3,3-trifluoro-2-hydroxypropyl]-2,3- dihydropyridazine-4-carboxamide |
| 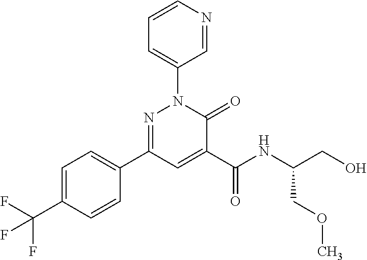 [SMILES: COC[C@@H](CO)NC(=O)c1cc(-c2ccc(C(F)(F)F)cc2)nn(-c2cccnc2)c1=O] | N-[(2R)-1-Hydroxy-3-methoxypropan-2-yl]-3-oxo-2-(pyridin-3- yl)-6-[4-(trifluoromethyl)phenyl]-2,3-dihydropyridazine-4- carboxamide |
| 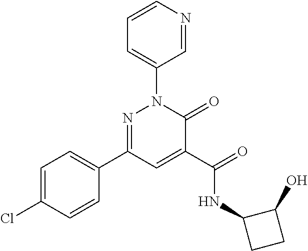 [SMILES: O=C(N[C@@H]1CC[C@@H]1O)c1cc(-c2ccc(Cl)cc2)nn(-c2cccnc2)c1=O] | 6-(4-Chlorophenyl)-N-cis-2-hydroxycyclobutyl-3-oxo-2- (pyridin-3-yl)-2,3-dihydropyridazine-4-carboxamide |
| 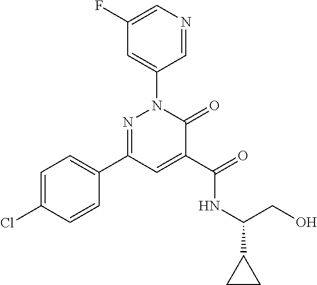 [SMILES: O=C(N[C@H](CO)C1CC1)c1cc(-c2ccc(Cl)cc2)nn(-c2cncc(F)c2)c1=O] | 6-(4-Chlorophenyl)-N-[(1S)-1-cyclopropyl-2-hydroxyethyl]-2- (5-fluoropyridin-3-yl)-3-oxo-2,3-dihydropyridazine-4- carboxamide |
| 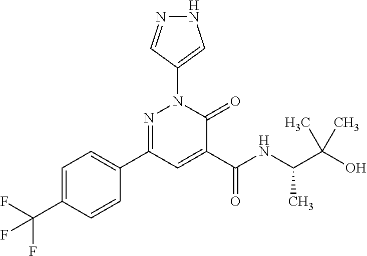 [SMILES: C[C@H](NC(=O)c1cc(-c2ccc(C(F)(F)F)cc2)nn(-c2cn[nH]c2)c1=O)C(C)(C)O] | N-[(2S)-3-Hydroxy-3-methylbutan-2-yl]-3-oxo-2-(1H-pyrazol-4- yl)-6-[4-(trifluoromethyl)phenyl]-2,3-dihydropyridazine-4- carboxamide |
| 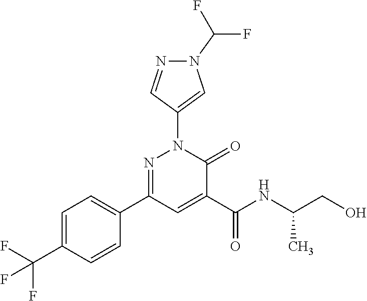 [SMILES: C[C@@H](CO)NC(=O)c1cc(-c2ccc(C(F)(F)F)cc2)nn(-c2cnn(C(F)F)c2)c1=O] | 2-[1-(Difluoromethyl)-1H-pyrazol-4-yl]-N-[(2S)-1- hydroxypropan-2-yl]-3-oxo-6-[4-(trifluoromethyl)phenyl]-2,3- dihydropyridazine-4-carboxamide |
| 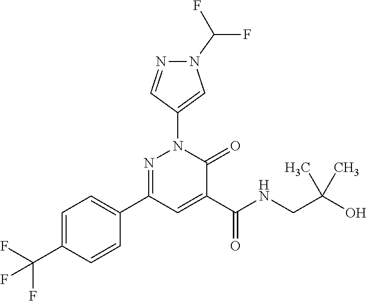 [SMILES: CC(C)(O)CNC(=O)c1cc(-c2ccc(C(F)(F)F)cc2)nn(-c2cnn(C(F)F)c2)c1=O] | 2-[1-(Difluoromethyl)-1H-pyrazol-4-yl]-N-(2-hydroxy-2- methylpropyl)-3-oxo-6-[4-(trifluoromethyl)phenyl]-2,3- dihydropyridazine-4-carboxamide |
| 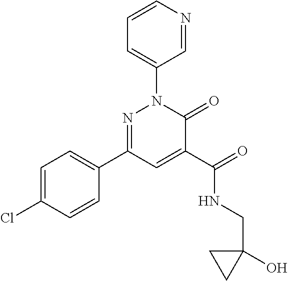 [SMILES: O=C(NCC1(O)CC1)c1cc(-c2ccc(Cl)cc2)nn(-c2cccnc2)c1=O] | 6-(4-Chlorophenyl)-N-[(1-hydroxycyclopropyl)methyl]-3-oxo-2- (pyridin-3-yl)-2,3-dihydropyridazine-4-carboxamide |
| 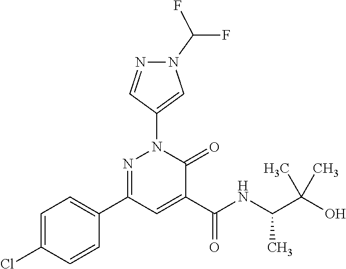 [SMILES: C[C@H](NC(=O)c1cc(-c2ccc(Cl)cc2)nn(-c2cnn(C(F)F)c2)c1=O)C(C)(C)O] | 6-(4-Chlorophenyl)-2-[1-(difluoromethyl)-1H-pyrazol-4-yl]-N- [(2S)-3-hydroxy-3-methylbutan-2-yl]-3-oxo-2,3- dihydropyridazine-4-carboxamide |
| 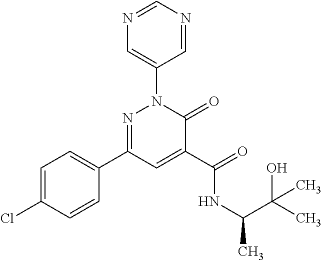 [SMILES: C[C@@H](NC(=O)c1cc(-c2ccc(Cl)cc2)nn(-c2cncnc2)c1=O)C(C)(C)O] | 6-(4-Chlorophenyl)-N-[(2R)-3-hydroxy-3-methylbutan-2-yl]-3- oxo-2-(pyrimidin-5-yl)-2,3-dihydropyridazine-4-carboxamide |
| 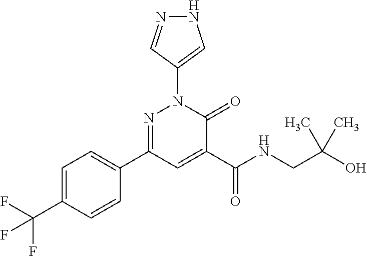 [SMILES: CC(C)(O)CNC(=O)c1cc(-c2ccc(C(F)(F)F)cc2)nn(-c2cn[nH]c2)c1=O] | N-(2-Hydroxy-2-methylpropyl)-3-oxo-2-(1H-pyrazol-4-yl)-6-[4- (trifluoromethyl)phenyl]-2,3-dihydropyridazine-4-carboxamide |
| 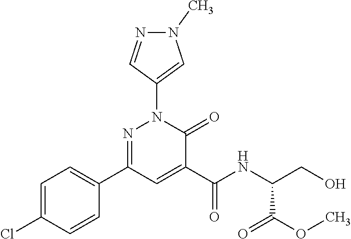 [SMILES: COC(=O)[C@@H](CO)NC(=O)c1cc(-c2ccc(Cl)cc2)nn(-c2cnn(C)c2)c1=O] | Methyl N-{[6-(4-chlorophenyl)-2-(1-methyl-1H-pyrazol-4-yl)-3- oxo-2,3-dihydropyridazin-4-yl]carbonyl}-D-serinate |
| 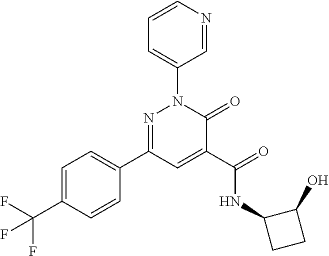 [SMILES: O=C(N[C@@H]1CC[C@@H]1O)c1cc(-c2ccc(C(F)(F)F)cc2)nn(-c2cccnc2)c1=O] | (−)-N-cis-2-Hydroxycyclobutyl-3-oxo-2-(pyridin-3-yl)-6-[4- (trifluoromethyl)phenyl]-2,3-dihydropyridazine-4-carboxamide, enantiomer 2 |
| 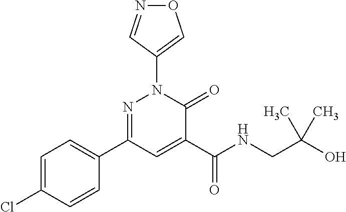 [SMILES: CC(C)(O)CNC(=O)c1cc(-c2ccc(Cl)cc2)nn(-c2cnoc2)c1=O] | 6-(4-Chlorophenyl)-N-(2-hydroxy-2-methylpropyl)-2-(1,2- oxazol-4-yl)-3-oxo-2,3-dihydropyridazine-4-carboxamide |
| 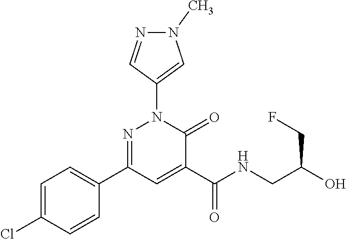 [SMILES: Cn1cc(-n2nc(-c3ccc(Cl)cc3)cc(C(=O)NC[C@H](O)CF)c2=O)cn1] | 6-(4-Chlorophenyl)-N-[(2S)-3-fluoro-2-hydroxypropyl]-2-(1- methyl-1H-pyrazol-4-yl)-3-oxo-2,3-dihydropyridazine-4- carboxamide |
| 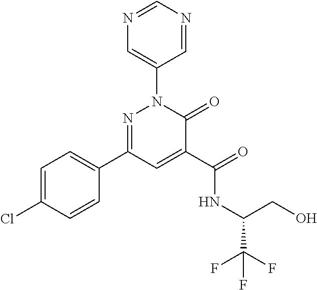 [SMILES: O=C(N[C@H](CO)C(F)(F)F)c1cc(-c2ccc(Cl)cc2)nn(-c2cncnc2)c1=O] | 6-(4-Chlorophenyl)-3-oxo-2-(pyrimidin-5-yl)-N-[(2R)-1,1,1- trifluoro-3-hydroxypropan-2-yl]-2,3-dihydropyridazine-4- carboxamide |
| 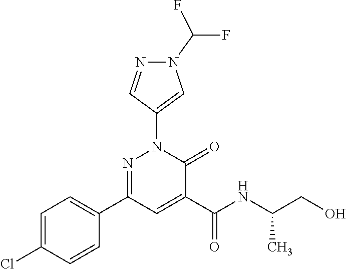 [SMILES: C[C@@H](CO)NC(=O)c1cc(-c2ccc(Cl)cc2)nn(-c2cnn(C(F)F)c2)c1=O] | 6-(4-Chlorophenyl)-2-[1-(difluoromethyl)-1H-pyrazol-4-yl]-N- [(2S)-1-hydroxypropan-2-yl]-3-oxo-2,3-dihydropyridazine-4- carboxamide |
| 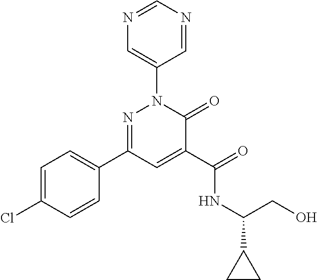 [SMILES: O=C(N[C@H](CO)C1CC1)c1cc(-c2ccc(Cl)cc2)nn(-c2cncnc2)c1=O] | 6-(4-Chlorophenyl)-N-[(1S)-1-cyclopropyl-2-hydroxyethyl]-3- oxo-2-(pyrimidin-5-yl)-2,3-dihydropyridazine-4-carboxamide |
| 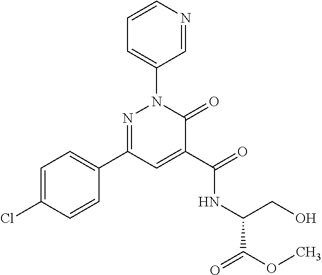 [SMILES: COC(=O)[C@@H](CO)NC(=O)c1cc(-c2ccc(Cl)cc2)nn(-c2cccnc2)c1=O] | Methyl N-{[6-(4-chlorophenyl)-3-oxo-2-(pyridin-3-yl)-2,3- dihydropyridazin-4-yl]carbonyl}-D-serinate |
| 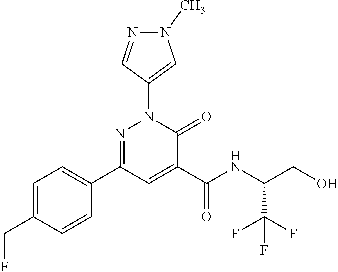 [SMILES: Cn1cc(-n2nc(-c3ccc(CF)cc3)cc(C(=O)N[C@H](CO)C(F)(F)F)c2=O)cn1] | 6-[4-(Fluoromethyl)phenyl]-2-(1-methyl-1H-pyrazol-4-yl)-3- oxo-N-[(2R)-1,1,1-trifluoro-3-hydroxypropan-2-yl]-2,3- dihydropyridazine-4-carboxamide |
| 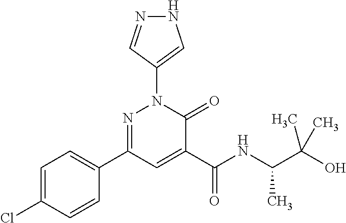 [SMILES: C[C@H](NC(=O)c1cc(-c2ccc(Cl)cc2)nn(-c2cn[nH]c2)c1=O)C(C)(C)O] | 6-(4-Chlorophenyl)-N-[(2S)-3-hydroxy-3-methylbutan-2-yl]-3- oxo-2-(1H-pyrazol-4-yl)-2,3-dihydropyridazine-4-carboxamide |
| 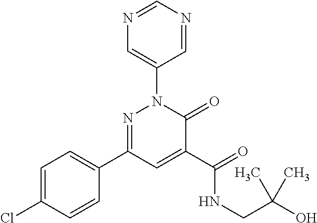 [SMILES: CC(C)(O)CNC(=O)c1cc(-c2ccc(Cl)cc2)nn(-c2cncnc2)c1=O] | 6-(4-Chlorophenyl)-N-(2-hydroxy-2-methylpropyl)-3-oxo-2- (pyrimidin-5-yl)-2,3-dihydropyridazine-4-carboxamide |
| 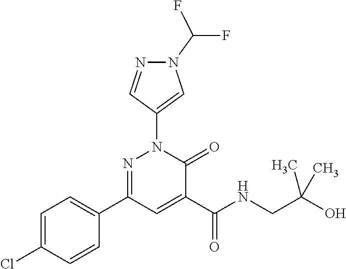 [SMILES: CC(C)(O)CNC(=O)c1cc(-c2ccc(Cl)cc2)nn(-c2cnn(C(F)F)c2)c1=O] | 6-(4-Chlorophenyl)-2-[1-(difluoromethyl)-1H-pyrazol-4-yl]-N- (2-hydroxy-2-methylpropyl)-3-oxo-2,3-dihydropyridazine-4- carboxamide |
| 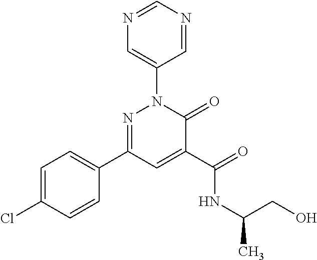 [SMILES: C[C@H](CO)NC(=O)c1cc(-c2ccc(Cl)cc2)nn(-c2cncnc2)c1=O] | 6-(4-Chlorophenyl)-N-[(2R)-1-hydroxypropan-2-yl]-3-oxo-2- (pyrimidin-5-yl)-2,3-dihydropyridazine-4-carboxamide |
| 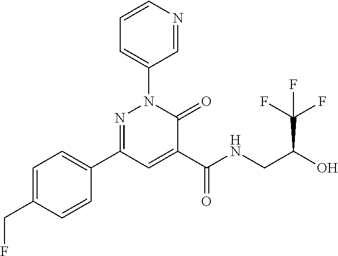 [SMILES: O=C(NC[C@H](O)C(F)(F)F)c1cc(-c2ccc(CF)cc2)nn(-c2cccnc2)c1=O] | 6-[4-(Fluoromethyl)phenyl]-3-oxo-2-(pyridin-3-yl)-N-[(2S)- 3,3,3-trifluoro-2-hydroxypropyl]-2,3-dihydropyridazine-4- carboxamide |
| 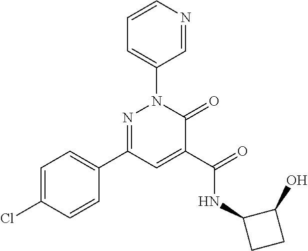 [SMILES: O=C(N[C@@H]1CC[C@@H]1O)c1cc(-c2ccc(Cl)cc2)nn(-c2cccnc2)c1=O] | (−)-6-(4-Chlorophenyl)-N-cis-2-hydroxycyclobutyl-3-oxo-2- (pyridin-3-yl)-2,3-dihydropyridazine-4-carboxamide, enantiomer 2 |
| 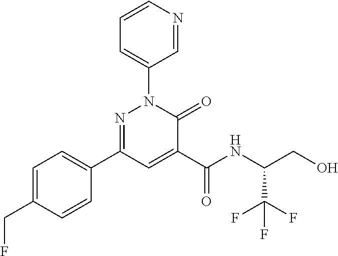 [SMILES: O=C(N[C@H](CO)C(F)(F)F)c1cc(-c2ccc(CF)cc2)nn(-c2cccnc2)c1=O] | 6-[4-(Fluoromethyl)phenyl]-3-oxo-2-(pyridin-3-yl)-N-[(2R)- 1,1,1-trifluoro-3-hydroxypropan-2-yl]-2,3-dihydropyridazine-4- carboxamide |
| 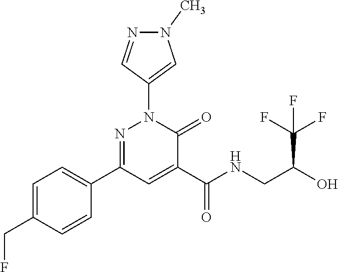 [SMILES: Cn1cc(-n2nc(-c3ccc(CF)cc3)cc(C(=O)NC[C@H](O)C(F)(F)F)c2=O)cn1] | 6-[4-(Fluoromethyl)phenyl]-2-(1-methyl-1H-pyrazol-4-yl)-3- oxo-N-[(2S)-3,3,3-trifluoro-2-hydroxypropyl]-2,3- dihydropyridazine-4-carboxamide |
| 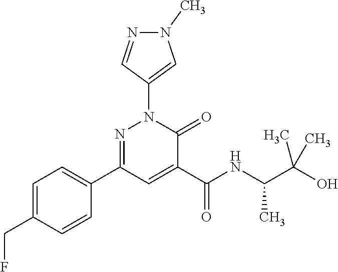 [SMILES: C[C@H](NC(=O)c1cc(-c2ccc(CF)cc2)nn(-c2cnn(C)c2)c1=O)C(C)(C)O] | 6-[4-(Fluoromethyl)phenyl]-N-[(2S)-3-hydroxy-3-methylbutan- 2-yl]-2-(1-methyl-1H-pyrazol-4-yl)-3-oxo-2,3- dihydropyridazine-4-carboxamide |
| 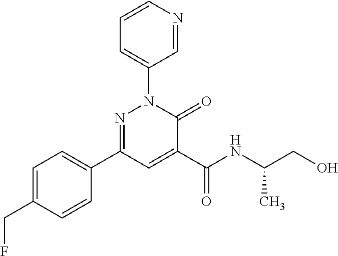 [SMILES: C[C@@H](CO)NC(=O)c1cc(-c2ccc(CF)cc2)nn(-c2cccnc2)c1=O] | 6-[4-(Fluoromethyl)phenyl]-N-[(2S)-1-hydroxypropan-2-yl]-3- oxo-2-(pyridin-3-yl)-2,3-dihydropyridazine-4-carboxamide |
| 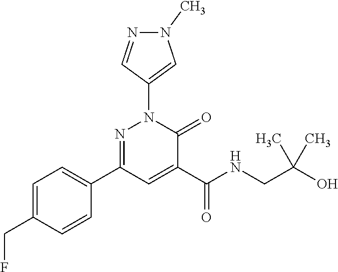 [SMILES: Cn1cc(-n2nc(-c3ccc(CF)cc3)cc(C(=O)NCC(C)(C)O)c2=O)cn1] | 6-[4-(Fluoromethyl)phenyl]-N-(2-hydroxy-2-methylpropyl)-3- oxo-2-(pyridin-3-yl)-2,3-dihydropyridazine-4-carboxamide |
| 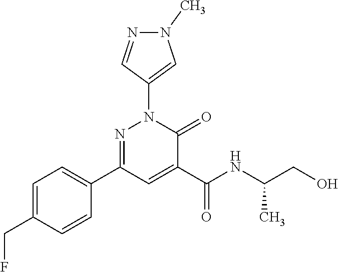 [SMILES: C[C@@H](CO)NC(=O)c1cc(-c2ccc(CF)cc2)nn(-c2cnn(C)c2)c1=O] | 6-[4-(Fluoromethyl)phenyl]-N-[(2S)-1-hydroxypropan-2-yl]-2- (1-methyl-1H-pyrazol-4-yl)-3-oxo-2,3-dihydropyridazine-4- carboxamide |
| 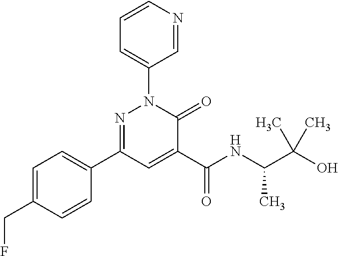 [SMILES: C[C@H](NC(=O)c1cc(-c2ccc(CF)cc2)nn(-c2cccnc2)c1=O)C(C)(C)O] | 6-[4-(Fluoromethyl)phenyl]-N-[(2S)-3-hydroxy-3-methylbutan- 2-yl]-3-oxo-2-(pyridin-3-yl)-2,3-dihydropyridazine-4- carboxamide |
| 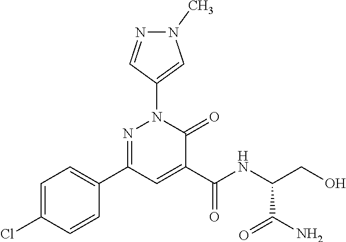 [SMILES: Cn1cc(-n2nc(-c3ccc(Cl)cc3)cc(C(=O)N[C@H](CO)C(N)=O)c2=O)cn1] | N-[(2R)-1-Amino-3-hydroxy-1-oxopropan-2-yl]-6-(4- chlorophenyl)-2-(1-methyl-1H-pyrazol-4-yl)-3-oxo-2,3- dihydropyridazine-4-carboxamide |
| 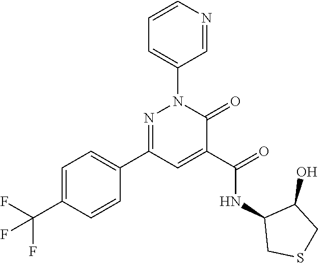 [SMILES: O=C(N[C@@H]1CSC[C@@H]1O)c1cc(-c2ccc(C(F)(F)F)cc2)nn(-c2cccnc2)c1=O] | N-cis-4-Hydroxytetrahydrothiophen-3-yl-3-oxo-2-(pyridin-3-yl)- 6-[4-(trifluoromethyl)phenyl]-2,3-dihydropyridazine-4- carboxamide |
| 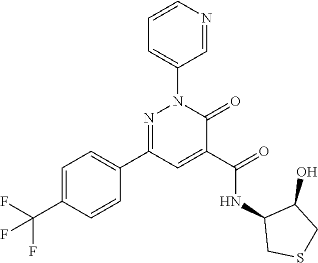 [SMILES: O=C(N[C@@H]1CSC[C@@H]1O)c1cc(-c2ccc(C(F)(F)F)cc2)nn(-c2cccnc2)c1=O] | (−)-N-cis-4-hydroxytetrahydrothiophen-3-yl-3-oxo-2-(pyridin-3- yl)-6-[4-(trifluoromethyl)phenyl]-2,3-dihydropyridazine-4- carboxamide, enantiomer 2 |
| 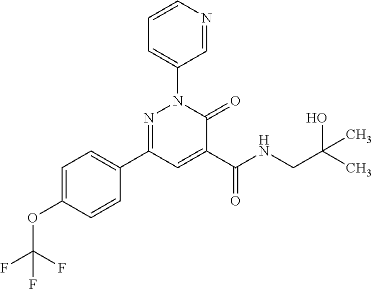 [SMILES: CC(C)(O)CNC(=O)c1cc(-c2ccc(OC(F)(F)F)cc2)nn(-c2cccnc2)c1=O] | N-(2-Hydroxy-2-methylpropyl)-3-oxo-2-(pyridin-3-yl)-6-[4- (trifluoromethoxy)phenyl]-2,3-dihydropyridazine-4- carboxamide |
| 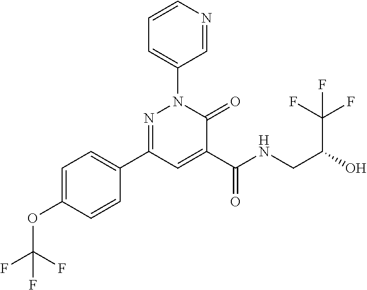 [SMILES: O=C(NC[C@@H](O)C(F)(F)F)c1cc(-c2ccc(OC(F)(F)F)cc2)nn(-c2cccnc2)c1=O] | 3-Oxo-2-(pyridin-3-yl)-N-[(2R)-3,3,3-trifluoro-2-hydroxypropyl]- 6-[4-(trifluoromethoxy)phenyl]-2,3-dihydropyridazine-4- carboxamide |
| 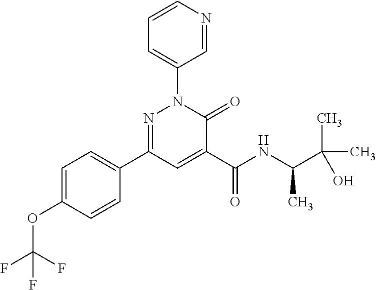 [SMILES: C[C@@H](NC(=O)c1cc(-c2ccc(OC(F)(F)F)cc2)nn(-c2cccnc2)c1=O)C(C)(C)O] | N-[(2R)-3-Hydroxy-3-methylbutan-2-yl]-3-oxo-2-(pyridin-3-yl)- 6-[4-(trifluoromethoxy)phenyl]-2,3-dihydropyridazine-4- carboxamide |
| 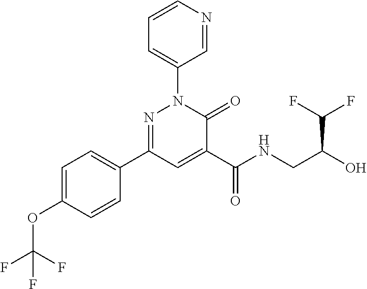 [SMILES: O=C(NC[C@H](O)C(F)F)c1cc(-c2ccc(OC(F)(F)F)cc2)nn(-c2cccnc2)c1=O] | N-[(2S)-3,3-Difluoro-2-hydroxypropyl]-3-oxo-2-(pyridin-3-yl)-6- [4-(trifluoromethoxy)phenyl]-2,3-dihydropyridazine-4- carboxamide |
| 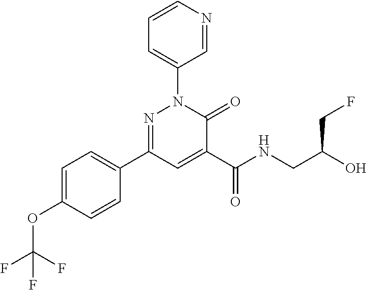 [SMILES: O=C(NC[C@H](O)CF)c1cc(-c2ccc(OC(F)(F)F)cc2)nn(-c2cccnc2)c1=O] | N-[(2S)-3-Fluoro-2-hydroxypropyl]-3-oxo-2-(pyridin-3-yl)-6-[4- (trifluoromethoxy)phenyl]-2,3-dihydropyridazine-4- carboxamide |
| 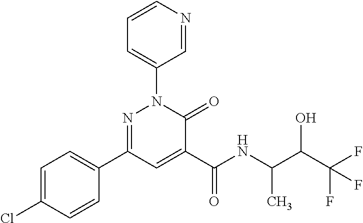 [SMILES: CC(NC(=O)c1cc(-c2ccc(Cl)cc2)nn(-c2cccnc2)c1=O)C(O)C(F)(F)F] | 6-(4-Chlorophenyl)-3-oxo-2-(pyridin-3-yl)-N-(4,4,4-trifluoro-3- hydroxybutan-2-yl)-2,3-dihydropyridazine-4-carboxamide Isomer 1 |
| 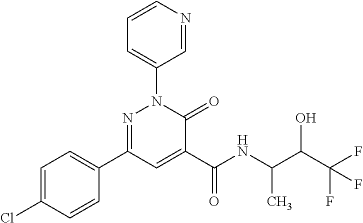 [SMILES: CC(NC(=O)c1cc(-c2ccc(Cl)cc2)nn(-c2cccnc2)c1=O)C(O)C(F)(F)F] | 6-(4-Chlorophenyl)-3-oxo-2-(pyridin-3-yl)-N-(4,4,4-trifluoro-3- hydroxybutan-2-yl)-2,3-dihydropyridazine-4-carboxamide Isomer 2 |
| 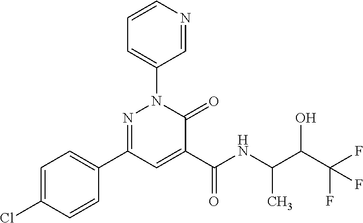 [SMILES: CC(NC(=O)c1cc(-c2ccc(Cl)cc2)nn(-c2cccnc2)c1=O)C(O)C(F)(F)F] | 6-(4-Chlorophenyl)-3-oxo-2-(pyridin-3-yl)-N-(4,4,4-trifluoro-3- hydroxybutan-2-yl)-2,3-dihydropyridazine-4-carboxamide Isomer 3 |
| 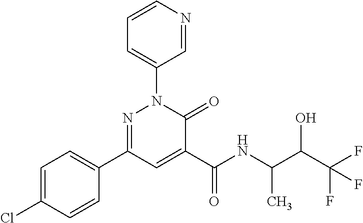 [SMILES: CC(NC(=O)c1cc(-c2ccc(Cl)cc2)nn(-c2cccnc2)c1=O)C(O)C(F)(F)F] | 6-(4-Chlorophenyl)-3-oxo-2-(pyridin-3-yl)-N-(4,4,4-trifluoro-3- hydroxybutan-2-yl)-2,3-dihydropyridazine-4-carboxamide Isomer 4 |
| 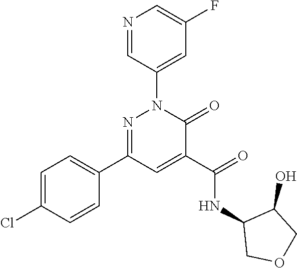 [SMILES: O=C(N[C@@H]1COC[C@@H]1O)c1cc(-c2ccc(Cl)cc2)nn(-c2cncc(F)c2)c1=O] | 6-(4-Chlorophenyl)-2-(5-fluoropyridin-3-yl)-N-[(cis)-4- hydroxytetrahydrofuran-3-yl]-3-oxo-2,3-dihydropyridazine-4- carboxamide |
| 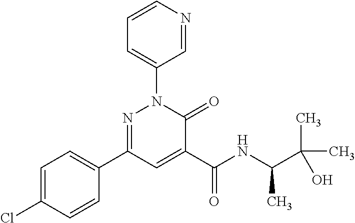 [SMILES: C[C@@H](NC(=O)c1cc(-c2ccc(Cl)cc2)nn(-c2cccnc2)c1=O)C(C)(C)O] | 6-(4-Chlorophenyl)-N-[(2R)-3-hydroxy-3-methylbutan-2-yl]-3- oxo-2-(pyridin-3-yl)-2,3-dihydropyridazine-4-carboxamide |
| 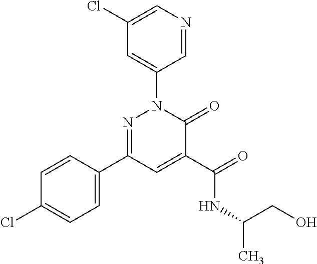 [SMILES: C[C@@H](CO)NC(=O)c1cc(-c2ccc(Cl)cc2)nn(-c2cncc(Cl)c2)c1=O] | 6-(4-Chlorophenyl)-2-(5-chloropyridin-3-yl)-N-[(2S)-1- hydroxypropan-2-yl]-3-oxo-2,3-dihydropyridazine-4- carboxamide |
| 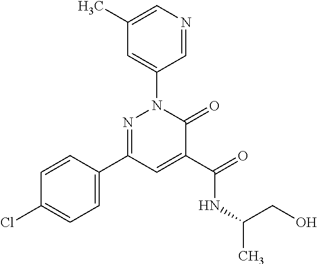 [SMILES: Cc1cncc(-n2nc(-c3ccc(Cl)cc3)cc(C(=O)N[C@@H](C)CO)c2=O)c1] | 6-(4-Chlorophenyl)-N-[(2S)-1-hydroxypropan-2-yl]-2-(5- methylpyridin-3-yl)-3-oxo-2,3-dihydropyridazine-4- carboxamide |
| 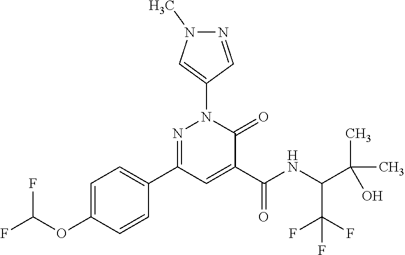 [SMILES: Cn1cc(-n2nc(-c3ccc(OC(F)F)cc3)cc(C(=O)NC(C(C)(C)O)C(F)(F)F)c2=O)cn1] | 6-[4-(Difluoromethoxy)phenyl]-2-(1-methyl-1H-pyrazol-4-yl)-3- oxo-N-(1,1,1-trifluoro-3-hydroxy-3-methylbutan-2-yl)-2,3- dihydropyridazine-4-carboxamide |
| 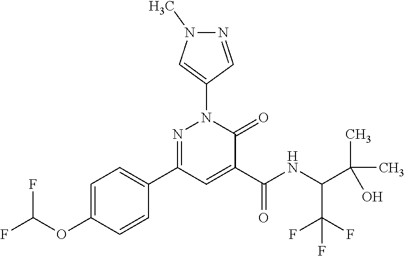 [SMILES: Cn1cc(-n2nc(-c3ccc(OC(F)F)cc3)cc(C(=O)NC(C(C)(C)O)C(F)(F)F)c2=O)cn1] | 6-[4-(Difluoromethoxy)phenyl]-2-(1-methyl-1H-pyrazol-4-yl)-3- oxo-N-(1,1,1-trifluoro-3-hydroxy-3-methylbutan-2-yl)-2,3- dihydropyridazine-4-carboxamide Isomer 1 |
| 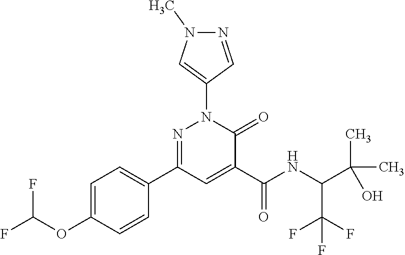 [SMILES: Cn1cc(-n2nc(-c3ccc(OC(F)F)cc3)cc(C(=O)NC(C(C)(C)O)C(F)(F)F)c2=O)cn1] | 6-[4-(Difluoromethoxy)phenyl]-2-(1-methyl-1H-pyrazol-4-yl)-3- oxo-N-(1,1,1-trifluoro-3-hydroxy-3-methylbutan-2-yl)-2,3- dihydropyridazine-4-carboxamide Isomer 2 |
| 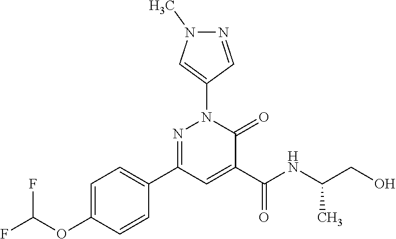 [SMILES: C[C@@H](CO)NC(=O)c1cc(-c2ccc(OC(F)F)cc2)nn(-c2cnn(C)c2)c1=O] | 6-[4-(Difluoromethoxy)phenyl]-N-[(2S)-1-hydroxypropan-2-yl]- 2-(1-methyl-1H-pyrazol-4-yl)-3-oxo-2,3-dihydropyridazine-4- carboxamide |
| 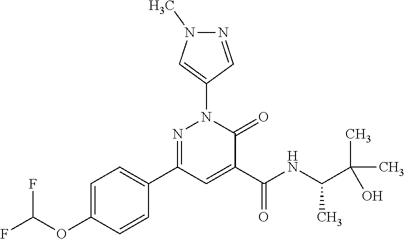 [SMILES: C[C@H](NC(=O)c1cc(-c2ccc(OC(F)F)cc2)nn(-c2cnn(C)c2)c1=O)C(C)(C)O] | 6-[4-(Difluoromethoxy)phenyl]-N-[(2S)-3-hydroxy-3- methylbutan-2-yl]-2-(1-methyl-1H-pyrazol-4-yl)-3-oxo-2,3- dihydropyridazine-4-carboxamide |
| 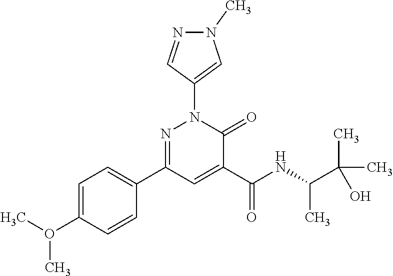 | 6-[4-(Dimethylamino)phenyl]-N-[(2S)-3-hydroxy-3- methylbutan-2-yl]-2-(1-methyl-1H-pyrazol-4-yl)-3-oxo-2,3- dihydropyridazine-4-carboxamide |
| 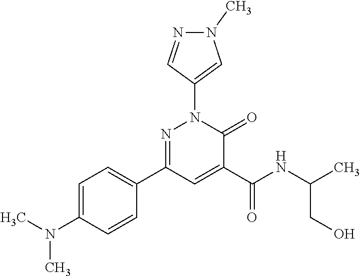 [SMILES: CC(CO)NC(=O)c1cc(-c2ccc(N(C)C)cc2)nn(-c2cnn(C)c2)c1=O] | 6-[4-(Dimethylamino)phenyl]-N-[(2S)-1-hydroxypropan-2-yl]-2- (1-methyl-1H-pyrazol-4-yl)-3-oxo-2,3-dihydropyridazine-4- carboxamide |
| 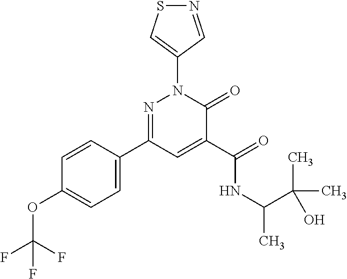 [SMILES: CC(NC(=O)c1cc(-c2ccc(OC(F)(F)F)cc2)nn(-c2cnsc2)c1=O)C(C)(C)O] | N-[(2S)-3-Hydroxy-3-methylbutan-2-yl]-3-oxo-2-(1,2-thiazol-4- yl)-6-[4-(trifluoromethoxy)phenyl]-2,3-dihydropyridazine-4- carboxamide |
| 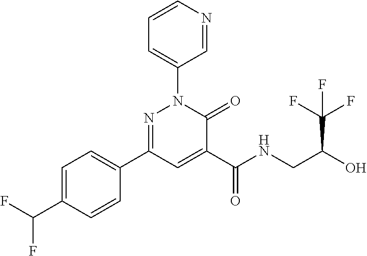 [SMILES: O=C(NC[C@H](O)C(F)(F)F)c1cc(-c2ccc(C(F)F)cc2)nn(-c2cccnc2)c1=O] | 6-[4-(Difluoromethyl)phenyl]-3-oxo-2-(pyridin-3-yl)-N-[(2S)- 3,3,3-trifluoro-2-hydroxypropyl]-2,3-dihydropyridazine-4- carboxamide |
| 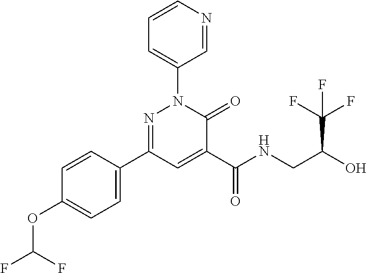 [SMILES: O=C(NC[C@H](O)C(F)(F)F)c1cc(-c2ccc(OC(F)F)cc2)nn(-c2cccnc2)c1=O] | 6-[4-(Difluoromethoxy)phenyl]-3-oxo-2-(pyridin-3-yl)-N-[(2S)- 3,3,3-trifluoro-2-hydroxypropyl]-2,3-dihydropyridazine-4- carboxamide |
| 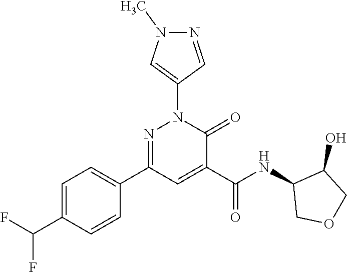 [SMILES: Cn1cc(-n2nc(-c3ccc(C(F)F)cc3)cc(C(=O)N[C@@H]3COC[C@@H]3O)c2=O)cn1] | 6-[4-(Difluoromethyl)phenyl]-N-[(cis)-4- hydroxytetrahydrofuran-3-yl]-2-(1-methyl-1H-pyrazol-4-yl)-3- oxo-2,3-dihydropyridazine-4-carboxamide Isomere 1 |
| 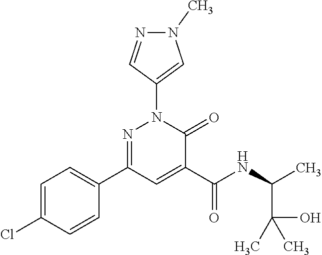 [SMILES: C[C@H](NC(=O)c1cc(-c2ccc(Cl)cc2)nn(-c2cnn(C)c2)c1=O)C(C)(C)O] | 6-(4-Chlorophenyl)-N-[(2S)-3-hydroxy-3-methylbutan-2-yl]-2- (1-methyl-1H-pyrazol-4-yl)-3-oxo-2,3-dihydropyridazine-4- carboxamide |
| 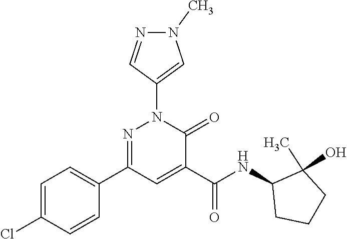 [SMILES: Cn1cc(-n2nc(-c3ccc(Cl)cc3)cc(C(=O)N[C@@H]3CCC[C@]3(C)O)c2=O)cn1] | 6-(4-Chlorophenyl)-N-[(cis)-2-hydroxy-2-methylcyclopentyl]-2- (1-methyl-1H-pyrazol-4-yl)-3-oxo-2,3-dihydropyridazine-4- carboxamide |
| 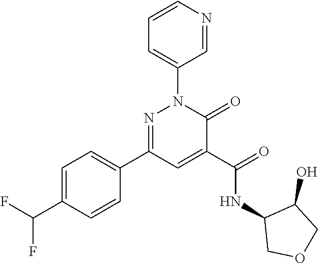 [SMILES: O=C(N[C@@H]1COC[C@@H]1O)c1cc(-c2ccc(C(F)F)cc2)nn(-c2cccnc2)c1=O] | 6-[4-(Difluoromethyl)phenyl]-N-[(cis)-4- hydroxytetrahydrofuran-3-yl]-3-oxo-2-(pyridin-3-yl)-2,3- dihydropyridazine-4-carboxamide Isomere 1 |
| 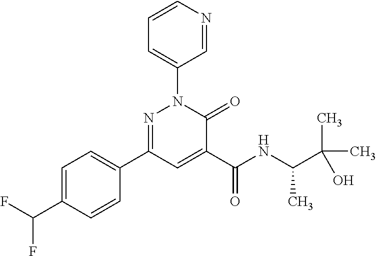 [SMILES: C[C@H](NC(=O)c1cc(-c2ccc(C(F)F)cc2)nn(-c2cccnc2)c1=O)C(C)(C)O] | 6-[4-(Difluoromethyl)phenyl]-N-[(2S)-3-hydroxy-3- methylbutan-2-yl]-3-oxo-2-(pyridin-3-yl)-2,3-dihydropyridazine- 4-carboxamide |
| 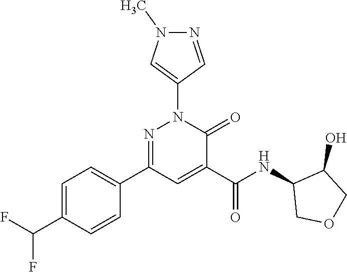 [SMILES: Cn1cc(-n2nc(-c3ccc(C(F)F)cc3)cc(C(=O)N[C@@H]3COC[C@@H]3O)c2=O)cn1] | 6-[4-(Difluoromethyl)phenyl]-N-[(cis)-4- hydroxytetrahydrofuran-3-yl]-2-(1-methyl-1H-pyrazol-4-yl)-3- oxo-2,3-dihydropyridazine-4-carboxamide Isomere 2 |
| 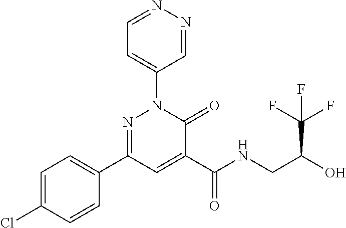 [SMILES: O=C(NC[C@H](O)C(F)(F)F)c1cc(-c2ccc(Cl)cc2)nn(-c2ccnnc2)c1=O] | 3-(4-Chlorophenyl)-6-oxo-N-[(2S)-3,3,3-trifluoro-2- hydroxypropyl]-6H-1,4′-bipyridazine-5-carboxamide |
| 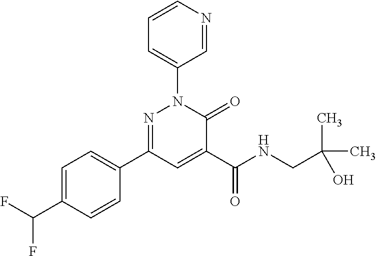 [SMILES: CC(C)(O)CNC(=O)c1cc(-c2ccc(C(F)F)cc2)nn(-c2cccnc2)c1=O] | 6-[4-(Difluoromethyl)phenyl]-N-(2-hydroxy-2-methylpropyl)-3- oxo-2-(pyridin-3-yl)-2,3-dihydropyridazine-4-carboxamide |
| 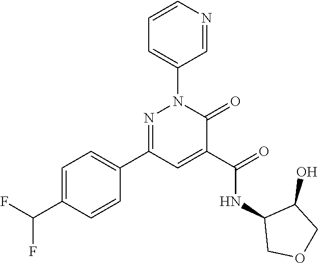 [SMILES: O=C(N[C@@H]1COC[C@@H]1O)c1cc(-c2ccc(C(F)F)cc2)nn(-c2cccnc2)c1=O] | 6-[4-(Difluoromethyl)phenyl]-N-[(cis)-4- hydroxytetrahydrofuran-3-yl]-3-oxo-2-(pyridin-3-yl)-2,3- dihydropyridazine-4-carboxamide Isomere 2 |
| 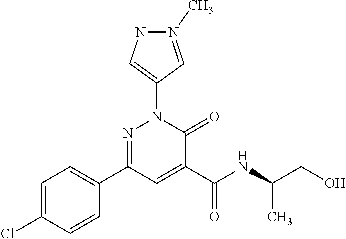 | 6-(4-Chlorophenyl)-N-[(2R)-1-hydroxypropan-2-yl]-2-(1- methyl-1H-pyrazol-4-yl)-3-oxo-2,3-dihydropyridazine-4- carboxamide |
| 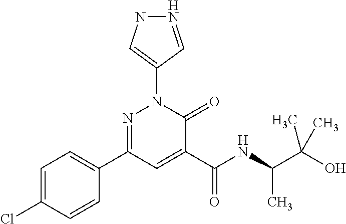 | 6-(4-Chlorophenyl)-N-[(2R)-3-hydroxy-3-methylbutan-2-yl]-3- oxo-2-(1H-pyrazol-4-yl)-2,3-dihydropyridazine-4-carboxamide |

| Structure | IUPAC name |
|---|---|
| 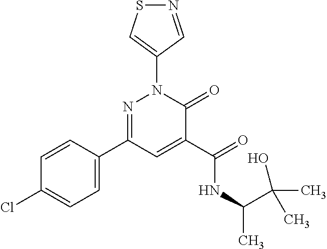 [SMILES: C[C@@H](NC(=O)c1cc(-c2ccc(Cl)cc2)nn(-c2cnsc2)c1=O)C(C)(C)O] | 6-(4-Chlorophenyl)-N-[(2R)-3-hydroxy-3-methylbutan-2-yl]-3- oxo-2-(1,2-thiazol-4-yl)-2,3-dihydropyridazine-4-carboxamide |
| 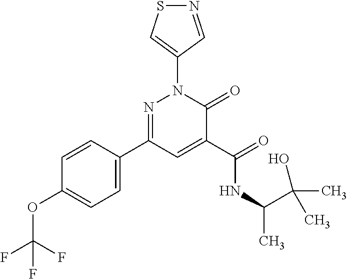 [SMILES: C[C@@H](NC(=O)c1cc(-c2ccc(OC(F)(F)F)cc2)nn(-c2cnsc2)c1=O)C(C)(C)O] | N-[(2R)-3-Hydroxy-3-methylbutan-2-yl]-3-oxo-2-(1,2-thiazol-4- yl)-6-[4-(trifluoromethoxy)phenyl]-2,3-dihydropyridazine-4- carboxamide |
| 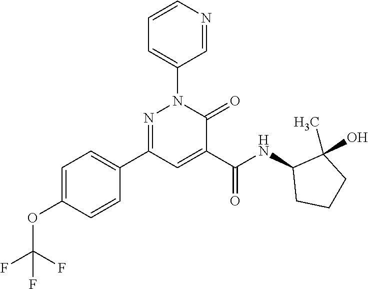 [SMILES: C[C@]1(O)CCC[C@H]1NC(=O)c1cc(-c2ccc(OC(F)(F)F)cc2)nn(-c2cccnc2)c1=O] | N-[(cis)-2-Hydroxy-2-methylcyclopentyl]-3-oxo-2-(pyridin-3-yl)- 6-[4-(trifluoromethoxy)phenyl]-2,3-dihydropyridazine-4- carboxamide |
| 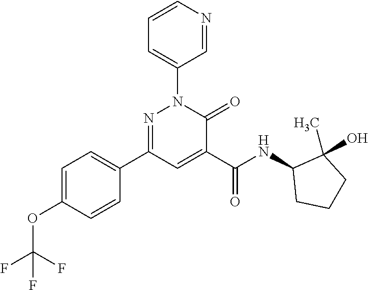 [SMILES: C[C@]1(O)CCC[C@H]1NC(=O)c1cc(-c2ccc(OC(F)(F)F)cc2)nn(-c2cccnc2)c1=O] | N-[(cis)-2-hydroxy-2-methylcyclopentyl]-3-oxo-2-(pyridin-3-yl)- 6-[4-(trifluoromethoxy)phenyl]-2,3-dihydropyridazine-4- carboxamide, Isomer 1 |
| 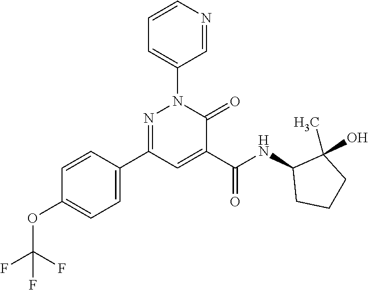 [SMILES: C[C@]1(O)CCC[C@H]1NC(=O)c1cc(-c2ccc(OC(F)(F)F)cc2)nn(-c2cccnc2)c1=O] | N-[(cis)-2-hydroxy-2-methylcyclopentyl]-3-oxo-2-(pyridin-3-yl)- 6-[4-(trifluoromethoxy)phenyl]-2,3-dihydropyridazine-4- carboxamide, Isomer 2 |
| 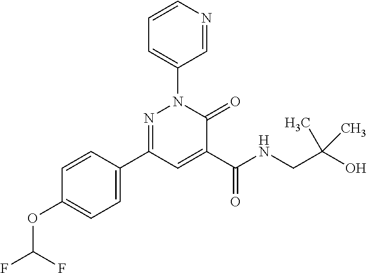 [SMILES: CC(C)(O)CNC(=O)c1cc(-c2ccc(OC(F)F)cc2)nn(-c2cccnc2)c1=O] | 6-[4-(Difluoromethoxy)phenyl]-N-(2-hydroxy-2-methylpropyl)- 3-oxo-2-(pyridin-3-yl)-2,3-dihydropyridazine-4-carboxamide |
| 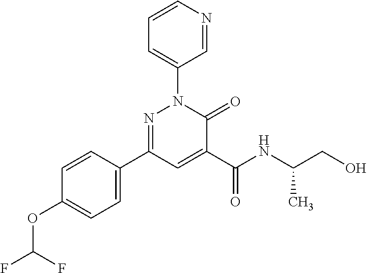 [SMILES: C[C@@H](CO)NC(=O)c1cc(-c2ccc(OC(F)F)cc2)nn(-c2cccnc2)c1=O] | 6-[4-(Difluoromethoxy)phenyl]-N-[(2S)-1-hydroxypropan-2-yl]- 3-oxo-2-(pyridin-3-yl)-2,3-dihydropyridazine-4-carboxamide |
| 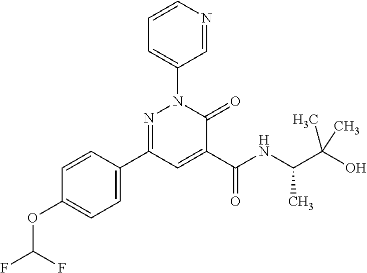 [SMILES: C[C@H](NC(=O)c1cc(-c2ccc(OC(F)F)cc2)nn(-c2cccnc2)c1=O)C(C)(C)O] | 6-[4-(Difluoromethoxy)phenyl]-N-[(2S)-3-hydroxy-3- methylbutan-2-yl]-3-oxo-2-(pyridin-3-yl)-2,3-dihydropyridazine- 4-carboxamide |
| 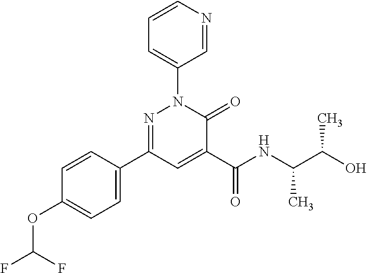 [SMILES: C[C@H](O)[C@H](C)NC(=O)c1cc(-c2ccc(OC(F)F)cc2)nn(-c2cccnc2)c1=O] | 6-[4-(Difluoromethoxy)phenyl]-N-[(2S,3S)-3-hydroxybutan-2- yl]-3-oxo-2-(pyridin-3-yl)-2,3-dihydropyridazine-4-carboxamide |
| 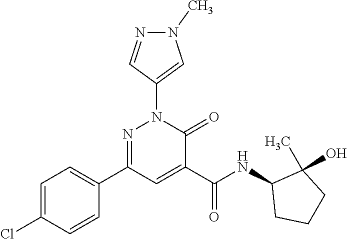 [SMILES: Cn1cc(-n2nc(-c3ccc(Cl)cc3)cc(C(=O)N[C@@H]3CCC[C@]3(C)O)c2=O)cn1] | 6-(4-Chlorophenyl)-N-[(cis)-2-hydroxy-2-methylcyclopentyl]-2- (1-methyl-1H-pyrazol-4-yl)-3-oxo-2,3-dihydropyridazine-4- carboxamide; Isomer 1 |
| 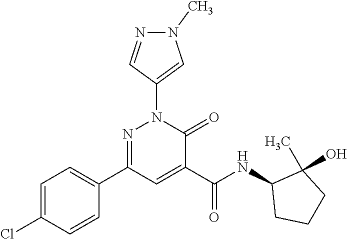 [SMILES: Cn1cc(-n2nc(-c3ccc(Cl)cc3)cc(C(=O)N[C@@H]3CCC[C@]3(C)O)c2=O)cn1] | 6-(4-Chlorophenyl)-N-[(cis)-2-hydroxy-2-methylcyclopentyl]-2- (1-methyl-1H-pyrazol-4-yl)-3-oxo-2,3-dihydropyridazine-4- carboxamide; Isomer 2 |
| 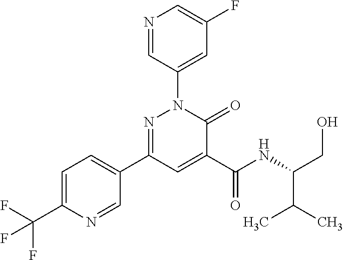 [SMILES: CC(C)[C@@H](CO)NC(=O)c1cc(-c2ccc(C(F)(F)F)nc2)nn(-c2cncc(F)c2)c1=O] | 2-(5-Fluoropyridin-3-yl)-N-[(2S)-1-hydroxy-3-methylbutan-2- yl]-3-oxo-6-[6-(trifluoromethyl)pyridin-3-yl]-2,3- dihydropyridazine-4-carboxamide |
| 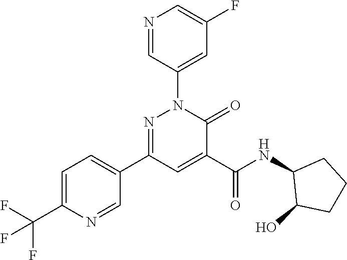 [SMILES: O=C(N[C@H]1CCC[C@H]1O)c1cc(-c2ccc(C(F)(F)F)nc2)nn(-c2cncc(F)c2)c1=O] | 2-(5-Fluoropyridin-3-yl)-N-[(1S,2R)-2-hydroxycyclopentyl]-3- oxo-6-[6-(trifluoromethyl)pyridin-3-yl]-2,3-dihydropyridazine-4- carboxamide |
| 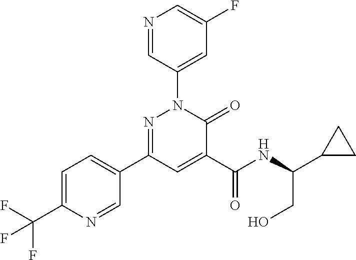 [SMILES: O=C(N[C@H](CO)C1CC1)c1cc(-c2ccc(C(F)(F)F)nc2)nn(-c2cncc(F)c2)c1=O] | N-[(1S)-1-Cyclopropyl-2-hydroxyethyl]-2-(5-fluoropyridin-3-yl)- 3-oxo-6-[6-(trifluoromethyl)pyridin-3-yl]-2,3-dihydropyridazine- 4-carboxamide |
| 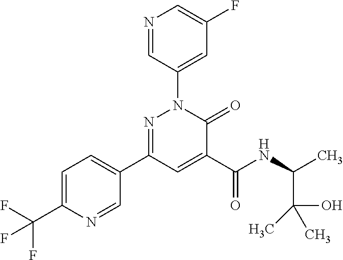 [SMILES: C[C@H](NC(=O)c1cc(-c2ccc(C(F)(F)F)nc2)nn(-c2cncc(F)c2)c1=O)C(C)(C)O] | 2-(5-Fluoropyridin-3-yl)-N-[(2S)-3-hydroxy-3-methylbutan-2- yl]-3-oxo-6-[6-(trifluoromethyl)pyridin-3-yl]-2,3- dihydropyridazine-4-carboxamide |
| 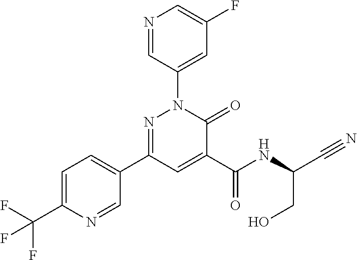 [SMILES: N#C[C@@H](CO)NC(=O)c1cc(-c2ccc(C(F)(F)F)nc2)nn(-c2cncc(F)c2)c1=O] | N-[(1S)-1-Cyano-2-hydroxyethyl]-2-(5-fluoropyridin-3-yl)-3- oxo-6-[6-(trifluoromethyl)pyridin-3-yl]-2,3-dihydropyridazine-4- carboxamide |
| 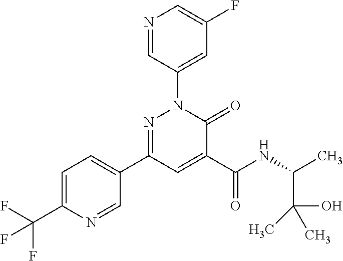 [SMILES: C[C@@H](NC(=O)c1cc(-c2ccc(C(F)(F)F)nc2)nn(-c2cncc(F)c2)c1=O)C(C)(C)O] | 2-(5-Fluoropyridin-3-yl)-N-[(2R)-3-hydroxy-3-methylbutan-2- yl]-3-oxo-6-[6-(trifluoromethyl)pyridin-3-yl]-2,3- dihydropyridazine-4-carboxamide |
| 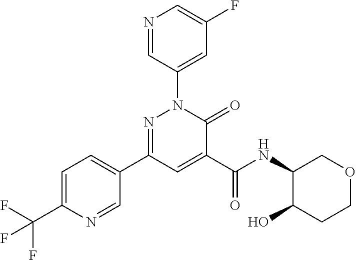 [SMILES: O=C(N[C@H]1COCC[C@H]1O)c1cc(-c2ccc(C(F)(F)F)nc2)nn(-c2cncc(F)c2)c1=O] | 1,5-Anhydro-2,4-dideoxy-2-[({2-(5-fluoropyridin-3-yl)-3-oxo-6- [6-(trifluoromethyl)pyridin-3-yl]-2,3-dihydropyridazine-4- yl}carbonyl)amino]-D-erythro-pentitol |
| 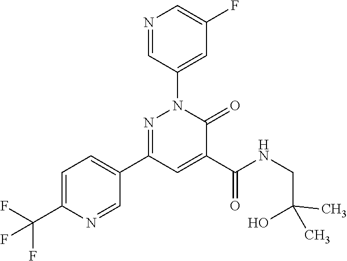 [SMILES: CC(C)(O)CNC(=O)c1cc(-c2ccc(C(F)(F)F)nc2)nn(-c2cncc(F)c2)c1=O] | 2-(5-Fluoropyridin-3-yl)-N-(2-hydroxy-2-methylpropyl)-3-oxo-6- [6-(trifluoromethyl)pyridin-3-yl]-2,3-dihydropyridazine-4- carboxamide |
| 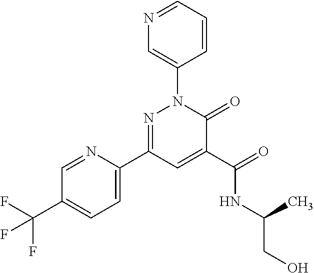 [SMILES: C[C@@H](CO)NC(=O)c1cc(-c2ccc(C(F)(F)F)cn2)nn(-c2cccnc2)c1=O] | N-[(2S)-1-Hydroxypropan-2-yl]-3-oxo-2-(pyridin-3-yl)-6-[5- (trifluoromethyl)pyridin-2-yl]-2,3-dihydropyridazine-4- carboxamide |

Therefore, in a first aspect, the present invention provides combinations of at least two components, component A and component B, component A being 6-(4-Chlorophenyl)-N-[(2S)-1-hydroxypropan-2-yl]-2-(1-methyl-1H-pyrazol-4-yl)-3-oxo-2,3-dihydropyridazine-4-carboxamide, an AhR inhibitor, or the polymorphs, enantiomeres, diastereomeres, racemates, tautomeres, N-oxides, hydrates and solvates, as well as the physiological acceptable salts and solvates of the salts, as well as mixtures of the same, and component B being an antibody.

Preferred is an antibody that is an immune-checkpoint-inhibitor (PD-1 inhibitor).

Preferred antibodies are nivolumab and pembrolizumab.

The most preferred antibody is pembrolizumab.

In accordance with a second aspect, the present invention covers combinations of at least two components A and B, component A being an AhR inhibitor as defined herein, and component B being pembrolizumab or nivolumab.

In accordance with a third aspect, the present invention comprises combinations of at least two components A and B, component A being an AhR inhibitor or polymorphs, enantiomeres, diastereomeres, racemates, tautomeres, N-oxides, hydrates and solvates thereof, as well as the physiological acceptable salts and solvates of the salts, as well as mixtures of the same, and component B being pembrolizumab or nivolumab.

The combinations comprising at least two components A and B, as described and defined herein, are also referred to as “combinations of the present invention”.

Further, the present invention relates to a kit comprising a combination of:

Component A: one or more AhR inhibitors as described supra and infra, or polymorphs, enantiomeres, diastereomeres, racemates, tautomeres, N-oxides, hydrates and solvates thereof, as well as the physiological acceptable salts and solvates of the salts, as well as mixtures of the same, thereof;
Component B: pembrolizumab or nivolumab;
and optionally,
Component C: one or more further pharmaceutical agents; in which optionally either or both of said components A and B in any of the above mentioned combinations are in the form of a pharmaceutical formulation/composition which is ready for use to be administered simultaneously, concurrently, separately or sequentially. The components may be administered independently of one another by the oral, intravenous, topical, local installations, intraperitoneal or nasal route.

In accordance with another aspect, the present invention covers the combinations as described supra for the treatment or prophylaxis of a disease, in particular cancer, but are not limited to solid tumors, such as for example cancers of the breast, respiratory tract, brain, reproductive organs, digestive tract, urinary tract, eye, liver, skin, head and neck, thyroid, parathyroid, and their distant metastases. Those disorders also include lymphomas, sarcomas and leukemias.

Tumors of the digestive tract include, but are not limited to anal, colon, colorectal, esophageal, gallbladder, gastric, pancreatic, rectal, small-intestine, and salivary gland cancers.

Examples of esophageal cancer include, but are not limited to esophageal cell carcinomas and Adenocarcinomas, as well as squamous cell carcinomas, Leiomyosarcoma, Malignant melanoma, rhabdomyosarcoma and Lymphoma.

Examples of gastric cancer include, but are not limited to intestinal type and diffuse type gastric adenocarcinoma.

Examples of pancreatic cancer include, but are not limited to ductal adenocarcinoma, adenosquamous carcinomas and pancreatic endocrine tumors.

Examples of breast cancer include, but are not limited to triple negative breast cancer, invasive ductal carcinoma, invasive lobular carcinoma, ductal carcinoma in situ, and lobular carcinoma in situ.

Examples of cancers of the respiratory tract include, but are not limited to small-cell and non-small-cell lung carcinoma, as well as bronchial adenoma and pleuropulmonary blastoma.

Examples of brain cancers include, but are not limited to brain stem and hypophtalmic glioma, cerebellar and cerebral astrocytoma, glioblastoma, medulloblastoma, ependymoma, as well as neuroectodermal and pineal tumor.

Tumors of the male reproductive organs include, but are not limited to prostate and testicular cancer. Tumors of the female reproductive organs include, but are not limited to endometrial, cervical, ovarian, vaginal and vulvar cancer, as well as sarcoma of the uterus.

Examples of ovarian cancer include, but are not limited to serous tumour, endometrioid tumor, mucinous cystadenocarcinoma, granulosa cell tumor, Sertoli-Leydig cell tumor and arrhenoblastoma.

Examples of cervical cancer include, but are not limited to squamous cell carcinoma, adenocarcinoma, adenosquamous carcinoma, small cell carcinoma, neuroendocrine tumour, glassy cell carcinoma and villoglandular adenocarcinoma.

Tumors of the urinary tract include, but are not limited to bladder, penile, kidney, renal pelvis, ureter, urethral, and hereditary and sporadic papillary renal cancers.

Examples of kidney cancer include, but are not limited to renal cell carcinoma, urothelial cell carcinoma, juxtaglomerular cell tumor (reninoma), angiomyolipoma, renal oncocytoma, Bellini duct carcinoma, clear-cell sarcoma of the kidney, mesoblastic nephroma and Wilms' tumor. Examples of bladder cancer include, but are not limited to transitional cell carcinoma, squamous cell carcinoma, adenocarcinoma, sarcoma and small cell carcinoma.

Eye cancers include, but are not limited to intraocular melanoma and retinoblastoma.

Examples of liver cancers include, but are not limited to hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to squamous cell carcinoma, Kaposi's sarcoma, malignant melanoma, Merkel cell skin cancer, and non-melanoma skin cancer.

Head-and-neck cancers include, but are not limited to squamous cell cancer of the head and neck, laryngeal, hypopharyngeal, nasopharyngeal, oropharyngeal cancer, salivary gland cancer, lip and oral cavity cancer, and squamous cell cancer.

Lymphomas include, but are not limited to AIDS-related lymphoma, non-Hodgkin's lymphoma, cutaneous T-cell lymphoma, Burkitt lymphoma, Hodgkin's disease, and lymphoma of the central nervous system.

Sarcomas include, but are not limited to sarcoma of the soft tissue, osteosarcoma, malignant fibrous histiocytoma, lymphosarcoma, and rhabdomyosarcoma.

Leukemias include, but are not limited to acute myeloid leukemia, acute lymphoblastic leukemia, chronic lymphocytic leukemia, chronic myelogenous leukemia, and hairy cell leukemia.

### DETAILED DESCRIPTION OF THE INVENTION

### Component A of the Combination

Component A can be selected from inhibitors of AhR, which are specifically or generically disclosed in the International Patent Application No. PCT/EP2018/052627, published as WO 2018/146010 A1 which are mentioned above and are incorporated herein by reference.

Where there is a discrepancy between the chemical name and the chemical structure depicted, the chemical structure depicted takes precedence over the chemical name given.

The synthesis of the compounds listed above is described in International Patent Application No. PCT/EP2018/052627, published as WO 2018/146010 A1, which is hereby incorporated herein in its entirety by reference.

Said component A may be in the form of a pharmaceutical formulation which is ready for use to be administered simultaneously, concurrently, separately or sequentially.

The components may be administered independently of one another by the oral, intravenous, topical, local installations, intraperitoneal or nasal route.

The AhR-inhibitors mentioned in the prior art as well as in the lists above have been disclosed for the treatment or prophylaxis of different diseases, especially cancer.

The specific compounds of the lists as disclosed above are preferred as being component A of the combination, most preferred is the compound used in the experimental section.

The synergistic behavior of a combination of the present invention is demonstrated herein with one of the AhR inhibitors specifically disclosed in the Examples section as component A.

In addition, a combination of the present invention comprising component A as mentioned above and an antibody as compound B.

The preferred antibody is pembrolizumab or is nivolumab as component B and is a preferred aspect of the invention.

In another aspect a combination of the present invention comprises the AhR inhibitor as defined above, or a pharmaceutically acceptable salt thereof as mentioned above as component A and pembrolizumab or nivolumab as component B.

It is to be understood that the present invention relates also to any combination of the embodiments of component A described above.

### Component B of the Combination

Component B is pembrolizumab or is nivolumab.

“Pembrolizumab”, developed by MERCK, (trade name “KEYTRUDA”, also known as lambrolizumab, and MK-3475) is a humanized monoclonal IgG4 antibody directed against human cell surface receptor PD-1. Pembrolizumab is described, for example, in U.S. Pat. No. 8,900,587. It is for example indicated for the treatment of patients with unresectable or metastatic melanoma, as a single agent for the first-line treatment of patients with metastatic NSCLC whose tumors have high PD-L1 expression [(Tumor Proportion Score (TPS)≥50%)] as determined by an FDA-approved test, with no EGFR or ALK genomic tumor aberrations and also for the treatment of patients with recurrent or metastatic HNSCC with disease progression on or after platinum-containing chemotherapy.

Pembrolizumab was purchased from Selleckchem (Catalog No. A2005).

“Nivolumab”, developed by Bristol-Myers Squibb, (trade name “OPDIVO”; formerly designated 5C4, BMS-936558, MDX-1106, or ONO-4538) is a fully human IgG4 (S228P) PD-1 immune checkpoint inhibitor antibody that selectively prevents interaction with PD-1 ligands (PD-L1 and PD-L2), thereby blocking the down-regulation of antitumor T-cell functions (U.S. Pat. No. 8,008,449). For example it is used as a first line treatment for inoperable or metastatic melanoma in combination with ipilimumab if the cancer does not have a mutation in BRAF, as a second-line treatment following treatment with ipilimumab and if the cancer has a mutation in BRAF, with a BRAF inhibitor, as a second-line treatment for squamous non-small cell lung cancer, and as a second-line treatment for renal cell carcinoma.

Nivolumab was generated in-house (TPP-2596, PPB2479).

In accordance with an embodiment, the present invention relates to a combination of any AhR inhibitor as component A mentioned herein with any component B mentioned herein, optionally with any component C mentioned herein.

In one embodiment component A of the combination is the compound used in the experimental section and component B is pembrolizumab being used in the experimental section.

In a particular embodiment, the present invention relates to a combination of a component A with a component B, optionally with a component C, as mentioned in the Examples Section herein.

Especially, the combination comprising as component A one or more compounds as defined herein; as components B pembrolizumab; and, optionally, one or more further pharmaceutical agents C; in which optionally both or either of said components A and B are in the form of a pharmaceutical formulation which is ready for use to be administered simultaneously, concurrently, separately or sequentially.

Further, the present invention relates to a kit comprising a combination of:

component A: one or more AhR inhibitors, or polymorphs, enantiomeres, diastereomeres, racemates, tautomeres, N-oxides, hydrates and solvates thereof, as well as the physiological acceptable salts and solvates of the salts, as well as mixtures of the same, thereof;
component B: pembrolizumab or nivolumab;
and, optionally,
component C: one or more further pharmaceutical agents;
in which optionally either or both of said components A and B in any of the above mentioned combinations are in the form of a pharmaceutical formulation which is ready for use to be administered simultaneously, concurrently, separately or sequentially.

The term “component C” being at least one pharmaceutical agent includes the effective compound itself as well as its pharmaceutically acceptable salts, solvates, hydrates or stereoisomers as well as any composition or pharmaceutical formulation comprising such effective compound or its pharmaceutically acceptable salts, solvates, hydrates or stereoisomers. A list of such readily available agents is being provided further below.

The components may be administered independently of one another by the oral, intravenous, topical, local installations, intraperitoneal or nasal route.

Component A is administered intravenously, intraperitoneally, preferably it is administered orally.

Component B is administered intravenously, intraperitoneally, preferably it is administered orally.

Component C being administered as the case may be.

The term “pharmaceutically acceptable” is used synonymously to the term “physiologically acceptable”.

The term “pharmaceutically or physiologically acceptable salt” of component A refers to a relatively non-toxic, inorganic or organic acid addition salt of a compound of the present invention. For example, see S. M. Berge, et al. “Pharmaceutical Salts,” J. Pharm. Sci. 1977, 66, 1-19. Pharmaceutically acceptable salts include those obtained by reacting the main compound, functioning as a base, with an inorganic or organic acid to form a salt, for example, salts of hydrochloric acid, sulfuric acid, phosphoric acid, methane sulfonic acid, camphor sulfonic acid, oxalic acid, maleic acid, succinic acid and citric acid. Pharmaceutically acceptable salts also include those in which the main compound functions as an acid and is reacted with an appropriate base to form, e.g., sodium, potassium, calcium, magnesium, ammonium, and chorine salts. Those skilled in the art will further recognize that acid addition salts of the claimed compounds may be prepared by reaction of the compounds with the appropriate inorganic or organic acid via any of a number of known methods. Alternatively, alkali and alkaline earth metal salts of acidic compounds of the invention are prepared by reacting the compounds of the invention with the appropriate base via a variety of known methods. Representative salts of a component A of this invention include the conventional non toxic salts and the quaternary ammonium salts which are formed, for example, from inorganic or organic acids or bases by means well known in the art. For example, such acid addition salts include acetate, adipate, alginate, ascorbate, aspartate, benzoate, benzenesulfonate, bisulfate, butyrate, citrate, camphorate, camphorsulfonate, cinnamate, cyclopentanepropionate, digluconate, dodecylsulfate, ethanesulfonate, fumarate, glucoheptanoate, glycerophosphate, hemisulfate, heptanoate, hexanoate, chloride, bromide, iodide, 2-hydroxyethanesulfonate, itaconate, lactate, maleate, mandelate, methanesulfonate, 2-naphthalenesulfonate, nicotinate, nitrate, oxalate, pamoate, pectinate, persulfate, 3-phenylpropionate, picrate, pivalate, propionate, succinate, sulfonate, sulfate, tartrate, thiocyanate, tosylate, and undecanoate.

Base salts include alkali metal salts such as potassium and sodium salts, alkaline earth metal salts such as calcium and magnesium salts, and ammonium salts with organic bases such as dicyclohexylamine and N-methyl-D-glucamine. Additionally, basic nitrogen containing groups may be quaternized with such agents as lower alkyl halides such as methyl, ethyl, propyl, or butyl chlorides, bromides and iodides; dialkyl sulfates like dimethyl, diethyl, dibutyl sulfate, or diamyl sulfates, long chain halides such as decyl, lauryl, myristyl and strearyl chlorides, bromides and iodides, aralkyl halides like benzyl and phenethyl bromides and others.

A solvate for the purpose of this invention is a complex of a solvent and a compound of the invention in the solid state. Exemplary solvates would include, but are not limited to, complexes of a compound of the invention with ethanol or methanol. Hydrates are a specific form of solvate wherein the solvent is water.

Components of this invention can be tableted with conventional tablet bases such as lactose, sucrose and cornstarch in combination with binders such as acacia, corn starch or gelatin, disintegrating agents intended to assist the break-up and dissolution of the tablet following administration such as potato starch, alginic acid, corn starch, and guar gum, gum tragacanth, acacia, lubricants intended to improve the flow of tablet granulation and to prevent the adhesion of tablet material to the surfaces of the tablet dies and punches, for example talc, stearic acid, or magnesium, calcium or zinc stearate, dyes, coloring agents, and flavoring agents such as peppermint, oil of wintergreen, or cherry flavoring, intended to enhance the aesthetic qualities of the tablets and make them more acceptable to the patient. Suitable excipients for use in oral liquid dosage forms include dicalcium phosphate and diluents such as water and alcohols, for example, ethanol, benzyl alcohol, and polyethylene alcohols, either with or without the addition of a pharmaceutically acceptable surfactant, suspending agent or emulsifying agent. Various other materials may be present as coatings or to otherwise modify the physical form of the dosage unit. For instance tablets, pills or capsules may be coated with shellac, sugar or both.

Dispersible powders and granules are suitable for the preparation of an aqueous suspension. They provide the active ingredient in admixture with a dispersing or wetting agent, a suspending agent and one or more preservatives. Suitable dispersing or wetting agents and suspending agents are exemplified by those already mentioned above. Additional excipients, for example those sweetening, flavoring and coloring gents described above, may also be present.

Components of this invention can also be in the form of oil-in-water emulsions. The oily phase may be a vegetable oil such as liquid paraffin or a mixture of vegetable oils.

Suitable emulsifying agents may be (1) naturally occurring gums such as gum acacia and gum tragacanth, (2) naturally occurring phosphatides such as soy bean and lecithin, (3) esters or partial esters derived form fatty acids and hexitol anhydrides, for example, sorbitan monooleate, (4) condensation products of said partial esters with ethylene oxide, for example, polyoxyethylene sorbitan monooleate. The emulsions may also contain sweetening and flavoring agents.

Oily suspensions can be formulated by suspending the active ingredient in a vegetable oil such as, for example, arachis oil, olive oil, sesame oil or coconut oil, or in a mineral oil such as liquid paraffin. The oily suspensions may contain a thickening agent such as, for example, beeswax, hard paraffin, or cetyl alcohol. The suspensions may also contain one or more preservatives, for example, ethyl or n-propyl p-hydroxybenzoate; one or more coloring agents; one or more flavoring agents; and one or more sweetening agents such as sucrose or saccharin.

Syrups and elixirs can be formulated with sweetening agents such as, for example, glycerol, propylene glycol, sorbitol or sucrose. Such formulations may also contain a demulcent, and preservative, such as methyl and propyl parabens and flavoring and coloring agents.

Components of this invention can also be administered parenterally, that is, subcutaneously, intravenously, intraocularly, intrasynovially, intramuscularly, or interperitoneally, as injectable dosages of the compound in preferably a physiologically acceptable diluent with a pharmaceutical carrier which can be a sterile liquid or mixture of liquids such as water, saline, aqueous dextrose and related sugar solutions, an alcohol such as ethanol, isopropanol, or hexadecyl alcohol, glycols such as propylene glycol or polyethylene glycol, glycerol ketals such as 2,2-dimethyl-1,1-dioxolane-4methanol, ethers such as poly(ethylene glycol) 400, an oil, a fatty acid, a fatty acid ester or, a fatty acid glyceride, or an acetylated fatty acid glyceride, with or without the addition of a pharmaceutically acceptable surfactant such as a soap or a detergent, suspending agent such as pectin, carbomers, methycellulose, hydroxypropylmethylcellulose, or carboxymethylcellulose, or emulsifying agent and other pharmaceutical adjuvants.

Illustrative of oils which can be used in the parenteral formulations of this invention are those of petroleum, animal, vegetable, or synthetic origin, for example, peanut oil, soybean oil, sesame oil, cottonseed oil, corn oil, olive oil, petrolatum and mineral oil.

Suitable fatty acids include oleic acid, stearic acid, isostearic acid and myristic acid. Suitable fatty acid esters are, for example, ethyl oleate and isopropyl myristate.

Suitable soaps include fatty acid alkali metal, ammonium, and triethanolamine salts and suitable detergents include cationic detergents, for example dimethyl dialkyl ammonium halides, alkyl pyridinium halides, and alkylamine acetates; anionic detergents, for example, alkyl, aryl, and olefin sulfonates, alkyl, olefin, ether, and monoglyceride sulfates, and sulfosuccinates; non-ionic detergents, for example, fatty amine oxides, fatty acid alkanolamides, and poly(oxyethylene-oxypropylene)s or ethylene oxide or propylene oxide copolymers; and amphoteric detergents, for example, alkyl-beta-aminopropionates, and 2-alkylimidazoline quarternary ammonium salts, as well as mixtures.

The parenteral compositions of this invention will typically contain from about 0.5% to about 25% by weight of the active ingredient in solution. Preservatives and buffers may also be used advantageously. In order to minimize or eliminate irritation at the site of injection, such compositions may contain a non-ionic surfactant having a hydrophile lipophile balance (HLB) preferably of from about 12 to about 17. The quantity of surfactant in such formulation preferably ranges from about 5% to about 15% by weight. The surfactant can be a single component having the above HLB or can be a mixture of two or more components having the desired HLB.

Illustrative of surfactants used in parenteral formulations are the class of polyethylene sorbitan fatty acid esters, for example, sorbitan monooleate and the high molecular weight adducts of ethylene oxide with a hydrophobic base, formed by the condensation of propylene oxide with propylene glycol.

The pharmaceutical compositions can be in the form of sterile injectable aqueous suspensions. Such suspensions may be formulated according to known methods using suitable dispersing or wetting agents and suspending agents such as, for example, sodium carboxymethylcellulose, methylcellulose, hydroxypropylmethyl-cellulose, sodium alginate, polyvinylpyrrolidone, gum tragacanth and gum acacia; dispersing or wetting agents which may be a naturally occurring phosphatide such as lecithin, a condensation product of an alkylene oxide with a fatty acid, for example, polyoxyethylene stearate, a condensation product of ethylene oxide with a long chain aliphatic alcohol, for example, heptadeca-ethyleneoxycetanol, a condensation product of ethylene oxide with a partial ester derived form a fatty acid and a hexitol such as polyoxyethylene sorbitol monooleate, or a condensation product of an ethylene oxide with a partial ester derived from a fatty acid and a hexitol anhydride, for example polyoxyethylene sorbitan monooleate.

The sterile injectable preparation can also be a sterile injectable solution or suspension in a non-toxic parenterally acceptable diluent or solvent. Diluents and solvents that may be employed are, for example, water, Ringer's solution, isotonic sodium chloride solutions and isotonic glucose solutions. In addition, sterile fixed oils are conventionally employed as solvents or suspending media. For this purpose, any bland, fixed oil may be employed including synthetic mono- or diglycerides. In addition, fatty acids such as oleic acid can be used in the preparation of injectables.

Components of the invention can also be administered in the form of suppositories for rectal administration of the drug. These components can be prepared by mixing the drug with a suitable non-irritation excipient which is solid at ordinary temperatures but liquid at the rectal temperature and will therefore melt in the rectum to release the drug. Such materials are, for example, cocoa butter and polyethylene glycol.

Another formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art (see, e.g., U.S. Pat. No. 5,023,252, issued Jun. 11, 1991, incorporated herein by reference). Such patches may be constructed for continuous, pulsatile, or on demand delivery of pharmaceutical agents.

Controlled release formulations for parenteral administration include liposomal, polymeric microsphere and polymeric gel formulations that are known in the art. It can be desirable or necessary to introduce a component of the present invention to the patient via a mechanical delivery device. The construction and use of mechanical delivery devices for the delivery of pharmaceutical agents is well known in the art.

Direct techniques for, for example, administering a drug directly to the brain usually involve placement of a drug delivery catheter into the patient's ventricular system to bypass the blood-brain barrier. One such implantable delivery system, used for the transport of agents to specific anatomical regions of the body, is described in U.S. Pat. No. 5,011,472, issued Apr. 30, 1991.

The compositions of the invention can also contain other conventional pharmaceutically acceptable compounding ingredients, generally referred to as carriers or diluents, as necessary or desired. Conventional procedures for preparing such compositions in appropriate dosage forms can be utilized. Such ingredients and procedures include those described in the following references, each of which is incorporated herein by reference: Powell, M. F. et al, “Compendium of Excipients for Parenteral Formulations” PDA Journal of Pharmaceutical Science & Technology 1998, 52(5), 238-311; Strickley, R. G “Parenteral Formulations of Small Molecule Therapeutics Marketed in the United States (1999)—Part-1” PDA Journal of Pharmaceutical Science & Technology 1999, 53(6), 324-349; and Nema, S. et al, “Excipients and Their Use in Injectable Products” PDA Journal of Pharmaceutical Science & Technology 1997, 51(4), 166-171.

Commonly used pharmaceutical ingredients that can be used as appropriate to formulate the composition for its intended route of administration include:

acidifying agents (examples include but are not limited to acetic acid, citric acid, fumaric acid, hydrochloric acid, nitric acid);
alkalinizing agents (examples include but are not limited to ammonia solution, ammonium carbonate, diethanolamine, monoethanolamine, potassium hydroxide, sodium borate, sodium carbonate, sodium hydroxide, triethanolamine, trolamine);
adsorbents (examples include but are not limited to powdered cellulose and activated charcoal); aerosol propellants (exmples include but are not limited to carbon dioxide, CCl2F2, F2ClC—CClF2 and CClF3);
air displacement agents (examples include but are not limited to nitrogen and argon);
antifungal preservatives (examples include but are not limited to benzoic acid, butylparaben, ethylparaben, methylparaben, propylparaben, sodium benzoate);
antimicrobial preservatives (examples include but are not limited to benzalkonium chloride, benzethonium chloride, benzyl alcohol, cetylpyridinium chloride, chlorobutanol, phenol, phenylethyl alcohol, phenylmercuric nitrate and thimerosal);
antioxidants (examples include but are not limited to ascorbic acid, ascorbyl palmitate, butylated hydroxyanisole, butylated hydroxytoluene, hypophosphorus acid, monothioglycerol, propyl gallate, sodium ascorbate, sodium bisulfite, sodium formaldehyde sulfoxylate, sodium metabisulfite);
binding materials (examples include but are not limited to block polymers, natural and synthetic rubber, polyacrylates, polyurethanes, silicones, polysiloxanes and styrene butadiene copolymers);
buffering agents (examples include but are not limited to potassium metaphosphate, dipotassium phosphate, sodium acetate, sodium citrate anhydrous and sodium citrate dihydrate);
carrying agents (examples include but are not limited to acacia syrup, aromatic syrup, aromatic elixir, cherry syrup, cocoa syrup, orange syrup, syrup, corn oil, mineral oil, peanut oil, sesame oil, bacteriostatic sodium chloride injection and bacteriostatic water for injection);
chelating agents (examples include but are not limited to edetate disodium and edetic acid) colorants (examples include but are not limited to FD&C Red No. 3, FD&C Red No. 20, FD&C Yellow No. 6, FD&C Blue No. 2, D&C Green No. 5, D&C Orange No. 5, D&C Red No. 8, caramel and ferric oxide red);
clarifying agents (examples include but are not limited to bentonite); emulsifying agents (examples include but are not limited to acacia, cetomacrogol, cetyl alcohol, glyceryl monostearate, lecithin, sorbitan monooleate, polyoxyethylene 50 monostearate);
encapsulating agents (examples include but are not limited to gelatin and cellulose acetate phthalate);
flavorants (examples include but are not limited to anise oil, cinnamon oil, cocoa, menthol, orange oil, peppermint oil and vanillin);
humectants (examples include but are not limited to glycerol, propylene glycol and sorbitol); levigating agents (examples include but are not limited to mineral oil and glycerin);
oils (examples include but are not limited to arachis oil, mineral oil, olive oil, peanut oil, sesame oil and vegetable oil);
ointment bases (examples include but are not limited to lanolin, hydrophilic ointment, polyethylene glycol ointment, petrolatum, hydrophilic petrolatum, white ointment, yellow ointment, and rose water ointment);
penetration enhancers (transdermal delivery) (examples include but are not limited to monohydroxy or polyhydroxy alcohols, mono- or polyvalent alcohols, saturated or unsaturated fatty alcohols, saturated or unsaturated fatty esters, saturated or unsaturated dicarboxylic acids, essential oils, phosphatidyl derivatives, cephalin, terpenes, amides, ethers, ketones and ureas);
plasticizers (examples include but are not limited to diethyl phthalate and glycerol);
solvents (examples include but are not limited to ethanol, corn oil, cottonseed oil, glycerol, isopropanol, mineral oil, oleic acid, peanut oil, purified water, water for injection, sterile water for injection and sterile water for irrigation);
stiffening agents (examples include but are not limited to cetyl alcohol, cetyl esters wax, microcrystalline wax, paraffin, stearyl alcohol, white wax and yellow wax);
suppository bases (examples include but are not limited to cocoa butter and polyethylene glycols (mixtures));
surfactants (examples include but are not limited to benzalkonium chloride, nonoxynol 10, oxtoxynol 9, polysorbate 80, sodium lauryl sulfate and sorbitan mono-palmitate);
suspending agents (examples include but are not limited to agar, bentonite, carbomers, carboxymethylcellulose sodium, hydroxyethyl cellulose, hydroxypropyl cellulose, hydroxypropyl methylcellulose, kaolin, methylcellulose, tragacanth and veegum);
sweetening agents (examples include but are not limited to aspartame, dextrose, glycerol, mannitol, propylene glycol, saccharin sodium, sorbitol and sucrose);
tablet anti-adherents (examples include but are not limited to magnesium stearate and talc); tablet binders (examples include but are not limited to acacia, alginic acid, carboxymethylcellulose sodium, compressible sugar, ethylcellulose, gelatin, liquid glucose, methylcellulose, non-crosslinked polyvinyl pyrrolidone, and pregelatinized starch);
tablet and capsule diluents (examples include but are not limited to dibasic calcium phosphate, kaolin, lactose, mannitol, microcrystalline cellulose, powdered cellulose, precipitated calcium carbonate, sodium carbonate, sodium phosphate, sorbitol and starch);
tablet coating agents (examples include but are not limited to liquid glucose, hydroxyethyl cellulose, hydroxypropyl cellulose, hydroxypropyl methylcellulose, methylcellulose, ethylcellulose, cellulose acetate phthalate and shellac);
tablet direct compression excipients (examples include but are not limited to dibasic calcium phosphate);
tablet disintegrants (examples include but are not limited to alginic acid, carboxymethylcellulose calcium, microcrystalline cellulose, polacrillin potassium, cross linked polyvinylpyrrolidone, sodium alginate, sodium starch glycollate and starch);
tablet qlidants (examples include but are not limited to colloidal silica, corn starch and talc);
tablet lubricants (examples include but are not limited to calcium stearate, magnesium stearate, mineral oil, stearic acid and zinc stearate);
tablet/capsule opaquants (examples include but are not limited to titanium dioxide);
tablet polishing agents (examples include but are not limited to carnuba wax and white wax);
thickening agents (examples include but are not limited to beeswax, cetyl alcohol and paraffin);
tonicity aqents (examples include but are not limited to dextrose and sodium chloride);
viscosity increasing agents (examples include but are not limited to alginic acid, bentonite, carbomers, carboxymethylcellulose sodium, methylcellulose, polyvinyl pyrrolidone, sodium alginate and tragacanth);
and
wetting agents (examples include but are not limited to heptadecaethylene oxycetanol, lecithins, sorbitol monooleate, polyoxyethylene sorbitol monooleate, and polyoxyethylene stearate).

Pharmaceutical compositions according to the present invention can be illustrated as follows:

Sterile i.v. Solution:

A 5 mg/mL solution of the desired compound of this invention can be made using sterile, injectable water, and the pH is adjusted if necessary. The solution is diluted for administration to 1-2 mg/mL with sterile 5% dextrose and is administered as an i.v. infusion over about 60 minutes.

Lyophilized Powder for i.v. Administration:

A sterile preparation can be prepared with
(i) 100-1000 mg of the desired compound of this invention as a lypholized powder,
(ii) 32-327 mg/mL sodium citrate, and
(iii) 300-3000 mg Dextran 40. The formulation is reconstituted with sterile, injectable saline or dextrose 5% to a concentration of 10 to 20 mg/mL, which is further diluted i.v. infusion over 15-60 minutes.

### Intramuscular Suspension:

The following solution or suspension can be prepared, for

### Intramuscular Injection:

50 mg/mL of the desired, water-insoluble compound of this invention
5 mg/mL sodium carboxymethylcellulose
4 mg/mL TWEEN 80
9 mg/mL sodium chloride
9 mg/mL benzyl alcohol.

Hard Shell Capsules: A large number of unit capsules are prepared by filling standard two-piece hard galantine capsules each with 100 mg of powdered active ingredient, 150 mg of lactose, 50 mg of cellulose and 6 mg of magnesium stearate.

Soft Gelatin Capsules: A mixture of active ingredient in a digestible oil such as soybean oil, cottonseed oil or olive oil is prepared and injected by means of a positive displacement pump into molten gelatin to form soft gelatin capsules containing 100 mg of the active ingredient. The capsules are washed and dried. The active ingredient can be dissolved in a mixture of polyethylene glycol, glycerin and sorbitol to prepare a water miscible medicine mix.

Tablets: A large number of tablets are prepared by conventional procedures so that the dosage unit is 100 mg of active ingredient, 0.2 mg. of colloidal silicon dioxide, 5 mg of magnesium stearate, 275 mg of microcrystalline cellulose, 11 mg. of starch, and 98.8 mg of lactose. Appropriate aqueous and non-aqueous coatings may be applied to increase palatability, improve elegance and stability or delay absorption.

Immediate Release Tablets/Capsules: These are solid oral dosage forms made by conventional and novel processes. These units are taken orally without water for immediate dissolution and delivery of the medication. The active ingredient is mixed in a liquid containing ingredient such as sugar, gelatin, pectin and sweeteners. These liquids are solidified into solid tablets or caplets by freeze drying and solid state extraction techniques. The drug compounds may be compressed with viscoelastic and thermoelastic sugars and polymers or effervescent components to produce porous matrices intended for immediate release, without the need of water.

### Commercial Utility

### Component A

The compounds of formula (A) and (I) and the stereoisomers thereof according to the combination as referred to above are components A. The compounds according to the combination have valuable pharmaceutical properties, which make them commercially utilizable. In particular, they inhibit AhR.

### Component B

Due to the mechanism as discussed in the introductory section component B is especially suitable to have effects on tumor diseases, especially those where it is approved such as for the treatment of select patients with advanced melanoma, NSCLC, HNSCC, esophageal, urothelial bladder, kidney, gastric, hepatocellular carcinoma, cervical and merkel cell carcinoma as well as MSI high cancer, classical Hodgkin Lymphoma and primary mediastinal B cell lymphoma.

### Combination

The combinations of the present invention thus can be used for the treatment or prophylaxis of diseases of uncontrolled cell growth, proliferation and/or survival, inappropriate cellular immune responses, or inappropriate cellular inflammatory responses, or diseases which are accompanied with uncontrolled cell growth, proliferation and/or survival, inappropriate cellular immune responses, or inappropriate cellular inflammatory responses, particularly in which the uncontrolled cell growth, proliferation and/or survival, inappropriate cellular immune responses, or inappropriate cellular inflammatory responses, such as, for example, haematological tumours and/or metastases thereof, solid tumours, and/or metastases thereof, e.g. leukaemias, multiple myeloma thereof and myelodysplastic syndrome, malignant lymphomas, breast tumours including and bone metastases thereof, tumours of the thorax including non-small cell and small cell lung tumours and bone metastases thereof, gastrointestinal tumours, endocrine tumours, mammary and other gynaecological tumours and bone metastases thereof, urological tumours including renal, bladder and prostate tumours, skin tumours, and sarcomas, and/or metastases thereof.

One embodiment relates to the use of a combination as defined herein for the preparation of a medicament for the treatment or prophylaxis of a cancer, in particular disorders and conditions suitable for treatment with a composition of the present inventions can be, but are not limited to solid tumors, such as for example cancers of the breast, respiratory tract, brain, reproductive organs, digestive tract, urinary tract, eye, liver, skin, head and neck, thyroid, parathyroid, and their distant metastases. Those disorders also include lymphomas, sarcomas and leukemias.

Tumors of the digestive tract include, but are not limited to anal, colon, colorectal, esophageal, gallbladder, gastric, pancreatic, rectal, small-intestine, and salivary gland cancers.

Examples of esophageal cancer include but are not limited to esophageal cell carcinomas and Adenocarcinomas, as well as squamous cell carcinomas, Leiomyosarcoma, Malignant melanoma, rhabdomyosarcoma and Lymphoma.

Examples of gastric cancer include but are not limited to intestinal type and diffuse type gastric adenocarcinoma.

Examples of pancreatic cancer include, but are not limited to ductal adenocarcinoma, adenosquamous carcinomas and pancreatic endocrine tumors.

Examples of breast cancer include, but are not limited to triple negative breast cancer, invasive ductal carcinoma, invasive lobular carcinoma, ductal carcinoma in situ, and lobular carcinoma in situ.

Examples of cancers of the respiratory tract include but are not limited to small-cell and non-small-cell lung carcinoma, as well as bronchial adenoma and pleuropulmonary blastoma.

Examples of brain cancers include but are not limited to brain stem and hypothalamic glioma, cerebellar and cerebral astrocytoma, glioblastoma, medulloblastoma, ependymoma, as well as neuroectodermal and pineal tumor.

Tumors of the male reproductive organs include but are not limited to prostate and testicular cancer. Tumors of the female reproductive organs include, but are not limited to endometrial, cervical, ovarian, vaginal and vulvar cancer, as well as sarcoma of the uterus.

Examples of ovarian cancer include, but are not limited to serous tumour, endometrioid tumor, mucinous cystadenocarcinoma, granulosa cell tumor, Sertoli-Leydig cell tumor and arrhenoblastoma.

Examples of cervical cancer include, but are not limited to squamous cell carcinoma, adenocarcinoma, adenosquamous carcinoma, small cell carcinoma, neuroendocrine tumor, glassy cell carcinoma and villoglandular adenocarcinoma.

Tumors of the urinary tract include, but are not limited to bladder, penile, kidney, renal pelvis, ureter, urethral, and hereditary and sporadic papillary renal cancers.

Examples of kidney cancer include, but are not limited to renal cell carcinoma, urothelial cell carcinoma, juxtaglomerular cell tumor (reninoma), angiomyolipoma, renal oncocytoma, Bellini duct carcinoma, clear-cell sarcoma of the kidney, mesoblastic nephroma and Wilms' tumor. Examples of bladder cancer include, but are not limited to transitional cell carcinoma, squamous cell carcinoma, adenocarcinoma, sarcoma and small cell carcinoma.

Eye cancers include but are not limited to intraocular melanoma and retinoblastoma.

Examples of liver cancers include but are not limited to hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to squamous cell carcinoma, Kaposi's sarcoma, malignant melanoma, Merkel cell skin cancer, and non-melanoma skin cancer.

Head-and-neck cancers include but are not limited to squamous cell cancer of the head and neck, laryngeal, hypopharyngeal, nasopharyngeal, oropharyngeal cancer, salivary gland cancer, lip and oral cavity cancer, and squamous cell cancer.

Lymphomas include, but are not limited to AIDS-related lymphoma, non-Hodgkin's lymphoma, cutaneous T-cell lymphoma, Burkitt lymphoma, Hodgkin's disease, and lymphoma of the central nervous system.

Sarcomas include but are not limited to sarcoma of the soft tissue, osteosarcoma, malignant fibrous histiocytoma, lymphosarcoma, and rhabdomyosarcoma.

Leukemias include, but are not limited to acute myeloid leukemia, acute lymphoblastic leukemia, chronic lymphocytic leukemia, chronic myelogenous leukemia, and hairy cell leukemia.

In one embodiment the invention relates to combinations comprising component A or a pharmaceutically acceptable salt thereof and component B being intravenously, intraperitoneally, preferably it is administered orally.

The term “inappropriate” within the context of the present invention, in particular in the context of “inappropriate cellular immune responses, or inappropriate cellular inflammatory responses”, as used herein, is to be understood as preferably meaning a response which is less than, or greater than normal, and which is associated with, responsible for, or results in, the pathology of said diseases.

Combinations of the present invention might be utilized to inhibit, block, reduce, decrease, etc., cell proliferation and/or cell division, and/or produce apoptosis.

This invention includes a method comprising administering to a mammal in need thereof, including a human, an amount of a component A and an amount of component B of this invention, or a pharmaceutically acceptable salt, isomer, polymorph, metabolite, hydrate, solvate or ester thereof; etc. which is effective to treat the disorder.

Hyper-proliferative disorders include but are not limited, e.g., psoriasis, keloids, and other hyperplasias affecting the skin, benign prostate hyperplasia (BPH), as well as malignant neoplasia. Examples of malignant neoplasia treatable with the compounds according to the present invention include solid and hematological tumors. Solid tumors can be exemplified by tumors of the breast, bladder, bone, brain, central and peripheral nervous system, colon, anum, endocrine glands (e.g. thyroid and adrenal cortex), esophagus, endometrium, germ cells, head and neck, kidney, liver, lung, larynx and hypopharynx, mesothelioma, ovary, pancreas, prostate, rectum, renal, small intestine, soft tissue, testis, stomach, skin, ureter, vagina and vulva. Malignant neoplasias include inherited cancers exemplified by Retinoblastoma and Wilms tumor.

In addition, malignant neoplasias include primary tumors in said organs and corresponding secondary tumors in distant organs (“tumor metastases”). Hematological tumors can be exemplified by aggressive and indolent forms of leukemia and lymphoma, namely non-Hodgkins disease, chronic and acute myeloid leukemia (CML/AML), acute lymphoblastic leukemia (ALL), Hodgkins disease, multiple myeloma and T-cell lymphoma. Also included are myelodysplastic syndrome, plasma cell neoplasia, paraneoplastic syndromes, and cancers of unknown primary site as well as AIDS related malignancies.

Examples of breast cancer include, but are not limited to invasive ductal carcinoma, invasive lobular carcinoma, ductal carcinoma in situ, and lobular carcinoma in situ, particularly with bone metastases.

Examples of cancers of the respiratory tract include but are not limited to small-cell and non-small-cell lung carcinoma, as well as bronchial adenoma and pleuropulmonary blastoma.

Examples of brain cancers include, but are not limited to brain stem and hypothalamic glioma, cerebellar and cerebral astrocytoma, medulloblastoma, ependymoma, as well as neuroectodermal and pineal tumor.

Tumors of the male reproductive organs include but are not limited to prostate and testicular cancer. Tumors of the female reproductive organs include, but are not limited to endometrial, cervical, ovarian, vaginal, and vulvar cancer, as well as sarcoma of the uterus.

Tumors of the digestive tract include, but are not limited to anal, colon, colorectal, esophageal, gallbladder, gastric, pancreatic, rectal, small-intestine, and salivary gland cancers.

Tumors of the urinary tract include, but are not limited to bladder, penile, kidney, renal pelvis, ureter, urethral and human papillary renal cancers.

Eye cancers include but are not limited to intraocular melanoma and retinoblastoma.

Examples of liver cancers include but are not limited to hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to squamous cell carcinoma, Kaposi's sarcoma, malignant melanoma, Merkel cell skin cancer, and non-melanoma skin cancer. Head-and-neck cancers include, but are not limited to laryngeal, hypopharyngeal, nasopharyngeal, oropharyngeal cancer, lip and oral cavity cancer and squamous cell.

Lymphomas include, but are not limited to AIDS-related lymphoma, non-Hodgkin's lymphoma, cutaneous T-cell lymphoma, Burkitt lymphoma, Hodgkin's disease, and lymphoma of the central nervous system. Sarcomas include but are not limited to sarcoma of the soft tissue, osteosarcoma, malignant fibrous histiocytoma, lymphosarcoma, and rhabdomyosarcoma.

Leukemias include, but are not limited to acute myeloid leukemia, acute lymphoblastic leukemia, chronic lymphocytic leukemia, chronic myelogenous leukemia, and hairy cell leukemia.

These disorders have been well characterized in humans, but also exist with a similar etiology in other mammals and can be treated by administering pharmaceutical compositions of the present invention.

The term “treating” or “treatment” as stated throughout this document is used conventionally, e.g., the management or care of a subject for the purpose of combating, alleviating, reducing, relieving, improving the condition of, etc., of a disease or disorder, such as a carcinoma.

Combinations of the present invention might also be used for treating disorders and diseases associated with excessive and/or abnormal angiogenesis.

Inappropriate and ectopic expression of angiogenesis can be deleterious to an organism. A number of pathological conditions are associated with the growth of extraneous blood vessels.

These include, e.g., diabetic retinopathy, ischemic retinalvein occlusion, and retinopathy of prematurity [Aiello et al. New Engl. J. Med. 1994, 331, 1480; Peer et al. Lab. Invest. 1995, 72, 638], age-related macular degeneration [AMD; see, Lopez et al. Invest. Opththalmol. Vis. Sci. 1996, 37, 855], neovascular glaucoma, psoriasis, retrolental fibroplasias, angiofibroma, inflammation, rheumatoid arthritis (RA), restenosis, in-stent restenosis, vascular graft restenosis, etc. In addition, the increased blood supply associated with cancerous and neoplastic tissue, encourages growth, leading to rapid tumor enlargement and metastasis. Moreover, the growth of new blood and lymph vessels in a tumor provides an escape route for renegade cells, encouraging metastasis and the consequence spread of the cancer.

Thus, combinations of the present invention can be utilized to treat and/or prevent any of the aforementioned angiogenesis disorders, e.g., by inhibiting and/or reducing blood vessel formation; by inhibiting, blocking, reducing, decreasing, etc. endothelial cell proliferation or other types involved in angiogenesis, as well as causing cell death or apoptosis of such cell types.

### Dose and Administration

### Component a and Component B

Based upon standard laboratory techniques known to evaluate compounds useful for the treatment of hyper-proliferative disorders and angiogenic disorders, by standard toxicity tests and by standard pharmacological assays for the determination of treatment of the conditions identified above in mammals, and by comparison of these results with the results of known medicaments that are used to treat these conditions, the effective dosage of the compounds of this invention can readily be determined for treatment of each desired indication. The amount of the active ingredients to be administered in the treatment of one of these conditions can vary widely according to such considerations as the particular component and dosage unit employed, the mode of administration, the period of treatment, the age and sex of the patient treated, and the nature and extent of the condition treated.

The total amount of the active ingredients to be administered will generally range from about 0.001 mg/kg to about 200 mg/kg body weight per day, and preferably from about 0.01 mg/kg to about 20 mg/kg body weight per day. Clinically useful dosing schedules of a compound will range from one to three times a day dosing to once every four weeks dosing. In addition, “drug holidays” in which a patient is not dosed with a drug for a certain period of time, may be beneficial to the overall balance between pharmacological effect and tolerability. A unit dosage may contain from about 0.5 mg to about 1500 mg of active ingredient and can be administered one or more times per day or less than once a day. The average daily dosage for administration by injection, including intravenous, intramuscular, subcutaneous and parenteral injections, and use of infusion techniques will preferably be from 0.01 to 200 mg/kg of total body weight. The average daily rectal dosage regimen will preferably be from 0.01 to 200 mg/kg of total body weight. The average daily vaginal dosage regimen will preferably be from 0.01 to 200 mg/kg of total body weight. The average daily topical dosage regimen will preferably be from 0.1 to 200 mg administered between one to four times daily. The transdermal concentration will preferably be that required to maintain a daily dose of from 0.01 to 200 mg/kg. The average daily inhalation dosage regimen will preferably be from 0.01 to 100 mg/kg of total body weight.

Of course, the specific initial and continuing dosage regimen for each patient will vary according to the nature and severity of the condition as determined by the attending diagnostician, the activity of the specific compounds employed, the age and general condition of the patient, time of administration, route of administration, rate of excretion of the drug, drug combinations, and the like. The desired mode of treatment and number of doses of a compound of the present invention or a pharmaceutically acceptable salt or ester or composition thereof can be ascertained by those skilled in the art using conventional treatment tests.

### Combinations of the Present Invention

The combinations of the present invention can be used in particular in therapy and prevention, i.e. prophylaxis, of tumor growth and metastases, including solid and haematological tumors of all indications and stages with or without pre-treatment of the tumor growth.

Methods of testing for a particular pharmacological or pharmaceutical property are well known to persons skilled in the art.

The combinations of component A and component B of this invention can be administered as the sole pharmaceutical agent or in combination with one or more further pharmaceutical agents C where the resulting combination of components A, B and C causes no unacceptable adverse effects. For example, the combinations of components A and B of this invention can be combined with component C, i.e. one or more further pharmaceutical agents, such as known anti-angiogenesis, anti-hyper-proliferative, antiinflammatory, analgesic, immunoregulatory, diuretic, antiarrhytmic, anti-hypercholsterolemia, anti-dyslipidemia, anti-diabetic or antiviral agents, and the like, as well as with admixtures and combinations thereof.

Component C, can be one or more pharmaceutical agents such as 131I-chTNT, abarelix, abemaciclib, abiraterone, acalabrutinib, aclarubicin, adalimumab, ado-trastuzumab emtansine, afatinib, aflibercept, aldesleukin, alectinib, alemtuzumab, alendronic acid, alitretinoin, altretamine, amifostine, aminoglutethimide, hexyl aminolevulinate, amrubicin, amsacrine, anastrozole, ancestim, anethole dithiolethione, anetumab ravtansine, angiotensin II, antithrombin Ill, apalutamide, aprepitant, arcitumomab, arglabin, arsenic trioxide, asparaginase, atezolizumab, avelumab, axicabtagene ciloleucel, axitinib, azacitidine, basiliximab, belotecan, bendamustine, besilesomab, belinostat, bevacizumab, bexarotene, bicalutamide, bisantrene, bleomycin, blinatumomab, bortezomib, bosutinib, buserelin, brentuximab vedotin, brigatinib, busulfan, cabazitaxel, cabozantinib, calcitonine, calcium folinate, calcium levofolinate, capecitabine, capromab, carbamazepine carboplatin, carboquone, carfilzomib, carmofur, carmustine, catumaxomab, celecoxib, celmoleukin, ceritinib, cetuximab, chlorambucil, chlormadinone, chlormethine, cidofovir, cinacalcet, cisplatin, cladribine, clodronic acid, clofarabine, cobimetinib, copanlisib, crisantaspase, crizotinib, cyclophosphamide, cyproterone, cytarabine, dacarbazine, dactinomycin, daratumumab, darbepoetin alfa, dabrafenib, dasatinib, daunorubicin, decitabine, degarelix, denileukin diftitox, denosumab, depreotide, deslorelin, dianhydrogalactitol, dexrazoxane, dibrospidium chloride, dianhydrogalactitol, diclofenac, dinutuximab, docetaxel, dolasetron, doxifluridine, doxorubicin, doxorubicin+estrone, dronabinol, durvalumab, eculizumab, edrecolomab, elliptinium acetate, elotuzumab, eltrombopag, Enasidenib, endostatin, enocitabine, enzalutamide, epirubicin, epitiostanol, epoetin alfa, epoetin beta, epoetin zeta, eptaplatin, eribulin, erlotinib, esomeprazole, estradiol, estramustine, ethinylestradiol, etoposide, everolimus, exemestane, fadrozole, fentanyl, filgrastim, fluoxymesterone, floxuridine, fludarabine, fluorouracil, flutamide, folinic acid, formestane, fosaprepitant, fotemustine, fulvestrant, gadobutrol, gadoteridol, gadoteric acid meglumine, gadoversetamide, gadoxetic acid, gallium nitrate, ganirelix, gefitinib, gemcitabine, gemtuzumab, Glucarpidase, glutoxim, GM-CSF, goserelin, granisetron, granulocyte colony stimulating factor, histamine dihydrochloride, histrelin, hydroxycarbamide, I-125 seeds, lansoprazole, ibandronic acid, ibritumomab tiuxetan, ibrutinib, idarubicin, ifosfamide, imatinib, imiquimod, improsulfan, indisetron, incadronic acid, ingenol mebutate, inotuzumab ozogamicin, interferon alfa, interferon beta, interferon gamma, iobitridol, iobenguane (I231), iomeprol, ipilimumab, irinotecan, Itraconazole, ixabepilone, ixazomib, lanreotide, lansoprazole, lapatinib, lasocholine, lenalidomide, lenvatinib, lenograstim, lentinan, letrozole, leuprorelin, levamisole, levonorgestrel, levothyroxine sodium, lisuride, lobaplatin, lomustine, lonidamine, lutetium Lu 177 dotatate, masoprocol, medroxyprogesterone, megestrol, melarsoprol, melphalan, mepitiostane, mercaptopurine, mesna, methadone, methotrexate, methoxsalen, methylaminolevulinate, methylprednisolone, methyltestosterone, metirosine, midostaurin, mifamurtide, miltefosine, miriplatin, mitobronitol, mitoguazone, mitolactol, mitomycin, mitotane, mitoxantrone, mogamulizumab, molgramostim, mopidamol, morphine hydrochloride, morphine sulfate, mvasi, nabilone, nabiximols, nafarelin, naloxone+pentazocine, naltrexone, nartograstim, necitumumab, nedaplatin, nelarabine, neratinib, neridronic acid, netupitant/palonosetron, pentetreotide, nilotinib, nilutamide, nimorazole, nimotuzumab, nimustine, nintedanib, niraparib, nitracrine, obinutuzumab, octreotide, ofatumumab, olaparib, olaratumab, omacetaxine mepesuccinate, omeprazole, ondansetron, oprelvekin, orgotein, orilotimod, osimertinib, oxaliplatin, oxycodone, oxymetholone, ozogamicine, p53 gene therapy, paclitaxel, palbociclib, palifermin, palladium-103 seed, palonosetron, pamidronic acid, panitumumab, panobinostat, pantoprazole, pazopanib, pegaspargase, PEG-epoetin beta (methoxy PEG-epoetin beta), pegfilgrastim, peginterferon alfa-2b, pemetrexed, pentazocine, pentostatin, peplomycin, Perflubutane, perfosfamide, Pertuzumab, picibanil, pilocarpine, pirarubicin, pixantrone, plerixafor, plicamycin, poliglusam, polyestradiol phosphate, polyvinylpyrrolidone+sodium hyaluronate, polysaccharide-K, pomalidomide, ponatinib, porfimer sodium, pralatrexate, prednimustine, prednisone, procarbazine, procodazole, propranolol, quinagolide, rabeprazole, racotumomab, radium-223 chloride, radotinib, raloxifene, raltitrexed, ramosetron, ramucirumab, ranimustine, rasburicase, razoxane, refametinib, regorafenib, ribociclib, risedronic acid, rhenium-186 etidronate, rituximab, rolapitant, romidepsin, romiplostim, romurtide, rucaparib, samarium (153Sm) lexidronam, sargramostim, sarilumab, satumomab, secretin, siltuximab, sipuleucel-T, sizofiran, sobuzoxane, sodium glycididazole, sonidegib, sorafenib, stanozolol, streptozocin, sunitinib, talaporfin, talimogene laherparepvec, tamibarotene, tamoxifen, tapentadol, tasonermin, teceleukin, technetium (99mTc) nofetumomab merpentan, 99mTc-HYNIC-[Tyr3]-octreotide, tegafur, tegafur+gimeracil+oteracil, temoporfin, temozolomide, temsirolimus, teniposide, testosterone, tetrofosmin, thalidomide, thiotepa, thymalfasin, thyrotropin alfa, tioguanine, tisagenlecleucel, tocilizumab, topotecan, toremifene, tositumomab, trabectedin, trametinib, tramadol, trastuzumab, trastuzumab emtansine, treosulfan, tretinoin, trifluridine+tipiracil, trilostane, triptorelin, trametinib, trofosfamide, thrombopoietin, tryptophan, ubenimex, valatinib, valrubicin, vandetanib, vapreotide, vemurafenib, vinblastine, vincristine, vindesine, vinflunine, vinorelbine, vismodegib, vorinostat, vorozole, yttrium-90 glass microspheres, zinostatin, zinostatin stimalamer, zoledronic acid, zorubicin, or combinations thereof.

Alternatively, said component C can be one or more further pharmaceutical agents selected from gemcitabine, paclitaxel, cisplatin, carboplatin, sodium butyrate, 5-FU, doxirubicin, tamoxifen, etoposide, trastumazab, gefitinib, intron A, rapamycin, 17-AAG, U0126, insulin, an insulin derivative, a PPAR ligand, a sulfonylurea drug, an α-glucosidase inhibitor, a biguanide, a PTP-1B inhibitor, a DPP-IV inhibitor, a 11-beta-HSD inhibitor, GLP-1, a GLP-1 derivative, GIP, a GIP derivative, PACAP, a PACAP derivative, secretin or a secretin derivative.

Optional anti-hyper-proliferative agents which can be added as component C to the combination of components A and B of the present invention include but are not limited to compounds listed on the cancer chemotherapy drug regimens in the 11th Edition of the Merck Index, (1996), which is hereby incorporated by reference, such as asparaginase, bleomycin, carboplatin, carmustine, chlorambucil, cisplatin, colaspase, cyclophosphamide, cytarabine, dacarbazine, dactinomycin, daunorubicin, doxorubicin (adriamycine), epirubicin, etoposide, 5-fluorouracil, hexamethylmelamine, hydroxyurea, ifosfamide, irinotecan, leucovorin, lomustine, mechlorethamine, 6mercaptopurine, mesna, methotrexate, mitomycin C, mitoxantrone, prednisolone, prednisone, procarbazine, raloxifen, streptozocin, tamoxifen, thioguanine, topotecan, vinblastine, vincristine, and vindesine.

Other anti-hyper-proliferative agents suitable for use as component C with the combination of components A and B of the present invention include but are not limited to those compounds acknowledged to be used in the treatment of neoplastic diseases in Goodman and Gilman's The Pharmacological Basis of Therapeutics (Ninth Edition), editor Molinoff et al., publ. by McGraw-Hill, pages 1225-1287, (1996), which is hereby incorporated by reference, such as aminoglutethimide, L-asparaginase, azathioprine, 5-azacytidine cladribine, busulfan, diethylstilbestrol, 2′,2′difluorodeoxycytidine, docetaxel, erythrohydroxynonyl adenine, ethinyl estradiol, fluorodeoxyuridine, 5-fluorodeoxyuridine monophosphate, fludarabine phosphate, fluoxymesterone, flutamide, hydroxyprogesterone caproate, idarubicin, interferon, medroxyprogesterone acetate, megestrol acetate, melphalan, mitotane, paclitaxel (when component B is not itself paclitaxel), pentostatin, N-phosphonoacetyl-Laspartate (PALA), plicamycin, semustine, teniposide, testosterone propionate, thiotepa, trimethylmelamine, uridine, and vinorelbine.

Other anti-hyper-proliferative agents suitable for use as component C with the combination of components A and B of the present invention include but are not limited to other anti-cancer agents such as epothilone and its derivatives, irinotecan, raloxifen and topotecan.

Generally, the use of cytotoxic and/or cytostatic agents as component C in combination with a combination of components A and B of the present invention will serve to:
(1) yield better efficacy in reducing the growth of a tumor and/or metastasis or even eliminate the tumor and/or metastasis as compared to administration of either agent alone,
(2) provide for the administration of lesser amounts of the administered chemo therapeutic agents,
(3) provide for a chemotherapeutic treatment that is well tolerated in the patient with fewer deleterious pharmacological complications than observed with single agent chemotherapies and certain other combined therapies,
(4) provide for treating a broader spectrum of different cancer types in mammals, especially humans,
(5) provide for a higher response rate among treated patients,
(6) provide for a longer survival time among treated patients compared to standard chemotherapy treatments,
(7) provide a longer time for tumor progression, and/or
(8) yield efficacy and tolerability results at least as good as those of the agents used alone, compared to known instances where other cancer agent combinations produce antagonistic effects.

### FIGURES

FIG. 1 shows the experimental results of the combination of component A with pembrolizumab.

FIG. 2 shows the experimental results of the combination of component A with nivolumab.

### EXPERIMENTAL SECTION

Examples demonstrating the synergistic effect of the combinations of components A and B of the present invention

### Component A:

In this Experimental section and in the Figures, the term “compound A” is an example of component A and is compound Example 17 of WO 2018/146010 A1 as shown herein:

it is 6-(4-Chlorophenyl)-N-[(2S)-1-hydroxypropan-2-yl]-2-(1-methyl-1H-pyrazol-4-yl)-3-oxo-2,3-dihydropyridazine-4-carboxamide, of structure:

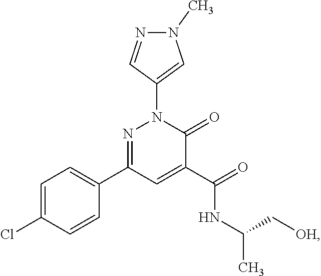 [SMILES: C[C@@H](CO)NC(=O)c1cc(-c2ccc(Cl)cc2)nn(-c2cnn(C)c2)c1=O]

or a polymorph, enantiomer, diastereomer, racemate, tautomer, N-oxide, hydrate and solvate thereof, as well as the physiological acceptable salts and solvates of the salts, as well as mixtures of the same thereof.

### Component B:

Component B is pembrolizumab or is nivolumab.

### Mixed Lymphocyte Reaction

Whole blood samples from healthy donors were collected and PBMCs were isolated using density gradient centrifugation for 15 min at 800×g at room temperature with Biocoll separation solution (density 1.977 g/ml; Biochrom, #L6115). The supernatants were discarded and the PBMCs were poured into 50 ml tubes, washed with PBS without Ca⁺/Mg⁺, counted and resuspended in MACS separation buffer (Miltenyi Biotec). The monocytes were isolated from PBMCs using the Monocyte Isolation Kit II (Miltenyi Biotec, Cat #130-091-153) according to manufacturer's instructions. On the day of co-culture, T cells were isolated from PBMC preparations obtained from whole blood of different donors using the Pan T Cell Isolation Kit (Auto MACS Pro, Cat #130-096-535).

Monocytes were cultured in RPMI 1640+Glutamax (Invitrogen, Cat #61870-044) medium supplemented with 10% fetal calf serum (FCS) Superior (Biochrome, Cat #S0615) and 50 U/ml or μg/ml penicillin/streptomycin (Biochrom, Cat #A2213). Monocytes were differentiated into monocyte-derived dendritic cells (moDCs) using 100 ng/ml GM-CSF (R&D Systems, Cat #215-GM) and 100 ng/ml rhIL4 (R&D Systems, Cat #204-IL) for 6 days at 37° C., 5% CO₂. To produce mature DCs, moDCs were further matured for 24 h with IFNγ (R&D Systems, Cat #285-IF) and lipopolysaccharide (LPS) from Escherichia coli O127: B8 (Sigma, Cat #L4516).

For the co-culture assay, matured DCs and T cells from different donors were used. The cells were seeded in 96-well plates (Costar, Cat #3585) at 1×10⁴ cells/well for matured DCs and 5×10⁴ cells/well for T cells. A serial dilution of component A (3× dilution steps) in combination with anti-programmed cell death 1 antibody (as component B: aPD-1; 1 μg/ml pembrolizumab or 3 μg/ml nivolumab) or respective concentrations of hIgG4 (isotype control TPP10747, PPB11084) was added and the cells were incubated for 3 days at 37° C., 5% CO₂.

IL-2 cytokine levels were measured in aliquots of supernatants using the Human IL-2 Tissue Culture kit (Meso Scale Discovery, Cat #K151AHB-4).

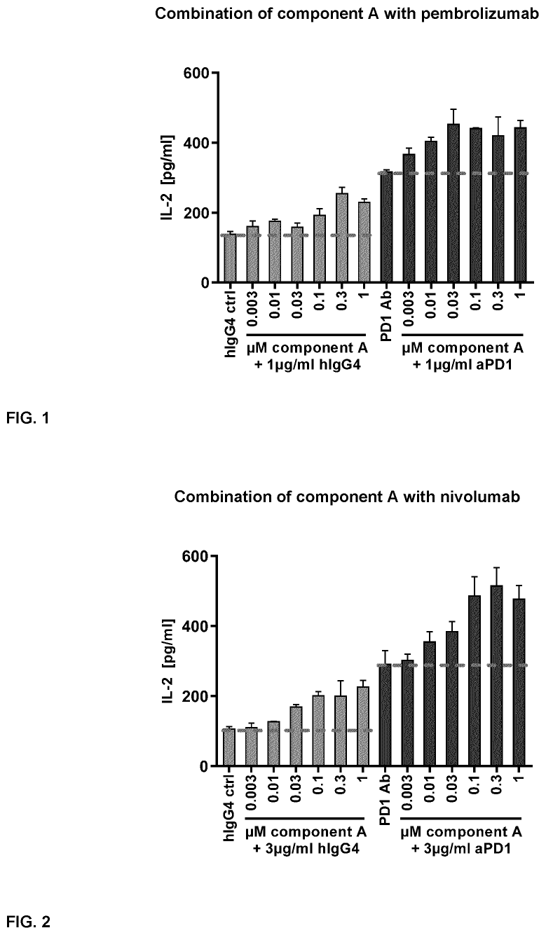

The results of the combination of component A with pembrolizumab and the combination of component A with nivolumab are shown in FIG. 1 and FIG. 2.

### CONCLUSIONS

The results clearly demonstrate that component A enhances T cell activity, alone and in combination with PD1 antibodies, as observed by increased IL-2 production from T cells in human T cell/DC co-cultures. In particular, IL-2 shows a dose-dependent response upon treatment with component A alone (component A+hIgG4 control). Furthermore, component A combines with PD1 antibodies (component A+aPD1); (Component B: pembrolizumab or nivolumab) resulting in higher IL-2 levels as compared to treatment with aPD-1 (first blue bar) or component A alone (component A+hIgG4).

The combination of 6-(4-Chlorophenyl)-N-[(2S)-1-hydroxypropan-2-yl]-2-(1-methyl-1H-pyrazol-4-yl)-3-oxo-2,3-dihydropyridazine-4-carboxamide and pembrolizumab enhanced the anti-proliferative effect leading to very strong synergy.

In summary, the data indicate synergistic effects of the AhR inhibitor 6-(4-Chlorophenyl)-N-[(2S)-1-hydroxypropan-2-yl]-2-(1-methyl-1H-pyrazol-4-yl)-3-oxo-2,3-dihydropyridazine-4-carboxamide and pembrolizumab or nivolumab in inhibiting tumor cell proliferation and warrant further clinical evaluation of this promising combination therapy for the treatment of cancer, including cancers of the breast, respiratory tract, brain, reproductive organs, digestive tract, urinary tract, eye, liver, skin, kidney, head and neck, thyroid, parathyroid, and their distant metastases, lymphomas, sarcomas and leukemias.

1: A combination comprising at least two components, component A and component B, wherein
component A is an Aryl Hydrocarbon Receptor (AhR) inhibitor or a polymorph, enantiomer, diastereomer, racemate, tautomer, N-oxide, hydrate, solvate, physiologically acceptable salt, solvate of a physiologically acceptable salt thereof, or a mixture of any of the foregoing; and
component B is a PD-1 inhibitor antibody.

2: The combination of claim 1, wherein component A is a compound of formula (I) 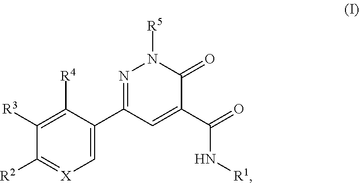
wherein
R¹ is C₂-C₆-hydroxyalkyl optionally substituted once with cyano, —COOR¹⁰, —CONR¹¹R¹², C₁-C₄-alkoxy, or C₃-C₆-cycloalkyl and optionally substituted one to three times with halogen, or C₃-C₆-cycloalkyl substituted once with hydroxy and optionally substituted once with C₁-C₃-alkyl and/or one to three times with halogen, or C₃-C₆-cycloalkyl-C₁-C₃-alkyl substituted once with hydroxy and optionally substituted once with C₁-C₃-alkyl and/or one to three times with halogen, or (C₃-C₆-cycloalkyl)₂-C₁-C₃-alkyl substituted once with hydroxy and optionally once with C₁-C₃-alkyl and/or one to three times with halogen, or 4- to 6-membered heterocycloalkyl substituted once with hydroxy and optionally once with C₁-C₃-alkyl and/or one to three times with halogen;
R² is chloro, cyano, dimethylamino, methyl, fluoromethyl, difluoromethyl, trifluoromethyl, methoxy, difluoromethoxy, or trifluoromethoxy;
R³ is hydrogen, fluoro, chloro, or methyl;
R⁴ is hydrogen or fluoro;
R⁵ is monocyclic heteroaryl, optionally substituted one to three times with R⁶;
each R⁶ is independently C₁-C₄-alkyl, C₁-C₄-haloalkyl, C₃-C₆-cycloalkyl, C₁-C₄-alkoxy, halogen, or cyano;
X is CH or N;
R¹⁰ is C₁-C₄-alkyl; and
R¹¹ and R¹² are independently from each other, hydrogen or C₁-C₃-alkyl, or
together with the nitrogen atom to which they are attached form a 4- to 6-membered nitrogen containing heterocyclic ring, said ring optionally containing one additional heteroatom selected from O, S, NH, NR^{a} in which R^{a} represents a C₁-C₄-alkyl group,
or a polymorph, enantiomer, diastereomer, racemate, tautomer, N oxide, hydrate, solvate, physiologically acceptable salt, or a solvate of a physiologically acceptable salt thereof, or a mixture of any of the foregoing.

3: The combination according to claim 2, wherein component A is a compound of formula (I), wherein
R¹ is C₂-C₆-hydroxyalkyl optionally substituted once with cyano, —COOR¹⁰, —CONR¹¹R¹², C₁-C₄-alkoxy, or
C₃-C₆-cycloalkyl and optionally substituted one to three times with halogen, or
C₃-C₆-cycloalkyl substituted once with hydroxy and optionally one to three times with halogen, or
C₃-C₆-cycloalkyl-C₁-C₃-alkyl substituted once with hydroxy and optionally one to three times with halogen, or
(C₃-C₆-cycloalkyl)₂-C₁-C₃-alkyl substituted once with hydroxy and optionally one to three times with halogen, or
4- to 6-membered heterocycloalkyl substituted once with hydroxy and optionally one to three times with halogen;
R² is chloro, cyano, dimethylamino, methyl, fluoromethyl, difluoromethyl, trifluoromethyl, methoxy, difluoromethoxy, or trifluoromethoxy;
R³ is hydrogen, fluoro, chloro, or methyl;
R⁴ is hydrogen or fluoro;
R⁵ is monocyclic heteroaryl optionally substituted one to three times with R⁶;
each R⁶ is independently C₁-C₄-alkyl, C₁-C₄-haloalkyl, C₃-C₆-cycloalkyl, C₁-C₄-alkoxy, halogeno or cyano;
X is CH or N;
R¹⁰ is C₁-C₄-alkyl; and
R¹¹ and R¹² are independently from each other, hydrogen or C₁-C₃-alkyl, or
together with the nitrogen atom to which they are attached form a 4- to 6-membered nitrogen containing heterocyclic ring, said ring optionally containing one additional heteroatom selected from O, S, NH, NR^{a} in which R^{a} represents a C₁-C₄-alkyl group,
or a polymorph, enantiomer, diastereomer, racemate, tautomer, N oxide, hydrate, solvate, physiologically acceptable salt, or a solvate of a physiologically acceptable salt thereof, or a mixture of any of the foregoing.

4: The combination according to claim 2, wherein said component A is a compound of formula (I), wherein
R¹ is C₂-C₆-hydroxyalkyl optionally substituted once with cyano, —COOR¹⁰, —CONR¹¹R¹², C₁-C₄-alkoxy, or
C₃-C₆-cycloalkyl and optionally one to three times with halogen, or
C₃-C₆-cycloalkyl substituted once with hydroxy and optionally once with C₁-C₃-alkyl and/or one to three times with halogen, or
C₃-C₆-cycloalkyl-C₁-C₃-alkyl substituted once with hydroxy and optionally once with C₁-C₃-alkyl and/or one to three times with halogen, or
(C₃-C₆-cycloalkyl)₂-C₁-C₃-alkyl substituted once with hydroxy and optionally once with C₁-C₃-alkyl and/or one to three times with halogen, or
4- to 6-membered heterocycloalkyl substituted once with hydroxy and optionally once with C₁-C₃-alkyl and/or one to three times with halogen;
R² is chloro, cyano, dimethylamino, methyl, fluoromethyl, difluoromethyl, trifluoromethyl, methoxy, difluoromethoxy, or trifluoromethoxy;
R³ is hydrogen, fluoro, chloro, or methyl;
R⁴ is hydrogen or fluoro;
R⁵ is monocyclic heteroaryl is optionally substituted one to three times with R⁶;
each R⁶ is independently C₁-C₄-alkyl, C₃-C₆-cycloalkyl, C₁-C₄-alkoxy, halogen or cyano; and
X is CH or N,
or a polymorph, enantiomer, diastereomer, racemate, tautomer, N oxide, hydrate, solvate, physiologically acceptable salt, or a solvate of a physiologically acceptable salt thereof, or a mixture of any of the foregoing.

5: The combination according to claim 2, wherein component A is a compound of formula (I), wherein
R¹ is C₂-C₅-hydroxyalkyl optionally substituted once with cyano, —COOCH₃, —CONH₂, methoxy, or cyclopropyl and optionally substituted one to three times with fluoro, or
C₄-C₆-cycloalkyl substituted once with hydroxy and optionally once with methyl and/or one to two times with fluoro, or
C₃-C₄-cycloalkyl-methyl substituted once with hydroxy, or
5- or 6-membered heterocycloalkyl substituted once with hydroxy, wherein the heterocycloalkyl contains one oxygen atom;
R² is chloro, cyano, dimethylamino, methyl, fluoromethyl, difluoromethyl, trifluoromethyl, methoxy, difluoromethoxy, or trifluoromethoxy;
R³ is hydrogen or fluoro;
R⁴ is hydrogen or fluoro;
R⁵ is monocyclic heteroaryl optionally substituted one to three times, independently, with R⁶;
each R⁶ is methyl, difluoromethyl, methoxy, halogen, or cyano;
X is CH or N;
R¹⁰ is C₁-C₄-alkyl; and
R¹¹ and R¹² are independently from each other, hydrogen or C₁-C₃-alkyl, or
together with the nitrogen atom to which they are attached form a 4- to 6-membered nitrogen containing heterocyclic ring, said ring optionally containing one additional heteroatom selected from O, S, NH, NR^{a} in which R^{a} represents a C₁-C₄-alkyl group,
or a polymorph, enantiomer, diastereomer, racemate, tautomer, N oxide, hydrate, solvate, physiologically acceptable salt, or a solvate of a physiologically acceptable salt thereof, or a mixture of any of the foregoing.

6: The combination according to claim 2, wherein component A is a compound of formula (I), wherein
R¹ is C₂-C₅-hydroxyalkyl optionally substituted once with cyano, —COOCH₃, —CONH₂, methoxy or cyclopropyl and optionally substituted one to three times with fluoro, or
C₄-C₆-cycloalkyl substituted once with hydroxy and optionally substituted once with methyl and/or one to two times with fluoro, or
C₃-C₄-cycloalkyl-methyl substituted once with hydroxy, or
5- or 6-membered heterocycloalkyl substituted once with hydroxy, said wherein the heterocycloalkyl contains one oxygen atom;
R² is chloro, dimethylamino, methyl, fluoromethyl, difluoromethyl, trifluoromethyl, difluoromethoxy, or trifluoromethoxy;
R³ is hydrogen;
R⁴ is hydrogen or fluoro;
R⁵ is 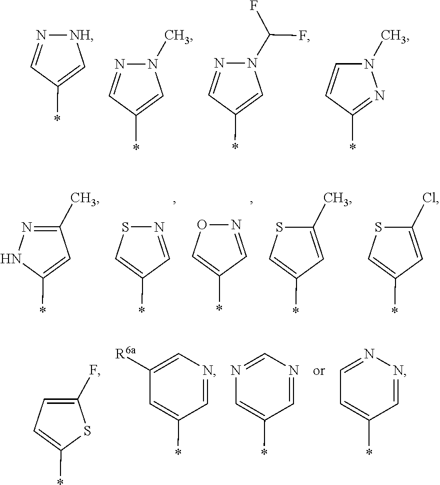
wherein * indicates the point of attachment of said group with the rest of the molecule;
R^{6a} is hydrogen, methyl, fluoro or chloro; and
X is CH or N;
or a polymorph, enantiomer, diastereomer, racemate, tautomer, N oxide, hydrate, solvate, physiologically acceptable salt, or a solvate of a physiologically acceptable salt thereof, or a mixture of any of the foregoing.

7: The combination according to claim 1, wherein component A is a compound of formula (Ia), 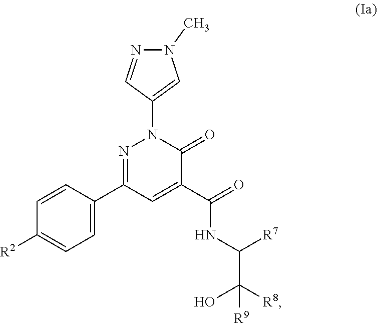
wherein
R² is chloro, dimethylamino, methyl, fluoromethyl, difluoromethyl, trifluoromethyl, difluoromethoxy, or trifluoromethoxy;
R⁷ is hydrogen, methyl, fluoromethyl, difluoromethyl, trifluoromethyl, hydroxymethyl, methoxymethyl, ethyl, isopropyl, cyclopropyl, cyano, —COOCH₃, or —CONH₂;
R⁸ is hydrogen, methyl, fluoromethyl, difluoromethyl, trifluoromethyl, hydroxymethyl, or methoxymethyl,
wherein one of R⁷ and R⁸ is different from hydrogen, or
together R⁷ and R⁸ form a cyclopentyl or cyclohexyl ring, which is optionally substituted one to two times with fluoro, or a heterocycloalkyl ring, which contains one oxygen atom; and
R⁹ is hydrogen or methyl, or
together R⁸ and R⁹ form a cyclopropyl or cyclobutyl ring;
or a polymorph, enantiomer, diastereomer, racemate, tautomer, N oxide, hydrate, solvate, physiologically acceptable salt, or a solvate of a physiologically acceptable salt thereof, or a mixture of any of the foregoing.

8: The combination according to claim 1, wherein component A is a compound of formula (Ib), 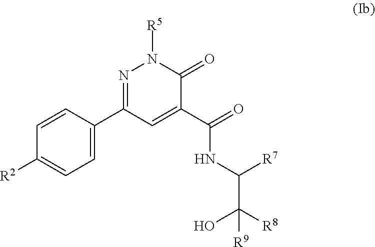
wherein
R² is chloro, dimethylamino, methyl, fluoromethyl, difluoromethyl, trifluoromethyl, difluoromethoxy, or trifluoromethoxy;
R⁵ is 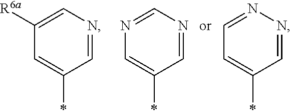
wherein * indicates the point of attachment of said group with the rest of the molecule;
R^{6a} is hydrogen, methyl, fluoro, or chloro;
R⁷ is hydrogen, methyl, fluoromethyl, difluoromethyl, trifluoromethyl, hydroxymethyl, methoxymethyl, ethyl, isopropyl, or cyclopropyl;
R⁸ is hydrogen, methyl, fluoromethyl, difluoromethyl, trifluoromethyl, hydroxymethyl, or methoxymethyl,
wherein one of R⁷ and R⁸ is different from hydrogen, or
together R⁷ and R⁸ form a cyclopentyl or cyclohexyl ring, which is optionally substituted one to two times with fluoro, or a heterocycloalkyl ring, which contains one oxygen or sulphur atom; and
R⁹ is hydrogen or methyl, or
together R⁸ and R⁹ form a cyclopropyl or cyclobutyl ring;
or a polymorph, enantiomer, diastereomer, racemate, tautomer, N oxide, hydrate, solvate, physiologically acceptable salt, or a solvate of a physiologically acceptable salt thereof, or a mixture of any of the foregoing.

9: The combination according to claim 2, wherein component A is a compound of formula (I), wherein
R¹ is C₂-C₆-hydroxyalkyl optionally substituted once with cyano, —COOR¹⁰, —CONR¹¹R¹², C₁-C₄-alkoxy, or
C₃-C₆-cycloalkyl and optionally substituted one to three times with halogen, or
C₃-C₆-cycloalkyl substituted once with hydroxy and optionally once with C₁-C₃-alkyl and/or one to three times with halogen, or
C₃-C₆-cycloalkyl-C₁-C₃-alkyl substituted once with hydroxy and optionally substituted once with C₁-C₃-alkyl and/or one to three times with halogen, or
(C₃-C₆-cycloalkyl)₂-C₁-C₃-alkyl substituted once with hydroxy and optionally substituted once with C₁-C₃-alkyl and/or one to three times with halogen, or
4- to 6-membered heterocycloalkyl substituted once with hydroxy and optionally substituted once with C₁-C₃-alkyl and/or one to three times with halogen,
or a polymorph, enantiomer, diastereomer, racemate, tautomer, N oxide, hydrate, solvate, physiologically acceptable salt, or a solvate of a physiologically acceptable salt thereof, or a mixture of any of the foregoing.

10: The combination according to claim 2, wherein component A is a compound of formula (I), wherein
R¹ is C₂-C₅-hydroxyalkyl optionally substituted once with cyano, —COOCH₃, —CONH₂, methoxy, or cyclopropyl and optionally substituted one to three times with fluoro, or
C₄-C₆-cycloalkyl substituted once with hydroxy and optionally substituted once with methyl and/or one to two times with fluoro, or
C₃-C₄-cycloalkyl-methyl substituted once with hydroxy, or
5- or 6-membered heterocycloalkyl substituted once with hydroxy, wherein the heterocycloalkyl contains one oxygen atom,
or a polymorph, enantiomer, diastereomer, racemate, tautomer, N oxide, hydrate, solvate, physiologically acceptable salt, or a solvate of a physiologically acceptable salt thereof, or a mixture of any of the foregoing.

11: The combination according to claim 2, wherein component A is a compound of formula (I), wherein
R¹ is C₂-C₆-hydroxyalkyl optionally substituted once with cyano, —COOR¹⁰, —CONR¹¹R¹², C₁-C₄-alkoxy, or C₃-C₆-cycloalkyl and optionally substituted one to three times with halogen, or
C₃-C₆-cycloalkyl substituted once with hydroxy and optionally substituted one to three times with halogen, or
C₃-C₆-cycloalkyl-C₁-C₃-alkyl substituted once with hydroxy and optionally substituted one to three times with halogen, or
(C₃-C₆-cycloalkyl)₂-C₁-C₃-alkyl substituted once with hydroxy and optionally substituted one to three times with halogen, or
4- to 6-membered heterocycloalkyl substituted once with hydroxy and optionally one to three times with halogen,
or a polymorph, enantiomer, diastereomer, racemate, tautomer, N oxide, hydrate, solvate, physiologically acceptable salt, or a solvate of a physiologically acceptable salt thereof, or a mixture of any of the foregoing.

12: The combination according to claim 2, wherein component A is a compound of formula (I), wherein
R¹ is C₂-C₆-hydroxyalkyl optionally substituted once with C₁-C₄-alkoxy or C₃-C₆-cycloalkyl and optionally substituted one to three times with halogen, or
C₃-C₆-cycloalkyl substituted once with hydroxy and optionally substituted once with methyl and/or one to three times with halogen, or
C₃-C₆-cycloalkyl-C₁-C₃-alkyl substituted once with hydroxy and optionally substituted one to three times with halogen, or
(C₃-C₆-cycloalkyl)₂-C₁-C₃-alkyl substituted once with hydroxy and optionally substituted one to three times with halogen, or
4- to 6-membered heterocycloalkyl substituted once with hydroxy and optionally substituted one to three times with halogen;
or a polymorph, enantiomer, diastereomer, racemate, tautomer, N oxide, hydrate, solvate, physiologically acceptable salt, or a solvate of a physiologically acceptable salt thereof, or a mixture of any of the foregoing.

13: The combination according to claim 2, wherein component A is a compound of formula (I), wherein
R¹ is C₂-C₆-hydroxyalkyl optionally substituted once with C₁-C₂-alkoxy or cyclopropyl and optionally substituted one to three times with halogen, or
C₄-C₆-cycloalkyl substituted once with hydroxy and optionally substituted once with methyl and/or one to three times with halogen, or
C₃-C₆-cycloalkyl-methyl substituted once with hydroxy and optionally substituted one to three times with halogen, or
(C₃-C₆-cycloalkyl)₂-methyl substituted once with hydroxy and optionally substituted one to three times with halogen, or
5- or 6-membered heterocycloalkyl substituted once with hydroxy and optionally substituted one to three times with halogen,
or a polymorph, enantiomer, diastereomer, racemate, tautomer, N oxide, hydrate, solvate, physiologically acceptable salt, or a solvate of a physiologically acceptable salt thereof, or a mixture of any of the foregoing.

14: The combination according to claim 2, wherein component A is a compound of formula (I), wherein
R¹ is C₂-C₅-hydroxyalkyl optionally substituted once with methoxy or cyclopropyl and optionally substituted one to three times with fluoro, or
C₄-C₆-cycloalkyl substituted once with hydroxy and optionally substituted once with methyl and/or one to two times with fluoro, or
C₃-C₄-cycloalkyl-methyl substituted once with hydroxy, or
5- or 6-membered heterocycloalkyl substituted once with hydroxy, said heterocycloalkyl contains one oxygen atom,
or a polymorph, enantiomer, diastereomer, racemate, tautomer, N oxide, hydrate, solvate, physiologically acceptable salt, or a solvate of a physiologically acceptable salt thereof, or a mixture of any of the foregoing.

15: The combination according to claim 2, wherein said component A is a compound of formula (I), wherein
R¹ is C₂-C₅-hydroxyalkyl, wherein said C₂-C₅-hydroxyalkyl groups are optionally substituted once with methoxy or cyclopropyl and optionally substituted one to three times with fluoro, or
C₄-C₆-cycloalkyl substituted once with hydroxy and optionally substituted once with methyl or one to two times with fluoro, or
C₃-C₄-cycloalkyl-methyl substituted once with hydroxy, or
5- or 6-membered heterocycloalkyl substituted once with hydroxy, wherein the heterocycloalkyl contains one oxygen atom,
or a polymorph, enantiomer, diastereomer, racemate, tautomer, N oxide, hydrate, solvate, physiologically acceptable salt, or a solvate of a physiologically acceptable salt thereof, or a mixture of any of the foregoing.

16: The combination according to claim 2, wherein component A is a compound of formula (I), wherein
R⁵ is 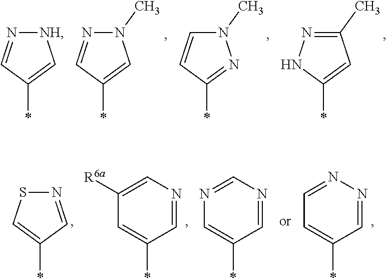
wherein * indicates the point of attachment of said group with the rest of the molecule,
or a polymorph, enantiomer, diastereomer, racemate, tautomer, N oxide, hydrate, solvate, physiologically acceptable salt, or a solvate of a physiologically acceptable salt thereof, or a mixture of any of the foregoing.

17: The combination according to claim 2, wherein component A is a compound of formula (I), wherein
R⁵ is 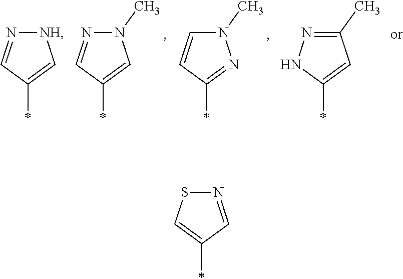
wherein * indicates the point of attachment of said group with the rest of the molecule,
or a polymorph, enantiomer, diastereomer, racemate, tautomer, N oxide, hydrate, solvate, physiologically acceptable salt, or a solvate of a physiologically acceptable salt thereof, or a mixture of any of the foregoing.

18: The combination according to claim 2, wherein component A is a compound of formula (I), wherein
R⁵ is 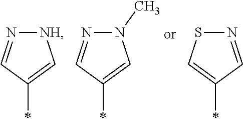
wherein * indicates the point of attachment of said group with the rest of the molecule,
or a polymorph, enantiomer, diastereomer, racemate, tautomer, N oxide, hydrate, solvate, physiologically acceptable salt, or a solvate of a physiologically acceptable salt thereof, or a mixture of any of the foregoing.

19: The combination according to claim 2, wherein component A is a compound of formula (I), wherein
R⁵ is 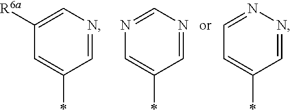
wherein * indicates the point of attachment of said group with the rest of the molecule,
or a polymorph, enantiomer, diastereomer, racemate, tautomer, N oxide, hydrate, solvate, physiologically acceptable salt, or a solvate of a physiologically acceptable salt thereof, or a mixture of any of the foregoing.

20: The combination according to claim 1, wherein component A is:
N-[(2S)-1-hydroxybutan-2-yl]-6-(4-methylphenyl)-2-(1-methyl-1H-pyrazol-4-yl)-3-oxo-2,3-dihydropyridazine-4-carboxamide;
N-(1-hydroxypropan-2-yl)-6-(4-methylphenyl)-2-(1-methyl-1H-pyrazol-4-yl)-3-oxo-2,3-dihydropyridazine-4-carboxamide;
N-[(2R)-1-hydroxypropan-2-yl]-6-(4-methylphenyl)-2-(1-methyl-1H-pyrazol-4-yl)-3-oxo-2,3-dihydropyridazine-4-carboxamide;
N-[(2S)-1-hydroxypropan-2-yl]-6-(4-methylphenyl)-2-(1-methyl-1H-pyrazol-4-yl)-3-oxo-2,3-dihydropyridazine-4-carboxamide;
6-(4-methylphenyl)-2-(1-methyl-1H-pyrazol-4-yl)-3-oxo-N-(3,3,3-trifluoro-2-hydroxypropyl)-2,3-dihydropyridazine-4-carboxamide;
6-(4-methylphenyl)-2-(1-methyl-1H-pyrazol-4-yl)-3-oxo-N-[(2R)-3,3,3-trifluoro-2-hydroxypropyl]-2,3-dihydropyridazine-4-carboxamide;
6-(4-methylphenyl)-2-(1-methyl-1H-pyrazol-4-yl)-3-oxo-N-[(2S)-3,3,3-trifluoro-2-hydroxypropyl]-2,3-dihydropyridazine-4-carboxamide;
N-(3,3-difluoro-2-hydroxypropyl)-6-(4-methylphenyl)-2-(1-methyl-1H-pyrazol-4-yl)-3-oxo-2,3-dihydropyridazine-4-carboxamide;
N-[(2R)-3,3-difluoro-2-hydroxypropyl]-6-(4-methylphenyl)-2-(1-methyl-1H-pyrazol-4-yl)-3-oxo-2,3-dihydropyridazine-4-carboxamide;
N-[(2S)-3,3-difluoro-2-hydroxypropyl]-6-(4-methylphenyl)-2-(1-methyl-1H-pyrazol-4-yl)-3-oxo-2,3-dihydropyridazine-4-carboxamide;
6-(4-chlorophenyl)-N-(1-hydroxy-3-methylbutan-2-yl)-2-(1-methyl-1H-pyrazol-4-yl)-3-oxo-2,3-dihydropyridazine-4-carboxamide;
6-(4-chlorophenyl)-N-[(2S)-1-hydroxy-3-methylbutan-2-yl]-2-(1-methyl-1H-pyrazol-4-yl)-3-oxo-2,3-dihydropyridazine-4-carboxamide;
6-(4-chlorophenyl)-N-(1-hydroxypropan-2-yl)-2-(1-methyl-1H-pyrazol-4-yl)-3-oxo-2,3-dihydropyridazine-4-carboxamide;
6-(4-chlorophenyl)-N-[(2S)-1-hydroxypropan-2-yl]-2-(1-methyl-1H-pyrazol-4-yl)-3-oxo-2,3-dihydropyridazine-4-carboxamide;
6-(4-chlorophenyl)-2-(1-methyl-1H-pyrazol-4-yl)-3-oxo-N-(3,3,3-trifluoro-2-hydroxypropyl)-2,3-dihydropyridazine-4-carboxamide;
6-(4-chlorophenyl)-2-(1-methyl-1H-pyrazol-4-yl)-3-oxo-N-[(2R)-3,3,3-trifluoro-2-hydroxypropyl]-2,3-dihydropyridazine-4-carboxamide;
6-(4-chlorophenyl)-2-(1-methyl-1H-pyrazol-4-yl)-3-oxo-N-[(2S)-3,3,3-trifluoro-2-hydroxypropyl]-2,3-dihydropyridazine-4-carboxamide;
6-(4-chlorophenyl)-N-(3,3-difluoro-2-hydroxypropyl)-2-(1-methyl-1H-pyrazol-4-yl)-3-oxo-2,3-dihydropyridazine-4-carboxamide;
6-(4-chlorophenyl)-N-[(2R)-3,3-difluoro-2-hydroxypropyl]-2-(1-methyl-1H-pyrazol-4-yl)-3-oxo-2,3-dihydropyridazine-4-carboxamide;
6-(4-chlorophenyl)-N-[(2S)-3,3-difluoro-2-hydroxypropyl]-2-(1-methyl-1H-pyrazol-4-yl)-3-oxo-2,3-dihydropyridazine-4-carboxamide;
6-(4-chlorophenyl)-N-(2-hydroxy-3-methoxypropyl)-2-(1-methyl-1H-pyrazol-4-yl)-3-oxo-2,3-dihydropyridazine-4-carboxamide;
6-(4-chlorophenyl)-N-[(2R)-2-hydroxy-3-methoxypropyl]-2-(1-methyl-1H-pyrazol-4-yl)-3-oxo-2,3-dihydropyridazine-4-carboxamide;
6-(4-chlorophenyl)-N-[(2S)-2-hydroxy-3-methoxypropyl]-2-(1-methyl-1H-pyrazol-4-yl)-3-oxo-2,3-dihydropyridazine-4-carboxamide;
6-(4-chlorophenyl)-N-(2,3-dihydroxypropyl)-2-(1-methyl-1H-pyrazol-4-yl)-3-oxo-2,3-dihydropyridazine-4-carboxamide;
6-(4-chlorophenyl)-N-[(2R)-2,3-dihydroxypropyl]-2-(1-methyl-1H-pyrazol-4-yl)-3-oxo-2,3-dihydropyridazine-4-carboxamide;
6-(4-chlorophenyl)-N-[(2S)-2,3-dihydroxypropyl]-2-(1-methyl-1H-pyrazol-4-yl)-3-oxo-2,3-dihydropyridazine-4-carboxamide;
6-(4-chlorophenyl)-N-(1-hydroxy-3-methoxypropan-2-yl)-2-(1-methyl-1H-pyrazol-4-yl)-3-oxo-2,3-dihydropyridazine-4-carboxamide;
6-(4-chlorophenyl)-N-[(2R)-1-hydroxy-3-methoxypropan-2-yl]-2-(1-methyl-1H-pyrazol-4-yl)-3-oxo-2,3-dihydropyridazine-4-carboxamide;
6-(4-chlorophenyl)-N-[(2S)-1-hydroxy-3-methoxypropan-2-yl]-2-(1-methyl-1H-pyrazol-4-yl)-3-oxo-2,3-dihydropyridazine-4-carboxamide;
6-(4-chlorophenyl)-N-(1,3-dihydroxypropan-2-yl)-2-(1-methyl-1H-pyrazol-4-yl)-3-oxo-2,3-dihydropyridazine-4-carboxamide;
6-(4-chlorophenyl)-N-(2-hydroxy-2-methylpropyl)-2-(1-methyl-1H-pyrazol-4-yl)-3-oxo-2,3-dihydropyridazine-4-carboxamide;
6-(4-chlorophenyl)-2-(1-methyl-1H-pyrazol-4-yl)-3-oxo-N-(1,1,1-trifluoro-3-hydroxypropan-2-yl)-2,3-dihydropyridazine-4-carboxamide;
6-(4-chlorophenyl)-2-(1-methyl-1H-pyrazol-4-yl)-3-oxo-N-[(2R)-1,1,1-trifluoro-3-hydroxypropan-2-yl]-2,3-dihydropyridazine-4-carboxamide;
6-(4-chlorophenyl)-2-(1-methyl-1H-pyrazol-4-yl)-3-oxo-N-[(2S)-1,1,1-trifluoro-3-hydroxypropan-2-yl]-2,3-dihydropyridazine-4-carboxamide;
2-(1-methyl-1H-pyrazol-4-yl)-3-oxo-N-(1,1,1-trifluoro-3-hydroxypropan-2-yl)-6-[4-(trifluoromethyl)phenyl]-2,3-dihydropyridazine-4-carboxamide;
2-(1-methyl-1H-pyrazol-4-yl)-3-oxo-N-[(2R)-1,1,1-trifluoro-3-hydroxypropan-2-yl]-6-[4-(trifluoromethyl)phenyl]-2,3-dihydropyridazine-4-carboxamide;
2-(1-methyl-1H-pyrazol-4-yl)-3-oxo-N-(3,3,3-trifluoro-2-hydroxypropyl)-6-[4-(trifluoromethyl)phenyl]-2,3-dihydropyridazine-4-carboxamide;
2-(1-methyl-1H-pyrazol-4-yl)-3-oxo-N-[(2S)-3,3,3-trifluoro-2-hydroxypropyl]-6-[4-(trifluoromethyl)phenyl]-2,3-dihydropyridazine-4-carboxamide;
N-(1-hydroxypropan-2-yl)-2-(1-methyl-1H-pyrazol-4-yl)-3-oxo-6-[4-(trifluoromethyl)phenyl]-2,3-dihydropyridazine-4-carboxamide;
N-[(2R)-1-hydroxypropan-2-yl]-2-(1-methyl-1H-pyrazol-4-yl)-3-oxo-6-[4-(trifluoromethyl)phenyl]-2,3-dihydropyridazine-4-carboxamide;
N-[(2S)-1-hydroxypropan-2-yl]-2-(1-methyl-1H-pyrazol-4-yl)-3-oxo-6-[4-(trifluoromethyl)phenyl]-2,3-dihydropyridazine-4-carboxamide;
6-(4-chlorophenyl)-2-(1-cyclobutyl-1H-pyrazol-4-yl)-3-oxo-N-(3,3,3-trifluoro-2-hydroxypropyl)-2,3-dihydropyridazine-4-carboxamide;
6-(4-chlorophenyl)-2-(1-cyclobutyl-1H-pyrazol-4-yl)-3-oxo-N-[(2S)-3,3,3-trifluoro-2-hydroxypropyl]-2,3-dihydropyridazine-4-carboxamide;
6-(4-chlorophenyl)-2-(1-cyclobutyl-1H-pyrazol-4-yl)-3-oxo-N-(1,1,1-trifluoro-3-hydroxypropan-2-yl)-2,3-dihydropyridazine-4-carboxamide;
6-(4-chlorophenyl)-2-(1-cyclobutyl-1H-pyrazol-4-yl)-3-oxo-N-[(2R)-1,1,1-trifluoro-3-hydroxypropan-2-yl]-2,3-dihydropyridazine-4-carboxamide;
6-(4-chlorophenyl)-2-(1-cyclobutyl-1H-pyrazol-4-yl)-N-(1-hydroxypropan-2-yl)-3-oxo-2,3-dihydropyridazine-4-carboxamide;
6-(4-chlorophenyl)-2-(1-cyclobutyl-1H-pyrazol-4-yl)-N-[(2S)-1-hydroxypropan-2-yl]-3-oxo-2,3-dihydropyridazine-4-carboxamide;
6-(4-chlorophenyl)-N-[(cis)-4-hydroxytetrahydrofuran-3-yl]-2-(1-methyl-1H-pyrazol-4-yl)-3-oxo-2,3-dihydropyridazine-4-carboxamide;
6-(4-chlorophenyl)-N-[(3R,4R)-4-hydroxytetrahydrofuran-3-yl]-2-(1-methyl-1H-pyrazol-4-yl)-3-oxo-2,3-dihydropyridazine-4-carboxamide;
6-(4-chlorophenyl)-N-[(3S,4S)-4-hydroxytetrahydrofuran-3-yl]-2-(1-methyl-1H-pyrazol-4-yl)-3-oxo-2,3-dihydropyridazine-4-carboxamide;
N-[(cis)-2-hydroxycyclohexyl]-2-(1-methyl-1H-pyrazol-4-yl)-3-oxo-6-[4-(trifluoromethoxy)phenyl]-2,3-dihydropyridazine-4-carboxamide;
N-[(1S,2R)-2-hydroxycyclohexyl]-2-(1-methyl-1H-pyrazol-4-yl)-3-oxo-6-[4-(trifluoromethoxy)phenyl]-2,3-dihydropyridazine-4-carboxamide;
2-(1-methyl-1H-pyrazol-4-yl)-3-oxo-N-(1,1,1-trifluoro-3-hydroxypropan-2-yl)-6-[4-(trifluoromethoxy)phenyl]-2,3-dihydropyridazine-4-carboxamide;
2-(1-methyl-1H-pyrazol-4-yl)-3-oxo-N-[(2R)-1,1,1-trifluoro-3-hydroxypropan-2-yl]-6-[4-(trifluoromethoxy)phenyl]-2,3-dihydropyridazine-4-carboxamide;
6-(4-chlorophenyl)-N-[(cis)-2-hydroxycyclohexyl]-2-(1-methyl-1H-pyrazol-4-yl)-3-oxo-2,3-dihydropyridazine-4-carboxamide;
6-(4-chlorophenyl)-N-[(1S,2R)-2-hydroxycyclohexyl]-2-(1-methyl-1H-pyrazol-4-yl)-3-oxo-2,3-dihydropyridazine-4-carboxamide;
6-(4-chlorophenyl)-N-[(trans)-2-hydroxycyclopentyl]-2-(1-methyl-1H-pyrazol-4-yl)-3-oxo-2,3-dihydropyridazine-4-carboxamide;
6-(4-chlorophenyl)-N-[(1S,2S)-2-hydroxycyclopentyl]-2-(1-methyl-1H-pyrazol-4-yl)-3-oxo-2,3-dihydropyridazine-4-carboxamide;
6-(4-chlorophenyl)-N-[(cis)-2-hydroxycyclopentyl]-2-(1-methyl-1H-pyrazol-4-yl)-3-oxo-2,3-dihydropyridazine-4-carboxamide;
6-(4-chlorophenyl)-N-[(1S,2R)-2-hydroxycyclopentyl]-2-(1-methyl-1H-pyrazol-4-yl)-3-oxo-2,3-dihydropyridazine-4-carboxamide;
6-[4-(difluoromethyl)phenyl]-N-[(trans)-2-hydroxycyclopentyl]-2-(1-methyl-1H-pyrazol-4-yl)-3-oxo-2,3-dihydropyridazine-4-carboxamide;
N-(1-hydroxypropan-2-yl)-2-(1-methyl-1H-pyrazol-4-yl)-3-oxo-6-[4-(trifluoromethoxy)phenyl]-2,3-dihydropyridazine-4-carboxamide;
N-[(2S)-1-hydroxypropan-2-yl]-2-(1-methyl-1H-pyrazol-4-yl)-3-oxo-6-[4-(trifluoromethoxy)phenyl]-2,3-dihydropyridazine-4-carboxamide;
6-[6-(difluoromethyl)pyridin-3-yl]-N-[(cis)-2-hydroxycyclohexyl]-2-(1-methyl-1H-pyrazol-4-yl)-3-oxo-2,3-dihydropyridazine-4-carboxamide;
6-[6-(difluoromethyl)pyridin-3-yl]-N-[(1S,2R)-2-hydroxycyclohexyl]-2-(1-methyl-1H-pyrazol-4-yl)-3-oxo-2,3-dihydropyridazine-4-carboxamide;
N-[(cis)-4-hydroxytetrahydrofuran-3-yl]-2-(1-methyl-1H-pyrazol-4-yl)-3-oxo-6-[4-(trifluoromethoxy)phenyl]-2,3-dihydropyridazine-4-carboxamide;
N-[(3R,4R)-4-hydroxytetrahydrofuran-3-yl]-2-(1-methyl-1H-pyrazol-4-yl)-3-oxo-6-[4-(trifluoromethoxy)phenyl]-2,3-dihydropyridazine-4-carboxamide;
N-[(3S,4S)-4-hydroxytetrahydrofuran-3-yl]-2-(1-methyl-1H-pyrazol-4-yl)-3-oxo-6-[4-(trifluoromethoxy)phenyl]-2,3-dihydropyridazine-4-carboxamide;
6-[4-(difluoromethyl)phenyl]-N-(1-hydroxypropan-2-yl)-2-(1-methyl-1H-pyrazol-4-yl)-3-oxo-2,3-dihydropyridazine-4-carboxamide;
6-[4-(difluoromethyl)phenyl]-N-[(2S)-1-hydroxypropan-2-yl]-2-(1-methyl-1H-pyrazol-4-yl)-3-oxo-2,3-dihydropyridazine-4-carboxamide;
N-(2-hydroxy-2-methylpropyl)-2-(1-methyl-1H-pyrazol-4-yl)-3-oxo-6-[4-(trifluoromethoxy)phenyl]-2,3-dihydropyridazine-4-carboxamide;
6-[4-(difluoromethyl)phenyl]-N-[(cis)-4-hydroxytetrahydrofuran-3-yl]-2-(1-methyl-1H-pyrazol-4-yl)-3-oxo-2,3-dihydropyridazine-4-carboxamide;
1,5-anhydro-2-({[6-(4-chlorophenyl)-2-(1-methyl-1H-pyrazol-4-yl)-3-oxo-2,3-dihydropyridazin-4-yl]carbonyl}amino)-2,4-dideoxy-D-erythro-pentitol;
6-[4-(difluoromethyl)phenyl]-N-(2-hydroxy-2-methylpropyl)-2-(1-methyl-1H-pyrazol-4-yl)-3-oxo-2,3-dihydropyridazine-4-carboxamide;
N-[(trans)-4-hydroxytetrahydrofuran-3-yl]-2-(1-methyl-1H-pyrazol-4-yl)-3-oxo-6-[4-(trifluoromethoxy)phenyl]-2,3-dihydropyridazine-4-carboxamide;
N-[(3R,4S)-4-hydroxytetrahydrofuran-3-yl]-2-(1-methyl-1H-pyrazol-4-yl)-3-oxo-6-[4-(trifluoromethoxy)phenyl]-2,3-dihydropyridazine-4-carboxamide;
N-[(3S,4R)-4-hydroxytetrahydrofuran-3-yl]-2-(1-methyl-1H-pyrazol-4-yl)-3-oxo-6-[4-(trifluoromethoxy)phenyl]-2,3-dihydropyridazine-4-carboxamide;
6-[6-(difluoromethyl)pyridin-3-yl]-N-(1-hydroxypropan-2-yl)-2-(1-methyl-1H-pyrazol-4-yl)-3-oxo-2,3-dihydropyridazine-4-carboxamide;
6-[6-(difluoromethyl)pyridin-3-yl]-N-[(2S)-1-hydroxypropan-2-yl]-2-(1-methyl-1H-pyrazol-4-yl)-3-oxo-2,3-dihydropyridazine-4-carboxamide;
N-(2-hydroxy-2-methylpropyl)-2-(1-methyl-1H-pyrazol-4-yl)-3-oxo-6-[4-(trifluoromethyl)phenyl]-2,3-dihydropyridazine-4-carboxamide;
6-(4-chlorophenyl)-N-(1-hydroxypropan-2-yl)-3-oxo-2-(pyridin-3-yl)-2,3-dihydropyridazine-4-carboxamide;
6-(4-chlorophenyl)-N-[(2R)-1-hydroxypropan-2-yl]-3-oxo-2-(pyridin-3-yl)-2,3-dihydropyridazine-4-carboxamide;
6-(4-chlorophenyl)-N-[(2S)-1-hydroxypropan-2-yl]-3-oxo-2-(pyridin-3-yl)-2,3-dihydropyridazine-4-carboxamide;
6-(4-chlorophenyl)-N-(1,3-dihydroxypropan-2-yl)-3-oxo-2-(pyridin-3-yl)-2,3-dihydropyridazine-4-carboxamide;
6-(4-chlorophenyl)-N-(2-hydroxy-2-methylpropyl)-3-oxo-2-(pyridin-3-yl)-2,3-dihydropyridazine-4-carboxamide;
6-(4-chlorophenyl)-N-(2,3-dihydroxypropyl)-3-oxo-2-(pyridin-3-yl)-2,3-dihydropyridazine-4-carboxamide;
6-(4-chlorophenyl)-N-[(2R)-2,3-dihydroxypropyl]-3-oxo-2-(pyridin-3-yl)-2,3-dihydropyridazine-4-carboxamide;
6-(4-chlorophenyl)-N-[(2S)-2,3-dihydroxypropyl]-3-oxo-2-(pyridin-3-yl)-2,3-dihydropyridazine-4-carboxamide;
6-(4-chlorophenyl)-N-(1-hydroxy-3-methoxypropan-2-yl)-3-oxo-2-(pyridin-3-yl)-2,3-dihydropyridazine-4-carboxamide;
6-(4-chlorophenyl)-N-[(2R)-1-hydroxy-3-methoxypropan-2-yl]-3-oxo-2-(pyridin-3-yl)-2,3-dihydropyridazine-4-carboxamide;
6-(4-chlorophenyl)-N-[(2S)-1-hydroxy-3-methoxypropan-2-yl]-3-oxo-2-(pyridin-3-yl)-2,3-dihydropyridazine-4-carboxamide;
6-(4-chlorophenyl)-3-oxo-2-(pyridin-3-yl)-N-(1,1,1-trifluoro-3-hydroxypropan-2-yl)-2,3-dihydropyridazine-4-carboxamide;
6-(4-chlorophenyl)-3-oxo-2-(pyridin-3-yl)-N-[(2R)-1,1,1-trifluoro-3-hydroxypropan-2-yl]-2,3-dihydropyridazine-4-carboxamide;
6-(4-chlorophenyl)-3-oxo-2-(pyridin-3-yl)-N-[(2S)-1,1,1-trifluoro-3-hydroxypropan-2-yl]-2,3-dihydropyridazine-4-carboxamide;
6-(4-chlorophenyl)-N-(1-cyclopropyl-2-hydroxyethyl)-3-oxo-2-(pyridin-3-yl)-2,3-dihydropyridazine-4-carboxamide;
6-(4-chlorophenyl)-N-[(1R)-1-cyclopropyl-2-hydroxyethyl]-3-oxo-2-(pyridin-3-yl)-2,3-dihydropyridazine-4-carboxamide;
6-(4-chlorophenyl)-N-[(1S)-1-cyclopropyl-2-hydroxyethyl]-3-oxo-2-(pyridin-3-yl)-2,3-dihydropyridazine-4-carboxamide;
6-(4-chlorophenyl)-3-oxo-2-(pyridin-3-yl)-N-(3,3,3-trifluoro-2-hydroxypropyl)-2,3-dihydropyridazine-4-carboxamide;
6-(4-chlorophenyl)-3-oxo-2-(pyridin-3-yl)-N-[(2R)-3,3,3-trifluoro-2-hydroxypropyl]-2,3-dihydropyridazine-4-carboxamide;
6-(4-chlorophenyl)-3-oxo-2-(pyridin-3-yl)-N-[(2S)-3,3,3-trifluoro-2-hydroxypropyl]-2,3-dihydropyridazine-4-carboxamide;
6-(4-chlorophenyl)-N-(3,3-difluoro-2-hydroxypropyl)-3-oxo-2-(pyridin-3-yl)-2,3-dihydropyridazine-4-carboxamide;
6-(4-chlorophenyl)-N-[(2R)-3,3-difluoro-2-hydroxypropyl]-3-oxo-2-(pyridin-3-yl)-2,3-dihydropyridazine-4-carboxamide;
6-(4-chlorophenyl)-N-[(2S)-3,3-difluoro-2-hydroxypropyl]-3-oxo-2-(pyridin-3-yl)-2,3-dihydropyridazine-4-carboxamide;
6-(4-chlorophenyl)-2-(5-fluoropyridin-3-yl)-N-(1-hydroxypropan-2-yl)-3-oxo-2,3-dihydropyridazine-4-carboxamide;
6-(4-chlorophenyl)-2-(5-fluoropyridin-3-yl)-N-[(2R)-1-hydroxypropan-2-yl]-3-oxo-2,3-dihydropyridazine-4-carboxamide;
6-(4-chlorophenyl)-2-(5-fluoropyridin-3-yl)-N-[(2S)-1-hydroxypropan-2-yl]-3-oxo-2,3-dihydropyridazine-4-carboxamide;
6-(4-chlorophenyl)-2-(5-fluoropyridin-3-yl)-N-(2-hydroxy-2-methylpropyl)-3-oxo-2,3-dihydropyridazine-4-carboxamide;
6-(4-chlorophenyl)-2-(5-fluoropyridin-3-yl)-N-(1-hydroxy-3-methylbutan-2-yl)-3-oxo-2,3-dihydropyridazine-4-carboxamide;
6-(4-chlorophenyl)-2-(5-fluoropyridin-3-yl)-N-[(2S)-1-hydroxy-3-methylbutan-2-yl]-3-oxo-2,3-dihydropyridazine-4-carboxamide;
6-(4-chlorophenyl)-N-(1,3-dihydroxypropan-2-yl)-2-(5-fluoropyridin-3-yl)-3-oxo-2,3-dihydropyridazine-4-carboxamide;
6-(4-chlorophenyl)-2-(5-fluoropyridin-3-yl)-N-(1-hydroxy-3-methoxypropan-2-yl)-3-oxo-2,3-dihydropyridazine-4-carboxamide;
6-(4-chlorophenyl)-2-(5-fluoropyridin-3-yl)-N-[(2R)-1-hydroxy-3-methoxypropan-2-yl]-3-oxo-2,3-dihydropyridazine-4-carboxamide;
6-(4-chlorophenyl)-2-(5-fluoropyridin-3-yl)-N-[(2S)-1-hydroxy-3-methoxypropan-2-yl]-3-oxo-2,3-dihydropyridazine-4-carboxamide;
6-(4-chlorophenyl)-N-(1-fluoro-3-hydroxypropan-2-yl)-3-oxo-2-(pyridin-3-yl)-2,3-dihydropyridazine-4-carboxamide;
6-(4-chlorophenyl)-N-[(2R)-1-fluoro-3-hydroxypropan-2-yl]-3-oxo-2-(pyridin-3-yl)-2,3-dihydropyridazine-4-carboxamide;
N-(1-cyclopropyl-2-hydroxyethyl)-3-oxo-2-(pyridin-3-yl)-6-[4-(trifluoromethyl)phenyl]-2,3-dihydropyridazine-4-carboxamide;
N-[(1S)-1-cyclopropyl-2-hydroxyethyl]-3-oxo-2-(pyridin-3-yl)-6-[4-(trifluoromethyl)phenyl]-2,3-dihydropyridazine-4-carboxamide;
6-(4-chlorophenyl)-N-(1-cyclopropyl-2-hydroxyethyl)-2-(5-fluoropyridin-3-yl)-3-oxo-2,3-dihydropyridazine-4-carboxamide;
6-(4-chlorophenyl)-N-(2,3-dihydroxypropyl)-2-(5-fluoropyridin-3-yl)-3-oxo-2,3-dihydropyridazine-4-carboxamide;
6-(4-chlorophenyl)-N-[(2R)-2,3-dihydroxypropyl]-2-(5-fluoropyridin-3-yl)-3-oxo-2,3-dihydropyridazine-4-carboxamide;
6-(4-chlorophenyl)-N-[(2S)-2,3-dihydroxypropyl]-2-(5-fluoropyridin-3-yl)-3-oxo-2,3-dihydropyridazine-4-carboxamide;
6-(4-chlorophenyl)-2-(5-fluoropyridin-3-yl)-3-oxo-N-(1,1,1-trifluoro-3-hydroxypropan-2-yl)-2,3-dihydropyridazine-4-carboxamide;
6-(4-chlorophenyl)-2-(5-fluoropyridin-3-yl)-3-oxo-N-[(2R)-1,1,1-trifluoro-3-hydroxypropan-2-yl]-2,3-dihydropyridazine-4-carboxamide;
N-(3,3-difluoro-2-hydroxypropyl)-3-oxo-2-(pyridin-3-yl)-6-[4-(trifluoromethyl)phenyl]-2,3-dihydropyridazine-4-carboxamide;
N-(2-hydroxycyclopentyl)-3-oxo-2-(pyridin-3-yl)-6-[4-(trifluoromethyl)phenyl]-2,3-dihydropyridazine-4-carboxamide;
N-[(1S,2S)-2-hydroxycyclopentyl]-3-oxo-2-(pyridin-3-yl)-6-[4-(trifluoromethyl)phenyl]-2,3-dihydropyridazine-4-carboxamide;
N-[(1R,2R)-2-hydroxycyclopentyl]-3-oxo-2-(pyridin-3-yl)-6-[4-(trifluoromethyl)phenyl]-2,3-dihydropyridazine-4-carboxamide;
N-[(1R,2S)-2-hydroxycyclopentyl]-3-oxo-2-(pyridin-3-yl)-6-[4-(trifluoromethyl)phenyl]-2,3-dihydropyridazine-4-carboxamide;
N-[(1S,2R)-2-hydroxycyclopentyl]-3-oxo-2-(pyridin-3-yl)-6-[4-(trifluoromethyl)phenyl]-2,3-dihydropyridazine-4-carboxamide;
N-[(cis)-2-hydroxycyclohexyl]-3-oxo-2-(pyridin-3-yl)-6-[4-(trifluoromethyl)phenyl]-2,3-dihydropyridazine-4-carboxamide;
N-[(1S,2R)-2-hydroxycyclohexyl]-3-oxo-2-(pyridin-3-yl)-6-[4-(trifluoromethyl)phenyl]-2,3-dihydropyridazine-4-carboxamide;
N-[(cis)-4-hydroxytetrahydrofuran-3-yl]-3-oxo-2-(pyridin-3-yl)-6-[4-(trifluoromethyl)phenyl]-2,3-dihydropyridazine-4-carboxamide;
3-oxo-2-(pyridin-3-yl)-N-(3,3,3-trifluoro-2-hydroxypropyl)-6-[4-(trifluoromethyl)phenyl]-2,3-dihydropyridazine-4-carboxamide;
3-oxo-2-(pyridin-3-yl)-N-[(2S)-3,3,3-trifluoro-2-hydroxypropyl]-6-[4-(trifluoromethyl)phenyl]-2,3-dihydropyridazine-4-carboxamide;
3-oxo-2-(pyridin-3-yl)-N-(1,1,1-trifluoro-3-hydroxypropan-2-yl)-6-[4-(trifluoromethyl)phenyl]-2,3-dihydropyridazine-4-carboxamide;
3-oxo-2-(pyridin-3-yl)-N-(1,1,1-trifluoro-3-hydroxypropan-2-yl)-6-[4-(trifluoromethyl)phenyl]-2,3-dihydropyridazine-4-carboxamide;
3-oxo-2-(pyridin-3-yl)-N-[(2R)-1,1,1-trifluoro-3-hydroxypropan-2-yl]-6-[4-(trifluoromethyl)phenyl]-2,3-dihydropyridazine-4-carboxamide;
N-(1-hydroxy-3-methoxypropan-2-yl)-3-oxo-2-(pyridin-3-yl)-6-[4-(trifluoromethyl)phenyl]-2,3-dihydropyridazine-4-carboxamide;
N-[(2S)-1-hydroxy-3-methoxypropan-2-yl]-3-oxo-2-(pyridin-3-yl)-6-[4-(trifluoromethyl)phenyl]-2,3-dihydropyridazine-4-carboxamide;
N-(2,3-dihydroxypropyl)-3-oxo-2-(pyridin-3-yl)-6-[4-(trifluoromethyl)phenyl]-2,3-dihydropyridazine-4-carboxamide;
N-[(2R)-2,3-dihydroxypropyl]-3-oxo-2-(pyridin-3-yl)-6-[4-(trifluoromethyl)phenyl]-2,3-dihydropyridazine-4-carboxamide;
N-[(2S)-2,3-dihydroxypropyl]-3-oxo-2-(pyridin-3-yl)-6-[4-(trifluoromethyl)phenyl]-2,3-dihydropyridazine-4-carboxamide;
N-(2-hydroxy-2-methylpropyl)-3-oxo-2-(pyridin-3-yl)-6-[4-(trifluoromethyl)phenyl]-2,3-dihydropyridazine-4-carboxamide;
N-(1,3-dihydroxypropan-2-yl)-3-oxo-2-(pyridin-3-yl)-6-[4-(trifluoromethyl)phenyl]-2,3-dihydropyridazine-4-carboxamide;
N-(1-hydroxypropan-2-yl)-3-oxo-2-(pyridin-3-yl)-6-[4-(trifluoromethyl)phenyl]-2,3-dihydropyridazine-4-carboxamide;
N-[(2R)-1-hydroxypropan-2-yl]-3-oxo-2-(pyridin-3-yl)-6-[4-(trifluoromethyl)phenyl]-2,3-dihydropyridazine-4-carboxamide;
N-[(2S)-1-hydroxypropan-2-yl]-3-oxo-2-(pyridin-3-yl)-6-[4-(trifluoromethyl)phenyl]-2,3-dihydropyridazine-4-carboxamide;
6-(4-chlorophenyl)-N-(1-hydroxypropan-2-yl)-3-oxo-2-(pyrimidin-5-yl)-2,3-dihydropyridazine-4-carboxamide;
6-(4-chlorophenyl)-N-[(2S)-1-hydroxypropan-2-yl]-3-oxo-2-(pyrimidin-5-yl)-2,3-dihydropyridazine-4-carboxamide;
6-(4-chlorophenyl)-3-oxo-2-(pyridin-3-yl)-N-(1,1,1-trifluoro-3-hydroxy-3-methylbutan-2-yl)-2,3-dihydropyridazine-4-carboxamide;
6-(4-chlorophenyl)-3-oxo-2-(pyridin-3-yl)-N-[(2R)-1,1,1-trifluoro-3-hydroxy-3-methylbutan-2-yl]-2,3-dihydropyridazine-4-carboxamide;
6-(4-chlorophenyl)-3-oxo-2-(pyridin-3-yl)-N-[(2S)-1,1,1-trifluoro-3-hydroxy-3-methylbutan-2-yl]-2,3-dihydropyridazine-4-carboxamide;
6-(4-chlorophenyl)-2-(5-fluoropyridin-3-yl)-N-[(cis)-2-hydroxycyclohexyl]-3-oxo-2,3-dihydropyridazine-4-carboxamide;
6-(4-chlorophenyl)-2-(5-fluoropyridin-3-yl)-N-[(1S,2R)-2-hydroxycyclohexyl]-3-oxo-2,3-dihydropyridazine-4-carboxamide;
6-(4-chlorophenyl)-2-(5-fluoropyridin-3-yl)-N-[(cis)-2-hydroxycyclohexyl]-3-oxo-2,3-dihydropyridazine-4-carboxamide;
6-(4-chlorophenyl)-2-(5-fluoropyridin-3-yl)-N-[(1S,2R)-2-hydroxycyclohexyl]-3-oxo-2,3-dihydropyridazine-4-carboxamide;
N-[(cis)-2-hydroxycyclohexyl]-3-oxo-2-(pyridin-3-yl)-6-[4-(trifluoromethoxy)phenyl]-2,3-dihydropyridazine-4-carboxamide;
N-[(1S,2R)-2-hydroxycyclohexyl]-3-oxo-2-(pyridin-3-yl)-6-[4-(trifluoromethoxy)phenyl]-2,3-dihydropyridazine-4-carboxamide;
6-[4-(difluoromethyl)phenyl]-3-oxo-2-(pyridin-3-yl)-N-(1,1,1-trifluoro-3-hydroxypropan-2-yl)-2,3-dihydropyridazine-4-carboxamide;
6-[4-(difluoromethyl)phenyl]-3-oxo-2-(pyridin-3-yl)-N-[(2R)-1,1,1-trifluoro-3-hydroxypropan-2-yl]-2,3-dihydropyridazine-4-carboxamide;
6-(4-chlorophenyl)-2-(5-fluoropyridin-3-yl)-3-oxo-N-(3,3,3-trifluoro-2-hydroxypropyl)-2,3-dihydropyridazine-4-carboxamide;
6-(4-chlorophenyl)-2-(5-fluoropyridin-3-yl)-3-oxo-N-[(2S)-3,3,3-trifluoro-2-hydroxypropyl]-2,3-dihydropyridazine-4-carboxamide;
6-(4-chlorophenyl)-2-(5-fluoropyridin-3-yl)-N-[(cis)-2-hydroxycyclopentyl]-3-oxo-2,3-dihydropyridazine-4-carboxamide;
6-(4-chlorophenyl)-2-(5-fluoropyridin-3-yl)-N-[(1S,2R)-2-hydroxycyclopentyl]-3-oxo-2,3-dihydropyridazine-4-carboxamide;
6-(4-chlorophenyl)-N-[(trans)-2-hydroxycyclopentyl]-3-oxo-2-(pyridin-3-yl)-2,3-dihydropyridazine-4-carboxamide;
6-(4-chlorophenyl)-N-[(1S,2S)-2-hydroxycyclopentyl]-3-oxo-2-(pyridin-3-yl)-2,3-dihydropyridazine-4-carboxamide;
3-oxo-2-(pyridin-3-yl)-N-(3,3,3-trifluoro-2-hydroxypropyl)-6-[4-(trifluoromethoxy)phenyl]-2,3-dihydropyridazine-4-carboxamide;
3-oxo-2-(pyridin-3-yl)-N-[(2S)-3,3,3-trifluoro-2-hydroxypropyl]-6-[4-(trifluoromethoxy)phenyl]-2,3-dihydropyridazine-4-carboxamide;
N-[(trans)-2-hydroxycyclopentyl]-3-oxo-2-(pyridin-3-yl)-6-[4-(trifluoromethoxy)phenyl]-2,3-dihydropyridazine-4-carboxamide;
N-[(1S,2S)-2-hydroxycyclopentyl]-3-oxo-2-(pyridin-3-yl)-6-[4-(trifluoromethoxy)phenyl]-2,3-dihydropyridazine-4-carboxamide;
6-(4-chlorophenyl)-N-[(cis)-2-hydroxycyclohexyl]-3-oxo-2-(pyridin-3-yl)-2,3-dihydropyridazine-4-carboxamide;
3-oxo-2-(pyridin-3-yl)-N-(1,1,1-trifluoro-3-hydroxypropan-2-yl)-6-[4-(trifluoromethoxy)phenyl]-2,3-dihydropyridazine-4-carboxamide;
3-oxo-2-(pyridin-3-yl)-N-[(2R)-1,1,1-trifluoro-3-hydroxypropan-2-yl]-6-[4-(trifluoromethoxy)phenyl]-2,3-dihydropyridazine-4-carboxamide;
3-oxo-2-(pyridin-3-yl)-N-(1,1,1-trifluoro-3-hydroxy-3-methylbutan-2-yl)-6-[4-(trifluoromethoxy)phenyl]-2,3-dihydropyridazine-4-carboxamide;
3-oxo-2-(pyridin-3-yl)-N-[(2R)-1,1,1-trifluoro-3-hydroxy-3-methylbutan-2-yl]-6-[4-(trifluoromethoxy)phenyl]-2,3-dihydropyridazine-4-carboxamide;
3-oxo-2-(pyridin-3-yl)-N-[(2S)-1,1,1-trifluoro-3-hydroxy-3-methylbutan-2-yl]-6-[4-(trifluoromethoxy)phenyl]-2,3-dihydropyridazine-4-carboxamide;
6-(4-chlorophenyl)-N-[(cis)-2-hydroxycyclopentyl]-3-oxo-2-(pyridin-3-yl)-2,3-dihydropyridazine-4-carboxamide;
6-(4-chlorophenyl)-N-[(1R,2S)-2-hydroxycyclopentyl]-3-oxo-2-(pyridin-3-yl)-2,3-dihydropyridazine-4-carboxamide;
6-[4-(difluoromethyl)phenyl]-N-[(trans)-2-hydroxycyclopentyl]-3-oxo-2-(pyridin-3-yl)-2,3-dihydropyridazine-4-carboxamide;
6-[4-(difluoromethyl)phenyl]-N-[(1S,2S)-2-hydroxycyclopentyl]-3-oxo-2-(pyridin-3-yl)-2,3-dihydropyridazine-4-carboxamide;
N-[(trans)-3,3-difluoro-2-hydroxycyclohexyl]-3-oxo-2-(pyridin-3-yl)-6-[4-(trifluoromethoxy)phenyl]-2,3-dihydropyridazine-4-carboxamide;
N-[(1S,2R)-3,3-difluoro-2-hydroxycyclohexyl]-3-oxo-2-(pyridin-3-yl)-6-[4-(trifluoromethoxy)phenyl]-2,3-dihydropyridazine-4-carboxamide;
N-[(1R,2S)-3,3-difluoro-2-hydroxycyclohexyl]-3-oxo-2-(pyridin-3-yl)-6-[4-(trifluoromethoxy)phenyl]-2,3-dihydropyridazine-4-carboxamide;
6-(4-chlorophenyl)-N-[(trans)-3,3-difluoro-2-hydroxycyclohexyl]-3-oxo-2-(pyridin-3-yl)-2,3-dihydropyridazine-4-carboxamide;
6-(4-chlorophenyl)-N-[(1S,2R)-3,3-difluoro-2-hydroxycyclohexyl]-3-oxo-2-(pyridin-3-yl)-2,3-dihydropyridazine-4-carboxamide;
6-(4-chlorophenyl)-N-[(1R,2S)-3,3-difluoro-2-hydroxycyclohexyl]-3-oxo-2-(pyridin-3-yl)-2,3-dihydropyridazine-4-carboxamide;
N-[(cis)-2-hydroxycyclopentyl]-3-oxo-2-(pyridin-3-yl)-6-[4-(trifluoromethoxy)phenyl]-2,3-dihydropyridazine-4-carboxamide;
N-[(1S,2R)-2-hydroxycyclopentyl]-3-oxo-2-(pyridin-3-yl)-6-[4-(trifluoromethoxy)phenyl]-2,3-dihydropyridazine-4-carboxamide;
N-(1-cyclopropyl-2-hydroxyethyl)-6-[4-(difluoromethyl)phenyl]-3-oxo-2-(pyridin-3-yl)-2,3-dihydropyridazine-4-carboxamide;
N-[(1S)-1-cyclopropyl-2-hydroxyethyl]-6-[4-(difluoromethyl)phenyl]-3-oxo-2-(pyridin-3-yl)-2,3-dihydropyridazine-4-carboxamide;
6-[4-(difluoromethyl)phenyl]-N-(1-hydroxypropan-2-yl)-3-oxo-2-(pyridin-3-yl)-2,3-dihydropyridazine-4-carboxamide;
6-[4-(difluoromethyl)phenyl]-N-[(2S)-1-hydroxypropan-2-yl]-3-oxo-2-(pyridin-3-yl)-2,3-dihydropyridazine-4-carboxamide;
N-(1-hydroxypropan-2-yl)-6-(4-methylphenyl)-3-oxo-2-(pyridin-3-yl)-2,3-dihydropyridazine-4-carboxamide;
N-[(2S)-1-hydroxypropan-2-yl]-6-(4-methylphenyl)-3-oxo-2-(pyridin-3-yl)-2,3-dihydropyridazine-4-carboxamide;
N-(1-hydroxypropan-2-yl)-3-oxo-2-(pyridin-3-yl)-6-[4-(trifluoromethoxy)phenyl]-2,3-dihydropyridazine-4-carboxamide;
N-[(2S)-1-hydroxypropan-2-yl]-3-oxo-2-(pyridin-3-yl)-6-[4-(trifluoromethoxy)phenyl]-2,3-dihydropyridazine-4-carboxamide;
6-(4-chlorophenyl)-2-(5-fluoropyridin-3-yl)-N-[(trans)-4-hydroxytetrahydrofuran-3-yl]-3-oxo-2,3-dihydropyridazine-4-carboxamide;
6-(4-chlorophenyl)-2-(5-fluoropyridin-3-yl)-N-[(3S,4R)-4-hydroxytetrahydrofuran-3-yl]-3-oxo-2,3-dihydropyridazine-4-carboxamide;
6-(4-chlorophenyl)-2-(5-fluoropyridin-3-yl)-N-[(3R,4S)-4-hydroxytetrahydrofuran-3-yl]-3-oxo-2,3-dihydropyridazine-4-carboxamide;
N-[(cis)-4-hydroxytetrahydrofuran-3-yl]-3-oxo-2-(pyridin-3-yl)-6-[4-(trifluoromethoxy)phenyl]-2,3-dihydropyridazine-4-carboxamide;
N-[(3S,4S)-4-hydroxytetrahydrofuran-3-yl]-3-oxo-2-(pyridin-3-yl)-6-[4-(trifluoromethoxy)phenyl]-2,3-dihydropyridazine-4-carboxamide;
N-[(3R,4R)-4-hydroxytetrahydrofuran-3-yl]-3-oxo-2-(pyridin-3-yl)-6-[4-(trifluoromethoxy)phenyl]-2,3-dihydropyridazine-4-carboxamide;
N-[(trans)-4-hydroxytetrahydrofuran-3-yl]-3-oxo-2-(pyridin-3-yl)-6-[4-(trifluoromethoxy)phenyl]-2,3-dihydropyridazine-4-carboxamide;
N-[(3S,4R)-4-hydroxytetrahydrofuran-3-yl]-3-oxo-2-(pyridin-3-yl)-6-[4-(trifluoromethoxy)phenyl]-2,3-dihydropyridazine-4-carboxamide;
N-[(3R,4S)-4-hydroxytetrahydrofuran-3-yl]-3-oxo-2-(pyridin-3-yl)-6-[4-(trifluoromethoxy)phenyl]-2,3-dihydropyridazine-4-carboxamide;
6-(4-chloro-2-fluorophenyl)-N-(1-hydroxypropan-2-yl)-3-oxo-2-(pyridin-3-yl)-2,3-dihydropyridazine-4-carboxamide;
6-(4-chloro-2-fluorophenyl)-N-[(2S)-1-hydroxypropan-2-yl]-3-oxo-2-(pyridin-3-yl)-2,3-dihydropyridazine-4-carboxamide;
3-(4-chlorophenyl)-N-[(cis)-2-hydroxycyclohexyl]-6-oxo-6H-1,4′-bipyridazine-5-carboxamide;
3-(4-chlorophenyl)-N-[(1S,2R)-2-hydroxycyclohexyl]-6-oxo-6H-1,4′-bipyridazine-5-carboxamide;
6-[6-(difluoromethyl)pyridin-3-yl]-N-[(trans)-2-hydroxycyclopentyl]-3-oxo-2-(pyridin-3-yl)-2,3-dihydropyridazine-4-carboxamide;
6-[6-(difluoromethyl)pyridin-3-yl]-N-[(1S,2S)-2-hydroxycyclopentyl]-3-oxo-2-(pyridin-3-yl)-2,3-dihydropyridazine-4-carboxamide;
N-(1-hydroxypropan-2-yl)-3-oxo-2-(pyridin-3-yl)-6-[6-(trifluoromethyl)pyridin-3-yl]-2,3-dihydropyridazine-4-carboxamide;
N-[(2S)-1-hydroxypropan-2-yl]-3-oxo-2-(pyridin-3-yl)-6-[6-(trifluoromethyl)pyridin-3-yl]-2,3-dihydropyridazine-4-carboxamide;
3-(4-chlorophenyl)-N-[(cis)-2-hydroxycyclopentyl]-6-oxo-6H-1,4′-bipyridazine-5-carboxamide;
3-(4-chlorophenyl)-N-[(1S,2R)-2-hydroxycyclopentyl]-6-oxo-6H-1,4′-bipyridazine-5-carboxamide;
6-(4-chlorophenyl)-N-[(trans)-4-hydroxytetrahydrofuran-3-yl]-3-oxo-2-(pyridin-3-yl)-2,3-dihydropyridazine-4-carboxamide;
6-(4-chlorophenyl)-N-[(3S,4R)-4-hydroxytetrahydrofuran-3-yl]-3-oxo-2-(pyridin-3-yl)-2,3-dihydropyridazine-4-carboxamide;
6-(4-chlorophenyl)-N-[(3R,4S)-4-hydroxytetrahydrofuran-3-yl]-3-oxo-2-(pyridin-3-yl)-2,3-dihydropyridazine-4-carboxamide;
6-(4-chlorophenyl)-N-[(cis)-4-hydroxytetrahydrofuran-3-yl]-3-oxo-2-(pyridin-3-yl)-2,3-dihydropyridazine-4-carboxamide;
6-(4-chlorophenyl)-N-[(3R,4R)-4-hydroxytetrahydrofuran-3-yl]-3-oxo-2-(pyridin-3-yl)-2,3-dihydropyridazine-4-carboxamide;
6-(4-chlorophenyl)-N-[(3S,4S)-4-hydroxytetrahydrofuran-3-yl]-3-oxo-2-(pyridin-3-yl)-2,3-dihydropyridazine-4-carboxamide;
3-(4-chlorophenyl)-N-[(trans)-2-hydroxycyclopentyl]-6-oxo-6H-1,4′-bipyridazine-5-carboxamide;
3-(4-chlorophenyl)-N-[(1S,2S)-2-hydroxycyclopentyl]-6-oxo-6H-1,4′-bipyridazine-5-carboxamide;
1,5-anhydro-2-({[6-(4-chlorophenyl)-3-oxo-2-(pyridin-3-yl)-2,3-dihydropyridazin-4-yl]carbonyl}amino)-2,4-dideoxy-cis-pentitol;
1,5-anhydro-2-({[6-(4-chlorophenyl)-3-oxo-2-(pyridin-3-yl)-2,3-dihydropyridazin-4-yl]carbonyl}amino)-2,4-dideoxy-D-erythro-pentitol;
1,5-anhydro-2,4-dideoxy-2-[({3-oxo-2-(pyridin-3-yl)-6-[4-(trifluoromethoxy)phenyl]-2,3-dihydropyridazin-4-yl}carbonyl)amino]-cis-pentitol;
1,5-anhydro-2,4-dideoxy-2-[({3-oxo-2-(pyridin-3-yl)-6-[4-(trifluoromethoxy)phenyl]-2,3-dihydropyridazin-4-yl}carbonyl)amino]-D-erythro-pentitol;
3-(4-chlorophenyl)-N-(1-hydroxypropan-2-yl)-6-oxo-6H-1,4′-bipyridazine-5-carboxamide;
3-(4-chlorophenyl)-N-[(2S)-1-hydroxypropan-2-yl]-6-oxo-6H-1,4′-bipyridazine-5-carboxamide;
3-(4-chlorophenyl)-N-(2-hydroxy-2-methylpropyl)-6-oxo-6H-1,4′-bipyridazine-5-carboxamide;
6-(4-cyanophenyl)-N-(1-hydroxypropan-2-yl)-3-oxo-2-(pyridin-3-yl)-2,3-dihydropyridazine-4-carboxamide;
6-(4-cyanophenyl)-N-[(2S)-1-hydroxypropan-2-yl]-3-oxo-2-(pyridin-3-yl)-2,3-dihydropyridazine-4-carboxamide;
6-[6-(difluoromethyl)pyridin-3-yl]-N-(1-hydroxypropan-2-yl)-3-oxo-2-(pyridin-3-yl)-2,3-dihydropyridazine-4-carboxamide;
6-[6-(difluoromethyl)pyridin-3-yl]-N-[(2S)-1-hydroxypropan-2-yl]-3-oxo-2-(pyridin-3-yl)-2,3-dihydropyridazine-4-carboxamide;
3-(4-chlorophenyl)-N-[(cis)-4-hydroxytetrahydrofuran-3-yl]-6-oxo-6H-1,4′-bipyridazine-5-carboxamide;
6-[6-(difluoromethyl)pyridin-3-yl]-N-[(cis)-4-methyltetrahydrofuran-3-yl]-3-oxo-2-(pyridin-3-yl)-2,3-dihydropyridazine-4-carboxamide;
6-[6-(difluoromethyl)pyridin-3-yl]-N-[(3R,4S)-4-methyltetrahydrofuran-3-yl]-3-oxo-2-(pyridin-3-yl)-2,3-dihydropyridazine-4-carboxamide;
6-[6-(difluoromethyl)pyridin-3-yl]-N-[(3S,4R)-4-methyltetrahydrofuran-3-yl]-3-oxo-2-(pyridin-3-yl)-2,3-dihydropyridazine-4-carboxamide;
6-(4-chlorophenyl)-3-oxo-2-(pyrimidin-5-yl)-N-(1,1,1-trifluoro-3-hydroxy-3-methylbutan-2-yl)-2,3-dihydropyridazine-4-carboxamide;
6-(4-chlorophenyl)-3-oxo-2-(pyrimidin-5-yl)-N-[(2R)-1,1,1-trifluoro-3-hydroxy-3-methylbutan-2-yl]-2,3-dihydropyridazine-4-carboxamide;
6-(4-chlorophenyl)-3-oxo-2-(pyrimidin-5-yl)-N-[(2S)-1,1,1-trifluoro-3-hydroxy-3-methylbutan-2-yl]-2,3-dihydropyridazine-4-carboxamide;
N-[(1-hydroxycyclopropyl)methyl]-3-oxo-2-(pyridin-3-yl)-6-[4-(trifluoromethyl)phenyl]-2,3-dihydropyridazine-4-carboxamide;
N-[(1-hydroxycyclobutyl)methyl]-3-oxo-2-(pyridin-3-yl)-6-[4-(trifluoromethyl)phenyl]-2,3-dihydropyridazine-4-carboxamide;
6-(4-chlorophenyl)-N-(1-hydroxypropan-2-yl)-3-oxo-2-(1H-pyrazol-4-yl)-2,3-dihydropyridazine-4-carboxamide;
6-(4-chlorophenyl)-N-[(2S)-1-hydroxypropan-2-yl]-3-oxo-2-(1H-pyrazol-4-yl)-2,3-dihydropyridazine-4-carboxamide;
6-[4-(dimethylamino)phenyl]-N-(1-hydroxypropan-2-yl)-3-oxo-2-(pyridin-3-yl)-2,3-dihydropyridazine-4-carboxamide;
6-[4-(dimethylamino)phenyl]-N-[(2S)-1-hydroxypropan-2-yl]-3-oxo-2-(pyridin-3-yl)-2,3-dihydropyridazine-4-carboxamide;
6-(4-chlorophenyl)-N-(1-hydroxypropan-2-yl)-2-(1-methyl-1H-pyrazol-3-yl)-3-oxo-2,3-dihydropyridazine-4-carboxamide;
6-(4-chlorophenyl)-N-[(2S)-1-hydroxypropan-2-yl]-2-(1-methyl-1H-pyrazol-3-yl)-3-oxo-2,3-dihydropyridazine-4-carboxamide;
6-(4-chlorophenyl)-N-(1-hydroxypropan-2-yl)-2-(3-methyl-H-pyrazol-5-yl)-3-oxo-2,3-dihydropyridazine-4-carboxamide;
6-(4-chlorophenyl)-N-[(2S)-1-hydroxypropan-2-yl]-2-(3-methyl-1H-pyrazol-5-yl)-3-oxo-2,3-dihydropyridazine-4-carboxamide;
6-(4-chlorophenyl)-N-(1-hydroxypropan-2-yl)-3-oxo-2-(1,2-thiazol-4-yl)-2,3-dihydropyridazine-4-carboxamide;
6-(4-chlorophenyl)-N-[(2S)-1-hydroxypropan-2-yl]-3-oxo-2-(1,2-thiazol-4-yl)-2,3-dihydropyridazine-4-carboxamide;
N-[(1-Hydroxycyclobutyl)methyl]-3-oxo-2-(1,2-thiazol-4-yl)-6-[4-(trifluoromethyl)phenyl]-2,3-dihydropyridazine-4-carboxamide;
N-[(2S)-3-Hydroxy-3-methylbutan-2-yl]-3-oxo-2-(1,2-thiazol-4-yl)-6-[4-(trifluoromethyl)phenyl]-2,3-dihydropyridazine-4-carboxamide;
(+)-3-Oxo-2-(pyridin-3-yl)-N-(1,1,1-trifluoro-3-hydroxy-3-methylbutan-2-yl)-6-[4-(trifluoromethyl)phenyl]-2,3-dihydropyridazine-4-carboxamide enantiomer 2;
6-(4-Chlorophenyl)-3-oxo-2-(1,2-thiazol-4-yl)-N-[(2R)-1,1,1-trifluoro-3-hydroxypropan-2-yl]-2,3-dihydropyridazine-4-carboxamide;
6-(4-Chlorophenyl)-3-oxo-2-(1,2-thiazol-4-yl)-N-[(2S)-3,3,3-trifluoro-2-hydroxypropyl]-2,3-dihydropyridazine-4-carboxamide;
6-(4-Chlorophenyl)-N-[(1-hydroxycyclobutyl)methyl]-3-oxo-2-(1,2-thiazol-4-yl)-2,3-dihydropyridazine-4-carboxamide;
6-(4-Chlorophenyl)-N-[(2R)-1-fluoro-3-hydroxypropan-2-yl]-3-oxo-2-(1,2-thiazol-4-yl)-2,3-dihydropyridazine-4-carboxamide;
N-[(2R)-3-Hydroxy-3-methylbutan-2-yl]-2-(1-methyl-1H-pyrazol-4-yl)-3-oxo-6-[4-(trifluoromethyl)phenyl]-2,3-dihydropyridazine-4-carboxamide;
N-[(2R)-1-Fluoro-3-hydroxypropan-2-yl]-3-oxo-2-(1,2-thiazol-4-yl)-6-[4-(trifluoromethyl)phenyl]-2,3-dihydropyridazine-4-carboxamide;
N-[(1S,2R)-2-Hydroxycyclopentyl]-2-(1-methyl-1H-pyrazol-4-yl)-3-oxo-6-[4-(trifluoromethyl)phenyl]-2,3-dihydropyridazine-4-carboxamide;
N-[(2S)-1-Hydroxypropan-2-yl]-3-oxo-2-(1,2-thiazol-4-yl)-6-[4-(trifluoromethyl)phenyl]-2,3-dihydropyridazine-4-carboxamide;
N-[(2S)-3-Hydroxy-3-methylbutan-2-yl]-2-(1-methyl-1H-pyrazol-4-yl)-3-oxo-6-[4-(trifluoromethyl)phenyl]-2,3-dihydropyridazine-4-carboxamide;
N-[(1S)-1-Cyclopropyl-2-hydroxyethyl]-3-oxo-2-(1,2-thiazol-4-yl)-6-[4-(trifluoromethyl)phenyl]-2,3-dihydropyridazine-4-carboxamide;
N-(2-Hydroxy-2-methylpropyl)-3-oxo-2-(1,2-thiazol-4-yl)-6-[4-(trifluoromethyl)phenyl]-2,3-dihydropyridazine-4-carboxamide;
6-(4-Chlorophenyl)-N-[(2S)-3-hydroxy-3-methylbutan-2-yl]-3-oxo-2-(1,2-thiazol-4-yl)-2,3-dihydropyridazine-4-carboxamide;
N-[(1S,2S)-2-Hydroxycyclopentyl]-2-(1-methyl-1H-pyrazol-4-yl)-3-oxo-6-[4-(trifluoromethyl)phenyl]-2,3-dihydropyridazine-4-carboxamide;
N-[(2R)-1-Fluoro-3-hydroxypropan-2-yl]-2-(i-methyl-1H-pyrazol-4-yl)-3-oxo-6-[4-(trifluoromethyl)phenyl]-2,3-dihydropyridazine-4-carboxamide;
3-Oxo-2-(1H-pyrazol-4-yl)-N-[(2R)-1,1,1-trifluoro-3-hydroxypropan-2-yl]-6-[4-(trifluoromethyl)phenyl]-2,3-dihydropyridazine-4-carboxamide;
3-Oxo-2-(pyridin-3-yl)-N-(1,1,1-trifluoro-3-hydroxy-3-methylbutan-2-yl)-6-[4-(trifluoromethyl)phenyl]-2,3-dihydropyridazine-4-carboxamide;
2-[1-(Difluoromethyl)-1H-pyrazol-4-yl]-3-oxo-N-[(2R)-1,1,1-trifluoro-3-hydroxypropan-2-yl]-6-[4-(trifluoromethyl)phenyl]-2,3-dihydropyridazine-4-carboxamide;
N-cis-4-Hydroxytetrahydrofuran-3-yl-2-(1-methyl-1H-pyrazol-4-yl)-3-oxo-6-[4-(trifluoromethyl)phenyl]-2,3-dihydropyridazine-4-carboxamide;
3-Oxo-2-(1H-pyrazol-4-yl)-N-[(2S)-3,3,3-trifluoro-2-hydroxypropyl]-6-[4-(trifluoromethyl)phenyl]-2,3-dihydropyridazine-4-carboxamide;
(+)-N-cis-2-Hydroxycyclobutyl-3-oxo-2-(pyridin-3-yl)-6-[4-(trifluoromethyl)phenyl]-2,3-dihydropyridazine-4-carboxamide enantiomer 1;
(+)-N-cis-4-Hydroxytetrahydrofuran-3-yl-2-(1-methyl-H-pyrazol-4-yl)-3-oxo-6-[4-(trifluoromethyl)phenyl]-2,3-dihydropyridazine-4-carboxamide enantiomer 2;
N-[(2S)-3,3-Difluoro-2-hydroxypropyl]-2-(1-methyl-H-pyrazol-4-yl)-3-oxo-6-[4-(trifluoromethyl)phenyl]-2,3-dihydropyridazine-4-carboxamide;
6-(4-Chlorophenyl)-2-(5-fluoro-2-thienyl)-N-[(2S)-1-hydroxypropan-2-yl]-3-oxo-2,3-dihydropyridazine-4-carboxamide;
N-[(2R)-1-Fluoro-3-hydroxypropan-2-yl]-3-oxo-2-(pyridin-3-yl)-6-[4-(trifluoromethyl)phenyl]-2,3-dihydropyridazine-4-carboxamide;
6-(4-Chlorophenyl)-N-(2-hydroxy-2-methylpropyl)-3-oxo-2-(1,2-thiazol-4-yl)-2,3-dihydropyridazine-4-carboxamide;
2-[1-(Difluoromethyl)-1H-pyrazol-4-yl]-3-oxo-N-[(2S)-3,3,3-trifluoro-2-hydroxypropyl]-6-[4-(trifluoromethyl)phenyl]-2,3-dihydropyridazine-4-carboxamide;
N-cis-2-Hydroxycyclobutyl-3-oxo-2-(pyridin-3-yl)-6-[4-(trifluoromethyl)phenyl]-2,3-dihydropyridazine-4-carboxamide;
6-(4-Chlorophenyl)-2-(5-methyl-3-thienyl)-3-oxo-N-[(2S)-3,3,3-trifluoro-2-hydroxypropyl]-2,3-dihydropyridazine-4-carboxamide;
N-[(2S)-3-Hydroxy-3-methylbutan-2-yl]-3-oxo-2-(pyridin-3-yl)-6-[4-(trifluoromethyl)phenyl]-2,3-dihydropyridazine-4-carboxamide;
6-(4-Chlorophenyl)-N-[(1S)-1-cyano-2-hydroxyethyl]-2-(1-methyl-1H-pyrazol-4-yl)-3-oxo-2,3-dihydropyridazine-4-carboxamide;
2-[1-(Difluoromethyl)-1H-pyrazol-4-yl]-N-[(2S)-3-hydroxy-3-methylbutan-2-yl]-3-oxo-6-[4-(trifluoromethyl)phenyl]-2,3-dihydropyridazine-4-carboxamide;
(+)-N-cis-4-Hydroxytetrahydrofuran-3-yl-3-oxo-2-(pyridin-3-yl)-6-[4-(trifluoromethyl)phenyl]-2,3-dihydropyridazine-4-carboxamide enantiomer 1;
(−)-N-(3,3-Difluoro-2-hydroxypropyl)-3-oxo-2-(pyridin-3-yl)-6-[4-(trifluoromethyl)phenyl]-2,3-dihydropyridazine-4-carboxamide enantiomer 2;
(−)-N-cis-4-Hydroxytetrahydrofuran-3-yl-2-(1-methyl-1H-pyrazol-4-yl)-3-oxo-6-[4-(trifluoromethyl)phenyl]-2,3-dihydropyridazine-4-carboxamide enantiomer 1;
6-(4-Chlorophenyl)-2-(5-chloro-3-thienyl)-3-oxo-N-[(2S)-3,3,3-trifluoro-2-hydroxypropyl]-2,3-dihydropyridazine-4-carboxamide;
N-[(2S)-3-Hydroxy-3-methylbutan-2-yl]-3-oxo-2-(1,2-thiazol-4-yl)-6-[6-(trifluoromethyl)pyridin-3-yl]-2,3-dihydropyridazine-4-carboxamide;
N-[(2R)-3-Hydroxy-3-methylbutan-2-yl]-3-oxo-2-(pyridin-3-yl)-6-[4-(trifluoromethyl)phenyl]-2,3-dihydropyridazine-4-carboxamide;
6-(4-Chlorophenyl)-2-(1,2-oxazol-4-yl)-3-oxo-N-[(2S)-3,3,3-trifluoro-2-hydroxypropyl]-2,3-dihydropyridazine-4-carboxamide;
2-[1-(Difluoromethyl)-1H-pyrazol-4-yl]-3-oxo-N-[(2S)-3,3,3-trifluoro-2-hydroxypropyl]-6-[4-(trifluoromethyl)phenyl]-2,3-dihydropyridazine-4-carboxamide;
6-(4-Chlorophenyl)-3-oxo-2-(pyrimidin-5-yl)-N-[(2S)-3,3,3-trifluoro-2-hydroxypropyl]-2,3-dihydropyridazine-4-carboxamide;
N-[(1S)-1-Cyclopropyl-2-hydroxyethyl]-2-(1-methyl-1H-pyrazol-4-yl)-3-oxo-6-[4-(trifluoromethyl)phenyl]-2,3-dihydropyridazine-4-carboxamide;
N-[(1R,2S)-2-Hydroxycyclopentyl]-2-(1-methyl-1H-pyrazol-4-yl)-3-oxo-6-[4-(trifluoromethyl)phenyl]-2,3-dihydropyridazine-4-carboxamide;
N-(2-Hydroxy-2-methylpropyl)-3-oxo-2-(1,2-thiazol-4-yl)-6-[6-(trifluoromethyl)pyridin-3-yl]-2,3-dihydropyridazine-4-carboxamide;
(−)-3-Oxo-2-(pyridin-3-yl)-N-(1,1,1-trifluoro-3-hydroxy-3-methylbutan-2-yl)-6-[4-(trifluoromethyl)phenyl]-2,3-dihydropyridazine-4-carboxamide enantiomer 1;
6-(4-Chlorophenyl)-N-[(2S)-1-hydroxypropan-2-yl]-2-(5-methyl-3-thienyl)-3-oxo-2,3-dihydropyridazine-4-carboxamide;
6-(4-Chlorophenyl)-2-(5-chloro-3-thienyl)-N-[(2S)-1-hydroxypropan-2-yl]-3-oxo-2,3-dihydropyridazine-4-carboxamide;
N-[(2S)-1-Hydroxypropan-2-yl]-3-oxo-2-(1H-pyrazol-4-yl)-6-[4-(trifluoromethyl)phenyl]-2,3-dihydropyridazine-4-carboxamide;
N-[(2S)-3-Fluoro-2-hydroxypropyl]-2-(1-methyl-1H-pyrazol-4-yl)-3-oxo-6-[4-(trifluoromethyl)phenyl]-2,3-dihydropyridazine-4-carboxamide;
6-(4-Chlorophenyl)-N-(2-hydroxy-2-methylpropyl)-2-(5-methyl-3-thienyl)-3-oxo-2,3-dihydropyridazine-4-carboxamide;
N-(3,3-Difluoro-2-hydroxypropyl)-3-oxo-2-(pyridin-3-yl)-6-[4-(trifluoromethyl)phenyl]-2,3-dihydropyridazine-4-carboxamide enantiomer 1;
N-[(1R,2R)-2-Hydroxycyclopentyl]-2-(1-methyl-1H-pyrazol-4-yl)-3-oxo-6-[4-(trifluoromethyl)phenyl]-2,3-dihydropyridazine-4-carboxamide;
(+)-6-(4-Chlorophenyl)-N-cis-2-hydroxycyclobutyl-3-oxo-2-(pyridin-3-yl)-2,3-dihydropyridazine-4-carboxamide enantiomer 1;
6-(4-Chlorophenyl)-2-(5-chloro-3-thienyl)-N-(2-hydroxy-2-methylpropyl)-3-oxo-2,3-dihydropyridazine-4-carboxamide;
N-[(2S)-1-Hydroxypropan-2-yl]-3-oxo-2-(1,2-thiazol-4-yl)-6-[6-(trifluoromethyl)pyridin-3-yl]-2,3-dihydropyridazine-4-carboxamide;
(−)-N-cis-4-Hydroxytetrahydrofuran-3-yl-3-oxo-2-(pyridin-3-yl)-6-[4-(trifluoromethyl)phenyl]-2,3-dihydropyridazine-4-carboxamide, enantiomer 2;
6-(4-Chlorophenyl)-N-[(2S)-1-hydroxypropan-2-yl]-2-(1,2-oxazol-4-yl)-3-oxo-2,3-dihydropyridazine-4-carboxamide;
6-(4-Chlorophenyl)-N-[(1S)-1-cyano-2-hydroxyethyl]-3-oxo-2-(pyridin-3-yl)-2,3-dihydropyridazine-4-carboxamide;
N-[(2S)-3-Fluoro-2-hydroxypropyl]-3-oxo-2-(pyridin-3-yl)-6-[4-(trifluoromethyl)phenyl]-2,3-dihydropyridazine-4-carboxamide;
6-(4-Chlorophenyl)-2-[1-(difluoromethyl)-1H-pyrazol-4-yl]-3-oxo-N-[(2S)-3,3,3-trifluoro-2-hydroxypropyl]-2,3-dihydropyridazine-4-carboxamide;
N-[(2R)-1-Hydroxy-3-methoxypropan-2-yl]-3-oxo-2-(pyridin-3-yl)-6-[4-(trifluoromethyl)phenyl]-2,3-dihydropyridazine-4-carboxamide;
6-(4-Chlorophenyl)-N-cis-2-hydroxycyclobutyl-3-oxo-2-(pyridin-3-yl)-2,3-dihydropyridazine-4-carboxamide;
6-(4-Chlorophenyl)-N-[(1S)-1-cyclopropyl-2-hydroxyethyl]-2-(5-fluoropyridin-3-yl)-3-oxo-2,3-dihydropyridazine-4-carboxamide;
N-[(2S)-3-Hydroxy-3-methylbutan-2-yl]-3-oxo-2-(1H-pyrazol-4-yl)-6-[4-(trifluoromethyl)phenyl]-2,3-dihydropyridazine-4-carboxamide;
2-[1-(Difluoromethyl)-1H-pyrazol-4-yl]-N-[(2S)-1-hydroxypropan-2-yl]-3-oxo-6-[4-(trifluoromethyl)phenyl]-2,3-dihydropyridazine-4-carboxamide;
2-[1-(Difluoromethyl)-1H-pyrazol-4-yl]-N-(2-hydroxy-2-methylpropyl)-3-oxo-6-[4-(trifluoromethyl)phenyl]-2,3-dihydropyridazine-4-carboxamide;
6-(4-Chlorophenyl)-N-[(1-hydroxycyclopropyl)methyl]-3-oxo-2-(pyridin-3-yl)-2,3-dihydropyridazine-4-carboxamide;
6-(4-Chlorophenyl)-2-[1-(difluoromethyl)-1H-pyrazol-4-yl]-N-[(2S)-3-hydroxy-3-methylbutan-2-yl]-3-oxo-2,3-dihydropyridazine-4-carboxamide;
6-(4-Chlorophenyl)-N-[(2R)-3-hydroxy-3-methylbutan-2-yl]-3-oxo-2-(pyrimidin-5-yl)-2,3-dihydropyridazine-4-carboxamide;
N-(2-Hydroxy-2-methylpropyl)-3-oxo-2-(1H-pyrazol-4-yl)-6-[4-(trifluoromethyl)phenyl]-2,3-dihydropyridazine-4-carboxamide;
Methyl N-{[6-(4-chlorophenyl)-2-(1-methyl-1H-pyrazol-4-yl)-3-oxo-2,3-dihydropyridazin-4-yl]carbonyl}-D-serinate;
(−)-N-cis-2-Hydroxycyclobutyl-3-oxo-2-(pyridin-3-yl)-6-[4-(trifluoromethyl)phenyl]-2,3-dihydropyridazine-4-carboxamide enantiomer 2;
6-(4-Chlorophenyl)-N-(2-hydroxy-2-methylpropyl)-2-(1,2-oxazol-4-yl)-3-oxo-2,3-dihydropyridazine-4-carboxamide;
6-(4-Chlorophenyl)-N-[(2S)-3-fluoro-2-hydroxypropyl]-2-(1-methyl-1H-pyrazol-4-yl)-3-oxo-2,3-dihydropyridazine-4-carboxamide;
6-(4-Chlorophenyl)-3-oxo-2-(pyrimidin-5-yl)-N-[(2R)-1,1,1-trifluoro-3-hydroxypropan-2-yl]-2,3-dihydropyridazine-4-carboxamide;
6-(4-Chlorophenyl)-2-[1-(difluoromethyl)-1H-pyrazol-4-yl]-N-[(2S)-1-hydroxypropan-2-yl]-3-oxo-2,3-dihydropyridazine-4-carboxamide;
6-(4-Chlorophenyl)-N-[(1S)-1-cyclopropyl-2-hydroxyethyl]-3-oxo-2-(pyrimidin-5-yl)-2,3-dihydropyridazine-4-carboxamide;
Methyl N-{[6-(4-chlorophenyl)-3-oxo-2-(pyridin-3-yl)-2,3-dihydropyridazin-4-yl]carbonyl}-D-serinate;
6-[4-(Fluoromethyl)phenyl]-2-(1-methyl-1H-pyrazol-4-yl)-3-oxo-N-[(2R)-1,1,1-trifluoro-3-hydroxypropan-2-yl]-2,3-dihydropyridazine-4-carboxamide;
6-(4-Chlorophenyl)-N-[(2S)-3-hydroxy-3-methylbutan-2-yl]-3-oxo-2-(1H-pyrazol-4-yl)-2,3-dihydropyridazine-4-carboxamide;
6-(4-Chlorophenyl)-N-(2-hydroxy-2-methylpropyl)-3-oxo-2-(pyrimidin-5-yl)-2,3-dihydropyridazine-4-carboxamide;
6-(4-Chlorophenyl)-2-[1-(difluoromethyl)-1H-pyrazol-4-yl]-N-(2-hydroxy-2-methylpropyl)-3-oxo-2,3-dihydropyridazine-4-carboxamide;
6-(4-Chlorophenyl)-N-[(2R)-1-hydroxypropan-2-yl]-3-oxo-2-(pyrimidin-5-yl)-2,3-dihydropyridazine-4-carboxamide;
6-[4-(Fluoromethyl)phenyl]-3-oxo-2-(pyridin-3-yl)-N-[(2S)-3,3,3-trifluoro-2-hydroxypropyl]-2,3-dihydropyridazine-4-carboxamide;
(−)-6-(4-Chlorophenyl)-N-cis-2-hydroxycyclobutyl-3-oxo-2-(pyridin-3-yl)-2,3-dihydropyridazine-4-carboxamide, enantiomer 2;
6-[4-(Fluoromethyl)phenyl]-3-oxo-2-(pyridin-3-yl)-N-[(2R)-1,1,1-trifluoro-3-hydroxypropan-2-yl]-2,3-dihydropyridazine-4-carboxamide;
6-[4-(Fluoromethyl)phenyl]-2-(1-methyl-1H-pyrazol-4-yl)-3-oxo-N-[(2S)-3,3,3-trifluoro-2-hydroxypropyl]-2,3-dihydropyridazine-4-carboxamide;
6-[4-(Fluoromethyl)phenyl]-N-[(2S)-3-hydroxy-3-methylbutan-2-yl]-2-(1-methyl-1H-pyrazol-4-yl)-3-oxo-2,3-dihydropyridazine-4-carboxamide;
6-[4-(Fluoromethyl)phenyl]-N-[(2S)-1-hydroxypropan-2-yl]-3-oxo-2-(pyridin-3-yl)-2,3-dihydropyridazine-4-carboxamide;
6-[4-(Fluoromethyl)phenyl]-N-(2-hydroxy-2-methylpropyl)-3-oxo-2-(pyridin-3-yl)-2,3-dihydropyridazine-4-carboxamide;
6-[4-(Fluoromethyl)phenyl]-N-[(2S)-1-hydroxypropan-2-yl]-2-(1-methyl-1H-pyrazol-4-yl)-3-oxo-2,3-dihydropyridazine-4-carboxamide;
6-[4-(Fluoromethyl)phenyl]-N-[(2S)-3-hydroxy-3-methylbutan-2-yl]-3-oxo-2-(pyridin-3-yl)-2,3-dihydropyridazine-4-carboxamide;
N-[(2R)-1-Amino-3-hydroxy-1-oxopropan-2-yl]-6-(4-chlorophenyl)-2-(1-methyl-1H-pyrazol-4-yl)-3-oxo-2,3-dihydropyridazine-4-carboxamide;
N-cis-4-Hydroxytetrahydrothiophen-3-yl-3-oxo-2-(pyridin-3-yl)-6-[4-(trifluoromethyl)phenyl]-2,3-dihydropyridazine-4-carboxamide;
(−)-N-cis-4-Hydroxytetrahydrothiophen-3-yl-3-oxo-2-(pyridin-3-yl)-6-[4-(trifluoromethyl)phenyl]-2,3-dihydropyridazine-4-carboxamide, enantiomer 2;
N-(2-Hydroxy-2-methylpropyl)-3-oxo-2-(pyridin-3-yl)-6-[4-(trifluoromethoxy)phenyl]-2,3-dihydropyridazine-4-carboxamide;
3-Oxo-2-(pyridin-3-yl)-N-[(2R)-3,3,3-trifluoro-2-hydroxypropyl]-6-[4-(trifluoromethoxy)-phenyl]-2,3-dihydropyridazine-4-carboxamide;
N-[(2R)-3-Hydroxy-3-methylbutan-2-yl]-3-oxo-2-(pyridin-3-yl)-6-[4-(trifluoromethoxy)-phenyl]-2,3-dihydropyridazine-4-carboxamide;
N-[(2S)-3,3-Difluoro-2-hydroxypropyl]-3-oxo-2-(pyridin-3-yl)-6-[4-(trifluoromethoxy)phenyl]-2,3-dihydropyridazine-4-carboxamide;
N-[(2S)-3-Fluoro-2-hydroxypropyl]-3-oxo-2-(pyridin-3-yl)-6-[4-(trifluoromethoxy)phenyl]-2,3-dihydropyridazine-4-carboxamide;
6-(4-Chlorophenyl)-3-oxo-2-(pyridin-3-yl)-N-(4,4,4-trifluoro-3-hydroxybutan-2-yl)-2,3-dihydropyridazine-4-carboxamide Isomer 1;
6-(4-Chlorophenyl)-3-oxo-2-(pyridin-3-yl)-N-(4,4,4-trifluoro-3-hydroxybutan-2-yl)-2,3-dihydropyridazine-4-carboxamide Isomer 2;
6-(4-Chlorophenyl)-3-oxo-2-(pyridin-3-yl)-N-(4,4,4-trifluoro-3-hydroxybutan-2-yl)-2,3-dihydropyridazine-4-carboxamide Isomer 3;
6-(4-Chlorophenyl)-3-oxo-2-(pyridin-3-yl)-N-(4,4,4-trifluoro-3-hydroxybutan-2-yl)-2,3-dihydropyridazine-4-carboxamide Isomer 4;
6-(4-Chlorophenyl)-2-(5-fluoropyridin-3-yl)-N-[(cis)-4-hydroxytetrahydrofuran-3-yl]-3-oxo-2,3-dihydropyridazine-4-carboxamide;
6-(4-Chlorophenyl)-N-[(2R)-3-hydroxy-3-methylbutan-2-yl]-3-oxo-2-(pyridin-3-yl)-2,3-dihydropyridazine-4-carboxamide;
6-(4-Chlorophenyl)-2-(5-chloropyridin-3-yl)-N-[(2S)-1-hydroxypropan-2-yl]-3-oxo-2,3-dihydropyridazine-4-carboxamide;
6-(4-Chlorophenyl)-N-[(2S)-1-hydroxypropan-2-yl]-2-(5-methylpyridin-3-yl)-3-oxo-2,3-dihydropyridazine-4-carboxamide;
6-[4-(Difluoromethoxy)phenyl]-2-(1-methyl-1H-pyrazol-4-yl)-3-oxo-N-(1,1,1-trifluoro-3-hydroxy-3-methylbutan-2-yl)-2,3-dihydropyridazine-4-carboxamide;
6-[4-(Difluoromethoxy)phenyl]-2-(1-methyl-1H-pyrazol-4-yl)-3-oxo-N-(1,1,1-trifluoro-3-hydroxy-3-methylbutan-2-yl)-2,3-dihydropyridazine-4-carboxamide Isomer 1;
6-[4-(Difluoromethoxy)phenyl]-2-(1-methyl-1H-pyrazol-4-yl)-3-oxo-N-(1,1,1-trifluoro-3-hydroxy-3-methylbutan-2-yl)-2,3-dihydropyridazine-4-carboxamide Isomer 2;
6-[4-(Difluoromethoxy)phenyl]-N-[(2S)-1-hydroxypropan-2-yl]-2-(1-methyl-1H-pyrazol-4-yl)-3-oxo-2,3-dihydropyridazine-4-carboxamide;
6-[4-(Difluoromethoxy)phenyl]-N-[(2S)-3-hydroxy-3-methylbutan-2-yl]-2-(1-methyl-1H-pyrazol-4-yl)-3-oxo-2,3-dihydropyridazine-4-carboxamide;
6-[4-(Dimethylamino)phenyl]-N-[(2S)-3-hydroxy-3-methylbutan-2-yl]-2-(1-methyl-1H-pyrazol-4-yl)-3-oxo-2,3-dihydropyridazine-4-carboxamide;
6-[4-(Dimethylamino)phenyl]-N-[(2S)-1-hydroxypropan-2-yl]-2-(1-methyl-1H-pyrazol-4-yl)-3-oxo-2,3-dihydropyridazine-4-carboxamide;
N-[(2S)-3-Hydroxy-3-methylbutan-2-yl]-3-oxo-2-(1,2-thiazol-4-yl)-6-[4-(trifluoromethoxy)-phenyl]-2,3-dihydropyridazine-4-carboxamide;
6-[4-(Difluoromethyl)phenyl]-3-oxo-2-(pyridin-3-yl)-N-[(2S)-3,3,3-trifluoro-2-hydroxy-propyl]-2,3-dihydropyridazine-4-carboxamide;
6-[4-(Difluoromethoxy)phenyl]-3-oxo-2-(pyridin-3-yl)-N-[(2S)-3,3,3-trifluoro-2-hydroxypropyl]-2,3-dihydropyridazine-4-carboxamide;
6-[4-(Difluoromethyl)phenyl]-N-[(cis)-4-hydroxytetrahydrofuran-3-yl]-2-(1-methyl-1H-pyrazol-4-yl)-3-oxo-2,3-dihydropyridazine-4-carboxamide Isomer 1;
6-(4-Chlorophenyl)-N-[(2S)-3-hydroxy-3-methylbutan-2-yl]-2-(1-methyl-1H-pyrazol-4-yl)-3-oxo-2,3-dihydropyridazine-4-carboxamide;
6-(4-Chlorophenyl)-N-(2-hydroxy-2-methylcyclopentyl)-2-(1-methyl-1H-pyrazol-4-yl)-3-oxo-2,3-dihydropyridazine-4-carboxamide;
6-[4-(Difluoromethyl)phenyl]-N-[(cis)-4-hydroxytetrahydrofuran-3-yl]-3-oxo-2-(pyridin-3-yl)-2,3-dihydropyridazine-4-carboxamide Isomer 1;
6-[4-(Difluoromethyl)phenyl]-N-[(2S)-3-hydroxy-3-methylbutan-2-yl]-3-oxo-2-(pyridin-3-yl)-2,3-dihydropyridazine-4-carboxamide;
6-[4-(Difluoromethyl)phenyl]-N-[(cis)-4-hydroxytetrahydrofuran-3-yl]-2-(1-methyl-1H-pyrazol-4-yl)-3-oxo-2,3-dihydropyridazine-4-carboxamide Isomer 2;
3-(4-Chlorophenyl)-6-oxo-N-[(2S)-3,3,3-trifluoro-2-hydroxypropyl]-6H-1,4′-bipyridazine-5-carboxamide;
6-[4-(Difluoromethyl)phenyl]-N-(2-hydroxy-2-methylpropyl)-3-oxo-2-(pyridin-3-yl)-2,3-dihydropyridazine-4-carboxamide;
6-[4-(Difluoromethyl)phenyl]-N-[(cis)-4-hydroxytetrahydrofuran-3-yl]-3-oxo-2-(pyridin-3-yl)-2,3-dihydropyridazine-4-carboxamide Isomer 2;
6-(4-Chlorophenyl)-N-[(2R)-1-hydroxypropan-2-yl]-2-(1-methyl-1H-pyrazol-4-yl)-3-oxo-2,3-dihydropyridazine-4-carboxamide;
6-(4-Chlorophenyl)-N-[(2R)-3-hydroxy-3-methylbutan-2-yl]-3-oxo-2-(1H-pyrazol-4-yl)-2,3-dihydropyridazine-4-carboxamide;
6-(4-Chlorophenyl)-N-[(2R)-3-hydroxy-3-methylbutan-2-yl]-3-oxo-2-(1,2-thiazol-4-yl)-2,3-dihydropyridazine-4-carboxamide;
N-[(2R)-3-Hydroxy-3-methylbutan-2-yl]-3-oxo-2-(1,2-thiazol-4-yl)-6-[4-(trifluoromethoxy)-phenyl]-2,3-dihydropyridazine-4-carboxamide;
N-[(cis)-2-hydroxy-2-methylcyclopentyl]-3-oxo-2-(pyridin-3-yl)-6-[4-(trifluoromethoxy)-phenyl]-2,3-dihydropyridazine-4-carboxamide;
N-[(cis)-2-hydroxy-2-methylcyclopentyl]-3-oxo-2-(pyridin-3-yl)-6-[4-(trifluoromethoxy)-phenyl]-2,3-dihydropyridazine-4-carboxamide, isomer 1;
N-[(cis)-2-hydroxy-2-methylcyclopentyl]-3-oxo-2-(pyridin-3-yl)-6-[4-(trifluoromethoxy)-phenyl]-2,3-dihydropyridazine-4-carboxamide, isomer 2;
6-[4-(difluoromethoxy)phenyl]-N-(2-hydroxy-2-methylpropyl)-3-oxo-2-(pyridin-3-yl)-2,3-dihydropyridazine-4-carboxamide;
6-[4-(difluoromethoxy)phenyl]-N-[(2S)-1-hydroxypropan-2-yl]-3-oxo-2-(pyridin-3-yl)-2,3-dihydropyridazine-4-carboxamide;
6-[4-(difluoromethoxy)phenyl]-N-[(2S)-3-hydroxy-3-methylbutan-2-yl]-3-oxo-2-(pyridin-3-yl)-2,3-dihydropyridazine-4-carboxamide;
6-[4-(difluoromethoxy)phenyl]-N-[(2S,3S)-3-hydroxybutan-2-yl]-3-oxo-2-(pyridin-3-yl)-2,3-dihydropyridazine-4-carboxamide;
6-(4-chlorophenyl)-N-[(cis)-2-hydroxy-2-methylcyclopentyl]-2-(1-methyl-1H-pyrazol-4-yl)-3-oxo-2,3-dihydropyridazine-4-carboxamide; isomer 1;
6-(4-Chlorophenyl)-N-[(cis)-2-hydroxy-2-methylcyclopentyl]-2-(1-methyl-1H-pyrazol-4-yl)-3-oxo-2,3-dihydropyridazine-4-carboxamide; Isomer 2;
2-(5-fluoropyridin-3-yl)-N-[(2S)-1-hydroxy-3-methylbutan-2-yl]-3-oxo-6-[6-(trifluoro-methyl)pyridin-3-yl]-2,3-dihydropyridazine-4-carboxamide;
2-(5-fluoropyridin-3-yl)-N-[(1S,2R)-2-hydroxycyclopentyl]-3-oxo-6-[6-(trifluoromethyl)-pyridin-3-yl]-2,3-dihydropyridazine-4-carboxamide;
N-[(1S)-1-cyclopropyl-2-hydroxyethyl]-2-(5-fluoropyridin-3-yl)-3-oxo-6-[6-(trifluoromethyl)-pyridin-3-yl]-2,3-dihydropyridazine-4-carboxamide;
2-(5-fluoropyridin-3-yl)-N-[(2S)-3-hydroxy-3-methylbutan-2-yl]-3-oxo-6-[6-(trifluoro-methyl)pyridin-3-yl]-2,3-dihydropyridazine-4-carboxamide;
N-[(1S)-1-cyano-2-hydroxyethyl]-2-(5-fluoropyridin-3-yl)-3-oxo-6-[6-(trifluoromethyl)-pyridin-3-yl]-2,3-dihydropyridazine-4-carboxamide;
2-(5-fluoropyridin-3-yl)-N-[(2R)-3-hydroxy-3-methylbutan-2-yl]-3-oxo-6-[6-(trifluoro-methyl)pyridin-3-yl]-2,3-dihydropyridazine-4-carboxamide;
1,5-anhydro-2,4-dideoxy-2-[({2-(5-fluoropyridin-3-yl)-3-oxo-6-[6-(trifluoromethyl)pyridin-3-yl]-2,3-dihydropyridazine-4-yl}carbonyl)amino]-D-erythro-pentitol;
2-(5-fluoropyridin-3-yl)-N-(2-hydroxy-2-methylpropyl)-3-oxo-6-[6-(trifluoromethyl)pyridin-3-yl]-2,3-dihydropyridazine-4-carboxamide; and
N-[(2S)-1-hydroxypropan-2-yl]-3-oxo-2-(pyridin-3-yl)-6-[5-(trifluoromethyl)pyridin-2-yl]-2,3-dihydropyridazine-4-carboxamide,
or a polymorph, enantiomer, diastereomer, racemate, tautomer, N oxide, hydrate, solvate, physiologically acceptable salt, or a solvate of a physiologically acceptable salt thereof, or a mixture of any of the foregoing.

21: The combination according to claim 1, wherein component A is:
N-(1-Hydroxy-3-methylbutan-2-yl)-6-(4-methylphenyl)-2-(1-methyl-1H-pyrazol-4-yl)-3-oxo-2,3-dihydropyridazine-4-carboxamide;
N-(1-Hydroxy-3-methylbutan-2-yl)-6-(4-methylphenyl)-2-(1-methyl-1H-pyrazol-4-yl)-3-oxo-2,3-dihydropyridazine-4-carboxamide, Enantiomer 1;
N-(1-Hydroxy-3-methylbutan-2-yl)-6-(4-methylphenyl)-2-(1-methyl-1H-pyrazol-4-yl)-3-oxo-2,3-dihydropyridazine-4-carboxamide, Enantiomer 2;
N-(1-Hydroxybutan-2-yl)-6-(4-methylphenyl)-2-(1-methyl-1H-pyrazol-4-yl)-3-oxo-2,3-dihydropyridazine-4-carboxamide;
N-(1-Hydroxybutan-2-yl)-6-(4-methylphenyl)-2-(1-methyl-1H-pyrazol-4-yl)-3-oxo-2,3-dihydropyridazine-4-carboxamide, Enantiomer 1;
N-(1-Hydroxybutan-2-yl)-6-(4-methylphenyl)-2-(1-methyl-1H-pyrazol-4-yl)-3-oxo-2,3-dihydropyridazine-4-carboxamide, Enantiomer 2;
N-(1-Hydroxypropan-2-yl)-6-(4-methylphenyl)-2-(1-methyl-1H-pyrazol-4-yl)-3-oxo-2,3-dihydropyridazine-4-carboxamide;
N-[(2S)-1-Hydroxypropan-2-yl]-6-(4-methylphenyl)-2-(1-methyl-1H-pyrazol-4-yl)-3-oxo-2,3-dihydropyridazine-4-carboxamide;
N-[(2R)-1-Hydroxypropan-2-yl]-6-(4-methylphenyl)-2-(1-methyl-1H-pyrazol-4-yl)-3-oxo-2,3-dihydropyridazine-4-carboxamide;
6-(4-Methylphenyl)-2-(1-methyl-1H-pyrazol-4-yl)-3-oxo-N-(3,3,3-trifluoro-2-hydroxypropyl)-2,3-dihydropyridazine-4-carboxamide;
(−)-6-(4-Methylphenyl)-2-(1-methyl-1H-pyrazol-4-yl)-3-oxo-N-(3,3,3-trifluoro-2-hydroxypropyl)-2,3-dihydropyridazine-4-carboxamide, Enantiomer 1;
(+)-6-(4-Methylphenyl)-2-(1-methyl-1H-pyrazol-4-yl)-3-oxo-N-(3,3,3-trifluoro-2-hydroxypropyl)-2,3-dihydropyridazine-4-carboxamide, Enantiomer 2;
N-(3,3-Difluoro-2-hydroxypropyl)-6-(4-methylphenyl)-2-(1-methyl-1H-pyrazol-4-yl)-3-oxo-2,3-dihydropyridazine-4-carboxamide;
N-(3,3-Difluoro-2-hydroxypropyl)-6-(4-methylphenyl)-2-(1-methyl-1H-pyrazol-4-yl)-3-oxo-2,3-dihydropyridazine-4-carboxamide, Enantiomer 1;
N-(3,3-Difluoro-2-hydroxypropyl)-6-(4-methylphenyl)-2-(1-methyl-1H-pyrazol-4-yl)-3-oxo-2,3-dihydropyridazine-4-carboxamide, Enantiomer 2;
6-(4-Chlorophenyl)-N-[(2S)-1-hydroxy-3-methylbutan-2-yl]-2-(1-methyl-1H-pyrazol-4-yl)-3-oxo-2,3-dihydropyridazine-4-carboxamide;
6-(4-Chlorophenyl)-N-[(2S)-1-hydroxypropan-2-yl]-2-(1-methyl-1H-pyrazol-4-yl)-3-oxo-2,3-dihydropyridazine-4-carboxamide;
6-(4-Chlorophenyl)-2-(1-methyl-1H-pyrazol-4-yl)-3-oxo-N-(3,3,3-trifluoro-2-hydroxypropyl)-2,3-dihydropyridazine-4-carboxamide;
(−)-6-(4-Chlorophenyl)-2-(1-methyl-1H-pyrazol-4-yl)-3-oxo-N-(3,3,3-trifluoro-2-hydroxypropyl)-2,3-dihydropyridazine-4-carboxamide;
(+)-6-(4-Chlorophenyl)-2-(1-methyl-1H-pyrazol-4-yl)-3-oxo-N-(3,3,3-trifluoro-2-hydroxypropyl)-2,3-dihydropyridazine-4-carboxamide;
6-(4-Chlorophenyl)-N-(3,3-difluoro-2-hydroxypropyl)-2-(i-methyl-1H-pyrazol-4-yl)-3-oxo-2,3-dihydropyridazine-4-carboxamide;
(−)-6-(4-Chlorophenyl)-N-(3,3-difluoro-2-hydroxypropyl)-2-(i-methyl-1H-pyrazol-4-yl)-3-oxo-2,3-dihydropyridazine-4-carboxamide;
(+)-6-(4-Chlorophenyl)-N-(3,3-difluoro-2-hydroxypropyl)-2-(i-methyl-1H-pyrazol-4-yl)-3-oxo-2,3-dihydropyridazine-4-carboxamide;
6-(4-Chlorophenyl)-N-(2-hydroxy-3-methoxypropyl)-2-(1-methyl-1H-pyrazol-4-yl)-3-oxo-2,3-dihydropyridazine-4-carboxamide;
(−)-6-(4-Chlorophenyl)-N-(2-hydroxy-3-methoxypropyl)-2-(1-methyl-1H-pyrazol-4-yl)-3-oxo-2,3-dihydropyridazine-4-carboxamide;
(+)-6-(4-Chlorophenyl)-N-(2-hydroxy-3-methoxypropyl)-2-(1-methyl-1H-pyrazol-4-yl)-3-oxo-2,3-dihydropyridazine-4-carboxamide;
6-(4-Chlorophenyl)-N-[(2S)-2,3-dihydroxypropyl]-2-(1-methyl-1H-pyrazol-4-yl)-3-oxo-2,3-dihydropyridazine-4-carboxamide;
6-(4-Chlorophenyl)-N-[(2R)-2,3-dihydroxypropyl]-2-(1-methyl-1H-pyrazol-4-yl)-3-oxo-2,3-dihydropyridazine-4-carboxamide;
6-(4-Chlorophenyl)-N-[(2S)-1-hydroxy-3-methoxypropan-2-yl]-2-(1-methyl-1H-pyrazol-4-yl)-3-oxo-2,3-dihydropyridazine-4-carboxamide;
6-(4-Chlorophenyl)-N-[(2R)-1-hydroxy-3-methoxypropan-2-yl]-2-(1-methyl-1H-pyrazol-4-yl)-3-oxo-2,3-dihydropyridazine-4-carboxamide;
6-(4-Chlorophenyl)-N-(1,3-dihydroxypropan-2-yl)-2-(1-methyl-1H-pyrazol-4-yl)-3-oxo-2,3-dihydropyridazine-4-carboxamide;
6-(4-Chlorophenyl)-N-(2-hydroxy-2-methylpropyl)-2-(1-methyl-1H-pyrazol-4-yl)-3-oxo-2,3-dihydropyridazine-4-carboxamide;
6-(4-Chlorophenyl)-2-(1-methyl-1H-pyrazol-4-yl)-3-oxo-N-[(2RS)-1,1,1-trifluoro-3-hydroxypropan-2-yl]-2,3-dihydropyridazine-4-carboxamide;
(−)-6-(4-Chlorophenyl)-2-(1-methyl-1H-pyrazol-4-yl)-3-oxo-N-[(2S)-1,1,1-trifluoro-3-hydroxypropan-2-yl]-2,3-dihydropyridazine-4-carboxamide;
(+)-6-(4-Chlorophenyl)-2-(1-methyl-1H-pyrazol-4-yl)-3-oxo-N-[(2R)-1,1,1-trifluoro-3-hydroxypropan-2-yl]-2,3-dihydropyridazine-4-carboxamide;
2-(1-Methyl-1H-pyrazol-4-yl)-3-oxo-N-[(2R)-1,1,1-trifluoro-3-hydroxypropan-2-yl]-6-[4-(trifluoromethyl)phenyl]-2,3-dihydropyridazine-4-carboxamide;
2-(1-Methyl-1H-pyrazol-4-yl)-3-oxo-N-[(2S)-3,3,3-trifluoro-2-hydroxypropyl]-6-[4-(trifluoromethyl)phenyl]-2,3-dihydropyridazine-4-carboxamide;
(−)-N-[(2R)-1-Hydroxypropan-2-yl]-2-(1-methyl-1H-pyrazol-4-yl)-3-oxo-6-[4-(trifluoromethyl)phenyl]-2,3-dihydropyridazine-4-carboxamide;
(+)-N-[(2S)-1-Hydroxypropan-2-yl]-2-(1-methyl-1H-pyrazol-4-yl)-3-oxo-6-[4-(trifluoromethyl)phenyl]-2,3-dihydropyridazine-4-carboxamide;
6-(4-Chlorophenyl)-2-(1-cyclobutyl-1H-pyrazol-4-yl)-3-oxo-N-[(2S)-3,3,3-trifluoro-2-hydroxypropyl]-2,3-dihydropyridazine-4-carboxamide;
6-(4-Chlorophenyl)-2-(1-cyclobutyl-1H-pyrazol-4-yl)-3-oxo-N-[(2R)-1,1,1-trifluoro-3-hydroxypropan-2-yl]-2,3-dihydropyridazine-4-carboxamide;
6-(4-Chlorophenyl)-2-(1-cyclobutyl-1H-pyrazol-4-yl)-N-[(2S)-1-hydroxypropan-2-yl]-3-oxo-2,3-dihydropyridazine-4-carboxamide;
6-(4-Chlorophenyl)-N-[(cis)-4-hydroxytetrahydrofuran-3-yl]-2-(1-methyl-1H-pyrazol-4-yl)-3-oxo-2,3-dihydropyridazine-4-carboxamide;
6-(4-Chlorophenyl)-N-[(cis)-4-hydroxytetrahydrofuran-3-yl]-2-(1-methyl-1H-pyrazol-4-yl)-3-oxo-2,3-dihydropyridazine-4-carboxamide, Enantiomer 1;
6-(4-Chlorophenyl)-N-[(cis)-4-hydroxytetrahydrofuran-3-yl]-2-(1-methyl-1H-pyrazol-4-yl)-3-oxo-2,3-dihydropyridazine-4-carboxamide, Enantiomer 2;
N-[(1S,2R)-2-Hydroxycyclohexyl]-2-(1-methyl-1H-pyrazol-4-yl)-3-oxo-6-[4-(trifluoromethoxy)phenyl]-2,3-dihydropyridazine-4-carboxamide;
2-(1-Methyl-1H-pyrazol-4-yl)-3-oxo-N-[(2R)-1,1,1-trifluoro-3-hydroxypropan-2-yl]-6-[4-(trifluoromethoxy)phenyl]-2,3-dihydropyridazine-4-carboxamide;
6-(4-Chlorophenyl)-N-[(1S,2R)-2-hydroxycyclohexyl]-2-(1-methyl-1H-pyrazol-4-yl)-3-oxo-2,3-dihydropyridazine-4-carboxamide;
6-(4-Chlorophenyl)-N-[(1S,2S)-2-hydroxycyclopentyl]-2-(1-methyl-1H-pyrazol-4-yl)-3-oxo-2,3-dihydropyridazine-4-carboxamide;
6-(4-Chlorophenyl)-N-[(1S,2R)-2-hydroxycyclopentyl]-2-(1-methyl-1H-pyrazol-4-yl)-3-oxo-2,3-dihydropyridazine-4-carboxamide;
6-(4-Chlorophenyl)-N-[(trans)-2-hydroxycyclopentyl]-2-(1-methyl-1H-pyrazol-4-yl)-3-oxo-2,3-dihydropyridazine-4-carboxamide;
6-[4-(Difluoromethyl)phenyl]-N-[(trans)-2-hydroxycyclopentyl]-2-(1-methyl-1H-pyrazol-4-yl)-3-oxo-2,3-dihydropyridazine-4-carboxamide;
N-[(2S)-1-Hydroxypropan-2-yl]-2-(1-methyl-1H-pyrazol-4-yl)-3-oxo-6-[4-(trifluoromethoxy)phenyl]-2,3-dihydropyridazine-4-carboxamide;
6-[6-(Difluoromethyl)pyridin-3-yl]-N-[(1S,2R)-2-hydroxycyclohexyl]-2-(1-methyl-1H-pyrazol-4-yl)-3-oxo-2,3-dihydropyridazine-4-carboxamide;
N-[(cis)-4-Hydroxytetrahydrofuran-3-yl]-2-(1-methyl-1H-pyrazol-4-yl)-3-oxo-6-[4-(trifluoromethoxy)phenyl]-2,3-dihydropyridazine-4-carboxamide;
N-[(cis)-4-Hydroxytetrahydrofuran-3-yl]-2-(1-methyl-1H-pyrazol-4-yl)-3-oxo-6-[4-(trifluoromethoxy)phenyl]-2,3-dihydropyridazine-4-carboxamide, Enantiomer 1;
N-[(cis)-4-Hydroxytetrahydrofuran-3-yl]-2-(1-methyl-1H-pyrazol-4-yl)-3-oxo-6-[4-(trifluoromethoxy)phenyl]-2,3-dihydropyridazine-4-carboxamide, Enantiomer 2;
6-[4-(Difluoromethyl)phenyl]-N-[(2S)-1-hydroxypropan-2-yl]-2-(1-methyl-1H-pyrazol-4-yl)-3-oxo-2,3-dihydropyridazine-4-carboxamide;
N-(2-Hydroxy-2-methylpropyl)-2-(1-methyl-1H-pyrazol-4-yl)-3-oxo-6-[4-(trifluoromethoxy)phenyl]-2,3-dihydropyridazine-4-carboxamide;
6-[4-(Difluoromethyl)phenyl]-N-[(cis)-4-hydroxytetrahydrofuran-3-yl]-2-(1-methyl-1H-pyrazol-4-yl)-3-oxo-2,3-dihydropyridazine-4-carboxamide;
1,5-Anhydro-2-({[6-(4-chlorophenyl)-2-(1-methyl-1H-pyrazol-4-yl)-3-oxo-2,3-dihydropyridazin-4-yl]carbonyl}amino)-2,4-dideoxy-D-erythro-pentitol;
6-[4-(Difluoromethyl)phenyl]-N-(2-hydroxy-2-methylpropyl)-2-(1-methyl-1H-pyrazol-4-yl)-3-oxo-2,3-dihydropyridazine-4-carboxamide;
N-[(trans)-4-Hydroxytetrahydrofuran-3-yl]-2-(1-methyl-1H-pyrazol-4-yl)-3-oxo-6-[4-(trifluoromethoxy)phenyl]-2,3-dihydropyridazine-4-carboxamide;
N-[(trans)-4-Hydroxytetrahydrofuran-3-yl]-2-(1-methyl-1H-pyrazol-4-yl)-3-oxo-6-[4-(trifluoromethoxy)phenyl]-2,3-dihydropyridazine-4-carboxamide, Enantiomer 1;
N-[(trans)-4-Hydroxytetrahydrofuran-3-yl]-2-(1-methyl-1H-pyrazol-4-yl)-3-oxo-6-[4-(trifluoromethoxy)phenyl]-2,3-dihydropyridazine-4-carboxamide, Enantiomer 2;
6-[6-(Difluoromethyl)pyridin-3-yl]-N-[(2S)-1-hydroxypropan-2-yl]-2-(1-methyl-1H-pyrazol-4-yl)-3-oxo-2,3-dihydropyridazine-4-carboxamide;
N-(2-Hydroxy-2-methylpropyl)-2-(1-methyl-1H-pyrazol-4-yl)-3-oxo-6-[4-(trifluoromethyl)phenyl]-2,3-dihydropyridazine-4-carboxamide;
6-(4-Chlorophenyl)-N-[(2S)-1-hydroxypropan-2-yl]-3-oxo-2-(pyridin-3-yl)-2,3-dihydropyridazine-4-carboxamide;
6-(4-Chlorophenyl)-N-[(2R)-1-hydroxypropan-2-yl]-3-oxo-2-(pyridin-3-yl)-2,3-dihydropyridazine-4-carboxamide;
6-(4-Chlorophenyl)-N-(1,3-dihydroxypropan-2-yl)-3-oxo-2-(pyridin-3-yl)-2,3-dihydropyridazine-4-carboxamide;
6-(4-Chlorophenyl)-N-(2-hydroxy-2-methylpropyl)-3-oxo-2-(pyridin-3-yl)-2,3-dihydropyridazine-4-carboxamide;
(−)-6-(4-Chlorophenyl)-N-[(2S)-2,3-dihydroxypropyl]-3-oxo-2-(pyridin-3-yl)-2,3-dihydropyridazine-4-carboxamide;
(+)-6-(4-Chlorophenyl)-N-[(2R)-2,3-dihydroxypropyl]-3-oxo-2-(pyridin-3-yl)-2,3-dihydropyridazine-4-carboxamide;
(+)-6-(4-Chlorophenyl)-N-[(2S)-1-hydroxy-3-methoxypropan-2-yl]-3-oxo-2-(pyridin-3-yl)-2,3-dihydropyridazine-4-carboxamide;
(−)-6-(4-Chlorophenyl)-N-[(2R)-1-hydroxy-3-methoxypropan-2-yl]-3-oxo-2-(pyridin-3-yl)-2,3-dihydropyridazine-4-carboxamide;
6-(4-Chlorophenyl)-3-oxo-2-(pyridin-3-yl)-N-[(2RS)-1,1,1-trifluoro-3-hydroxypropan-2-yl]-2,3-dihydropyridazine-4-carboxamide;
6-(4-Chlorophenyl)-N-[(1RS)-1-cyclopropyl-2-hydroxyethyl]-3-oxo-2-(pyridin-3-yl)-2,3-dihydropyridazine-4-carboxamide;
6-(4-Chlorophenyl)-3-oxo-2-(pyridin-3-yl)-N-[(2RS)-3,3,3-trifluoro-2-hydroxypropyl]-2,3-dihydropyridazine-4-carboxamide;
6-(4-Chlorophenyl)-N-[(2RS)-3,3-difluoro-2-hydroxypropyl]-3-oxo-2-(pyridin-3-yl)-2,3-dihydropyridazine-4-carboxamide;
6-(4-Chlorophenyl)-2-(5-fluoropyridin-3-yl)-N-[(2S)-1-hydroxypropan-2-yl]-3-oxo-2,3-dihydropyridazine-4-carboxamide;
6-(4-Chlorophenyl)-2-(5-fluoropyridin-3-yl)-N-[(2R)-1-hydroxypropan-2-yl]-3-oxo-2,3-dihydropyridazine-4-carboxamide;
6-(4-Chlorophenyl)-2-(5-fluoropyridin-3-yl)-N-(2-hydroxy-2-methylpropyl)-3-oxo-2,3-dihydropyridazine-4-carboxamide;
6-(4-Chlorophenyl)-2-(5-fluoropyridin-3-yl)-N-[(2S)-1-hydroxy-3-methylbutan-2-yl]-3-oxo-2,3-dihydropyridazine-4-carboxamide;
6-(4-Chlorophenyl)-N-(1,3-dihydroxypropan-2-yl)-2-(5-fluoropyridin-3-yl)-3-oxo-2,3-dihydropyridazine-4-carboxamide;
6-(4-Chlorophenyl)-2-(5-fluoropyridin-3-yl)-N-[(2R)-1-hydroxy-3-methoxypropan-2-yl]-3-oxo-2,3-dihydropyridazine-4-carboxamide;
6-(4-Chlorophenyl)-2-(5-fluoropyridin-3-yl)-N-[(2S)-1-hydroxy-3-methoxypropan-2-yl]-3-oxo-2,3-dihydropyridazine-4-carboxamide;
(−)-6-(4-Chlorophenyl)-N-(3,3-difluoro-2-hydroxypropyl)-3-oxo-2-(pyridin-3-yl)-2,3-dihydropyridazine-4-carboxamide;
(+)-6-(4-Chlorophenyl)-N-(3,3-difluoro-2-hydroxypropyl)-3-oxo-2-(pyridin-3-yl)-2,3-dihydropyridazine-4-carboxamide;
(−)-6-(4-Chlorophenyl)-3-oxo-2-(pyridin-3-yl)-N-(3,3,3-trifluoro-2-hydroxypropyl)-2,3-dihydropyridazine-4-carboxamide;
(+)-6-(4-Chlorophenyl)-3-oxo-2-(pyridin-3-yl)-N-(3,3,3-trifluoro-2-hydroxypropyl)-2,3-dihydropyridazine-4-carboxamide;
(+)-6-(4-Chlorophenyl)-N-(1-cyclopropyl-2-hydroxyethyl)-3-oxo-2-(pyridin-3-yl)-2,3-dihydropyridazine-4-carboxamide;
(−)-6-(4-Chlorophenyl)-N-(1-cyclopropyl-2-hydroxyethyl)-3-oxo-2-(pyridin-3-yl)-2,3-dihydropyridazine-4-carboxamide;
(+)-6-(4-Chlorophenyl)-N-[(2R)-1-fluoro-3-hydroxypropan-2-yl]-3-oxo-2-(pyridin-3-yl)-2,3-dihydropyridazine-4-carboxamide;
N-[(1S)-1-Cyclopropyl-2-hydroxyethyl]-3-oxo-2-(pyridin-3-yl)-6-[4-(trifluoromethyl)phenyl]-2,3-dihydropyridazine-4-carboxamide;
(−)-6-(4-Chlorophenyl)-3-oxo-2-(pyridin-3-yl)-N-[(2S)-1,1,1-trifluoro-3-hydroxypropan-2-yl]-2,3-dihydropyridazine-4-carboxamide;
(+)-6-(4-Chlorophenyl)-3-oxo-2-(pyridin-3-yl)-N-[(2R)-1,1,1-trifluoro-3-hydroxypropan-2-yl]-2,3-dihydropyridazine-4-carboxamide;
6-(4-Chlorophenyl)-N-[(1RS)-1-cyclopropyl-2-hydroxyethyl]-2-(5-fluoropyridin-3-yl)-3-oxo-2,3-dihydropyridazine-4-carboxamide;
6-(4-Chlorophenyl)-N-[(2R)-2,3-dihydroxypropyl]-2-(5-fluoropyridin-3-yl)-3-oxo-2,3-dihydropyridazine-4-carboxamide;
6-(4-Chlorophenyl)-N-[(2S)-2,3-dihydroxypropyl]-2-(5-fluoropyridin-3-yl)-3-oxo-2,3-dihydropyridazine-4-carboxamide;
6-(4-Chlorophenyl)-2-(5-fluoropyridin-3-yl)-3-oxo-N-[(2R)-1,1,1-trifluoro-3-hydroxypropan-2-yl]-2,3-dihydropyridazine-4-carboxamide;
N-[(2RS)-3,3-Difluoro-2-hydroxypropyl]-3-oxo-2-(pyridin-3-yl)-6-[4-(trifluoromethyl)phenyl]-2,3-dihydropyridazine-4-carboxamide;
N-[(1S,2S)-2-Hydroxycyclopentyl]-3-oxo-2-(pyridin-3-yl)-6-[4-(trifluoromethyl)phenyl]-2,3-dihydropyridazine-4-carboxamide;
N-[(1R,2S)-2-Hydroxycyclopentyl]-3-oxo-2-(pyridin-3-yl)-6-[4-(trifluoromethyl)phenyl]-2,3-dihydropyridazine-4-carboxamide;
N-[(1R,2R)-2-Hydroxycyclopentyl]-3-oxo-2-(pyridin-3-yl)-6-[4-(trifluoromethyl)phenyl]-2,3-dihydropyridazine-4-carboxamide;
N-[(1S,2R)-2-Hydroxycyclopentyl]-3-oxo-2-(pyridin-3-yl)-6-[4-(trifluoromethyl)phenyl]-2,3-dihydropyridazine-4-carboxamide;
N-[(1S,2R)-2-Hydroxycyclohexyl]-3-oxo-2-(pyridin-3-yl)-6-[4-(trifluoromethyl)phenyl]-2,3-dihydropyridazine-4-carboxamide;
N-[(3RS,4RS)-4-Hydroxytetrahydrofuran-3-yl]-3-oxo-2-(pyridin-3-yl)-6-[4-(trifluoromethyl)-phenyl]-2,3-dihydropyridazine-4-carboxamide;
3-Oxo-2-(pyridin-3-yl)-N-[(2RS)-3,3,3-trifluoro-2-hydroxypropyl]-6-[4-(trifluoromethyl)phenyl]-2,3-dihydropyridazine-4-carboxamide;
N-[(1RS)-1-Cyclopropyl-2-hydroxyethyl]-3-oxo-2-(pyridin-3-yl)-6-[4-(trifluoromethyl)phenyl]-2,3-dihydropyridazine-4-carboxamide;
3-Oxo-2-(pyridin-3-yl)-N-[(2RS)-1,1,1-trifluoro-3-hydroxypropan-2-yl]-6-[4-(trifluoromethyl)phenyl]-2,3-dihydropyridazine-4-carboxamide;
N-[(2S)-1-Hydroxy-3-methoxypropan-2-yl]-3-oxo-2-(pyridin-3-yl)-6-[4-(trifluoromethyl)phenyl]-2,3-dihydropyridazine-4-carboxamide;
3-Oxo-2-(pyridin-3-yl)-N-[(2R)-1,1,1-trifluoro-3-hydroxypropan-2-yl]-6-[4-(trifluoromethyl)phenyl]-2,3-dihydropyridazine-4-carboxamide;
N-[(2R)-2,3-Dihydroxypropyl]-3-oxo-2-(pyridin-3-yl)-6-[4-(trifluoromethyl)phenyl]-2,3-dihydropyridazine-4-carboxamide;
N-[(2S)-2,3-Dihydroxypropyl]-3-oxo-2-(pyridin-3-yl)-6-[4-(trifluoromethyl)phenyl]-2,3-dihydropyridazine-4-carboxamide;
N-(2-Hydroxy-2-methylpropyl)-3-oxo-2-(pyridin-3-yl)-6-[4-(trifluoromethyl)phenyl]-2,3-dihydropyridazine-4-carboxamide;
N-(1,3-Dihydroxypropan-2-yl)-3-oxo-2-(pyridin-3-yl)-6-[4-(trifluoromethyl)phenyl]-2,3-dihydropyridazine-4-carboxamide;
3-Oxo-2-(pyridin-3-yl)-N-[(2S)-3,3,3-trifluoro-2-hydroxypropyl]-6-[4-(trifluoromethyl)phenyl]-2,3-dihydropyridazine-4-carboxamide;
N-[(2R)-1-Hydroxypropan-2-yl]-3-oxo-2-(pyridin-3-yl)-6-[4-(trifluoromethyl)phenyl]-2,3-dihydropyridazine-4-carboxamide;
N-[(2S)-1-Hydroxypropan-2-yl]-3-oxo-2-(pyridin-3-yl)-6-[4-(trifluoromethyl)phenyl]-2,3-dihydropyridazine-4-carboxamide;
6-(4-Chlorophenyl)-N-[(2S)-1-hydroxypropan-2-yl]-3-oxo-2-(pyrimidin-5-yl)-2,3-dihydropyridazine-4-carboxamide;
6-(4-Chlorophenyl)-3-oxo-2-(pyridin-3-yl)-N-(1,1,1-trifluoro-3-hydroxy-3-methylbutan-2-yl)-2,3-dihydropyridazine-4-carboxamide;
6-(4-Chlorophenyl)-3-oxo-2-(pyridin-3-yl)-N-(1,1,1-trifluoro-3-hydroxy-3-methylbutan-2-yl)-2,3-dihydropyridazine-4-carboxamide, Enantiomer 1;
6-(4-Chlorophenyl)-3-oxo-2-(pyridin-3-yl)-N-(1,1,1-trifluoro-3-hydroxy-3-methylbutan-2-yl)-2,3-dihydropyridazine-4-carboxamide, Enantiomer 2;
6-(4-Chlorophenyl)-2-(5-fluoropyridin-3-yl)-N-[(1S,2R)-2-hydroxycyclohexyl]-3-oxo-2,3-dihydropyridazine-4-carboxamide;
6-(4-Chlorophenyl)-N-[(1S,2R)-2-hydroxycyclohexyl]-3-oxo-2-(pyridin-3-yl)-2,3-dihydropyridazine-4-carboxamide;
N-[(1S,2R)-2-Hydroxycyclohexyl]-3-oxo-2-(pyridin-3-yl)-6-[4-(trifluoromethoxy)phenyl]-2,3-dihydropyridazine-4-carboxamide;
6-[4-(Difluoromethyl)phenyl]-3-oxo-2-(pyridin-3-yl)-N-[(2R)-1,1,1-trifluoro-3-hydroxypropan-2-yl]-2,3-dihydropyridazine-4-carboxamide;
6-(4-Chlorophenyl)-2-(5-fluoropyridin-3-yl)-3-oxo-N-[(2S)-3,3,3-trifluoro-2-hydroxypropyl]-2,3-dihydropyridazine-4-carboxamide;
6-(4-Chlorophenyl)-2-(5-fluoropyridin-3-yl)-N-[(1S,2R)-2-hydroxycyclopentyl]-3-oxo-2,3-dihydropyridazine-4-carboxamide;
6-(4-Chlorophenyl)-N-[(1S,2S)-2-hydroxycyclopentyl]-3-oxo-2-(pyridin-3-yl)-2,3-dihydropyridazine-4-carboxamide;
3-Oxo-2-(pyridin-3-yl)-N-[(2S)-3,3,3-trifluoro-2-hydroxypropyl]-6-[4-(trifluoromethoxy)phenyl]-2,3-dihydropyridazine-4-carboxamide;
N-[(1S,2S)-2-Hydroxycyclopentyl]-3-oxo-2-(pyridin-3-yl)-6-[4-(trifluoromethoxy)phenyl]-2,3-dihydropyridazine-4-carboxamide;
6-(4-Chlorophenyl)-N-[(cis)-2-hydroxycyclohexyl]-3-oxo-2-(pyridin-3-yl)-2,3-dihydropyridazine-4-carboxamide;
3-Oxo-2-(pyridin-3-yl)-N-[(2R)-1,1,1-trifluoro-3-hydroxypropan-2-yl]-6-[4-(trifluoromethoxy)-phenyl]-2,3-dihydropyridazine-4-carboxamide;
3-Oxo-2-(pyridin-3-yl)-N-[1,1,1-trifluoro-3-hydroxy-3-methylbutan-2-yl]-6-[4-(trifluoromethoxy)phenyl]-2,3-dihydropyridazine-4-carboxamide;
3-Oxo-2-(pyridin-3-yl)-N-[1,1,1-trifluoro-3-hydroxy-3-methylbutan-2-yl]-6-[4-(trifluoromethoxy)phenyl]-2,3-dihydropyridazine-4-carboxamide, Enantiomer 1;
3-Oxo-2-(pyridin-3-yl)-N-[1,1,1-trifluoro-3-hydroxy-3-methylbutan-2-yl]-6-[4-(trifluoromethoxy)phenyl]-2,3-dihydropyridazine-4-carboxamide, Enantiomer 2;
6-(4-Chlorophenyl)-N-[(1R,2S)-2-hydroxycyclopentyl]-3-oxo-2-(pyridin-3-yl)-2,3-dihydropyridazine-4-carboxamide;
6-[4-(Difluoromethyl)phenyl]-N-[(1S,2S)-2-hydroxycyclopentyl]-3-oxo-2-(pyridin-3-yl)-2,3-dihydropyridazine-4-carboxamide;
N-[(trans)-3,3-Difluoro-2-hydroxycyclohexyl]-3-oxo-2-(pyridin-3-yl)-6-[4-(trifluoromethoxy)-phenyl]-2,3-dihydropyridazine-4-carboxamide;
N-[(trans)-3,3-Difluoro-2-hydroxycyclohexyl]-3-oxo-2-(pyridin-3-yl)-6-[4-(trifluoromethoxy)-phenyl]-2,3-dihydropyridazine-4-carboxamide, Enantiomer 1;
N-[(trans)-3,3-Difluoro-2-hydroxycyclohexyl]-3-oxo-2-(pyridin-3-yl)-6-[4-(trifluoromethoxy)phenyl]-2,3-dihydropyridazine-4-carboxamide, Enantiomer 2;
6-(4-Chlorophenyl)-N-[(trans)-3,3-difluoro-2-hydroxycyclohexyl]-3-oxo-2-(pyridin-3-yl)-2,3-dihydropyridazine-4-carboxamide;
6-(4-Chlorophenyl)-N-[(trans)-3,3-difluoro-2-hydroxycyclohexyl]-3-oxo-2-(pyridin-3-yl)-2,3-dihydropyridazine-4-carboxamide, Enantiomer 1;
6-(4-Chlorophenyl)-N-[(trans)-3,3-difluoro-2-hydroxycyclohexyl]-3-oxo-2-(pyridin-3-yl)-2,3-dihydropyridazine-4-carboxamide, Enantiomer 2;
3-Oxo-2-(pyridin-3-yl)-N-[3,3,3-trifluoro-2-hydroxypropyl]-6-[4-(trifluoromethoxy)phenyl]-2,3-dihydropyridazine-4-carboxamide;
N-[(1S,2R)-2-Hydroxycyclopentyl]-3-oxo-2-(pyridin-3-yl)-6-[4-(trifluoromethoxy)phenyl]-2,3-dihydropyridazine-4-carboxamide;
6-(4-Chlorophenyl)-N-[(trans)-2-hydroxycyclopentyl]-3-oxo-2-(pyridin-3-yl)-2,3-dihydropyridazine-4-carboxamide;
3-Oxo-2-(pyridin-3-yl)-N-(1,1,1-trifluoro-3-hydroxypropan-2-yl)-6-[4-(trifluoromethoxy)phenyl]-2,3-dihydropyridazine-4-carboxamide;
N-[(1S)-1-Cyclopropyl-2-hydroxyethyl]-6-[4-(difluoromethyl)phenyl]-3-oxo-2-(pyridin-3-yl)-2,3-dihydropyridazine-4-carboxamide;
6-[4-(Difluoromethyl)phenyl]-N-[(2S)-1-hydroxypropan-2-yl]-3-oxo-2-(pyridin-3-yl)-2,3-dihydropyridazine-4-carboxamide;
N-[(2S)-1-Hydroxypropan-2-yl]-6-(4-methylphenyl)-3-oxo-2-(pyridin-3-yl)-2,3-dihydropyridazine-4-carboxamide;
N-[(2S)-1-Hydroxypropan-2-yl]-3-oxo-2-(pyridin-3-yl)-6-[4-(trifluoromethoxy)phenyl]-2,3-dihydropyridazine-4-carboxamide;
6-(4-Chlorophenyl)-2-(5-fluoropyridin-3-yl)-N-[(trans)-4-hydroxytetrahydrofuran-3-yl]-3-oxo-2,3-dihydropyridazine-4-carboxamide;
6-(4-Chlorophenyl)-2-(5-fluoropyridin-3-yl)-N-[(trans)-4-hydroxytetrahydrofuran-3-yl]-3-oxo-2,3-dihydropyridazine-4-carboxamide, Enantiomer 1;
6-(4-Chlorophenyl)-2-(5-fluoropyridin-3-yl)-N-[(trans)-4-hydroxytetrahydrofuran-3-yl]-3-oxo-2,3-dihydropyridazine-4-carboxamide, Enantiomer 2;
N-[(cis)-4-Hydroxytetrahydrofuran-3-yl]-3-oxo-2-(pyridin-3-yl)-6-[4-(trifluoromethoxy)phenyl]-2,3-dihydropyridazine-4-carboxamide;
N-[(cis)-4-Hydroxytetrahydrofuran-3-yl]-3-oxo-2-(pyridin-3-yl)-6-[4-(trifluoromethoxy)phenyl]-2,3-dihydropyridazine-4-carboxamide, Enantiomer 1;
N-[(cis)-4-Hydroxytetrahydrofuran-3-yl]-3-oxo-2-(pyridin-3-yl)-6-[4-(trifluoromethoxy)phenyl]-2,3-dihydropyridazine-4-carboxamide, Enantiomer 2;
N-[(trans)-4-Hydroxytetrahydrofuran-3-yl]-3-oxo-2-(pyridin-3-yl)-6-[4-(trifluoromethoxy)phenyl]-2,3-dihydropyridazine-4-carboxamide;
N-[(trans)-4-Hydroxytetrahydrofuran-3-yl]-3-oxo-2-(pyridin-3-yl)-6-[4-(trifluoromethoxy)phenyl]-2,3-dihydropyridazine-4-carboxamide, Enantiomer 1;
N-[(trans)-4-Hydroxytetrahydrofuran-3-yl]-3-oxo-2-(pyridin-3-yl)-6-[4-(trifluoromethoxy)phenyl]-2,3-dihydropyridazine-4-carboxamide, Enantiomer 2;
6-(4-Chloro-2-fluorophenyl)-N-[(2S)-1-hydroxypropan-2-yl]-3-oxo-2-(pyridin-3-yl)-2,3-dihydropyridazine-4-carboxamide;
3-(4-Chlorophenyl)-N-[(1S,2R)-2-hydroxycyclohexyl]-6-oxo-6H-1,4′-bipyridazine-5-carboxamide;
6-[6-(Difluoromethyl)pyridin-3-yl]-N-[(1S,2S)-2-hydroxycyclopentyl]-3-oxo-2-(pyridin-3-yl)-2,3-dihydropyridazine-4-carboxamide;
N-[(2S)-1-Hydroxypropan-2-yl]-3-oxo-2-(pyridin-3-yl)-6-[6-(trifluoromethyl)pyridin-3-yl]-2,3-dihydropyridazine-4-carboxamide;
3-(4-Chlorophenyl)-N-[(1S,2R)-2-hydroxycyclopentyl]-6-oxo-6H-1,4′-bipyridazine-5-carboxamide;
6-(4-Chlorophenyl)-N-[(trans)-4-hydroxytetrahydrofuran-3-yl]-3-oxo-2-(pyridin-3-yl)-2,3-dihydropyridazine-4-carboxamide;
6-(4-Chlorophenyl)-N-[(trans)-4-hydroxytetrahydrofuran-3-yl]-3-oxo-2-(pyridin-3-yl)-2,3-dihydropyridazine-4-carboxamide, Enantiomer 1;
6-(4-Chlorophenyl)-N-[(trans)-4-hydroxytetrahydrofuran-3-yl]-3-oxo-2-(pyridin-3-yl)-2,3-dihydropyridazine-4-carboxamide, Enantiomer 2;
6-(4-Chlorophenyl)-N-[(cis)-4-hydroxytetrahydrofuran-3-yl]-3-oxo-2-(pyridin-3-yl)-2,3-dihydropyridazine-4-carboxamide;
6-(4-Chlorophenyl)-N-[(cis)-4-hydroxytetrahydrofuran-3-yl]-3-oxo-2-(pyridin-3-yl)-2,3-dihydropyridazine-4-carboxamide, Enantiomer 1;
6-(4-Chlorophenyl)-N-[(cis)-4-hydroxytetrahydrofuran-3-yl]-3-oxo-2-(pyridin-3-yl)-2,3-dihydropyridazine-4-carboxamide, Enantiomer 2;
3-(4-Chlorophenyl)-N-[(1S,2S)-2-hydroxycyclopentyl]-6-oxo-6H-1,4′-bipyridazine-5-carboxamide;
1,5-Anhydro-2-({[6-(4-chlorophenyl)-3-oxo-2-(pyridin-3-yl)-2,3-dihydropyridazin-4-yl]carbonyl}amino)-2,4-dideoxy-D-erythro-pentitol;
1,5-Anhydro-2,4-dideoxy-2-[({3-oxo-2-(pyridin-3-yl)-6-[4-(trifluoromethoxy)phenyl]-2,3-dihydropyridazin-4-yl}carbonyl)amino]-D-erythro-pentitol;
3-(4-Chlorophenyl)-N-[(2S)-1-hydroxypropan-2-yl]-6-oxo-6H-1,4′-bipyridazine-5-carboxamide;
3-(4-Chlorophenyl)-N-(2-hydroxy-2-methylpropyl)-6-oxo-6H-1,4′-bipyridazine-5-carboxamide;
6-(4-Cyanophenyl)-N-[(2S)-1-hydroxypropan-2-yl]-3-oxo-2-(pyridin-3-yl)-2,3-dihydropyridazine-4-carboxamide;
6-[6-(Difluoromethyl)pyridin-3-yl]-N-[(2S)-1-hydroxypropan-2-yl]-3-oxo-2-(pyridin-3-yl)-2,3-dihydropyridazine-4-carboxamide;
3-(4-Chlorophenyl)-N-[(cis)-4-hydroxytetrahydrofuran-3-yl]-6-oxo-6H-1,4′-bipyridazine-5-carboxamide;
6-[6-(Difluoromethyl)pyridin-3-yl]-N-[(cis)-4-hydroxytetrahydrofuran-3-yl]-3-oxo-2-(pyridin-3-yl)-2,3-dihydropyridazine-4-carboxamide;
6-[6-(Difluoromethyl)pyridin-3-yl]-N-[(cis)-4-hydroxytetrahydrofuran-3-yl]-3-oxo-2-(pyridin-3-yl)-2,3-dihydropyridazine-4-carboxamide, Enantiomer 1;
6-[6-(Difluoromethyl)pyridin-3-yl]-N-[(cis)-4-hydroxytetrahydrofuran-3-yl]-3-oxo-2-(pyridin-3-yl)-2,3-dihydropyridazine-4-carboxamide, Enantiomer 2;
6-(4-Chlorophenyl)-3-oxo-2-(pyrimidin-5-yl)-N-(1,1,1-trifluoro-3-hydroxy-3-methylbutan-2-yl)-2,3-dihydropyridazine-4-carboxamide;
6-(4-Chlorophenyl)-3-oxo-2-(pyrimidin-5-yl)-N-(1,1,1-trifluoro-3-hydroxy-3-methylbutan-2-yl)-2,3-dihydropyridazine-4-carboxamide, Enantiomer 1;
6-(4-Chlorophenyl)-3-oxo-2-(pyrimidin-5-yl)-N-(1,1,1-trifluoro-3-hydroxy-3-methylbutan-2-yl)-2,3-dihydropyridazine-4-carboxamide, Enantiomer 2;
N-[(1-Hydroxycyclopropyl)methyl]-3-oxo-2-(pyridin-3-yl)-6-[4-(trifluoromethyl)phenyl]-2,3-dihydropyridazine-4-carboxamide;
N-[(1-Hydroxycyclobutyl)methyl]-3-oxo-2-(pyridin-3-yl)-6-[4-(trifluoromethyl)phenyl]-2,3-dihydropyridazine-4-carboxamide;
(+)-6-(4-Chlorophenyl)-N-[(2S)-1-hydroxypropan-2-yl]-3-oxo-2-(1H-pyrazol-4-yl)-2,3-dihydropyridazine-4-carboxamide;
6-[4-(Dimethylamino)phenyl]-N-[(2S)-1-hydroxypropan-2-yl]-3-oxo-2-(pyridin-3-yl)-2,3-dihydropyridazine-4-carboxamide;
6-(4-Chlorophenyl)-N-[(2S)-1-hydroxypropan-2-yl]-2-(1-methyl-1H-pyrazol-3-yl)-3-oxo-2,3-dihydropyridazine-4-carboxamide;
6-(4-Chlorophenyl)-N-[(2S)-1-hydroxypropan-2-yl]-2-(3-methyl-1H-pyrazol-5-yl)-3-oxo-2,3-dihydropyridazine-4-carboxamide;
(+)-6-(4-Chlorophenyl)-N-[(2S)-1-hydroxypropan-2-yl]-3-oxo-2-(1,2-thiazol-4-yl)-2,3-dihydropyridazine-4-carboxamide;
N-[(1-Hydroxycyclobutyl)methyl]-3-oxo-2-(1,2-thiazol-4-yl)-6-[4-(trifluoromethyl)phenyl]-2,3-dihydropyridazine-4-carboxamide;
N-[(2S)-3-Hydroxy-3-methylbutan-2-yl]-3-oxo-2-(1,2-thiazol-4-yl)-6-[4-(trifluoromethyl)phenyl]-2,3-dihydropyridazine-4-carboxamide;
(+)-3-Oxo-2-(pyridin-3-yl)-N-(1,1,1-trifluoro-3-hydroxy-3-methylbutan-2-yl)-6-[4-(trifluoromethyl)-phenyl]-2,3-dihydropyridazine-4-carboxamide, enantiomer 2;
6-(4-Chlorophenyl)-3-oxo-2-(1,2-thiazol-4-yl)-N-[(2R)-1,1,1-trifluoro-3-hydroxypropan-2-yl]-2,3-dihydropyridazine-4-carboxamide;
6-(4-Chlorophenyl)-3-oxo-2-(1,2-thiazol-4-yl)-N-[(2S)-3,3,3-trifluoro-2-hydroxypropyl]-2,3-dihydropyridazine-4-carboxamide;
6-(4-Chlorophenyl)-N-[(1-hydroxycyclobutyl)methyl]-3-oxo-2-(1,2-thiazol-4-yl)-2,3-dihydropyridazine-4-carboxamide;
6-(4-Chlorophenyl)-N-[(2R)-1-fluoro-3-hydroxypropan-2-yl]-3-oxo-2-(1,2-thiazol-4-yl)-2,3-dihydropyridazine-4-carboxamide;
N-[(2R)-3-Hydroxy-3-methylbutan-2-yl]-2-(1-methyl-1H-pyrazol-4-yl)-3-oxo-6-[4-(trifluoromethyl)-phenyl]-2,3-dihydropyridazine-4-carboxamide;
N-[(2R)-1-Fluoro-3-hydroxypropan-2-yl]-3-oxo-2-(1,2-thiazol-4-yl)-6-[4-(trifluoromethyl)-phenyl]-2,3-dihydropyridazine-4-carboxamide;
N-[(1S,2R)-2-Hydroxycyclopentyl]-2-(1-methyl-1H-pyrazol-4-yl)-3-oxo-6-[4-(trifluoromethyl)phenyl]-2,3-dihydropyridazine-4-carboxamide;
N-[(2S)-1-Hydroxypropan-2-yl]-3-oxo-2-(1,2-thiazol-4-yl)-6-[4-(trifluoromethyl)phenyl]-2,3-dihydropyridazine-4-carboxamide;
N-[(2S)-3-Hydroxy-3-methylbutan-2-yl]-2-(1-methyl-1H-pyrazol-4-yl)-3-oxo-6-[4-(trifluoromethyl)phenyl]-2,3-dihydropyridazine-4-carboxamide;
N-[(1S)-1-Cyclopropyl-2-hydroxyethyl]-3-oxo-2-(1,2-thiazol-4-yl)-6-[4-(trifluoromethyl)-phenyl]-2,3-dihydropyridazine-4-carboxamide;
N-(2-Hydroxy-2-methylpropyl)-3-oxo-2-(1,2-thiazol-4-yl)-6-[4-(trifluoromethyl)phenyl]-2,3-dihydropyridazine-4-carboxamide;
6-(4-Chlorophenyl)-N-[(2S)-3-hydroxy-3-methylbutan-2-yl]-3-oxo-2-(1,2-thiazol-4-yl)-2,3-dihydropyridazine-4-carboxamide;
N-[(1S,2S)-2-Hydroxycyclopentyl]-2-(1-methyl-1H-pyrazol-4-yl)-3-oxo-6-[4-(trifluoromethyl)-phenyl]-2,3-dihydropyridazine-4-carboxamide;
N-[(2R)-1-Fluoro-3-hydroxypropan-2-yl]-2-(1-methyl-1H-pyrazol-4-yl)-3-oxo-6-[4-(trifluoromethyl)phenyl]-2,3-dihydropyridazine-4-carboxamide;
3-Oxo-2-(1H-pyrazol-4-yl)-N-[(2R)-1,1,1-trifluoro-3-hydroxypropan-2-yl]-6-[4-(trifluoromethyl)-phenyl]-2,3-dihydropyridazine-4-carboxamide;
3-Oxo-2-(pyridin-3-yl)-N-(1,1,1-trifluoro-3-hydroxy-3-methylbutan-2-yl)-6-[4-(trifluoromethyl)-phenyl]-2,3-dihydropyridazine-4-carboxamide;
2-[1-(Difluoromethyl)-1H-pyrazol-4-yl]-3-oxo-N-[(2R)-1,1,1-trifluoro-3-hydroxypropan-2-yl]-6-[4-(trifluoromethyl)phenyl]-2,3-dihydropyridazine-4-carboxamide;
N-cis-4-Hydroxytetrahydrofuran-3-yl-2-(1-methyl-1H-pyrazol-4-yl)-3-oxo-6-[4-(trifluoromethyl)-phenyl]-2,3-dihydropyridazine-4-carboxamide;
3-Oxo-2-(1H-pyrazol-4-yl)-N-[(2S)-3,3,3-trifluoro-2-hydroxypropyl]-6-[4-(trifluoromethyl)phenyl]-2,3-dihydropyridazine-4-carboxamide;
(+)-N-cis-2-Hydroxycyclobutyl-3-oxo-2-(pyridin-3-yl)-6-[4-(trifluoromethyl)phenyl]-2,3-dihydropyridazine-4-carboxamide, enantiomer 1;
(+)-N-cis-4-Hydroxytetrahydrofuran-3-yl-2-(1-methyl-1H-pyrazol-4-yl)-3-oxo-6-[4-(trifluoromethyl)phenyl]-2,3-dihydropyridazine-4-carboxamide, enantiomer 2;
N-[(2S)-3,3-Difluoro-2-hydroxypropyl]-2-(1-methyl-1H-pyrazol-4-yl)-3-oxo-6-[4-(trifluoromethyl)-phenyl]-2,3-dihydropyridazine-4-carboxamide;
6-(4-Chlorophenyl)-2-(5-fluoro-2-thienyl)-N-[(2S)-1-hydroxypropan-2-yl]-3-oxo-2,3-dihydropyridazine-4-carboxamide;
N-[(2R)-1-Fluoro-3-hydroxypropan-2-yl]-3-oxo-2-(pyridin-3-yl)-6-[4-(trifluoromethyl)phenyl]-2,3-dihydropyridazine-4-carboxamide;
6-(4-Chlorophenyl)-N-(2-hydroxy-2-methylpropyl)-3-oxo-2-(1,2-thiazol-4-yl)-2,3-dihydropyridazine-4-carboxamide;
2-[1-(Difluoromethyl)-1H-pyrazol-4-yl]-3-oxo-N-[(2S)-3,3,3-trifluoro-2-hydroxypropyl]-6-[4-(trifluoromethyl)phenyl]-2,3-dihydropyridazine-4-carboxamide;
N-cis-2-Hydroxycyclobutyl-3-oxo-2-(pyridin-3-yl)-6-[4-(trifluoromethyl)phenyl]-2,3-dihydropyridazine-4-carboxamide;
6-(4-Chlorophenyl)-2-(5-methyl-3-thienyl)-3-oxo-N-[(2S)-3,3,3-trifluoro-2-hydroxypropyl]-2,3-dihydropyridazine-4-carboxamide;
N-[(2S)-3-Hydroxy-3-methylbutan-2-yl]-3-oxo-2-(pyridin-3-yl)-6-[4-(trifluoromethyl)phenyl]-2,3-dihydropyridazine-4-carboxamide;
6-(4-Chlorophenyl)-N-[(1S)-1-cyano-2-hydroxyethyl]-2-(1-methyl-1H-pyrazol-4-yl)-3-oxo-2,3-dihydropyridazine-4-carboxamide;
2-[1-(Difluoromethyl)-1H-pyrazol-4-yl]-N-[(2S)-3-hydroxy-3-methylbutan-2-yl]-3-oxo-6-[4-(trifluoromethyl)phenyl]-2,3-dihydropyridazine-4-carboxamide;
(+)-N-cis-4-Hydroxytetrahydrofuran-3-yl-3-oxo-2-(pyridin-3-yl)-6-[4-(trifluoromethyl)phenyl]-2,3-dihydropyridazine-4-carboxamide, enantiomer 1;
(−)-N-(3,3-Difluoro-2-hydroxypropyl)-3-oxo-2-(pyridin-3-yl)-6-[4-(trifluoromethyl)phenyl]-2,3-dihydropyridazine-4-carboxamide, enantiomer 2;
(−)-N-cis-4-Hydroxytetrahydrofuran-3-yl-2-(1-methyl-1H-pyrazol-4-yl)-3-oxo-6-[4-(trifluoromethyl)phenyl]-2,3-dihydropyridazine-4-carboxamide, enantiomer 1;
6-(4-Chlorophenyl)-2-(5-chloro-3-thienyl)-3-oxo-N-[(2S)-3,3,3-trifluoro-2-hydroxypropyl]-2,3-dihydropyridazine-4-carboxamide;
N-[(2S)-3-Hydroxy-3-methylbutan-2-yl]-3-oxo-2-(1,2-thiazol-4-yl)-6-[6-(trifluoromethyl)pyridin-3-yl]-2,3-dihydropyridazine-4-carboxamide;
N-[(2R)-3-Hydroxy-3-methylbutan-2-yl]-3-oxo-2-(pyridin-3-yl)-6-[4-(trifluoromethyl)phenyl]-2,3-dihydropyridazine-4-carboxamide;
6-(4-Chlorophenyl)-2-(1,2-oxazol-4-yl)-3-oxo-N-[(2S)-3,3,3-trifluoro-2-hydroxypropyl]-2,3-dihydropyridazine-4-carboxamide;
6-(4-Chlorophenyl)-2-[1-(difluoromethyl)-1H-pyrazol-4-yl]-3-oxo-N-[(2R)-1,1,1-trifluoro-3-hydroxypropan-2-yl]-2,3-dihydropyridazine-4-carboxamide;
6-(4-Chlorophenyl)-3-oxo-2-(pyrimidin-5-yl)-N-[(2S)-3,3,3-trifluoro-2-hydroxypropyl]-2,3-dihydropyridazine-4-carboxamide;
N-[(1S)-1-Cyclopropyl-2-hydroxyethyl]-2-(1-methyl-1H-pyrazol-4-yl)-3-oxo-6-[4-(trifluoromethyl)phenyl]-2,3-dihydropyridazine-4-carboxamide;
N-[(1R,2S)-2-Hydroxycyclopentyl]-2-(1-methyl-1H-pyrazol-4-yl)-3-oxo-6-[4-(trifluoromethyl)phenyl]-2,3-dihydropyridazine-4-carboxamide;
N-(2-Hydroxy-2-methylpropyl)-3-oxo-2-(1,2-thiazol-4-yl)-6-[6-(trifluoromethyl)pyridin-3-yl]-2,3-dihydropyridazine-4-carboxamide;
(−)-3-Oxo-2-(pyridin-3-yl)-N-(1,1,1-trifluoro-3-hydroxy-3-methylbutan-2-yl)-6-[4-(trifluoromethyl)phenyl]-2,3-dihydropyridazine-4-carboxamide, enantiomer 1;
6-(4-Chlorophenyl)-N-[(2S)-1-hydroxypropan-2-yl]-2-(5-methyl-3-thienyl)-3-oxo-2,3-dihydropyridazine-4-carboxamide;
6-(4-Chlorophenyl)-2-(5-chloro-3-thienyl)-N-[(2S)-1-hydroxypropan-2-yl]-3-oxo-2,3-dihydropyridazine-4-carboxamide;
N-[(2S)-1-Hydroxypropan-2-yl]-3-oxo-2-(1H-pyrazol-4-yl)-6-[4-(trifluoromethyl)phenyl]-2,3-dihydropyridazine-4-carboxamide;
N-[(2S)-3-Fluoro-2-hydroxypropyl]-2-(1-methyl-1H-pyrazol-4-yl)-3-oxo-6-[4-(trifluoromethyl)phenyl]-2,3-dihydropyridazine-4-carboxamide;
6-(4-Chlorophenyl)-N-(2-hydroxy-2-methylpropyl)-2-(5-methyl-3-thienyl)-3-oxo-2,3-dihydropyridazine-4-carboxamide;
(+)-N-(3,3-Difluoro-2-hydroxypropyl)-3-oxo-2-(pyridin-3-yl)-6-[4-(trifluoromethyl)phenyl]-2,3-dihydropyridazine-4-carboxamide, enantiomer 1;
N-[(1R,2R)-2-Hydroxycyclopentyl]-2-(1-methyl-1H-pyrazol-4-yl)-3-oxo-6-[4-(trifluoromethyl)phenyl]-2,3-dihydropyridazine-4-carboxamide;
(+)-6-(4-Chlorophenyl)-N-cis-2-hydroxycyclobutyl-3-oxo-2-(pyridin-3-yl)-2,3-dihydropyridazine-4-carboxamide, enantiomer 1;
6-(4-Chlorophenyl)-2-(5-chloro-3-thienyl)-N-(2-hydroxy-2-methylpropyl)-3-oxo-2,3-dihydropyridazine-4-carboxamide;
N-[(2S)-1-Hydroxypropan-2-yl]-3-oxo-2-(1,2-thiazol-4-yl)-6-[6-(trifluoromethyl)pyridin-3-yl]-2,3-dihydropyridazine-4-carboxamide;
(−)-N-cis-4-Hydroxytetrahydrofuran-3-yl-3-oxo-2-(pyridin-3-yl)-6-[4-(trifluoromethyl)phenyl]-2,3-dihydropyridazine-4-carboxamide, enantiomer 2;
6-(4-Chlorophenyl)-N-[(2S)-1-hydroxypropan-2-yl]-2-(1,2-oxazol-4-yl)-3-oxo-2,3-dihydropyridazine-4-carboxamide;
6-(4-Chlorophenyl)-N-[(1S)-1-cyano-2-hydroxyethyl]-3-oxo-2-(pyridin-3-yl)-2,3-dihydropyridazine-4-carboxamide;
N-[(2S)-3-Fluoro-2-hydroxypropyl]-3-oxo-2-(pyridin-3-yl)-6-[4-(trifluoromethyl)phenyl]-2,3-dihydropyridazine-4-carboxamide;
6-(4-Chlorophenyl)-2-[1-(difluoromethyl)-1H-pyrazol-4-yl]-3-oxo-N-[(2S)-3,3,3-trifluoro-2-hydroxypropyl]-2,3-dihydropyridazine-4-carboxamide;
N-[(2R)-1-Hydroxy-3-methoxypropan-2-yl]-3-oxo-2-(pyridin-3-yl)-6-[4-(trifluoromethyl)phenyl]-2,3-dihydropyridazine-4-carboxamide;
6-(4-Chlorophenyl)-N-cis-2-hydroxycyclobutyl-3-oxo-2-(pyridin-3-yl)-2,3-dihydropyridazine-4-carboxamide;
6-(4-Chlorophenyl)-N-[(1S)-1-cyclopropyl-2-hydroxyethyl]-2-(5-fluoropyridin-3-yl)-3-oxo-2,3-dihydropyridazine-4-carboxamide;
N-[(2S)-3-Hydroxy-3-methylbutan-2-yl]-3-oxo-2-(1H-pyrazol-4-yl)-6-[4-(trifluoromethyl)phenyl]-2,3-dihydropyridazine-4-carboxamide;
2-[1-(Difluoromethyl)-1H-pyrazol-4-yl]-N-[(2S)-1-hydroxypropan-2-yl]-3-oxo-6-[4-(trifluoromethyl)phenyl]-2,3-dihydropyridazine-4-carboxamide;
2-[1-(Difluoromethyl)-1H-pyrazol-4-yl]-N-(2-hydroxy-2-methylpropyl)-3-oxo-6-[4-(trifluoromethyl)phenyl]-2,3-dihydropyridazine-4-carboxamide;
6-(4-Chlorophenyl)-N-[(1-hydroxycyclopropyl)methyl]-3-oxo-2-(pyridin-3-yl)-2,3-dihydropyridazine-4-carboxamide;
6-(4-Chlorophenyl)-2-[1-(difluoromethyl)-1H-pyrazol-4-yl]-N-[(2S)-3-hydroxy-3-methylbutan-2-yl]-3-oxo-2,3-dihydropyridazine-4-carboxamide;
6-(4-Chlorophenyl)-N-[(2R)-3-hydroxy-3-methylbutan-2-yl]-3-oxo-2-(pyrimidin-5-yl)-2,3-dihydropyridazine-4-carboxamide;
N-(2-Hydroxy-2-methylpropyl)-3-oxo-2-(1H-pyrazol-4-yl)-6-[4-(trifluoromethyl)phenyl]-2,3-dihydropyridazine-4-carboxamide;
Methyl N-{[6-(4-chlorophenyl)-2-(1-methyl-1H-pyrazol-4-yl)-3-oxo-2,3-dihydropyridazin-4-yl]carbonyl}-D-serinate;
(−)-N-cis-2-Hydroxycyclobutyl-3-oxo-2-(pyridin-3-yl)-6-[4-(trifluoromethyl)phenyl]-2,3-dihydropyridazine-4-carboxamide, enantiomer 2;
6-(4-Chlorophenyl)-N-(2-hydroxy-2-methylpropyl)-2-(1,2-oxazol-4-yl)-3-oxo-2,3-dihydropyridazine-4-carboxamide;
6-(4-Chlorophenyl)-N-[(2S)-3-fluoro-2-hydroxypropyl]-2-(1-methyl-1H-pyrazol-4-yl)-3-oxo-2,3-dihydropyridazine-4-carboxamide;
6-(4-Chlorophenyl)-3-oxo-2-(pyrimidin-5-yl)-N-[(2R)-1,1,1-trifluoro-3-hydroxypropan-2-yl]-2,3-dihydropyridazine-4-carboxamide;
6-(4-Chlorophenyl)-2-[1-(difluoromethyl)-1H-pyrazol-4-yl]-N-[(2S)-1-hydroxypropan-2-yl]-3-oxo-2,3-dihydropyridazine-4-carboxamide;
6-(4-Chlorophenyl)-N-[(1S)-1-cyclopropyl-2-hydroxyethyl]-3-oxo-2-(pyrimidin-5-yl)-2,3-dihydropyridazine-4-carboxamide;
Methyl N-{[6-(4-chlorophenyl)-3-oxo-2-(pyridin-3-yl)-2,3-dihydropyridazin-4-yl]carbonyl}-D-serinate;
6-[4-(Fluoromethyl)phenyl]-2-(1-methyl-1H-pyrazol-4-yl)-3-oxo-N-[(2R)-1,1,1-trifluoro-3-hydroxypropan-2-yl]-2,3-dihydropyridazine-4-carboxamide;
6-(4-Chlorophenyl)-N-[(2S)-3-hydroxy-3-methylbutan-2-yl]-3-oxo-2-(1H-pyrazol-4-yl)-2,3-dihydropyridazine-4-carboxamide;
6-(4-Chlorophenyl)-N-(2-hydroxy-2-methylpropyl)-3-oxo-2-(pyrimidin-5-yl)-2,3-dihydropyridazine-4-carboxamide;
6-(4-Chlorophenyl)-2-[1-(difluoromethyl)-1H-pyrazol-4-yl]-N-(2-hydroxy-2-methylpropyl)-3-oxo-2,3-dihydropyridazine-4-carboxamide;
6-(4-Chlorophenyl)-N-[(2R)-1-hydroxypropan-2-yl]-3-oxo-2-(pyrimidin-5-yl)-2,3-dihydropyridazine-4-carboxamide;
6-[4-(Fluoromethyl)phenyl]-3-oxo-2-(pyridin-3-yl)-N-[(2S)-3,3,3-trifluoro-2-hydroxypropyl]-2,3-dihydropyridazine-4-carboxamide;
(−)-6-(4-Chlorophenyl)-N-cis-2-hydroxycyclobutyl-3-oxo-2-(pyridin-3-yl)-2,3-dihydropyridazine-4-carboxamide, enantiomer 2;
6-[4-(Fluoromethyl)phenyl]-3-oxo-2-(pyridin-3-yl)-N-[(2R)-1,1,1-trifluoro-3-hydroxypropan-2-yl]-2,3-dihydropyridazine-4-carboxamide;
6-[4-(Fluoromethyl)phenyl]-2-(1-methyl-1H-pyrazol-4-yl)-3-oxo-N-[(2S)-3,3,3-trifluoro-2-hydroxypropyl]-2,3-dihydropyridazine-4-carboxamide;
6-[4-(Fluoromethyl)phenyl]-N-[(2S)-3-hydroxy-3-methylbutan-2-yl]-2-(1-methyl-1H-pyrazol-4-yl)-3-oxo-2,3-dihydropyridazine-4-carboxamide;
6-[4-(Fluoromethyl)phenyl]-N-[(2S)-1-hydroxypropan-2-yl]-3-oxo-2-(pyridin-3-yl)-2,3-dihydropyridazine-4-carboxamide;
6-[4-(Fluoromethyl)phenyl]-N-(2-hydroxy-2-methylpropyl)-3-oxo-2-(pyridin-3-yl)-2,3-dihydropyridazine-4-carboxamide;
6-[4-(Fluoromethyl)phenyl]-N-[(2S)-1-hydroxypropan-2-yl]-2-(1-methyl-1H-pyrazol-4-yl)-3-oxo-2,3-dihydropyridazine-4-carboxamide;
6-[4-(Fluoromethyl)phenyl]-N-[(2S)-3-hydroxy-3-methylbutan-2-yl]-3-oxo-2-(pyridin-3-yl)-2,3-dihydropyridazine-4-carboxamide;
N-[(2R)-1-Amino-3-hydroxy-1-oxopropan-2-yl]-6-(4-chlorophenyl)-2-(1-methyl-1H-pyrazol-4-yl)-3-oxo-2,3-dihydropyridazine-4-carboxamide;
N-cis-4-Hydroxytetrahydrothiophen-3-yl-3-oxo-2-(pyridin-3-yl)-6-[4-(trifluoromethyl)-phenyl]-2,3-dihydropyridazine-4-carboxamide;
(−)-N-cis-4-hydroxytetrahydrothiophen-3-yl-3-oxo-2-(pyridin-3-yl)-6-[4-(trifluoromethyl)-phenyl]-2,3-dihydropyridazine-4-carboxamide, enantiomer 2;
N-(2-Hydroxy-2-methylpropyl)-3-oxo-2-(pyridin-3-yl)-6-[4-(trifluoromethoxy)phenyl]-2,3-dihydropyridazine-4-carboxamide;
3-Oxo-2-(pyridin-3-yl)-N-[(2R)-3,3,3-trifluoro-2-hydroxypropyl]-6-[4-(trifluoromethoxy)-phenyl]-2,3-dihydropyridazine-4-carboxamide;
N-[(2R)-3-Hydroxy-3-methylbutan-2-yl]-3-oxo-2-(pyridin-3-yl)-6-[4-(trifluoromethoxy)-phenyl]-2,3-dihydropyridazine-4-carboxamide;
N-[(2S)-3,3-Difluoro-2-hydroxypropyl]-3-oxo-2-(pyridin-3-yl)-6-[4-(trifluoromethoxy)-phenyl]-2,3-dihydropyridazine-4-carboxamide;
N-[(2S)-3-Fluoro-2-hydroxypropyl]-3-oxo-2-(pyridin-3-yl)-6-[4-(trifluoromethoxy)phenyl]-2,3-dihydropyridazine-4-carboxamide;
6-(4-Chlorophenyl)-3-oxo-2-(pyridin-3-yl)-N-(4,4,4-trifluoro-3-hydroxybutan-2-yl)-2,3-dihydropyridazine-4-carboxamide Isomer 1;
6-(4-Chlorophenyl)-3-oxo-2-(pyridin-3-yl)-N-(4,4,4-trifluoro-3-hydroxybutan-2-yl)-2,3-dihydropyridazine-4-carboxamide Isomer 2;
6-(4-Chlorophenyl)-3-oxo-2-(pyridin-3-yl)-N-(4,4,4-trifluoro-3-hydroxybutan-2-yl)-2,3-dihydropyridazine-4-carboxamide Isomer 3;
6-(4-Chlorophenyl)-3-oxo-2-(pyridin-3-yl)-N-(4,4,4-trifluoro-3-hydroxybutan-2-yl)-2,3-dihydropyridazine-4-carboxamide Isomer 4;
6-(4-Chlorophenyl)-2-(5-fluoropyridin-3-yl)-N-[(cis)-4-hydroxytetrahydrofuran-3-yl]-3-oxo-2,3-dihydropyridazine-4-carboxamide;
6-(4-Chlorophenyl)-N-[(2R)-3-hydroxy-3-methylbutan-2-yl]-3-oxo-2-(pyridin-3-yl)-2,3-dihydropyridazine-4-carboxamide;
6-(4-Chlorophenyl)-2-(5-chloropyridin-3-yl)-N-[(2S)-1-hydroxypropan-2-yl]-3-oxo-2,3-dihydropyridazine-4-carboxamide;
6-(4-Chlorophenyl)-N-[(2S)-1-hydroxypropan-2-yl]-2-(5-methylpyridin-3-yl)-3-oxo-2,3-dihydropyridazine-4-carboxamide;
6-[4-(Difluoromethoxy)phenyl]-2-(1-methyl-1H-pyrazol-4-yl)-3-oxo-N-(1,1,1-trifluoro-3-hydroxy-3-methylbutan-2-yl)-2,3-dihydropyridazine-4-carboxamide;
6-[4-(Difluoromethoxy)phenyl]-2-(1-methyl-1H-pyrazol-4-yl)-3-oxo-N-(1,1,1-trifluoro-3-hydroxy-3-methylbutan-2-yl)-2,3-dihydropyridazine-4-carboxamide Isomer 1;
6-[4-(Difluoromethoxy)phenyl]-2-(1-methyl-1H-pyrazol-4-yl)-3-oxo-N-(1,1,1-trifluoro-3-hydroxy-3-methylbutan-2-yl)-2,3-dihydropyridazine-4-carboxamide Isomer 2;
6-[4-(Difluoromethoxy)phenyl]-N-[(2S)-1-hydroxypropan-2-yl]-2-(1-methyl-1H-pyrazol-4-yl)-3-oxo-2,3-dihydropyridazine-4-carboxamide;
6-[4-(Difluoromethoxy)phenyl]-N-[(2S)-3-hydroxy-3-methylbutan-2-yl]-2-(1-methyl-1H-pyrazol-4-yl)-3-oxo-2,3-dihydropyridazine-4-carboxamide;
6-[4-(Dimethylamino)phenyl]-N-[(2S)-3-hydroxy-3-methylbutan-2-yl]-2-(1-methyl-1H-pyrazol-4-yl)-3-oxo-2,3-dihydropyridazine-4-carboxamide;
6-[4-(Dimethylamino)phenyl]-N-[(2S)-1-hydroxypropan-2-yl]-2-(1-methyl-1H-pyrazol-4-yl)-3-oxo-2,3-dihydropyridazine-4-carboxamide;
N-[(2S)-3-Hydroxy-3-methylbutan-2-yl]-3-oxo-2-(1,2-thiazol-4-yl)-6-[4-(trifluoromethoxy)-phenyl]-2,3-dihydropyridazine-4-carboxamide;
6-[4-(Difluoromethyl)phenyl]-3-oxo-2-(pyridin-3-yl)-N-[(2S)-3,3,3-trifluoro-2-hydroxypropyl]-2,3-dihydropyridazine-4-carboxamide;
6-[4-(Difluoromethoxy)phenyl]-3-oxo-2-(pyridin-3-yl)-N-[(2S)-3,3,3-trifluoro-2-hydroxypropyl]-2,3-dihydropyridazine-4-carboxamide;
6-[4-(Difluoromethyl)phenyl]-N-[(cis)-4-hydroxytetrahydrofuran-3-yl]-2-(1-methyl-1H-pyrazol-4-yl)-3-oxo-2,3-dihydropyridazine-4-carboxamide Isomer 1;
6-(4-Chlorophenyl)-N-[(2S)-3-hydroxy-3-methylbutan-2-yl]-2-(1-methyl-1H-pyrazol-4-yl)-3-oxo-2,3-dihydropyridazine-4-carboxamide;
6-(4-Chlorophenyl)-N-[(cis)-2-hydroxy-2-methylcyclopentyl]-2-(1-methyl-1H-pyrazol-4-yl)-3-oxo-2,3-dihydropyridazine-4-carboxamide;
6-[4-(Difluoromethyl)phenyl]-N-[(cis)-4-hydroxytetrahydrofuran-3-yl]-3-oxo-2-(pyridin-3-yl)-2,3-dihydropyridazine-4-carboxamide Isomer 1;
6-[4-(Difluoromethyl)phenyl]-N-[(2S)-3-hydroxy-3-methylbutan-2-yl]-3-oxo-2-(pyridin-3-yl)-2,3-dihydropyridazine-4-carboxamide;
6-[4-(Difluoromethyl)phenyl]-N-[(cis)-4-hydroxytetrahydrofuran-3-yl]-2-(1-methyl-1H-pyrazol-4-yl)-3-oxo-2,3-dihydropyridazine-4-carboxamide Isomer 2;
3-(4-Chlorophenyl)-6-oxo-N-[(2S)-3,3,3-trifluoro-2-hydroxypropyl]-6H-1,4′-bipyridazine-5-carboxamide;
6-[4-(Difluoromethyl)phenyl]-N-(2-hydroxy-2-methylpropyl)-3-oxo-2-(pyridin-3-yl)-2,3-dihydropyridazine-4-carboxamide;
6-[4-(Difluoromethyl)phenyl]-N-[(cis)-4-hydroxytetrahydrofuran-3-yl]-3-oxo-2-(pyridin-3-yl)-2,3-dihydropyridazine-4-carboxamide Isomer 2;
6-(4-Chlorophenyl)-N-[(2R)-1-hydroxypropan-2-yl]-2-(1-methyl-1H-pyrazol-4-yl)-3-oxo-2,3-dihydropyridazine-4-carboxamide;
6-(4-Chlorophenyl)-N-[(2R)-3-hydroxy-3-methylbutan-2-yl]-3-oxo-2-(1H-pyrazol-4-yl)-2,3-dihydropyridazine-4-carboxamide;
6-(4-Chlorophenyl)-N-[(2R)-3-hydroxy-3-methylbutan-2-yl]-3-oxo-2-(1,2-thiazol-4-yl)-2,3-dihydropyridazine-4-carboxamide;
N-[(2R)-3-Hydroxy-3-methylbutan-2-yl]-3-oxo-2-(1,2-thiazol-4-yl)-6-[4-(trifluoromethoxy)-phenyl]-2,3-dihydropyridazine-4-carboxamide;
N-[(cis)-2-Hydroxy-2-methylcyclopentyl]-3-oxo-2-(pyridin-3-yl)-6-[4-(trifluoromethoxy)-phenyl]-2,3-dihydropyridazine-4-carboxamide;
N-[(cis)-2-hydroxy-2-methylcyclopentyl]-3-oxo-2-(pyridin-3-yl)-6-[4-(trifluoromethoxy)-phenyl]-2,3-dihydropyridazine-4-carboxamide, Isomer 1;
N-[(cis)-2-hydroxy-2-methylcyclopentyl]-3-oxo-2-(pyridin-3-yl)-6-[4-(trifluoromethoxy)-phenyl]-2,3-dihydropyridazine-4-carboxamide, Isomer 2;
6-[4-(Difluoromethoxy)phenyl]-N-(2-hydroxy-2-methylpropyl)-3-oxo-2-(pyridin-3-yl)-2,3-dihydropyridazine-4-carboxamide;
6-[4-(Difluoromethoxy)phenyl]-N-[(2S)-1-hydroxypropan-2-yl]-3-oxo-2-(pyridin-3-yl)-2,3-dihydropyridazine-4-carboxamide;
6-[4-(Difluoromethoxy)phenyl]-N-[(2S)-3-hydroxy-3-methylbutan-2-yl]-3-oxo-2-(pyridin-3-yl)-2,3-dihydropyridazine-4-carboxamide;
6-[4-(Difluoromethoxy)phenyl]-N-[(2S,3S)-3-hydroxybutan-2-yl]-3-oxo-2-(pyridin-3-yl)-2,3-dihydropyridazine-4-carboxamide;
6-(4-Chlorophenyl)-N-[(cis)-2-hydroxy-2-methylcyclopentyl]-2-(1-methyl-1H-pyrazol-4-yl)-3-oxo-2,3-dihydropyridazine-4-carboxamide; Isomer 1;
6-(4-Chlorophenyl)-N-[(cis)-2-hydroxy-2-methylcyclopentyl]-2-(1-methyl-1H-pyrazol-4-yl)-3-oxo-2,3-dihydropyridazine-4-carboxamide; Isomer 2;
2-(5-Fluoropyridin-3-yl)-N-[(2S)-1-hydroxy-3-methylbutan-2-yl]-3-oxo-6-[6-(trifluoromethyl)pyridin-3-yl]-2,3-dihydropyridazine-4-carboxamide;
2-(5-Fluoropyridin-3-yl)-N-[(1S,2R)-2-hydroxycyclopentyl]-3-oxo-6-[6-(trifluoromethyl)-pyridin-3-yl]-2,3-dihydropyridazine-4-carboxamide;
N-[(1S)-1-Cyclopropyl-2-hydroxyethyl]-2-(5-fluoropyridin-3-yl)-3-oxo-6-[6-(trifluoromethyl)pyridin-3-yl]-2,3-dihydropyridazine-4-carboxamide;
2-(5-Fluoropyridin-3-yl)-N-[(2S)-3-hydroxy-3-methylbutan-2-yl]-3-oxo-6-[6-(trifluoromethyl)pyridin-3-yl]-2,3-dihydropyridazine-4-carboxamide;
N-[(1S)-1-Cyano-2-hydroxyethyl]-2-(5-fluoropyridin-3-yl)-3-oxo-6-[6-(trifluoromethyl)pyridin-3-yl]-2,3-dihydropyridazine-4-carboxamide;
2-(5-Fluoropyridin-3-yl)-N-[(2R)-3-hydroxy-3-methylbutan-2-yl]-3-oxo-6-[6-(trifluoromethyl)pyridin-3-yl]-2,3-dihydropyridazine-4-carboxamide;
1,5-Anhydro-2,4-dideoxy-2-[({2-(5-fluoropyridin-3-yl)-3-oxo-6-[6-(trifluoromethyl)pyridin-3-yl]-2,3-dihydropyridazine-4-yl}carbonyl)amino]-D-erythro-pentitol;
2-(5-Fluoropyridin-3-yl)-N-(2-hydroxy-2-methylpropyl)-3-oxo-6-[6-(trifluoromethyl)pyridin-3-yl]-2,3-dihydropyridazine-4-carboxamide; and
N-[(2S)-1-Hydroxypropan-2-yl]-3-oxo-2-(pyridin-3-yl)-6-[5-(trifluoromethyl)pyridin-2-yl]-2,3-dihydropyridazine-4-carboxamide,
or a polymorph, enantiomer, diastereomer, racemate, tautomer, N oxide, hydrate, solvate, physiologically acceptable salt, or a solvate of a physiologically acceptable salt thereof, or a mixture of any of the foregoing.

22: The combination of claim 1, wherein component B is pembrolizumab or nivolumab.

23: The combination of claim 1, wherein component B is pembrolizumab.

24: The combination of claim 1, wherein
component A is 6-(4-Chloro-phenyl)-N-[(2S)-1-hydroxypropan-2-yl]-2-(1-methyl-1H-pyrazol-4-yl)-3-oxo-2,3-dihydropyridazine-4-carboxamide,
or a polymorph, enantiomer, diastereomer, racemate, tautomer, N oxide, hydrate, solvate, physiologically acceptable salt, or a solvate of a physiologically acceptable salt thereof, or a mixture of any of the foregoing.

25-28. (canceled)

29: A method of treatment or prophylaxis of a cancer comprising administering to a subject in need thereof a therapeutically effective amount of a combination according to claim 1.

30: The combination of claim 1, wherein component B is pembrolizumab or nivolumab and at least one of components A and B are in the form of a pharmaceutical formulation.

31: A composition comprising the combination according to claim 1 and pharmaceutically acceptable ingredients.

32: A kit comprising a combination of component A, component B, and, optionally component C, wherein component A is one or more AhR inhibitors, or a polymorph, enantiomer, diastereomer, racemate, tautomer, N oxide, hydrate, solvate, physiologically acceptable salt, or a solvate of a physiologically acceptable salt thereof, or a mixture of any of the foregoing; component B is pembrolizumab or nivolumab; and component C is one or more further pharmaceutical agents.

33: The method according to claim 29, wherein the cancer is a cancer of the breast, respiratory tract, brain, reproductive organs, digestive tract, urinary tract, eye, liver, skin, kidney, head, neck, thyroid, or parathyroid, or a distant metastasis, lymphoma, sarcoma or leukemia of any of the foregoing.

34: The combination of claim 30, further comprising one or more additional pharmaceutical agents.

35: The combination of claim 30, wherein components A and B are both in the form of a pharmaceutical formulation and are suitable for administration to a subject in need thereof simultaneously, concurrently, separately, or sequentially.
